(19) United States
(12) Patent Application Publication
(10) Pub. No.: US 2008/0293712 A1
(43) Pub. Date: Nov. 27, 2008

(54) Benzo[C][2,7]Naphtyridine Derivatives, Methods of Making Thereof and Methods of Use Thereof

(75) Inventors: Allan WISSNER, Westchester, NY (US); Middleton Brawner FLOYD, JR., Suffern, NY (US); Russell DUSHIN, Garrison, NY (US); Heidi L. FRASER, Yorktown Heights, NY (US); Yongbo HU, River Edge, NJ (US); Andreas MADERNA, Stony Point, NY (US); Thomas NITTOLI, Pearl River, NY (US); Yanong Daniel WANG, Warren, NJ (US)

Correspondence Address: WYETH;PATENT LAW GROUP, 5 GIRALDA FARMS, MADISON, NJ, 07940, US

(73) Assignee: WYETH, Madison, NJ (US)

(21) Appl. No.: 12/042,128

(22) Filed: Mar. 4, 2008

Related U.S. Application Data
(60) Provisional application No. 60/905,087, filed on Mar. 5, 2007.

(51) Int. Cl. A61K 31/5377; C07D 471/02; A61K 31/4375; A61P 35/00; A61P 37/00; C07D 295/00; A61K 31/496

(52) U.S. Cl. 514/232.8; 546/81; 514/292; 544/361; 514/253.03; 544/126

(57) ABSTRACT

The present invention relates to Benzo[c][2,7]naphthyridine Derivatives, compositions comprising an effective amount of a Benzo[c][2,7]naphthyridine Derivative, methods for treating or preventing a proliferative disorder or an autoimmune disease, comprising administering to a subject in need thereof an effective amount of a Benzo[c][2,7]naphthyridine Derivative, methods for modulating PDK-1 activity, PKA activity, Akt activity, S6K activity, or PKC activity, comprising administering to a subject in need thereof an effective amount of a Benzo[c][2,7]naphthyridine Derivative. The invention also relates to processes for preparing a Benzo[c][2,7]naphthyridine Derivative.

### 1. REFERENCE TO RELATED APPLICATIONS

This application claims priority to U.S. Provisional Application No. 60/905,087, filed Mar. 5, 2007, the disclosure of which is incorporated by reference herein in its entirety.

### 2. FIELD OF THE INVENTION

The present invention relates to Benzo[c][2,7]naphthyridine Derivatives, methods of making thereof, compositions comprising an effective amount of a Benzo[c][2,7]naphthyridine Derivative and methods of treating or preventing a disease, e.g., a proliferative disorder, comprising administering a subject in need thereof an effective amount of a Benzo[c][2,7]naphthyridine Derivative.

### 3. BACKGROUND OF THE INVENTION

Cancer is second only to cardiovascular disease as the leading cause of death in the United States. The American Cancer Society estimated that 1.4 million new cancer cases would be diagnosed and 565,000 people would die of cancer in 2006 (American Cancer Society, Cancer Facts and Figures 2006, Atlanta, Ga.). The National Cancer Institute estimated that in January 2002, approximately 10.1 million living Americans had a history of cancer. The National Institutes of Health estimate direct medical costs of cancer as over $100 billion per year with an additional $100 billion in indirect costs due to lost productivity—the largest such costs of any major disease.

Cancer is a process by which the controlling mechanisms that regulate cell growth and differentiation are impaired, resulting in a failure to control cell turnover and growth. This lack of control causes a tumor to grow progressively, enlarging and occupying space in vital areas of the body. If the tumor invades surrounding tissue and is transported to distant sites, death of the individual can result.

The selective killing of cancer cells, while minimizing deleterious effects on normal cells, is a desired goal in cancer therapy. Modalities commonly used in the treatment of cancer include chemotherapy, radiation therapy, surgery and biological therapy (a broad category that includes gene-, protein- or cell-based treatments and immunotherapy). Despite the availability of a variety of anticancer agents, traditional chemotherapy has drawbacks. Many anticancer agents are toxic, and chemotherapy can cause significant, and often dangerous, side effects, including severe nausea, bone marrow depression, liver, heart and kidney damage, and immunosuppression. Additionally, many tumor cells eventually develop multi-drug resistance after being exposed to one or more anticancer agents. As such, single-agent chemotherapy is effective for only a very limited number of cancers. Many chemotherapeutic drugs are anti-proliferative agents, acting at different stages of the cell cycle. Because it is difficult to predict the pattern of sensitivity of a neoplastic cell population to anticancer drugs, or the current stage of the cell cycle that a cell happens to be in, it is common to use multi-drug regimens in the treatment of cancer.

Despite the significant research efforts and resources which have been directed towards the development of novel anticancer agents and improved methods for treating cancer, there remains a need in the art for novel compounds, compositions, and methods that are useful for treating cancer with improved therapeutic indices.

The interaction of insulin and growth factors with their receptors on the outside surface of a cell, leads to the activation of phosphatidylinositol 3-kinase (PI 3-kinase) and generation of the phosphatidylinositol 3,4,5-trisphosphate (PtdIns(3,4,5)P₃) second messenger at the inner surface of the cell membrane. One of the most studied signaling events controlled by PtdIns(3,4,5)P₃, comprises the activation of a group of cAMP-dependent protein kinase/protein kinase G/protein kinase C family (AGC family) protein kinases, including isoforms of protein kinase B (PKB, also known as Akt), p70 ribosomal S6 kinase (S6K), serum- and glucocorticoid-induced protein kinase (SGK) and protein kinase C (PKC), which are involved in regulating physiological processes relevant to metabolism, growth, proliferation and survival. Studies have shown that 3-phosphoinositide-dependent protein kinase-1 (PDK-1) phosphorylates and activates the AGC family kinase members regulated by PI 3-kinase (see, e.g., Mora et al., Semin Cell Dev Biol (2004) 15:161-170). Akt has at least three isoforms: Akt1, Akt2 and Akt3, which are also known as PKBα, PKBβ and PKBγ, respectively. PKC has at least 12 isoforms, which have distinct and in some cases opposing roles in cell growth and differentiation (see, e.g., Mackay et al., Endocr Relat Cancer (2003) 10:389-396). For example, PKCθ kinase has been implicated in signaling of T-cell activation, proliferation, and cytokine production. PKCθ-deficient mice immunized with the myelin oligodendrocyte glycoprotein (MOG) peptide MOG(35-55) were found to be resistant to the development of clinical experimental autoimmune encephalomyelitis compared with wild-type control mice (Tan et al., J Immunol (2006) 176:2872-9).

There remains a need in the art for novel compounds, compositions and methods that are useful for inhibition or modulation of PDK-1, S6K, PKA, an Akt isoform or a PKC isoform activity.

Citation of any reference in Section 2 is not an admission that the reference is prior art.

### 4. SUMMARY OF THE INVENTION

In one aspect, the invention provides a compound of Formula (I)

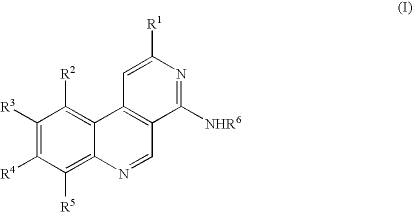

and pharmaceutically acceptable salts thereof,
wherein

R¹ is —H, -halo, —OH, —NH₂, —CN, —NO₂, —C₁-C₆ alkyl, —O—C₁-C₆ alkyl, —S—C₁-C₆ alkyl, —NH—C₁-C₆ alkyl, —N(C₁-C₆ alkyl)₂, -phenyl, -benzyl, —O-phenyl, —O-benzyl, —NH-phenyl, —NH-benzyl, NR¹²R¹³, —OR¹³, —SR¹³, —N(R¹²)(C(R¹²)₂)ₙ—R¹³, —O—(C(R¹²)₂)—R¹³, —S—(C(R¹²)₂)—R¹³, —(C(R¹²)₂)ₙ—R¹³, —(C(R¹²)₂)ₙ—R¹⁴, —(C(R¹²)₂)ₙ—N(R¹²)(C(R¹²)₂)—R¹³, —(C(R¹²)₂)—N(R¹²)(C(R¹²)₂)ₙ—R¹⁴, —(C(R¹²)₂)ₙ—O—(C(R¹²)₂)ₙ—R¹³, —(C(R¹²)₂)ₙ—O—(C(R¹²)₂)ₙ—R¹⁴, —(C(R¹²)₂)ₙ—S—(C(R¹²)₂)ₙ—R¹³, —(C(R¹²)₂)ₙ—S—(C(R¹²)₂)ₙ—R¹³, R¹³, or R¹⁴,

wherein each C₁-C₆ alkyl, -phenyl or -benzyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl, benzoyl, —S(O)₂NH₂, —S(O)₂NH—C₁-C₆ alkyl, or —S(O)₂N(C₁-C₆ alkyl)₂,

wherein n=1-4 at each occurrence, with the proviso that when the carbon chain of length n is between two heteroatoms, n=2-4;

R² and R⁵ are each independently —H, —OH, -halo, —CN, —N₃, —NH₂, —NH—C₁-C₄ alkyl, —N(C₁-C₂ alkyl)₂, —C₁-C₄ alkyl, —O—C₁-C₄ alkyl, or —S—C₁-C₄ alkyl,

wherein each C₁-C₂ alkyl or C₁-C₄ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R³ and R⁴ are each independently —H, —OH, -halo, —C₁-C₆ alkyl, —C₂-C₆ alkenyl, —C₂-C₆ alkynyl, —O—C₁-C₆ alkyl, —O—C₂-C₆ alkenyl, —O—C₂-C₆ alkynyl, —C(O)O—C₁-C₆ alkyl, —C(O)NH—C₁-C₆ alkyl, —C(O)N(C₁-C₆ alkyl)₂, —C₁-C₆-alkylene-O—C₁-C₆ alkyl, —O—C₁-C₆-alkylene-O—C₁-C₆ alkyl, —C₁-C₆-alkylene-C(O)O—C₁-C₆ alkyl, —C₁-C₆ alkylene-C(O)NH—C₁-C₆ alkyl, —OC(O)—C₁-C₆ alkyl, —OC(O)—C₂-C₆ alkenyl, —OC(O)—C₂-C₆ alkynyl, —C₁-C₆ alkylene-OC(O)—C₁-C₆ alkyl, —C₁-C₆ alkylene-OC(O)—C₂-C₆ alkenyl, —C₁-C₆ alkylene-OC(O)—C₂-C₆ alkynyl, —S—C₁-C₆ alkyl, —S(O)—C₁-C₆ alkyl, —S(O)₂—C₁-C₆ alkyl, —S(O)₂NH—C₁-C₆ alkyl, —S(O)₂NH—C₂-C₆ alkenyl, —S(O)₂NH—C₂-C₆ alkynyl, —C(O)-benzyl, —CN, —NO₂, —CHO, —COOH, —C(O)O—C₁-C₆ alkyl, —C(O)—C₁-C₆ alkyl, -phenyl, —O-phenyl, —S-phenyl, -benzyl, —O-benzyl, —CHO, —NHC(O)H, —NH₂, —NHOH, —NH—O—C₁-C₆ alkyl, —NH—C₁-C₆ alkyl, —N(C₁-C₆ alkyl)₂, —NHC(O)—C₁-C₆ alkyl, —N(C₁-C₆ alkyl)(C₂-C₆ alkenyl), —N(C₂-C₆ alkenyl)₂, —NH-phenyl, —NH-benzyl, —NHS(O)₂—C₁-C₆ alkyl, —NHS(O)₂-phenyl, —NHS(O)₂-benzyl,

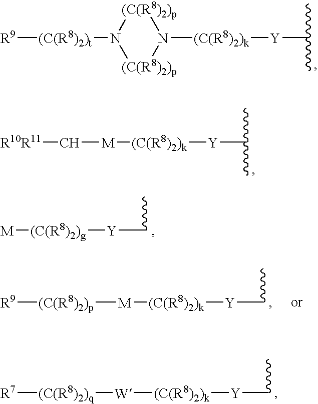

wherein

a=0-1;

g=1-6;

k=0-4;

p=2-4 at each occurrence;

q=0-4;

t=2-4;

M is a bond, —N(R⁸)—, —O—, —N((C(R⁸)₂)ₚN(R⁸)₂—, —N((C(R⁸)₂)ₚOR⁸)—, —CC—, —CH═CH—, or phenylene;
W′ is a bond, —N(R⁸)—, —O—, —CC—, —CH═CH— or phenylene;
Y is —(CH₂)ₐ—, —O—, —S—, —C(O)N(R⁸)—, —C(S)N(R⁸)—, or —N(R⁸)—;
when M is phenylene then p=0-4;
when M is —O— and R⁹ is —OR⁸ then p=1-4;
when M is —CC— or —CH═CH— and Y is —S—, —O— or —N(R⁸)— then k=1-4;
when M is —CC— or —CH═CH— then p=1-4;
when W′ is —CC— or —CH═CH— and R⁷ is bonded through a heteroatom then q=1-4;
when W′ is a bond and q=0 and R⁷ is bonded through a nitrogen atom and Y is —S—, —O— or —N(R⁸)— then k=2-4;
when W′ is a bond and k=0 and R⁷ is bonded through a nitrogen atom and Y is —S—, —O— or —N(R⁸)— then q=2-4;
when W′ is not a bond, —CC—, —CH═CH—, or -phenyl- and R⁷ is bonded through a nitrogen atom then q=2-4;
when Y is —N(R⁸)— and R⁹ is —N(R⁸)₂, —N(R⁸)₃⁺ or —NR⁸(OR⁸) then g=2-6;
when Y is —N(R⁸)— and M is —N(R⁸)—, —O—, —N((C(R⁸)₂)ₚN(R⁸)₂)— or —N((C(R⁸)₂)ₚ—OR⁸)— then k=1-4;
when Y is —N(R⁸)— and W′ is —N(R⁸)— or —O— then k=2-4;
when Y is —O— and either M or W′ is —O— then k=2-4;

wherein each —C₁-C₆ alkyl, —C₁-C₆ alkylene, —C₁-C₆ alkylene-O—C₁-C₆ alkyl, —C₂-C₆ alkenyl, —C₂-C₆ alkynyl, -phenyl, -benzyl, or phenylene is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R⁶ is —H, —C₁-C₄ alkyl, —OH, a 5- or 6-membered aromatic monocyclic heterocycle, a 3- to 7-membered non-aromatic monocyclic heterocycle, a —C₁-C₃ alkylene-5- or 6-membered aromatic monocyclic heterocycle or a —C₁-C₃ alkylene-3- to 7-membered non-aromatic monocyclic heterocycle

wherein each 5- or 6-membered aromatic monocyclic heterocycle or 3- to 7-membered non-aromatic monocyclic heterocycle is independently unsubstituted or substituted with one or more of —OH, -halo, —CN, —N₃, —NH₂, and —C₁-C₄ alkyl;

R⁷ is -phenyl, a 5- or 6-membered aromatic monocyclic heterocycle, or a 3- to 7-membered non-aromatic monocyclic heterocycle, wherein, each R⁷ is optionally mono or di-substituted on a carbon atom with —OH, —R⁸, —N(R⁸)₂, —OR⁸, —(C(R⁸)₂)ᵣOR⁸, —(C(R⁸)₂)ᵣN(R⁸)₂, or a 3- to 7-membered monocyclic heterocycle,

wherein r=1-6;

each R⁷ is optionally mono or di-substituted on a saturated carbon atom with —O(C(R⁸)₂)ᵣO—,

wherein r=1-6;

each R⁷ is optionally mono-substituted on a nitrogen atom with R⁸;

R⁸ is each independently —H, —C₁-C₆ alkyl, —C₂-C₆ alkenyl, —C₂-C₆ alkynyl, —C₃-C₈ monocyclic cycloalkyl, —C(O)—C₁-C₆ alkyl, —C(O)O—C₁-C₆ alkyl, or -phenyl, each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R⁹ is -halo, —N(R⁸)₂, —OR⁸, —N(R⁸)₃⁺, or —NR⁸(OR⁸);

R¹⁰ and R¹¹ are each independently —(C(R⁸)₂)ₙN(R⁸)₂ or —(C(R⁸)₂)ₙOR⁸,

wherein s=1-4.

R¹² is each independently —H, —C₁-C₆ alkyl, —C₂-C₆ alkenyl, —C₂-C₆ alkynyl, —C₃-C₈ monocyclic cycloalkyl, —C₃-C₈ monocyclic cycloalkenyl, -phenyl, -benzyl, a 5- or 6-membered aromatic monocyclic heterocycle, or a 3- to 7-membered non-aromatic monocyclic heterocycle, each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R¹³ is a 5- or 6-membered aromatic monocyclic heterocycle, a 3- to 7-membered non-aromatic monocyclic heterocycle, a 8- to 12-membered bicyclic heterocycle, -phenyl, -benzyl, or

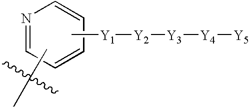

wherein:

Y₁ is a bond, —O—, —N(R¹⁶)—, —C((R¹⁶)₂)—, —C(R¹⁶)═C(R¹⁶), or —CC—;

Y₂ and Y₄ are each independently —(C(R¹⁶)₂)_{b}—, wherein b=0-4;

Y₃ is —C(R¹⁵R¹⁶)—;

Y₅ is absent, —H, -halo, —C₁-C₆ alkyl, —NH-halo-substituted C₁-C₄ alkyl, —O—C₁-C₆ alkyl, —O—C₁-C₆ alkyl, —OH, —NH₂, —NH—C₁-C₆ alkyl, —N(C₁-C₆ alkyl)₂, —OS(O)₂—C₁-C₆ alkyl, -phenyl, —O-phenyl, —NH-phenyl, -benzyl, —O-benzyl, —NR¹²R²⁸, —OR²⁸, —SR²⁸, —N(R¹²)(C(R¹²)₂)ₘ—R²⁸, —O(C(R¹²)₂)ₘ—R²⁸, —S(C(R¹²)₂)ₘ—R²⁸, —(C(R¹²)₂)ₘ—R²⁸, —(C(R¹²)₂)ₘ—R¹⁴, —(C(R¹²)₂)ₘ—N(R¹²)(C(R¹²)₂)ₘ—R²⁸, —(C(R¹²)₂)ₘ—N(R¹²)(C(R¹²)₂)ₘ—R¹⁴, —(C(R¹²)₂)ₘ—O—(C(R¹²)₂)ₘ—R²⁸, —(C(R¹²)₂)ₘ—O—(C(R¹²)₂)ₘ—R¹⁴, —(C(R¹²)₂)ₘ—S—(C(R¹²)₂)ₘ—R²⁸, —(C(R¹²)₂)ₘ—S—(C(R¹²)₂)ₘ—R¹⁴, R¹⁴, —N(C₁-C₆ alkyl)(S(O)₂R²⁷), a 5- or 6-membered aromatic monocyclic heterocycle, a 3- to 7-membered non-aromatic monocyclic heterocycle, or a 8- to 12-membered bicyclic heterocycle,

wherein m=1-4 with the proviso that when the carbon chain of length m is between two heteroatoms, m=2-4;

when Y₁ is —C((R¹⁶)₂)— the R¹⁶ substituents together with the intervening carbon atom or atoms can form a 3- to 7-membered ring;
when Y₁ is —C(R¹⁶)═C(R¹⁶)— the R¹⁶ substituents together with the intervening carbon atoms can form a 3- to 7-membered ring;

wherein each heterocycle can be substituted on a single saturated carbon atom not adjacent to a nitrogen atom with —O(CH₂)₂O—, —(CH₂)₃O— or —NH—; and

each heterocycle can optionally be benzo-fused; and

each heterocycle can optionally be mono- or di-substituted with —O—, —S—, —NH-benzoyl, R²⁸, —O—R²⁸, —O—C₁₋₄ alkylene-R²⁸, —NH—R²⁸, —NH—C₁-C₄ alkylene-R²⁸, —C(O)—R²⁸, or R¹⁸,

wherein each 5- or 6-membered aromatic monocyclic heterocycle, a 3- to 7-membered non-aromatic monocyclic heterocycle, a 8- to 12-membered bicyclic heterocycle, -phenyl, -benzyl, benzoyl or —C₁-C₆ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —O—C₂-C₆ alkenyl, —S—C₁-C₆ alkyl, —NH—C₁-C₄ alkyl, —N(C₁-C₄ alkyl)₂, -halo-substituted C₁-C₄ alkyl, -amino-substituted C₁-C₄ alkyl, hydroxy-substituted C₁-C₄ alkyl, -cyano-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, —NHC(O)—C₁-C₆ alkyl, —C(O)NH₂, —C(O)NH—C₁-C₆ alkylene-N(C₁-C₄ alkyl)₂, —C(NH)—NH—C₁-C₆ alkyl, —C(NH)—NH₂, -phenyl, -halo-substituted-phenyl, -amino-substituted phenyl, —O-phenyl, —S-phenyl, —NH-phenyl, —C(O)N(H)-phenyl, —O—C₁-C₄ alkylene-C(O)-phenyl, -benzyl, —O-benzyl, —S-benzyl, —NH-benzyl, —C(O)-benzyl, benzoyl, pyridyl or morpholinyl;

R¹⁴ is —N(R¹²)₂, —OR¹², —SR¹², —NR¹²—(C(R¹²)₂)ₙ—N(R¹²)₂, —NR¹²(C(R¹²)₂)ₙ—OR¹², —N((C(R¹²)₂)ₙ—N(R¹²)₂)₂, —N((C(R¹²)₂)ₙ—OR¹²)₂, or —N((C(R¹²)₂)ₙ—N(R¹²)₂)(C(R¹²)₂)ₙ—OR¹²);

wherein n=1-4 at each occurrence, with the proviso that when the carbon chain of length n is between two heteroatoms, n=2-4;

R¹⁵ is —OH or —N(R¹⁷)₂;

R¹⁶ is each independently an open valence, —H, —C₁-C₈ alkyl, —C₂-C₈ alkenyl, —C₂-C₈ alkynyl, C₃-C₈ monocyclic cycloalkyl, or —C₃-C₈ monocyclic cycloalkenyl, each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R¹⁶ on Y₃ together with R¹⁶ on Y₂ together with the intervening atoms can form a 3- to 7-membered ring;

R¹⁶ on Y₃ together with R¹⁶ on Y₁ together with the intervening atoms can form a 3- to 7-membered ring;

R¹⁶ on Y₁ together with R¹⁶ on Y₂ together with the intervening atoms can form a 3- to 7-membered ring;

R¹⁶ on Y₂ together with R¹⁶ on Y₄ together with the intervening atoms can form a 3- to 7-membered ring;

R¹⁶ on Y₃ together with R¹⁶ on Y₄ together with the intervening atoms can form a 3- to 7-membered ring;

when R¹⁵ is —N(R¹⁷)₂, R¹⁷ on Y₃ together with R¹⁶ on either Y₁, Y₂, or Y₄ together with the intervening atoms can form a 3- to 7-membered ring;

R¹⁷ is each independently an open valence, —H, —C(O)NH—C₁-C₆ alkyl, —C(NH)—NH₂, —C(NH)—NH—C₁-C₆ alkyl, —C(O)—O—C₁-C₆ alkyl, —C(O)—O-phenyl, —C(O)—O—C₁-C₃ alkylene-phenyl, —C(O)—O—C₂-C₃ alkenylene-phenyl, —C(O)—O—C₂-C₃ alkynylene-phenyl, —C(O)H, —C₁-C₉ alkyl, —C₂-C₈ alkenyl, —C₂-C₈ alkynyl, —C₃-C₈ monocyclic cycloalkyl, —C₃-C₈ monocyclic cycloalkenyl, —C₇-C₉ monocyclic cycloalkynyl, -phenyl, —C₁-C₃ alkylene-phenyl, —C₂-C₃ alkenylene-phenyl, or —C₂-C₃ alkynylene-phenyl,

each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R¹⁸ is —H, -halo, —OH, —CN, —NH₂, —N₃, —NO₂, —CHO, —COOH, —C₁-C₆ alkyl, —C₂-C₆ alkenyl, —C₂-C₆ alkynyl, —NHC(O)—C₁-C₆ alkyl, —C₁-C₆ alkylene-O—C₁-C₆ alkyl, —C₁-C₆ alkylene-C(O)O—C₁-C₆ alkyl, —O—C₁-C₆ alkyl, —S—C₁-C₆ alkyl, —C(O)O—C₁-C₆ alkyl, —C(O)—C₁-C₆ alkyl, -phenyl, —O-phenyl, —S-phenyl, -benzoyl, -benzyl, —C₁-C₆ alkylene-C(O)O-phenyl, —NH—C₁-C₆ alkyl, —N(C₁-C₆ alkyl)₂, —NH-phenyl, —NH-benzyl, —NH—C(O)—C₁-C₆ alkyl, —NH—C(O)—C₂-C₆ alkenyl, —NH—C(O)—C₂-C₆ alkynyl, —C₁-C₆ alkylene-COOH, —C₁-C₆ alkylene-CHO, —C₁-C₆ alkylene-NH—C₁-C₆ alkyl, —C₁-C₆ alkylene-N(C₁-C₆ alkyl)₂, —C(O)NH—C₁-C₆ alkyl, —C(O)N(C₁-C₆)₂ alkyl, —O—C₁-C₆ alkylene-NH—C₁-C₆ alkyl, —O—C₁-C₆ alkylene-N(C₁-C₆ alkyl)₂, —OS(O)₂—C₁-C₆ alkyl, or —SH,

wherein each —C₁-C₆ alkyl, —C₁-C₆ alkylene, —C₂-C₆ alkenyl, —C₂-C₆ alkynyl, -phenyl, -benzyl, or benzoyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R²⁷ is C₁-C₆ alkyl, -phenyl, —(C((R¹²)₂)ₚ)-phenyl, a 5- or 6-membered aromatic monocyclic heterocycle, a 3- to 7-membered non-aromatic monocyclic heterocycle, or a 8- to 12-membered bicyclic heterocycle, —(C((R¹²)₂)ₚ)-5- or 6-membered aromatic monocyclic heterocycle, —(C((R¹²)₂)ₚ)-3- to 7-membered non-aromatic monocyclic heterocycle, —(C((R¹²)₂)ₚ)-8- to 12-membered bicyclic heterocycle,

wherein p=1-4 with the proviso that when the carbon chain of length p is between two heteroatoms, p=2-4; and

R²⁸ is a 5- or 6-membered aromatic monocyclic heterocycle, a 3- to 7-membered non-aromatic monocyclic heterocycle, a 8- to 12-membered bicyclic heterocycle, a C₃-C₈ monocyclic cycloalkyl, -phenyl, -benzyl,

wherein each heterocycle can be substituted on a single saturated carbon atom not adjacent to a nitrogen atom with —O(CH₂)₂O—, —(CH₂)₃O— or —NH—;

each heterocycle can optionally be benzo-fused; and

each 5- or 6-membered aromatic monocyclic heterocycle, a 3- to 7-membered non-aromatic monocyclic heterocycle, a 8- to 12-membered bicyclic heterocycle, C₃-C₈ monocyclic cycloalkyl, -phenyl, -benzyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —O—C₂-C₆ alkenyl, —S—C₁-C₆ alkyl, —NH—C₁-C₄ alkyl, —N(C₁-C₄ alkyl)₂, -halo-substituted C₁-C₄ alkyl, -amino-substituted C₁-C₄ alkyl, -hydroxy-substituted-C₁-C₄ alkyl, -cyano-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, —NHC(O)—C₁-C₆ alkyl, —C(O)NH₂, —C(O)NH—C₁-C₆ alkylene-N(C₁-C₄ alkyl)₂, —C(NH)—NH—C₁-C₆ alkyl, —C(NH)—NH₂, -phenyl, -halo-substituted phenyl, -amino-substituted phenyl, —O-phenyl, —S-phenyl, —NH-phenyl, —C(O)N(H)-phenyl, -benzyl, —O-benzyl, —S-benzyl, —NH-benzyl, —C(O)-benzyl, benzoyl, pyridyl or morpholinyl.

In one embodiment of a compound of Formula (I),

R¹ is —H, -halo, —OH, —NH₂, —CN, —NO₂, —C₁-C₆ alkyl, —O—C₁-C₆ alkyl, —S—C₁-C₆ alkyl, —NH—C₁-C₆ alkyl, —N(C₁-C₆ alkyl)₂, -phenyl, -benzyl, —O-phenyl, —O-benzyl, —NH-phenyl, —NH-benzyl, NR¹²R¹³, —OR¹³, —SR¹³, —N(R¹²)(C(R¹²)₂)ₙ—R¹³, —O—(C(R¹²)₂)ₙ—R¹³, —S—(C(R¹²)₂)ₙ—R¹³, —(C(R¹²)₂)ₙ—R¹³, —(C(R¹²)₂)ₙ—R¹⁴, —(C(R¹²)₂)ₙ—N(R¹²)(C(R¹²)₂)—R¹³, —(C(R¹²)₂)—N(R¹²)(C(R¹²)₂)—R¹⁴, —(C(R¹²)₂)ₙ—O—(C(R¹²)₂)ₙ—R¹³, —(C(R¹²)₂)ₙ—O—(C(R¹²)₂)ₙ—R¹⁴, —(C(R¹²)₂)ₙ—S—(C(R¹²)₂)ₙ—R¹³, —(C(R¹²)₂)ₙ—S—(C(R¹²)₂)ₙ—R¹⁴, R¹³, or R¹⁴,

wherein each C₁-C₆ alkyl, -phenyl or -benzyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl, benzoyl, —S(O)₂NH₂, —S(O)₂NH—C₁-C₆ alkyl, or —S(O)₂N(C₁-C₆ alkyl)₂,

wherein n=1-4 at each occurrence, with the proviso that when the carbon chain of length n is between two heteroatoms, n=2-4;

R² and R⁵ are each independently —H, —OH, -halo, —CN, —N₃, —NH₂, —NH—C₁-C₄ alkyl, —N(C₁-C₂ alkyl)₂, —C₁-C₄ alkyl, —O—C₁-C₄ alkyl, or —S—C₁-C₄ alkyl,

wherein each C₁-C₂ alkyl or C₁-C₄ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R³ and R⁴ are each independently —H, —OH, -halo, —C₁-C₆ alkyl, —C₂-C₆ alkenyl, —C₂-C₆ alkynyl, —O—C₁-C₆ alkyl, —O—C₂-C₆ alkenyl, —O—C₂-C₆ alkynyl, —C(O)O—C₁-C₆ alkyl, —C(O)NH—C₁-C₆ alkyl, —C(O)N(C₁-C₆ alkyl)₂, —C₁-C₆— alkylene-O—C₁-C₆ alkyl, —O—C₁-C₆-alkylene-O—C₁-C₆ alkyl, —C₁-C₆-alkylene-C(O)O—C₁-C₆ alkyl, —C₁-C₆ alkylene-C(O)NH—C₁-C₆ alkyl, —OC(O)—C₁-C₆ alkyl, —OC(O)—C₂-C₆ alkenyl, —OC(O)—C₂-C₆ alkynyl, —C₁-C₆ alkylene-OC(O)—C₁-C₆ alkyl, —C₁-C₆ alkylene-OC(O)—C₂-C₆ alkenyl, —C₁-C₆ alkylene-OC(O)—C₂-C₆ alkynyl, —S—C₁-C₆ alkyl, —S(O)—C₁-C₆ alkyl, —S(O)₂—C₁-C₆ alkyl, —S(O)₂NH—C₁-C₆ alkyl, —S(O)₂NH—C₂-C₆ alkenyl, —S(O)₂NH—C₂-C₆ alkynyl, —C(O)-benzyl, —CN, —NO₂, —CHO, —COOH, —C(O)O—C₁-C₆ alkyl, —C(O)—C₁-C₆ alkyl, -phenyl, —O-phenyl, —S-phenyl, -benzyl, —O-benzyl, —CHO, —NHC(O)H, —NH₂, —NHOH, —NH—O—C₁-C₆ alkyl, —NH—C₁-C₆ alkyl, —N(C₁-C₆ alkyl)₂, —NHC(O)—C₁-C₆ alkyl, —N(C₁-C₆ alkyl)(C₂-C₆ alkenyl), —N(C₂-C₆ alkenyl)₂, —NH-phenyl, —NH-benzyl, —NHS(O)₂—C₁-C₆ alkyl, —NHS(O)₂-phenyl, —NHS(O)₂-benzyl,

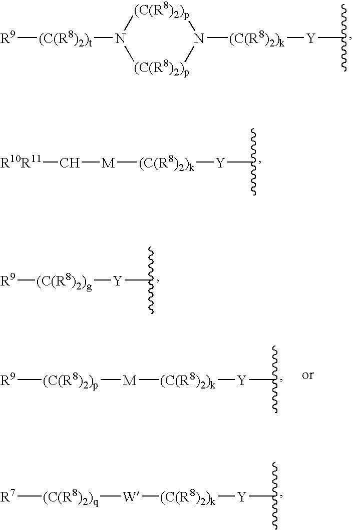

wherein

a=0-1;

g=1-6;

k=0-4;

p=2-4 at each occurrence;

q=0-4;

t=2-4;

M is a bond, —N(R⁸)—, —O—, —N((C(R⁸)₂)ₚN(R⁸)₂—, —N((C(R⁸)₂)ₚOR⁸)—, —CC—, —CH═CH—, or phenylene;
W′ is a bond, —N(R⁸)—, —O—, —CC—, —CH═CH— or phenylene;
Y is —(CH₂)ₐ—, —O—, —S—, —C(O)N(R⁸)—, —C(S)N(R⁸)—, or —N(R⁸)—;
when M is phenylene then p=0-4;
when M is —O— and R⁹ is —OR⁸ then p=1-4;
when M is —CC— or —CH═CH— and Y is —S—, —O— or —N(R⁸)— then k=1-4;
when M is —CC— or —CH═CH— then p=1-4;
when W′ is —CC— or —CH═CH— and R⁷ is bonded through a heteroatom then q=1-4;
when W′ is a bond and q=0 and R⁷ is bonded through a nitrogen atom and Y is —S—, —O— or —N(R⁸)— then k=2-4;
when W′ is a bond and k=0 and R⁷ is bonded through a nitrogen atom and Y is —S—, —O— or —N(R⁸)— then q=2-4;
when W′ is not a bond, —CC—, —CH═CH—, or -phenyl- and R⁷ is bonded through a nitrogen atom then q=2-4;
when Y is —N(R⁸)— and R⁹ is —N(R⁸)₂, —N(R⁸)₃⁺ or —NR⁸(OR⁸) then g=2-6;
when Y is —N(R⁸)— and M is —N(R⁸)—, —O—, —N((C(R⁸)₂)ₚN(R⁸)₂)— or —N((C(R⁸)₂)ₚ—OR⁸)— then k=1-4;
when Y is —N(R⁸)— and W′ is —N(R⁸)— or —O— then k=2-4;
when Y is —O— and either M or W′ is —O— then k=2-4;

wherein each —C₁-C₆ alkyl, —C₁-C₆ alkylene, —C₁-C₆ alkylene-O—C₁-C₆ alkyl, —C₂-C₆ alkenyl, —C₂-C₆ alkynyl, -phenyl, -benzyl, or phenylene is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R⁶ is —H, —C₁-C₄ alkyl, —OH, a 5- or 6-membered aromatic monocyclic heterocycle, a 3- to 7-membered non-aromatic monocyclic heterocycle, a —C₁-C₃ alkylene-5- or 6-membered aromatic monocyclic heterocycle or a —C₁-C₃ alkylene-3- to 7-membered non-aromatic monocyclic heterocycle;

wherein each 5- or 6-membered aromatic monocyclic heterocycle or 3- to 7-membered non-aromatic monocyclic heterocycle is independently unsubstituted or substituted with one or more of —OH, -halo, —CN, —N₃, —NH₂, and —C₁-C₄ alkyl;

R⁷ is -phenyl, a 5- or 6-membered aromatic monocyclic heterocycle, or a 3- to 7-membered non-aromatic monocyclic heterocycle, wherein, each R⁷ is optionally mono or di-substituted on a carbon atom with —OH, —R⁸, —N(R⁸)₂, —OR⁸, —(C(R⁸)₂)ᵣOR⁸, —(C(R⁸)₂)ᵣN(R⁸)₂, or a 3- to 7-membered monocyclic heterocycle,

wherein r=1-6;

each R⁷ is optionally mono or di-substituted on a saturated carbon atom with —O(C(R⁸)₂)ᵣO—,

wherein r=1-6;

each R⁷ is optionally mono-substituted on a nitrogen atom with R⁸;

R⁸ is each independently —H, —C₁-C₆ alkyl, —C₂-C₆ alkenyl, —C₂-C₆ alkynyl, —C₃-C₈ monocyclic cycloalkyl, —C(O)—C₁-C₆ alkyl, —C(O)O—C₁-C₆ alkyl, or -phenyl, each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R⁹ is -halo, —N(R⁸)₂, —OR⁸, —N(R⁸)₃⁺, or —NR⁸(OR⁸);

R¹⁰ and R¹¹ are each independently —(C(R⁸)₂)ₙN(R⁸)₂ or —(C(R⁸)₂)ₙOR⁸,

wherein s=1-4.

R¹² is each independently —H, —C₁-C₆ alkyl, —C₂-C₆ alkenyl, —C₂-C₆ alkynyl, —C₃-C₈ monocyclic cycloalkyl, —C₃-C₈ monocyclic cycloalkenyl, -phenyl, -benzyl, a 5- or 6-membered aromatic monocyclic heterocycle, or a 3- to 7-membered non-aromatic monocyclic heterocycle, each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R¹³ is a 5- or 6-membered aromatic monocyclic heterocycle, a 3- to 7-membered non-aromatic monocyclic heterocycle, a 8- to 12-membered bicyclic heterocycle, -phenyl, -benzyl, or

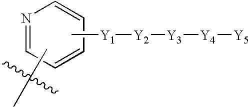

wherein:

Y₁ is a bond, —O—, —N(R¹⁶), —C(R¹⁶)₂—, —C(R¹⁶)═C(R¹⁶)—, or —CC—;

Y₂ and Y₄ are each independently —(C(R¹⁶)₂)_{b}—, wherein b=0-4;

Y₃ is —C(R¹⁵R¹⁶)—;

Y₅ is absent, —H, -halo, —C₁-C₆ alkyl, —O—C₁-C₆ alkyl, —O—C₁-C₆ alkyl, —OH, —NH₂, —NH—C₁-C₆ alkyl, —N(C₁-C₆ alkyl)₂, —OS(O)₂—C₁-C₆ alkyl, -phenyl, —O-phenyl, —NH-phenyl, -benzyl, —O-benzyl, —NR¹²R²⁸, —OR²⁸, —SR²⁸, —N(R¹²)(C(R¹²)₂)ₘ—R¹², —O(C(R¹²)₂)ₘ—R²⁸, —S(C(R¹²)₂)ₘR²⁸, (C(R¹²)₂)ₘ—R¹⁴, —(C(R¹²)₂)ₘ—R²⁸, —(C(R¹²)₂)ₘ—N(R¹²)(C(R¹²)₂)ₘ—R⁸, —(C(R¹²)₂)ₘ—N(R¹²)(C(R¹²)₂)ₘ—R¹⁴, —(C(R¹²)₂)ₘ—O—(C(R¹²)₂)ₘ—R¹², —(C(R¹²)₂)ₘ—O—(C(R¹²)₂)ₘ—R¹⁴, —(C(R¹²)₂)ₘ—S—(C(R¹²)₂)ₘ—R¹², —(C(R¹²)₂)ₘ—S—(C(R¹²)₂)ₘ—R¹⁴, R¹⁴, —N(C₁-C₆ alkyl)(S(O)₂R²⁷), a 5- or 6-membered aromatic monocyclic heterocycle, a 3- to 7-membered non-aromatic monocyclic heterocycle, or a 8- to 12-membered bicyclic heterocycle,

wherein m=1-4 with the proviso that when the carbon chain of length m is between two heteroatoms, m=2-4;

when Y₁ is —C((R¹⁶)₂)— the R¹⁶ substituents together with the intervening carbon atom or atoms can form a 3- to 7-membered ring;
when Y₁ is —C(R¹⁶)═C(R¹⁶)— the R¹⁶ substituents together with the intervening carbon atoms can form a 3- to 7-membered ring;

wherein each heterocycle can be substituted on a single saturated carbon atom not adjacent to a nitrogen atom with —O(CH₂)₂O— or —(CH₂)₃O—; and

each heterocycle can optionally be benzo-fused; and

each heterocycle can optionally be mono- or di-substituted with —O—, —S—, —NH-benzoyl, R²⁸, —O—R²⁸ or R¹⁸,

wherein each 5- or 6-membered aromatic monocyclic heterocycle, a 3- to 7-membered non-aromatic monocyclic heterocycle, a 8- to 12-membered bicyclic heterocycle, -phenyl, -benzyl, benzoyl or —C₁-C₆ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R¹⁴ is —N(R¹²)₂, —OR¹², —SR¹², —NR¹²—(C(R¹²)₂)ₙ—N(R¹²)₂, —NR¹²—(C(R¹²)₂)ₙ—OR¹², —N((C(R¹²)₂)ₙ—N(R¹²)₂)₂, —N((C(R¹²)₂)ₙ—OR¹²)₂, or —N((C(R¹²)₂)ₙ—N(R¹²)₂)(C(R¹²)₂)ₙ—OR¹²);

wherein n=1-4 at each occurrence, with the proviso that when the carbon chain of length n is between two heteroatoms, n=2-4;

R¹⁵ is —OH or —N(R¹⁷)₂;

R¹⁶ is each independently an open valence, —H, —C₁-C₈ alkyl, —C₂-C₈ alkenyl, —C₂-C₈ alkynyl, C₃-C₈ monocyclic cycloalkyl, or —C₃-C₈ monocyclic cycloalkenyl, each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R¹⁶ on Y₃ together with R¹⁶ on Y₂ together with the intervening atoms can form a 3- to 7-membered ring;

R¹⁶ on Y₃ together with R¹⁶ on Y₁ together with the intervening atoms can form a 3- to 7-membered ring;

R¹⁶ on Y₁ together with R¹⁶ on Y₂ together with the intervening atoms can form a 3- to 7-membered ring;

R¹⁶ on Y₂ together with R¹⁶ on Y₄ together with the intervening atoms can form a 3- to 7-membered ring;

R¹⁶ on Y₃ together with R¹⁶ on Y₄ together with the intervening atoms can form a 3- to 7-membered ring;

when R¹⁵ is —N(R¹⁷)₂, R¹⁷ on Y₃ together with R¹⁶ on either Y₁, Y₂, or Y₄ together with the intervening atoms can form a 3- to 7-membered ring;

R¹⁷ is each independently an open valence, —H, —C(O)NH—C₁-C₆ alkyl, —C(O)—O—C₁-C₆ alkyl, —C(O)—O-phenyl, —C(O)—O—C₁-C₃ alkylene-phenyl, —C(O)—O—C₂-C₃ alkenylene-phenyl, —C(O)—O—C₂-C₃ alkynylene-phenyl, —C(O)H, —C₁-C₉ alkyl, —C₂-C₉ alkenyl, —C₂-C₉ alkynyl, —C₃-C₈ monocyclic cycloalkyl, —C₃-C₈ monocyclic cycloalkenyl, —C₇-C₉ monocyclic cycloalkynyl, -phenyl, —C₁-C₃ alkylene-phenyl, —C₂-C₃ alkenylene-phenyl, or —C₂-C₃ alkynylene-phenyl,

each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R¹⁸ is —H, -halo, —OH, —CN, —NH₂, —N₃, —NO₂, —CHO, —COOH, —C₁-C₆ alkyl, —C₂-C₆ alkenyl, —C₂-C₆ alkynyl, —NHC(O)—C₁-C₆ alkyl, —C₁-C₆ alkylene-O—C₁-C₆ alkyl, —C₁-C₆ alkylene-C(O)O—C₁-C₆ alkyl, —O—C₁-C₆ alkyl, —S—C₁-C₆ alkyl, —C(O)O—C₁-C₆ alkyl, —C(O)—C₁-C₆ alkyl, -phenyl, —O-phenyl, —S-phenyl, -benzoyl, -benzyl, —C₁-C₆ alkylene-C(O)O-phenyl, —NH—C₁-C₆ alkyl, —N(C₁-C₆ alkyl)₂, —NH-phenyl, —NH-benzyl, —NH—C(O)—C₁-C₆ alkyl, —NH—C(O)—C₂-C₆ alkenyl, —NH—C(O)—C₂-C₆ alkynyl, —C₁-C₆ alkylene-COOH, —C₁-C₆ alkylene-CHO, —C₁-C₆ alkylene-NH—C₁-C₆ alkyl, —C₁-C₆ alkylene-N(C₁-C₆ alkyl)₂, —C(O)NH—C₁-C₆ alkyl, —C(O)N(C₁-C₆)₂ alkyl, —O—C₁-C₆ alkylene-NH—C₁-C₆ alkyl, —O—C₁-C₆ alkylene-N(C₁-C₆ alkyl)₂, —OS(O)₂—C₁-C₆ alkyl, or —SH,

wherein each —C₁-C₆ alkyl, —C₁-C₆ alkylene, —C₂-C₆ alkenyl, —C₂-C₆ alkynyl, -phenyl, -benzyl, or benzoyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

R²⁷ is C₁-C₆ alkyl, -phenyl, —(C((R¹²)₂)ₚ)-phenyl, a 5- or 6-membered aromatic monocyclic heterocycle, a 3- to 7-membered non-aromatic monocyclic heterocycle, or a 8- to 12-membered bicyclic heterocycle, —(C((R¹²)₂)ₚ)-5- or 6-membered aromatic monocyclic heterocycle, —(C((R¹²)₂)ₚ)-3- to 7-membered non-aromatic monocyclic heterocycle, —(C((R¹²)₂)ₚ)-8- to 12-membered bicyclic heterocycle,

wherein p=1-4 with the proviso that when the carbon chain of length p is between two heteroatoms, p=2-4; and

R²⁸ is a 5- or 6-membered aromatic monocyclic heterocycle, a 3- to 7-membered non-aromatic monocyclic heterocycle, a 8- to 12-membered bicyclic heterocycle, -phenyl, -benzyl,

wherein each heterocycle can be substituted on a single saturated carbon atom not adjacent to a nitrogen atom with —O(CH₂)₂O— or —(CH₂)₃O—;

each heterocycle can optionally be benzo-fused; and

each 5- or 6-membered aromatic monocyclic heterocycle, a 3- to 7-membered non-aromatic monocyclic heterocycle, a 8- to 12-membered bicyclic heterocycle, -phenyl, -benzyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl.

The invention provides compositions comprising an effective amount of a compound of Formula (I) or a pharmaceutically acceptable salt thereof and a physiologically acceptable carrier or vehicle.

The invention further provides methods for treating or preventing a proliferative disorder, e.g. cancer, comprising administering to a subject in need thereof an effective amount of a compound of Formula (I) or a pharmaceutically acceptable salt thereof.

The invention further provides methods for treating or preventing an autoimmune disease comprising administering to a subject in need thereof an effective amount of a compound of Formula (I) or a pharmaceutically acceptable salt thereof.

The invention provides methods for modulating activity of PDK-1 comprising administering to a subject in need thereof an effective amount of the compound or a pharmaceutically acceptable salt of the compound of Formula (I), wherein it is known that PDK-1 activity is related to a disease or condition.

The invention further provides methods for modulating activity of PKA comprising administering to a subject in need thereof an effective amount of the compound or a pharmaceutically acceptable salt of the compound of Formula (I), wherein it is known that PKA activity is related to a disease or condition. In one embodiment, the disease or condition is cancer.

The invention provides methods for modulating activity of an Akt isoform comprising administering to a subject in need thereof an effective amount of the compound or a pharmaceutically acceptable salt of the compound of Formula (I), wherein it is known that the Akt isoform activity is related to a disease or condition. In one embodiment, the disease or condition is cancer.

The invention further provides methods for modulating activity of a PKC isoform comprising administering to a subject in need thereof an effective amount of the compound or a pharmaceutically acceptable salt of the compound of Formula (I), wherein it is known that the PKC isoform activity is related to a disease or condition. In one embodiment, the disease or condition is cancer.

The invention provides methods for modulating activity of S6K (p70 ribosomal S6 kinase) comprising administering to a subject in need thereof an effective amount of the compound or a pharmaceutically acceptable salt of the compound of Formula (I), wherein it is known that S6K activity is related to a disease or condition. In one embodiment, the disease or condition is cancer.

A compound of Formula (I) or a pharmaceutically acceptable salt thereof (a “Benzo[c][2,7]naphthyridine Derivative”) is useful for treating or preventing a proliferative disorder.

A Benzo[c][2,7]naphthyridine Derivative is useful for treating or preventing an autoimmune disease.

A composition comprising an effective amount of a Benzo[c][2,7]naphthyridine Derivative and a physiologically acceptable carrier or vehicle is useful for treating or preventing a proliferative disorder.

A composition comprising an effective amount of a Benzo[c][2,7]naphthyridine Derivative and a physiologically acceptable carrier or vehicle is useful for treating or preventing an autoimmune disease.

The invention provides a process for preparing the compound of the Formula (IIc)

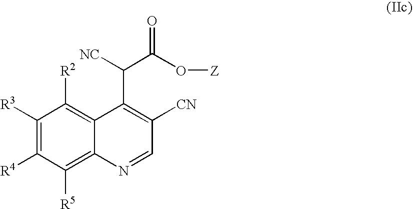

comprising reacting the compound of the Formula (IIa)

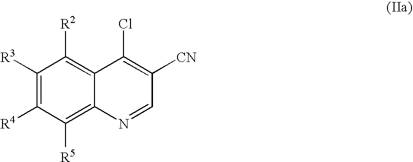

with the compound of the Formula (IIb)

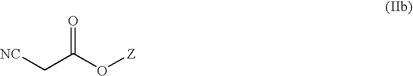

wherein

Z is —C(C₁-C₆ alkyl)₃,

R², R³, R⁴ and R⁵ are as defined above for the compounds of Formula (I).

The invention further provides a process for preparing the compound of the Formula (IIe)

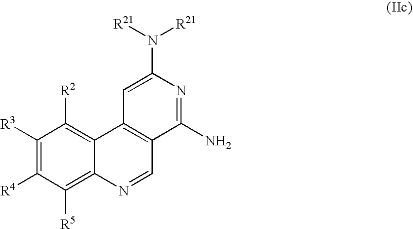

comprising reacting the compound of the Formula (IIc)

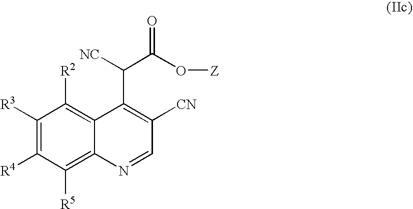

with the compound of the Formula (IId)

(R²¹)₂NH (IId)

wherein

Z is —C(C₁-C₆ alkyl)₃,

each R²¹ is independently R¹², R¹³ or —(C(R¹²)₂)ₙ—R¹³, wherein n=1-4, with the proviso that when the carbon chain of length n is between two heteroatoms, n=2-4; or

(R²¹)₂N— is —R^{13′},

R², R³, R⁴, R⁵, R¹² and R¹³ are as defined above for the compounds of Formula (I), and

R^{13′} is a nitrogen-containing 3- to 7-membered non-aromatic monocyclic heterocycle or a nitrogen-containing non-aromatic 8- to 12-membered bicyclic heterocycle, which is not a lactam, which can optionally be mono- or di-substituted with —O—, —S—, —NH-benzoyl, R²⁸, —O—R²⁸, or R¹⁸,

wherein each 5- or 6-membered aromatic monocyclic heterocycle, a 3- to 7-membered non-aromatic monocyclic heterocycle, a 8- to 12-membered bicyclic heterocycle, -phenyl, -benzyl, benzoyl or —C₁-C₆ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl. In one embodiment, one or more is one to three.

The invention provides a process for preparing the compound of the Formula (IIe)

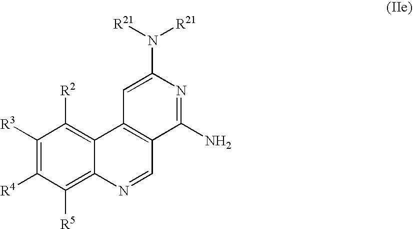

comprising decarboxylating the compound of the Formula (IIc)

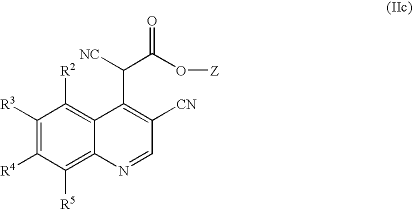

to provide the compound of the Formula (IIc′)

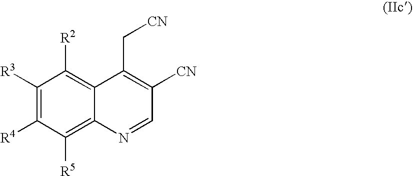

and then reacting the compound of the Formula (IIc′) with the compound of the Formula (IId)

(R²¹)₂NH (IId)

wherein

Z is —C(C₁-C₆ alkyl)₃,

each R²¹ is independently R¹², R¹³ or —(C(R¹²)₂)ₙ—R¹³, wherein n=3-4, with the proviso that when the carbon chain of length n is between two heteroatoms, n=2-4; or

(R²¹)₂N— is —R^{13′},

R², R³, R⁴, R⁵, R¹² and R¹³ are as defined above for the compounds of Formula (I), and

R^{13′} is a nitrogen-containing 3- to 7-membered non-aromatic monocyclic heterocycle or a nitrogen-containing non-aromatic 8- to 12-membered bicyclic heterocycle, which is not a lactam, which can optionally be mono- or di-substituted with —O—, —S—, —NH-benzoyl, R²⁸, —O—R²⁸, or R¹⁸,

wherein each 5- or 6-membered aromatic monocyclic heterocycle, a 3- to 7-membered non-aromatic monocyclic heterocycle, a 8- to 12-membered bicyclic heterocycle, -phenyl, -benzyl, benzoyl or —C₁-C₆ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl. In one embodiment, one or more is one to three.

The invention provides a process for preparing the compound of the Formula (IIIb)

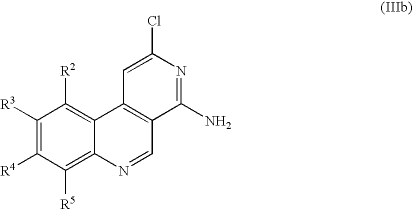

comprising reacting the compound of the Formula (IIIa)

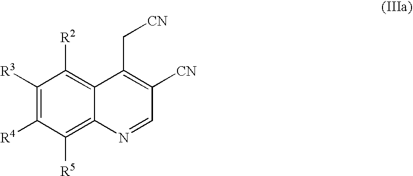

with hydrogen chloride,
wherein

R², R³, R⁴ and R⁵ are as defined above for the compounds of Formula (I).

The invention provides a process for preparing the compound of the Formula (IIIc)

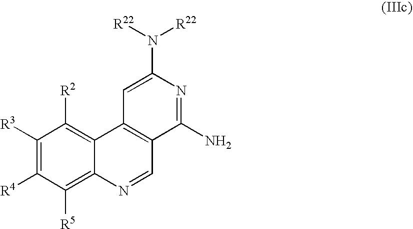

comprising reacting the compound of the Formula (IIb)

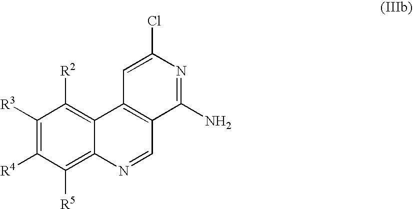

with the compound of formula H—N(R²²)₂,
wherein

(R²²)₂N— is —NR¹²R¹³, —NR¹³(C(R¹²)₂)ₙ—R¹³ wherein n=1-4, with the proviso that when the carbon chain of length n is between two heteroatoms, n=2-4; or

(R²²)₂N— is —R^{13″},

R², R³, R⁴, R⁵, R¹² and R¹³ are as defined above for the compounds of Formula (I), and

R^{13″} is a nitrogen-containing 3- to 7-membered non-aromatic monocyclic heterocycle or a nitrogen-containing non-aromatic 8- to 12-membered bicyclic heterocycle, which is not a lactam, which can optionally be mono- or di-substituted with —O—, —S—, —NH-benzoyl, R²⁸, —O—R²⁸, or R¹⁸,

wherein each 5- or 6-membered aromatic monocyclic heterocycle, a 3- to 7-membered non-aromatic monocyclic heterocycle, a 8- to 12-membered bicyclic heterocycle, -phenyl, -benzyl, benzoyl or —C₁-C₆ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl. In one embodiment, one or more is one to three.

The invention provides a process for preparing the compound of the Formula (IIId)

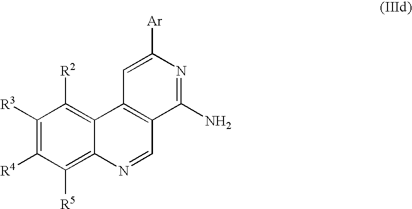

comprising reacting the compound of the Formula (IIb)

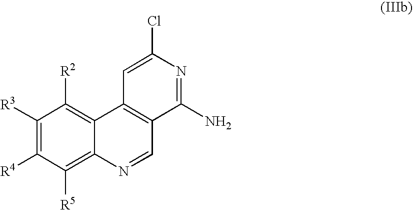

with the compound of formula Ar—B(OH)₂,
wherein

Ar is aryl or a heteroaryl group compound substituted on a carbon atom, and

R², R³, R⁴ and R⁵ are as defined above for the compounds of Formula (I).

The invention further provides a process for preparing the compound of the Formula (IVb)

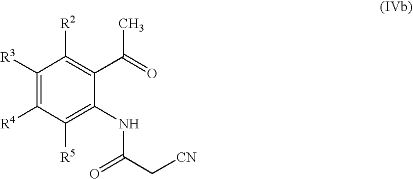

comprising reacting the compound of the Formula (IVa)

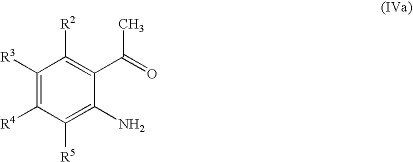

with 2-cyanoacetyl chloride,
wherein

R¹, R³, R⁴ and R⁵ are as defined above for the compounds of Formula (I).

The invention provides a process for preparing the compound of the Formula (IVc)

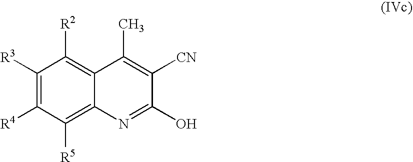

comprising cyclizing the compound of the Formula (IVb)

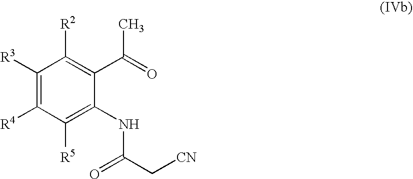

with a base,
wherein

R², R³, R⁴ and R⁵ are as defined above for the compounds of Formula (I).

The invention provides a process for preparing the compound of the Formula (IVc)

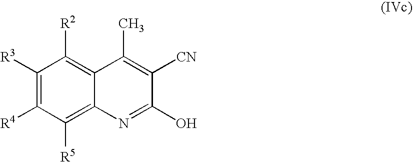

comprising reacting the compound of the Formula (IVa)

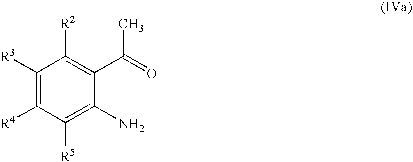

with ethyl cyanoacetate,
wherein

R², R³, R⁴ and R⁵ are as defined above for the compounds of Formula (I).

The invention provides a process for preparing the compound of the Formula (IVd)

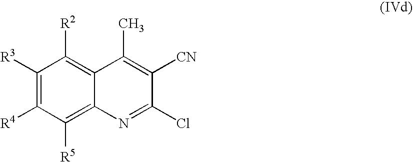

comprising reacting the compound of the Formula (IVc)

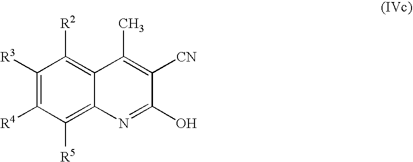

with a chlorinating reagent,
wherein

R², R³, R⁴ and R⁵ are as defined above for the compounds of Formula (I).

The invention provides a process for preparing the compound of the Formula (IVe)

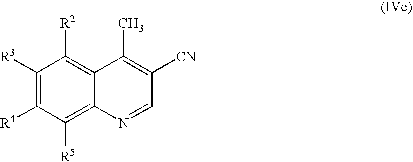

comprising removing the chlorine atom of the compound of Formula (IVd)

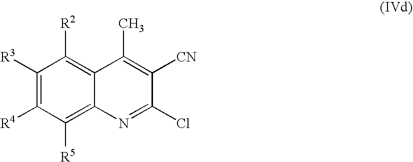

by hydrogenolysis with hydrogen and a palladium catalyst in the presence of a base,
wherein

R², R³, R⁴ and R⁵ are as defined above for the compounds of Formula (I).

The invention provides a process for preparing the compound of the Formula (IVg)

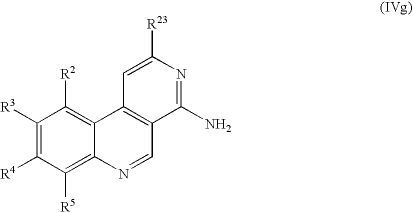

comprising reacting the compound of the Formula (IVf)

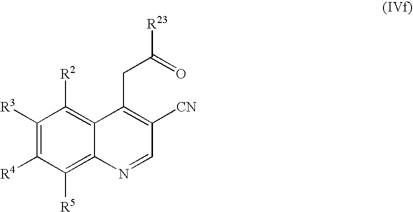

with ammonium acetate,
wherein

R²³ is —C₁-C₆ alkyl, -phenyl, -benzyl, —(C(R¹²)₂)ₙ—R¹³, —(C(R¹²)₂)ₙ—R¹⁴, —(C(R¹²)₂)ₙ—N(R¹²)(C(R¹²)₂)ₙ—R¹³, —(C(R¹²)₂)ₙ—N(R¹²)(C(R¹²)₂)ₙ—R¹⁴, —(C(R¹²)₂)—O—(C(R¹²)₂)—R¹³, —(C(R¹²)₂)ₙ—O—(C(R¹²)₂)ₙ—R¹⁴, —(C(R¹²)₂)ₙ—S—(C(R¹²)₂)ₙ—R¹³, —(C(R¹²)₂)ₙ—S—(C(R¹²)₂)ₙ—R¹⁴, R¹³, or R¹⁴,

wherein each C₁-C₆ alkyl, -phenyl or -benzyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;

wherein n=1-4 at each occurrence, with the proviso that when R¹³ or R¹⁴ is a heterocycle, n=2-4; and

R², R³, R⁴, R⁵, R¹², R¹³ and R¹⁴ are as defined above for the compounds of Formula (I). In one embodiment, one or more is one to three.

The invention provides a process for preparing the compound of the Formula (Vc)

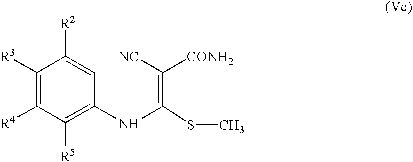

comprising reacting the compound of the Formula (Va)

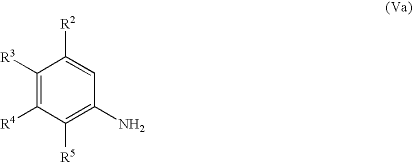

with the compound of the Formula (Vb)

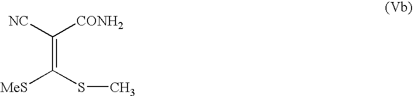

wherein

R², R³, R⁴ and R⁵ are as defined above for the compounds of Formula (I).

The invention provides a process for preparing the compound of the Formula (Vd)

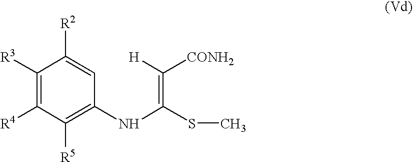

comprising heating the compound of the Formula (Vc)

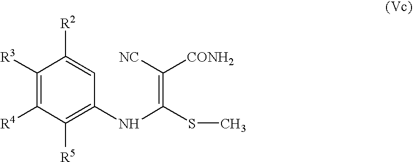

in the presence of N(C₁-C₆ alkyl)₃⁺,
wherein

R², R³, R⁴ and R⁵ are as defined above for the compounds of Formula (I).

The invention provides a process for preparing the compound of the Formula (Ve)

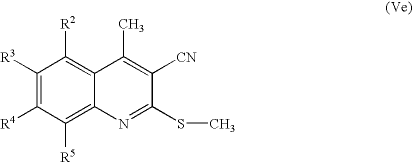

comprising reacting the compound of the Formula (Vd)

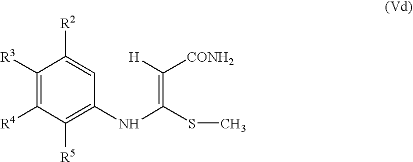

with dimethylacetamide,
wherein

R², R³, R⁴ and R⁵ are as defined above for the compounds of Formula (I).

The invention provides a process for preparing the compound of the Formula (Vf)

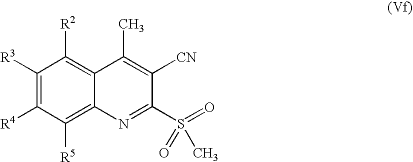

comprising oxidizing the compound of the Formula (Ve)

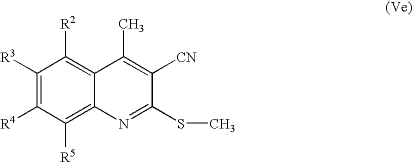

wherein

R², R³, R⁴ and R⁵ are as defined above for the compounds of Formula (I).

The invention provides a process for preparing the compound of the Formula (Vg)

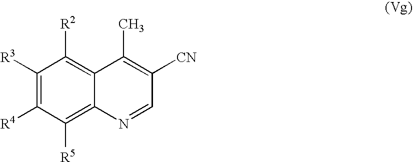

comprising removing the sulphone group of the compound of the Formula (Vf)

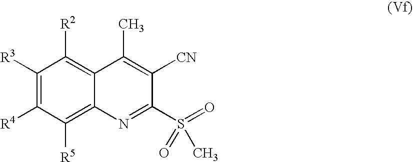

wherein

R², R³, R⁴ and R⁵ are as defined above for the compounds of Formula (I).

The invention provides a process for preparing the compound of the Formula (VIb)

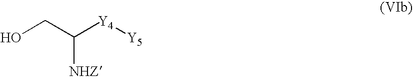

comprising reacting the compound of the Formula (VIa)

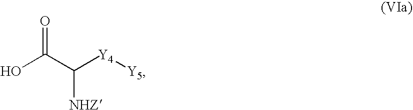

with a reducing agent capable of reducing the carboxylate group,
wherein

Z′ is a protecting group for amines, and

Y₄ and Y₅ are as defined above for the compounds of Formula (I).

The invention provides a process for preparing the compound of the Formula (VId)

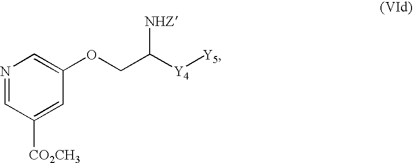

comprising reacting the compound of the Formula (VIb)

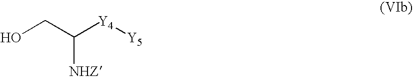

with the compound of the Formula (VIc)

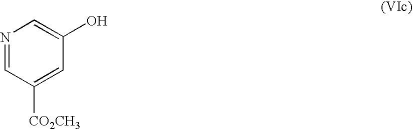

wherein

Z′ is a protecting group for amines, and

Y₄ and Y₅ are as defined above for the compounds of Formula (I).

The invention provides a process for preparing the compound of the Formula (VIIc)

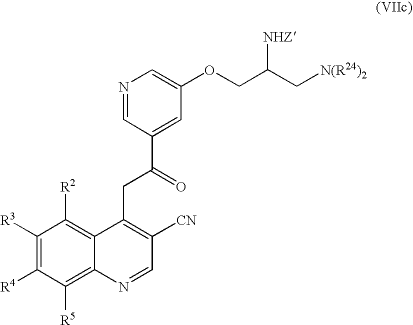

comprising reacting the compound of the Formula (VIIa)

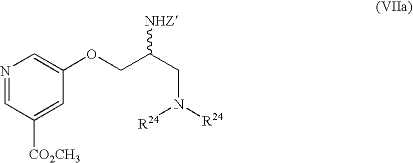

with the compound of the Formula (VIIb)

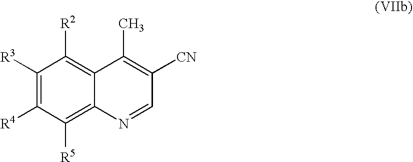

wherein

Z′ is a protecting group for amines,

—N(R²⁴)₂ is —NH₂, —NH—C₁-C₆ alkyl, —N(C₁-C₆ alkyl)₂, —NH-phenyl, —NR¹²R¹³, —N(R¹²)(C(R¹²)₂)ₘ—R¹³, —O(C(R¹²)₂)ₘ—R¹³, R¹³, or R^{14′};

R^{14′} is —N(R¹²)₂, —NR¹²—(C(R¹²)₂)ₙ—N(R¹²)₂, —NR¹²—(C(R¹²)₂)—OR¹², —N((C(R¹²)₂)ₙ—N(R¹²)₂)₂, —N((C(R¹²)₂)ₙ—OR¹²)₂, or —N((C(R¹²)₂)—N(R¹²))(C(R¹²)₂)ₙ—OR¹²); and

n, R², R³, R⁴, R⁵, R¹² and R¹³ are as defined above for the compounds of Formula (I).

The invention provides a process for preparing the compound of the Formula (VIId)

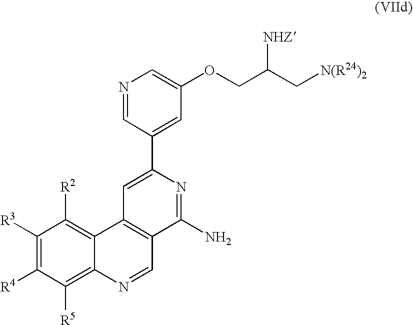

comprising reacting the compound of the Formula (VIIc)

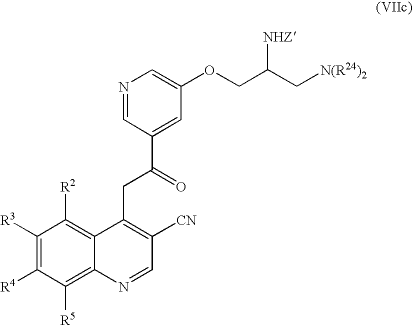

with ammonium acetate,
wherein

Z′ is a protecting group for amines,

—N(R¹⁴)₂ is —NH₂, —NH—C₁-C₆ alkyl, —N(C₁-C₆ alkyl)₂, —NH-phenyl, —NR¹²R¹³, —N(R¹²)(C(R¹²)₂)ₘ—R¹³, —O(C(R¹²)₂)ₘ—R¹³, R¹³, or R^{14′};

R^{14′} is —N(R¹²)₂, —NR¹²—(C(R¹²)₂)ₙ—N(R¹²)₂, —NR¹²—(C(R¹²)₂)—OR¹², —N((C(R¹²)₂)ₙ—N(R¹²)₂)₂, —N((C(R¹²)₂)ₙ—OR¹²)₂, or —N((C(R¹²)₂)—N(R¹²)₂)(C(R¹²)₂)ₙ—OR¹²); and

n, R², R³, R⁴, R⁵, R¹² and R¹³ are as defined above for the compounds of Formula (I).

The invention provides a process for preparing the compound of the Formula (VIIe)

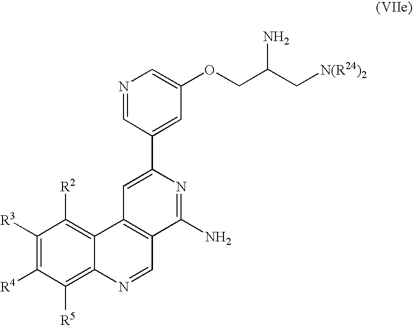

comprising removing Z′ from the compound of the Formula (VIId)

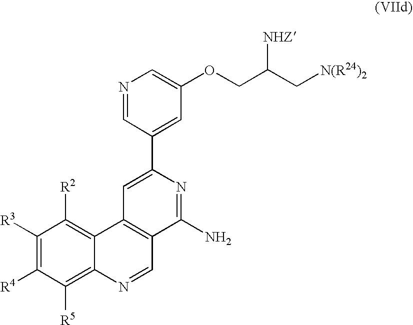

wherein

Z′ is a protecting group for amines,

—N(R¹⁴)₂ is —NH₂, —NH—C₁-C₆ alkyl, —N(C₁-C₆ alkyl)₂, —NH-phenyl, —NR¹²R¹³, —N(R¹²)(C(R¹²)₂)ₘ—R¹³, —O(C(R¹²)₂)ₘ—R¹³, R¹³, or R^{14′};

R^{14′} is —N(R¹²)₂, —NR¹²—(C(R¹²)₂)ₙ—N(R¹²)₂, —NR¹²—(C(R¹²)₂)ₙ—OR¹², —N((C(R¹²)₂)ₙ—N(R¹²)₂)₂, —N((C(R¹²)₂)ₙ—OR¹²)₂, or —N((C(R¹²)₂)—N(R¹²))(C(R¹²)₂)—OR¹²); and

n, R², R³, R⁴, R⁵, R¹² and R¹³ are as defined above for the compounds of Formula (I).

The invention provides a process for preparing the compound of the Formula (VIIc)

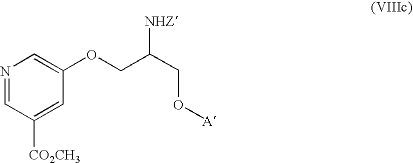

comprising reacting the compound of the Formula (VIIIa)

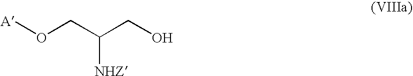

with the compound of the Formula (VIIIb)

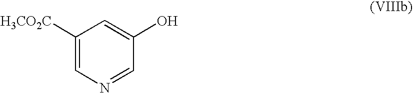

wherein

Z′ is a protecting group for amines; and

A′ is a silyl protecting group.

The invention provides a process for preparing the compound of the Formula (VIIIe)

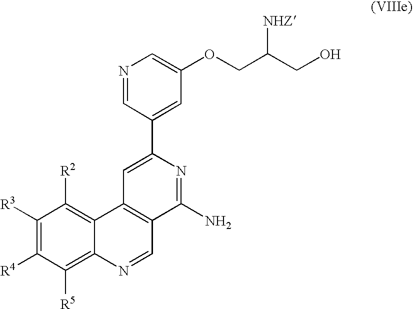

comprising reacting the compound of the Formula (VIIIc)

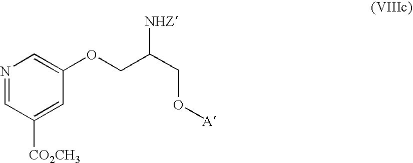

with the compound of the Formula (VIIId)

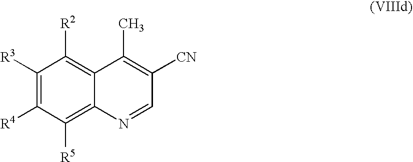

wherein

Z′ is a protecting group for amines,

A′ is a silyl protecting group, and

R², R³, R⁴, and R⁵ are as defined above for the compounds of Formula (I).

The invention provides a process for preparing the compound of the Formula (VIIIf)

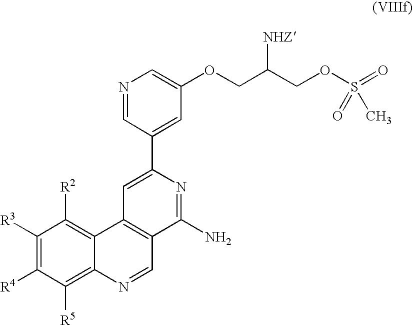

comprising reacting the compound of the Formula (VIIIe)

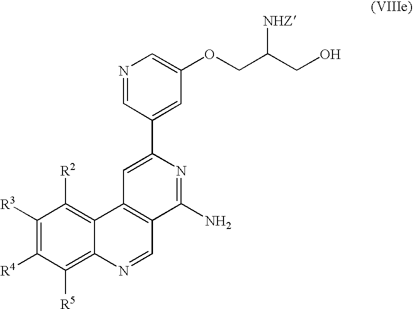

with a mesylate halide,
wherein

Z′ is a protecting group for amines,

R², R³, R⁴ and R⁵ are as defined above for the compounds of Formula (I).

The invention provides a process for preparing the compound of the Formula (VIIIg)

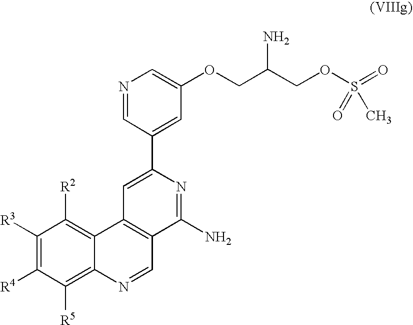

comprising removing Z′ from the compound of the Formula (VIIIf)

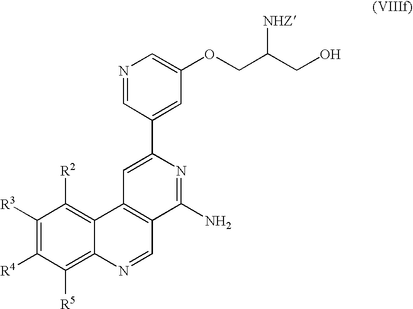

wherein

Z′ is a protecting group for amines, and

R², R³, R⁴ and R⁵ are as defined above for the compounds of Formula (I).

The invention provides a process for preparing the compound of the Formula (VIIIh)

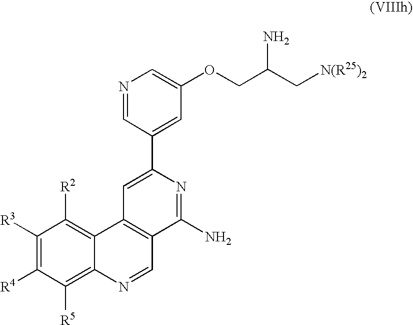

comprising reacting the compound of the Formula (VIIIg)

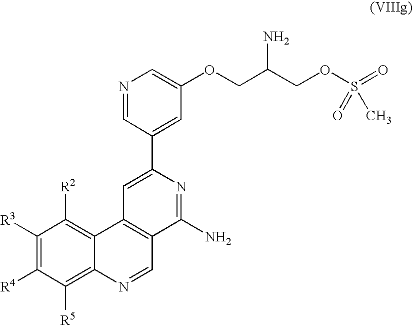

with the compound of formula H—N(R²⁵)₂,
wherein

—N(R²⁵)₂ is —NH₂, —NH—C₁-C₆ alkyl, —N(C₁-C₆ alkyl)₂, —NH-phenyl, —NR¹²R¹³, —N(R¹²)(C(R¹²)₂)ₘ—R¹³, —O(C(R¹²)₂)ₘ—R¹³, R¹³, or R^{14′};

R^{14′} is —N(R¹²)₂, —NR¹²—(C(R¹²)₂)ₙ—N(R¹²)₂, —NR¹²—(C(R¹²)₂)—OR¹², —N((C(R¹²)₂)ₙ—N(R¹²)₂)₂, —N((C(R¹²)₂)ₙ—OR¹²)₂, or —N((C(R¹²)₂)ₙ—N(R¹²))(C(R¹²)₂)—OR¹²); and

m, n, R², R³, R⁴, R⁵, R¹² and R¹³ are as defined above for the compounds of Formula (I).

The invention provides a process for preparing the compound of the Formula (IXc)

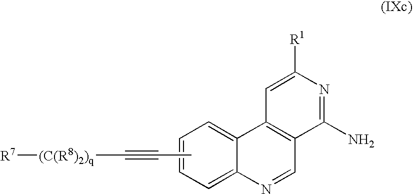

comprising reacting the compound of the Formula (IXa)

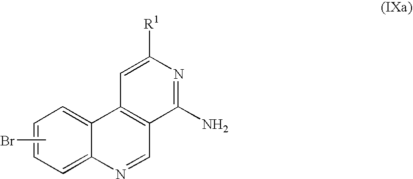

with the compound of the Formula (IXb)

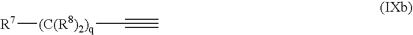

wherein

q, R¹, R⁷, and R⁸ are as defined above for the compounds of Formula (I).

The invention provides a process for preparing the compound of the Formula (IXe)

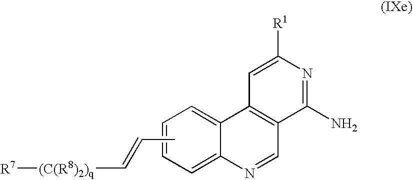

comprising reacting the compound of the Formula (IXa)

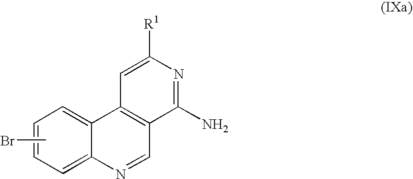

with the compound of the Formula (IXd)

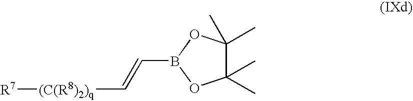

wherein

q, R¹, R⁷, and R⁸ are as defined above for the compounds of Formula (I).

The invention provides process for preparing the compound of the Formula (Xb)

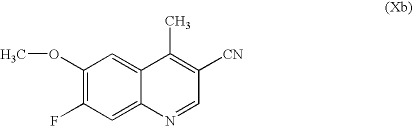

comprising reacting the compound of the Formula (Xa)

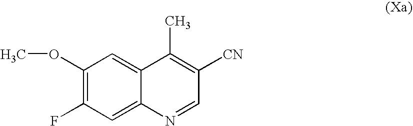

with methyl magnesium bromide.

The invention provides a process for preparing the compound of the Formula (Xd)

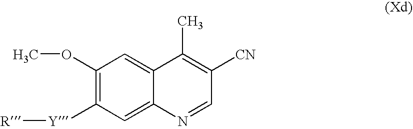

comprising reacting the compound of the Formula (Xb)

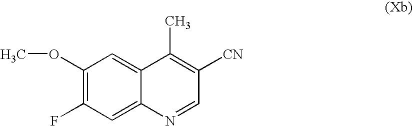

with the compound of the formula R′″—Y′″—H,
wherein

Y′″ is —S—, —O—, or —N(R⁸)—,

R²⁶ is

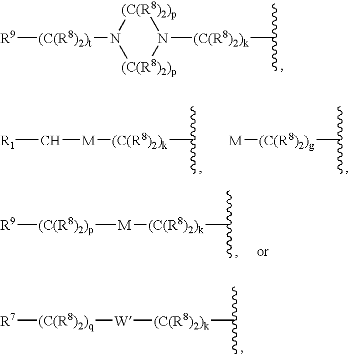

wherein

g, k, p, q, t, M, W′, R⁷, R⁸, R⁹, R¹⁰ and R¹¹ are as defined above for the compounds of Formula (I).

The invention provides a process for preparing the compound of the Formula (XIb)

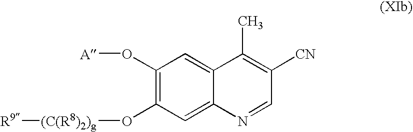

comprising hydrolyzing the Ac—O— group of the compound of the Formula (XIa)

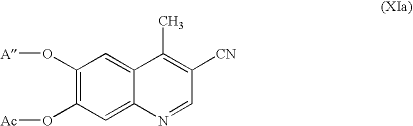

with a base and reacting it with the compound of the formula R^{9″}—(C(R⁸)₂)_{g}-J′,
wherein

A″ is —C₁-C₆ alkyl;

R^{9″} is -halo or —OR⁸;

J′ is —Br, —I, -mesylate, or -tosylate; and

g and R⁸ are as defined above for the compounds of Formula (I).

The invention provides a process for preparing the compound of the Formula (XIc)

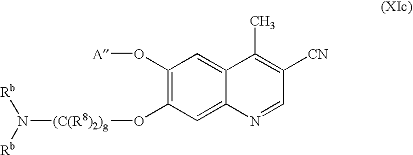

comprising reacting the compound of the Formula (XIb)

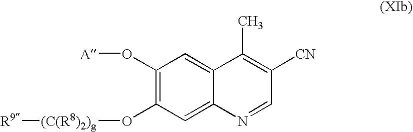

with the compound of the formula (R^{b})₂NH,
wherein

A″ is —C₁-C₆ alkyl;

R^{9″} is -halo or —OR⁵; and

R^{b} is —N(R⁸)₂, —N(R⁸)₃⁺, —NR⁸(OR⁸), a nitrogen-containing 5- or 6-membered aromatic monocyclic heterocycle, or a nitrogen-containing 3- to 7-membered non-aromatic monocyclic heterocycle, each of which is optionally mono or di-substituted on a carbon atom with —OH, —R⁸, —N(R⁸)₂, —OR⁸, —(C(R⁸)₂)ᵣOR⁸, —(C(R⁸)₂)ᵣN(R⁸)₂, or a 3- to 7-membered monocyclic heterocycle,

wherein r=1-6;

each R⁷ is optionally mono or di-substituted on a saturated carbon atom with —O(C(R⁸)₂)ᵣO—,

wherein r=1-6;

each R⁷ is optionally mono-substituted on a nitrogen atom with R⁸; and

g, r, R⁷, and R⁸ are as defined above for the compounds of Formula (I).

The invention provides a process for preparing the compound of the Formula (XId)

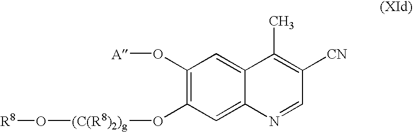

comprising reacting the compound of the Formula (XIb)

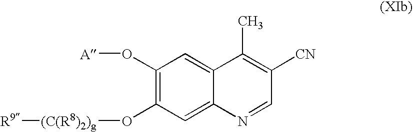

with the compound of the formula R⁸—OH,
wherein

A″ is —C₁-C₆ alkyl;

R^{9″} is -halo;

R^{b} is —N(R⁸)₂, —N(R⁸)₃⁺, —NR⁸(OR⁸), a nitrogen-containing 5- or 6-membered aromatic monocyclic heterocycle, or a nitrogen-containing 3- to 7-membered non-aromatic monocyclic heterocycle, each of which is optionally mono or di-substituted on a carbon atom with —OH, —R⁸, —N(R⁸)₂, —OR⁸, —(C(R⁸)₂)ᵣOR⁸, —(C(R⁸)₂)ᵣN(R⁸)₂, or a 3- to 7-membered monocyclic heterocycle,

wherein r=1-6;

each R⁷ is optionally mono or di-substituted on a saturated carbon atom with —O(C(R⁸)₂)ᵣO—,

wherein r=1-6;

each R⁷ is optionally mono-substituted on a nitrogen atom with R⁸; and

g, R⁷ and R⁸ are as defined above for the compounds of Formula (I).

The invention provides a process for preparing the compound of the Formula (XIIb)

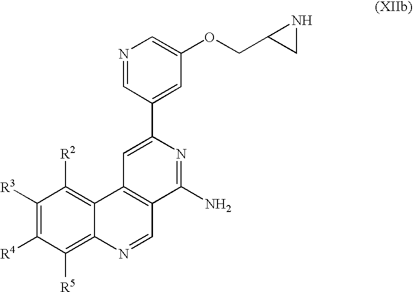

comprising reacting the compound of the Formula (XIIa)

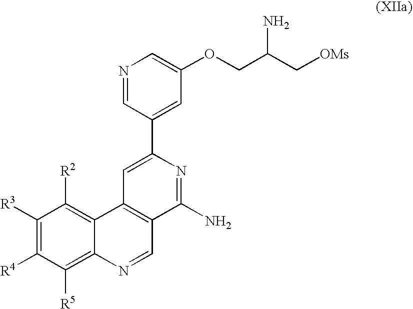

with a base,
wherein R², R³, R⁴, and R⁵ are as defined above for the compounds of Formula (I).

The invention provides a process for preparing the compound of the Formula (XIIIb)

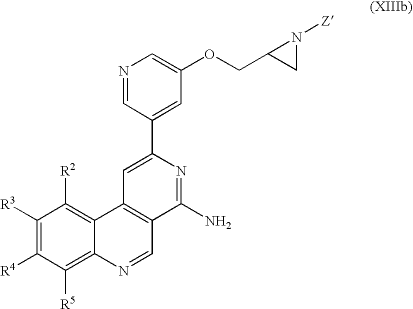

comprising protecting the compound of the Formula (XIIIa)

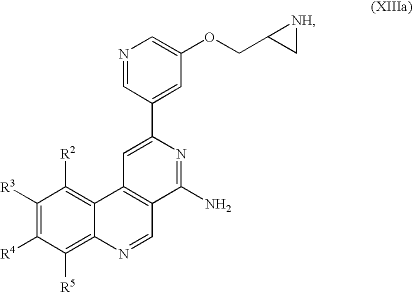

with Z′,
wherein Z′ is a protecting group for amines and R², R³, R⁴, and R⁵ are as defined above for the compounds of Formula (I).

The invention provides a process for preparing the compound of the Formula (XIVb)

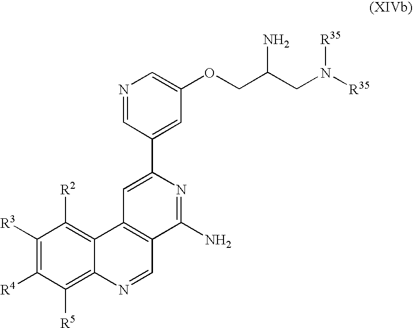

comprising
a) heating the compound of the formula (XIVa)

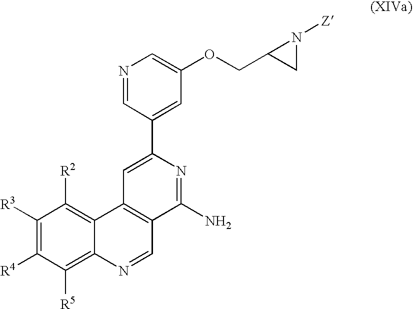

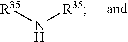

with the compound of the formula
b) removing Z′ group,
wherein Z′ is a protecting group for amines;

—N(R³⁵)₂ is —NH₂, —NH—C₁-C₆ alkyl, —N(C₁-C₆ alkyl)₂, —NH-phenyl, —NR¹²R¹³, —N(R¹²)(C(R¹²)₂)ₘ—R¹³, —O(C(R¹²)₂)ₘ—R¹³, R¹³, or R^{14′};

R^{14′} is —N(R¹²)₂, —NR¹²—(C(R¹²)₂)ₙ—N(R¹²)₂, —NR¹²—(C(R¹²)₂)—OR¹², —N((C(R¹²)₂)ₙ—N(R¹²)₂)₂, —N((C(R¹²)₂)ₙ—OR¹²)₂, or —N((C(R¹²)₂)ₙ—N(R¹²))(C(R¹²)₂)—OR¹²); and

m, n, R², R³, R⁴, R⁵, R¹² and R¹³ are as defined above for the compounds of Formula (I).

The invention provides a process for preparing the compound of the Formula (XVb)

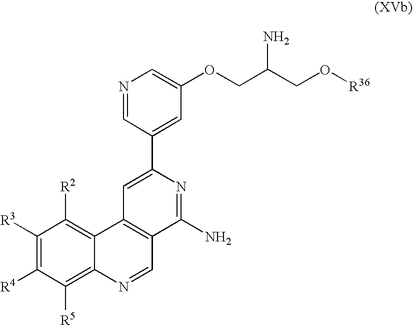

comprising
a) heating the compound of the formula (XVa)

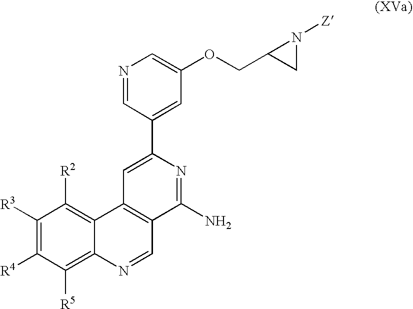

with the compound of the formula HO—R³⁶; and
b) removing Z′ group,
wherein Z′ is a protecting group for amines,

R³⁶ is a 5- or 6-membered aromatic monocyclic heterocycle, a 3- to 7-membered non-aromatic monocyclic heterocycle, a 8- to 12-membered bicyclic heterocycle, -phenyl, -benzyl,

wherein each heterocycle can be substituted on a single saturated carbon atom not adjacent to a nitrogen atom with —O(CH₂)₂O— or —(CH₂)₃O—;

each heterocycle can optionally be benzo-fused; and

each 5- or 6-membered aromatic monocyclic heterocycle, a 3- to 7-membered non-aromatic monocyclic heterocycle, a 8- to 12-membered bicyclic heterocycle, -phenyl, -benzyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl; and

R², R³, R⁴, and R⁵ are as defined above for the compounds of Formula (I).

The invention provides a process for preparing the compound of the Formula (XVIb)

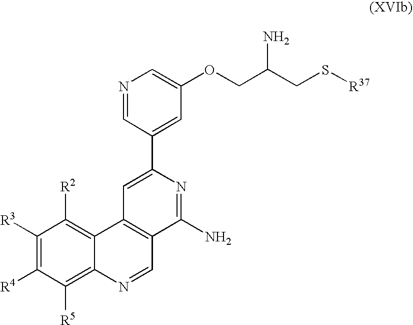

comprising
a) heating the compound of the formula (XVIa)

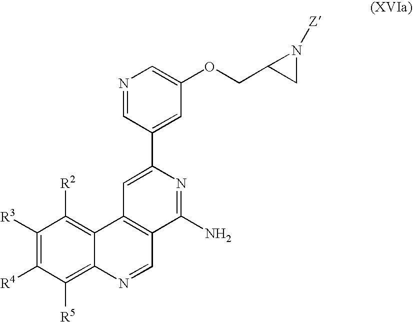

with the compound of the formula HS—R³⁷; and
b) removing Z′ group,
wherein Z′ is a protecting group for amines,

R³⁷ is a 5- or 6-membered aromatic monocyclic heterocycle, a 3- to 7-membered non-aromatic monocyclic heterocycle, a 8- to 12-membered bicyclic heterocycle, -phenyl, -benzyl,

wherein each heterocycle can be substituted on a single saturated carbon atom not adjacent to a nitrogen atom with —O(CH₂)₂O— or —(CH₂)₃O—;

each heterocycle can optionally be benzo-fused; and

each 5- or 6-membered aromatic monocyclic heterocycle, a 3- to 7-membered non-aromatic monocyclic heterocycle, a 8- to 12-membered bicyclic heterocycle, -phenyl, -benzyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl; and

R², R³, R⁴, and R⁵ are as defined above for the compounds of Formula (I).

The invention provides a process for preparing the compound of the Formula (XVIIb)

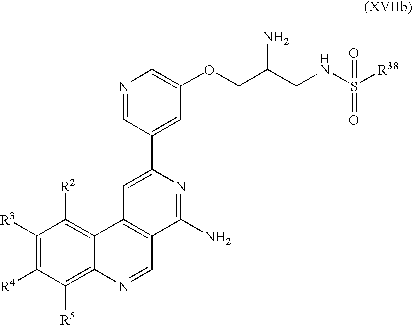

comprising
a) heating the compound of the formula (XVIIa)

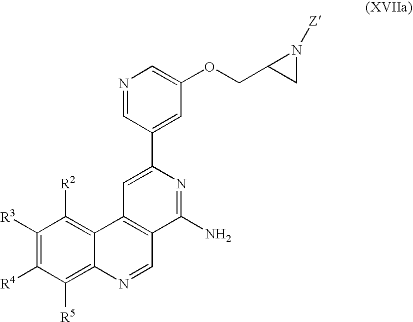

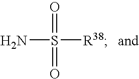

with the compound of the formula
b) removing Z′ group,
wherein Z′ is a protecting group for amines,

R³⁸ is C₁-C₆ alkyl, -phenyl, —(C((R¹²)₂)ₚ)-phenyl, a 5- or 6-membered aromatic monocyclic heterocycle, a 3- to 7-membered non-aromatic monocyclic heterocycle, or a 8- to 12-membered bicyclic heterocycle, —(C((R¹²)₂)ₚ)-5- or 6-membered aromatic monocyclic heterocycle, —(C((R¹²)₂)ₚ)-3- to 7-membered non-aromatic monocyclic heterocycle, —(C((R¹²)₂)ₚ)-8- to 12-membered bicyclic heterocycle;

and p, R², R³, R⁴, R⁵ and R¹² are as defined above for the compounds of Formula (I).

The details of the invention are set forth in the accompanying description below.

### 5. DETAILED DESCRIPTION OF THE INVENTION

### 5.1 Definitions and Abbreviations

The following definitions are used in connection with the Benzo[c][2,7]naphthyridine Derivatives:

“C₁-C₂ alkyl” refers to a straight chain saturated hydrocarbon containing 1-2 carbon atoms. Representative C₁-C₂ alkyl groups include methyl and ethyl. In one embodiment, the C₁-C₂ alkyl group is substituted with one or more of the following groups: -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl. In one embodiment, one or more is one to three.

“C₁-C₄ alkyl” refers to a straight or branched chain saturated hydrocarbon containing 1-4 carbon atoms. Representative C₁-C₄ alkyl groups include methyl, ethyl, propyl, isopropyl, butyl, tert-butyl, sec-butyl, and isobutyl. In one embodiment, the C₁-C₄ alkyl group is substituted with one or more of the following groups: -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl. In one embodiment, one or more is one to three.

“C₁-C₃ alkylene” refers to a straight or branched chain saturated hydrocarbon containing 1-3 carbon atoms, wherein two of the hydrocarbon's hydrogen atoms have been replaced by a single a bond. Representative C₁-C₃ alkylene groups include methylene, ethylene, propylene and isopropylene.

“C₁-C₆ alkylene” refers to a straight or branched chain saturated hydrocarbon containing 1-6 carbon atoms, wherein two of the hydrocarbon's hydrogen atoms have been replaced by a single a bond. Representative C₁-C₆ alkylene groups include, but are not limited to, methylene, ethylene, n-propylene, isopropylene, n-butylene, isobutylene, n-pentylene, isopentylene, and n-hexylene.

“C₁-C₄ alkylene” refers to a straight or branched chain saturated hydrocarbon containing 1-4 carbon atoms, wherein two of the hydrocarbon's hydrogen atoms have been replaced by a single a bond. Representative C₁-C₄ alkylene groups include, but are not limited to, methylene, ethylene, n-propylene, isopropylene, n-butylene, and isobutylene.

“C₂-C₃ alkenylene” refers to a straight or branched chain hydrocarbon containing 2-3 carbon atoms and at least one double bond, wherein two of the hydrocarbon's hydrogen atoms have been replaced by a single bond. Representative C₂-C₃ alkenylene groups include ethenylene, propenylene and ispropropenylene.

“C₂-C₃ alkynylene” refers to a straight or branched chain hydrocarbon containing 2-3 carbon atoms and at least one triple bond, wherein two of the hydrocarbon's hydrogen atoms have been replaced by a single bond. Representative C₂-C₃ alkynylene groups include ethynylene, propynylene and ispropropynylene.

“Halo-substituted C₁-C₄ alkyl” refers to a C₁-C₄ alkyl group, as defined above, wherein one or more of the C₁-C₄ alkyl group's hydrogen atoms has been replaced with —F, —Cl, —Br or —I. In one embodiment, one or more is one to three. Representative examples of a halo-substituted C₁-C₄ alkyl include, but are not limited to, —CH₂F, —CCl₃, —CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂I, —CH₂CH(Br)CH₃, —CH₂CH(Cl)CH₂CH₃, —CH(F)CH₂CH₃ and —C(CH₃)₂(CH₂Cl).

“Cyano-substituted C₁-C₄ alkyl” refers to a C₁-C₄ alkyl group, as defined above, wherein one or more of the C₁-C₄ alkyl group's hydrogen atoms has been replaced with —CN. In one embodiment, one or more is one to three. Representative examples of a cyano-substituted C₁-C₄ alkyl include, but are not limited to, —CH₂CN, —CH₂CH₂CN, —CH(CN)CH₃, —CH₂CH₂CH₂CN, —CH₂CH₂CH(CN)CH₃, —CH₂CH(CN)CH₂CH₃, —CH₂CH(CN)CH₂CH₃, —CH(CN)CH₂CH₃ and —C(CH₃)₂(CH₂CN).

“Amino-substituted C₁-C₄ alkyl” refers to a C₁-C₄ alkyl group, as defined above, wherein one or more of the C₁-C₄ alkyl group's hydrogen atoms has been replaced with —NH₂. In one embodiment, one or more is one to three. Representative examples of a cyano-substituted C₁-C₄ alkyl include, but are not limited to, —CH₂NH₂, —CH₂CH₂NH₂, —CH(NH₂)CH₃, —CH₂CH₂CH₂NH₂, —CH₂CH₂CH(NH₂)CH₃, —CH₂CH(NH₂)CH₂CH₃, —CH₂CH(NH₂)CH₂CH₃, —CH(NH₂)CH₂CH₃ and —C(CH₃)₂(CH₂NH₂).

“Halo-substituted phenyl” refers to a phenyl group, wherein one or more of the phenyl group's hydrogen atoms has been replaced with —F, —Cl, —Br or —I. In one embodiment, one or more is one to three.

“Cyano-substituted phenyl” refers to a phenyl group, wherein one or more of the phenyl group's hydrogen atoms has been replaced with —CN. In one embodiment, one or more is one to three.

“Hydroxy-substituted phenyl” refers to a phenyl group, wherein one or more of the phenyl group's hydrogen atoms has been replaced with —OH. In one embodiment, one or more is one to three.

“C₁-C₆ alkyl” refers to a straight or branched chain saturated hydrocarbon containing 1-6 carbon atoms. Representative C₁-C₆ alkyl groups include, but are not limited to, methyl, ethyl, propyl, isopropyl, butyl, tert-butyl, sec-butyl, isobutyl, pentyl, isopentyl, neopentyl, hexyl, isohexyl and neohexyl. In one embodiment, the C₁-C₆ alkyl group is substituted with one or more of the following groups: -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl. In one embodiment, one or more is one to three.

“C₁-C₉ alkyl” refers to a straight or branched chain non-cyclic hydrocarbon having from 1 to 9 carbon atoms, wherein one of the hydrocarbon's hydrogen atoms has been replaced by a single bond. Representative —C₁-C₉ alkyls include, methyl, ethyl, propyl, butyl, pentyl, hexyl, heptyl, octyl, nonyl, isopropyl, isobutyl, sec-butyl and tert-butyl, isopentyl, neopentyl, isohexyl, isoheptyl, isooctyl and isononyl.

“C₂-C₈ alkenyl” refers to a straight or branched chain non-cyclic hydrocarbon having from 2 to 9 carbon atoms and at least one double bond. Representative C₂-C₈ alkenyls include, ethenyl, propenyl, butenyl, pentenyl, hexenyl, heptenyl, octenyl, nonenyl, isopropenyl, isobutenyl, sec-butenyl and tert-butenyl, isopentenyl, neopentenyl, isohexenyl, isoheptenyl, isooctenyl and isononenyl.

“C₂-C₉ alkynyl” refers to a straight or branched chain non-cyclic hydrocarbon having from 2 to 9 carbon atoms and at least one triple bond. Representative C₂-C₉ alkynyls include, ethynyl, propynyl, butynyl, pentynyl, hexynyl, heptynyl, octynyl, nonynyl, isopropynyl, isobutynyl, sec-butynyl and tert-butynyl, isopentynyl, neopentynyl, isohexynyl, isoheptynyl, isooctynyl and isononynyl.

“C₂-C₆ alkenyl” refers to a straight or branched chain unsaturated hydrocarbon containing 2-6 carbon atoms and at least one double bond. Representative C₂-C₆ alkenyl groups include, but are not limited to, ethylene, propylene, 1-butylene, 2-butylene, isobutylene, sec-butylene, 1-pentene, 2-pentene, isopentene, 1-hexene, 2-hexene, 3-hexene and isohexene. In one embodiment, the C₂-C₆ alkenyl group is substituted with one or more of the following groups: -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl. In one embodiment, one or more is one to three.

“C₂-C₆ alkynyl” refers to a straight or branched chain unsaturated hydrocarbon containing 2-6 carbon atoms and at least one triple bond. Representative C₂-C₆ alkynyl groups include, but are not limited to, acetylene, propyne, 1-butyne, 2-butyne, isobutyne, sec-butyne, 1-pentyne, 2-pentyne, isopentyne, 1-hexyne, 2-hexyne, 3-hexyne and isohexyne. In one embodiment, the C₂-C₆ alkynyl group is substituted with one or more of the following groups: -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl. In one embodiment, one or more is one to three.

A “phenylene” is a substituted or unsubstituted phenyl group, wherein two of the phenyl group's hydrogen atoms have been replaced by a single a bond, e.g., 1,2- or 1,3- or 1,4-phenylene.

“Halo” refers to —F, —Cl, —Br or —I.

A “C₃-C₈ monocyclic cycloalkyl” is a non-aromatic, saturated hydrocarbon ring containing 3-8 carbon atoms. Representative C₃-C₈ monocyclic cycloalkyl groups include, but are not limited to, cyclopropyl, cyclobutyl, cyclopentyl, cyclohexyl, cycloheptyl and cyclooctyl. In one embodiment, the C₃-C₈ monocyclic cycloalkyl group is substituted with one or more of the following groups: -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl. In one embodiment, one or more is one to three.

A “C₃-C₈ monocyclic cycloalkenyl” is a non-aromatic hydrocarbon ring containing 3-8 carbon atoms and having at least one endocyclic double bond. Representative C₃-C₈ monocyclic cycloalkenyl groups include, but are not limited to, cyclopropenyl, cyclobutenyl, 1,3-cyclobutadienyl, cyclopentenyl, 1,3-cyclopentadienyl, cyclohexenyl, 1,3-cyclohexadienyl, cycloheptenyl, 1,3-cycloheptadienyl, 1,4-cycloheptadienyl, -1,3,5-cycloheptatrienyl, cyclooctenyl, 1,3-cyclooctadienyl, 1,4-cyclooctadienyl, -1,3,5-cyclooctatrienyl. In one embodiment, the C₃-C₈ monocyclic cycloalkenyl group is unsubstituted or substituted with one or more of the following groups: -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl. In one embodiment, one or more is one to three.

A “C₇-C₉ monocyclic cycloalkynyl” is a non-aromatic hydrocarbon ring containing 7-9 carbon atoms and having at least one endocyclic triple bond. Representative C₇-C₉ monocyclic cycloalkynyl groups include, but are not limited to, cycloheptynyl, cyclooctynyl, and cyclononynyl. In one embodiment, the C₇-C₉ monocyclic cycloalkynyl group is substituted with one or more of the following groups: -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl. In one embodiment, one or more is one to three.

The term “3- to 7-membered non-aromatic monocyclic heterocycle” refers to a 3-7 membered saturated or partially unsaturated ring having 1 to 3 heteroatoms independently selected from N, O and S, where any carbon ring atom may form a carbonyl or thiocarbonyl group, and where any ring nitrogen or sulfur atom may be oxidised. Included in this class are (1) a 3- or 4-membered non-aromatic monocyclic cycloalkyl in which one of the ring carbon atoms has been replaced with a N, O or S atom; or (2) a 5-, 6-, or 7-membered non-aromatic monocyclic cycloalkyl in which 1 to 3 of the ring carbon atoms has been independently replaced with an N, O or S atom. In one embodiment, the 3- to 7-membered non-aromatic monocyclic heterocycle is a 4- to 7-membered non-aromatic monocyclic heterocycle. In one embodiment, a carbon atom of a 4- to 7-membered non-aromatic monocyclic heterocycle is replaced with a carbonyl group. In one embodiment, a carbon atom of a 3- to 7-membered non-aromatic monocyclic heterocycle is replaced with a carbonyl group. In another embodiment, a carbon atom of a 3- to 7-membered non-aromatic monocyclic heterocycle is replaced with a thiocarbonyl group. A 3- to 7-membered non-aromatic monocyclic heterocycle can be attached via a ring nitrogen or ring carbon atom. Representative examples of a 3- to 7-membered non-aromatic monocyclic heterocycle group include, but are not limited to azepanyl, aziridinyl, 1,3-dioxolanyl, 1,4-dioxolanyl, imidazolidinyl, imidazolidin-2-one-yl, imidazolinyl, 1,4-oxazepanyl, morpholinyl, piperazinyl, N-methylpiperazinyl, piperidinyl, N-methylpiperidinyl, pyranyl, pyrazolidinyl, pyrazolinyl, pyrrolidinonyl, pyrrolidinyl, N-methylpyrrolidinyl, N-benzylpyrrolidinyl, pyrrolinyl, tetrahydrofuranyl, tetrahydrothiophenyl, tetrahydropyranyl, thiomorpholinyl, thiomorpholinyl-5-oxide, thiomorpholinyl-S,S-dioxide and trithianyl.

In one embodiment, the 3- to 7-membered non-aromatic monocyclic heterocycle group is independently substituted on one or more ring nitrogen or ring carbon atoms with one or more of the following groups: —R²⁸, —OR²⁸, —R¹⁴, —NH-benzoyl, wherein R²⁸ and R¹⁴ are as defined above for the compounds of Formula (I), (Ia) and (Ib). In another embodiment, the 3- to 7-membered non-aromatic monocyclic heterocycle group is substituted on a single saturated carbon atom not adjacent to a nitrogen atom with —O(CH₂)₂O— or —(CH₂)₃O—.

The term “nitrogen-containing 3- to 7-membered non-aromatic monocyclic heterocycle” refers to a 3- to 7-membered non-aromatic monocyclic heterocycle, defined above, that contains at least one nitrogen atom.

The term “5- or 6-membered aromatic monocyclic heterocycle” refers to a 5- or 6-membered aromatic monocyclic cycloalkyl in which from 1 to 4 of the ring carbon atoms has been replaced with an N, O or S atom. In one embodiment, the 5- or 6-membered aromatic monocyclic heterocycle is attached via a ring carbon atom. Representative examples of a 5- or 6-membered aromatic monocyclic heterocycle group include, but are not limited to furanyl, imidazolyl, isothiazolyl, isoxazolyl, oxazolyl, oxadiazolyl, pyrimidinyl, pyrazolyl, pyrazinyl, pyridazinyl, pyridinyl, pyrrolyl, thiazolyl, thiadiazolyl, thiophenyl, triazinyl, and triazolyl.

In one embodiment, the 5- or 6-membered aromatic monocyclic heterocycle group is independently substituted on one or more ring nitrogen or ring carbon atoms with one or more of the following groups: —R²⁸, —OR²⁸, —R¹⁴, —NH-benzoyl, wherein R²⁸ and R¹⁴ are as defined above for the compounds of Formula (I), (Ia) and (Ib). In a specific embodiment, the 5- to 6-membered aromatic heterocycle is substituted with 4-(2,3-dihydro-indol-1-yl) or 4-(1,3-dihydro-isoindol-2-yl).

The term “nitrogen-containing 5- or 6-membered aromatic monocyclic heterocycle” refers to a 5- or 6-membered aromatic monocyclic heterocycle, defined above, that contains at least one nitrogen atom.

The term “8- to 12-membered bicyclic heterocycle” refers to a bicyclic 8- to 12-membered aromatic or non-aromatic bicyclic cycloalkyl in which one or both of the of the rings of the bicyclic ring system have 1-4 of its ring carbon atoms independently replaced with a N, O or S atom. Included in this class are 3- to 7-membered monocyclic heterocycles that are fused to a benzene ring. A non-aromatic ring of an 8- to 12-membered bicyclic heterocycle is attached via a ring nitrogen or ring carbon atom. An aromatic 8- to 12-membered bicyclic heterocycle is attached via a ring carbon atom. Examples of 8- to 12-membered bicyclic heterocycles include, but are not limited to, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, cinnolinyl, decahydroquinolinyl, 1H-indazolyl, indolenyl, indolinyl, indolizinyl, indolyl, isobenzofuranyl, isoindazolyl, isoindolyl, isoindolinyl, isoquinolinyl, naphthyridinyl, octahydroisoquinolinyl, phthalazinyl, pteridinyl, purinyl, quinoxalinyl, tetrahydroisoquinolinyl, tetrahydroquinolinyl, xanthenyl, and imidazo[4,5-c]pyridine-1-yl. In one embodiment, each ring of the −8- to 12-membered bicyclic heterocycle group is independently substituted on one or more ring nitrogen or ring carbon atoms with one or more of the following groups: —R²⁸, —OR²⁸, —R¹⁴, —NH-benzoyl, wherein R²⁸ and R¹⁴ are as defined above for the Benzo[c][2,7]naphthyridine Derivatives of Formula (I), (Ia) and (Ib).

In another embodiment, the 8- to 12-membered bicyclic heterocycle group can be substituted on a single saturated carbon atom not adjacent to a nitrogen atom with —O(CH₂)₂O— or —(CH₂)₃O—.

In another embodiment, the 8- to 12-membered bicyclic heterocycle group can be substituted on a single saturated carbon atom not adjacent to a nitrogen atom with —O(CH₂)₂O— or —(CH₂)₃O—.

In one embodiment, a carbon atom of a 8- to 12-membered bicyclic heterocycle group is replaced with a carbonyl group. In another embodiment, a carbon atom of a −8- to 12-membered bicyclic heterocycle group is replaced with a thiocarbonyl group.

Unless indicated, the 8- to 12-membered bicyclic heterocycle group is unsubstituted.

The term “nitrogen-containing 8- to 12-membered bicyclic heterocycle” refers to a 8- to 12-membered bicyclic heterocycle, defined above, that contains at least one nitrogen atom.

The term “aryl or heteroaryl” refers to a phenyl, a benzyl, a 5- to 6-membered aromatic monocyclic heterocycle, an aromatic 8- to 12-membered bicyclic heterocycle, or a

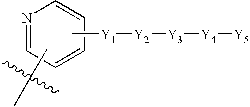

group, each of which is unsubstituted or substituted as provided above, provided the substituted aryl or heteroaryl retains its aromaticity, when Y₁, Y₂, Y₃, Y₄ and Y₅ are as defined for the compounds of Formula (I), (Ia) and (Ib).

The term “open valence” refers to an electron that is able to form a covalent bond with another open valence.

A “subject” is a mammal, e.g., a human, mouse, rat, guinea pig, dog, cat, horse, cow, pig, or non-human primate, such as a monkey, chimpanzee, baboon. In one embodiment, the monkey is a rhesus. In one embodiment, a subject is a human.

The phrase “pharmaceutically acceptable salt,” as used herein, is a salt formed from an acid and a basic nitrogen group of one of the Benzo[c][2,7]naphthyridine Derivatives. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, besylate, mesylate, camphor sulfonate, and pamoate (i.e., 1,1′-methylene-bis-(2-OH-3-naphthoate)) salts.

The term “pharmaceutically acceptable salt” also refers to a salt prepared from a Benzo[c][2,7]naphthyridine Derivative having an acidic functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, tris-(hydroxymethyl)methylamine, or 2-hydroxy-tert-butylamine, or N,N-di-lower alkylene-N-(hydroxy lower alkyl)-amines, such as N,N-dimethyl-N-(2-hydroxyethyl)amine or tri-(2-hydroxyethyl)amine; N-methyl-D-glucamine; and amino acids such as arginine, lysine, and the like, wherein ‘lower’ denotes 1 to 6 carbon atoms. A Benzo[c][2,7]naphthyridine Derivative can exist in a form of a hydrate.

An “effective amount” when used in connection with a Benzo[c][2,7]naphthyridine Derivative is an amount effective for treating or preventing a proliferative disorder.

An “effective amount” when used in connection with another anticancer agent is an amount that is effective for treating or preventing cancer alone or in combination with a Benzo[c][2,7]naphthyridine Derivative.

“In combination with” includes administration within the same composition and within separate compositions. In the latter instance, the anticancer agent is administered during a time when the Benzo[c][2,7]naphthyridine Derivative exerts its prophylactic or therapeutic effect, or vice versa.

A “protecting group for amines” refers to a chemical group that is introduced into a molecule by chemical modification of an amine group in order to obtain chemoselectivity in a subsequent chemical reaction. Examples of protecting groups for amines include, but are not limited to, carbobenzyloxy (Cbz) group, tert-butyloxycarbonyl (BOC) group, 9-fluorenylmethyloxycarbonyl (FMOC) group, benzyl (Bn) group, and p-methoxyphenyl (PMP) group. Further examples of a protecting group for amines can be found in T. W. Green and P. G. M. Wuts, Protective Groups in Organic Synthesis, Wiley-Interscience, New York, 1999, incorporated herein by reference in its entirety.

A “silyl protecting group” refers to a chemical group that is introduced into a molecule by chemical modification of a hydroxy group in order to obtain chemoselectivity in a subsequent chemical reaction. Examples of silyl protecting groups include, but are not limited to, silyl ethers such as trimethylsilyl (TMS), tert-butyldimethylsilyl (TBDMS), and triisopropylsilyl (TIPS). One of skill in the art will understand that other hydroxyl protecting groups can be used in place of a silyl protecting group. Examples of other hydroxyl protecting groups include, but are not limited to, acetyl (Ac), β-methoxyethoxymethyl ether (MEM), methoxymethyl ether (MOM), p-methoxybenzyl ether (PMB), methylthiomethyl ether, pivaloyl (Piv), and tetrahydropyran (THP). Further examples of silyl protecting groups and other hydroxyl protecting groups can be found in T. W. Green and P. G. M. Wuts, Protective Groups in Organic Synthesis, Wiley-Interscience, New York, 1999, incorporated herein by reference in its entirety.

Some chemical structures herein are depicted using bold and dashed lines to represent chemical bonds. These bold and dashed lines depict absolute stereochemistry. Unless otherwise indicated, a chiral center without any indication of stereochemistry represents (R) isomer, (S) isomer, or any mixture of the two.

Illustrative examples of the Benzo[c][2,7]naphthyridine Derivative can be described herein using both chemical structures and chemical names. It is to be understood that if a discrepancy exists between a chemical structure and its corresponding chemical name, the chemical structure predominates.

It is provided that open valences must occur in pairs that are connected.

It is provided that when connecting open valences that reside on unsaturated carbon atoms, rings cannot be created that have trans double bonds.

It is provided that when connecting open valences to create a 5- to 7-membered ring, no N—C′—N, N—C′—O, or N—C′—S atom grouping are formed where C′ represents a saturated carbon atom.

The compounds of this invention may contain one or more asymmetric carbon atoms. In such cases, the compounds of this invention include the individual diasteromers, the racemates, and the individual (R) and (S) entantiomers thereof. Some of the compounds of this invention may contain one or more double bonds. In such cases, the compounds of this invention include each of the possible configurational isomers as well as mixtures of these isomers. Some of the compounds of this invention may exist as separate tautomers. In such cases, the compounds of this invention include each tautomer and mixtures of these tautomers.

When a compound of this invention has a moiety that contains a heterocyclic ring, either mono, bicyclic, or tricyclic, such heterocyclic ring does not contain O—O, S—S, or S—O bonds in the ring.

The following abbreviations are used herein and have the indicated definitions: BSA is bovine serum albumin, DMF is N,N-dimethylformamide, DCM is dichloromethane, DME is dimethoxyethane, DMAP is 4-(dimethylamino)pyridine, DTT is dithiothreitol, FRET is fluorescence resonance energy transfer, ATP is adenosine triphosphate, THF is tetrahydrofuran, DMSO is dimethylsulfoxide, Ac is acyl, Et is ethyl, EtOAc is ethyl acetate, EtOH is ethanol, Me is methyl, MtOH is methanol, MS is mass spectrometry, Ph is phenyl, Ar is aryl, NOE is Nuclear Overhauser Effect, NMR is Nuclear Magnetic Resonance, HPLC is High Performance Liquid Chromatograpy, Ms is mesylate, Ac is acetyl, LiHDMS is lithium hexamethyldisilazane, TEA is triethylamine, TFA is trifluoroacetic acid, Rf is retention factor, HRMS is High Resolution Mass Spectrometry, mCPBA is m-chloroperbenzoic acid, NMP is N-methylpyrrolidinone, DIBAL is diisobutylaluminium hydride, TBDS-Cl is tert-butyldimethylsilyl chloride, TBAF is tetra n-butylammonium fluoride, DEAD is diethyl azodicarboxylate, and Boc (or BOC) is t-butyloxycarbonyl.

### 5.2 The Benzo[c][2,7]naphthyridine Derivatives of Formula (I)

The invention provides Benzo[c][2,7]naphthyridine Derivatives according to Formula (I):

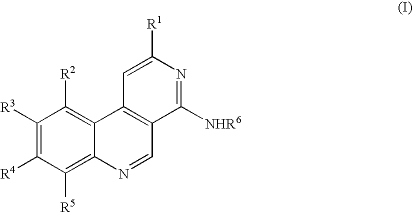

and pharmaceutically acceptable salts thereof,
wherein

R¹, R², R³, R⁴, R⁵ and R⁶, are as defined above for the compounds of Formula (I).

In one embodiment, R² is —H.

In another embodiment, R⁵ is —H.

In one embodiment, R² and R⁵ are —H.

In another embodiment, R⁶ is —H.

In yet another embodiment, R³ and R⁴ are —O—CH₃.

In a further embodiment, R⁴ is C₁-C₆ alkylene-O—C₁-C₆ alkyl.

In one embodiment, R⁴ is —Y—(C(R⁵)₂)ₖ—W′—C(R⁸)₂)_{q}—R⁷.

In another embodiment, R⁴ is —Y—(C(R⁸)₂)ₖ—W′—C(R⁸)₂)_{q}—R⁷ and Y is —O—.

In yet another embodiment, R⁴ is —Y—(C(R⁸)₂)ₖ—W′—C(R⁸)₂)_{q}—R⁷ and Y is —CC—.

In a further embodiment, R⁴ is —Y—(C(R⁸)₂)_{g}—R⁹.

In one embodiment, R⁴ is —Y—(C(R⁸)₂)_{g}—R⁹ and Y is —O—.

In another embodiment, R⁴ is —Y—(C(R⁸)₂)_{g}—R⁹ and g=1.

In yet another embodiment, Y₁ is —O— and R¹⁵ is —NH₂.

In a further embodiment, Y₁ is —O— and Y₅ is —N(CH₃)—CH₂-pyridyl.

In one embodiment, Y₁ is —O— and Y₅ is —OS(O)₂—C₁-C₆ alkyl.

In another embodiment, Y₁ is —O— and Y₅ is —C₁-C₆ alkyl.

In yet another embodiment Y₁ is —O— and Y₅ is phenyl or halo-substituted phenyl.

In a further embodiment, Y₁ is —O— and Y₅ is absent.

In one embodiment, R¹ is pyridyl-O-pyrrolidinyl.

In another embodiment, R¹ is imidazolyl.

In yet another embodiment, R¹ is

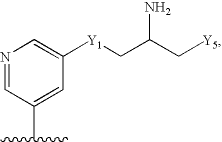

wherein Y₁ and Y₅ are as defined for the compounds of Formula (I).

In a further embodiment, R¹ is

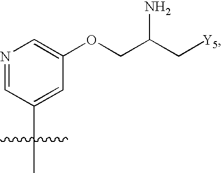

wherein Y₅ is as defined for the Benzo[c][2,7]naphthyridine Derivatives of Formula (I).

In a still further embodiment, R¹ is

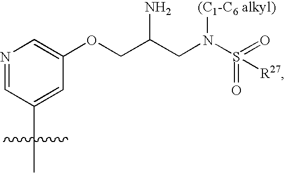

wherein C₁-C₆ alkyl and R²⁷ are as defined for the Benzo[c][2,7]naphthyridine Derivatives of Formula (I).

In various embodiments, R¹ is

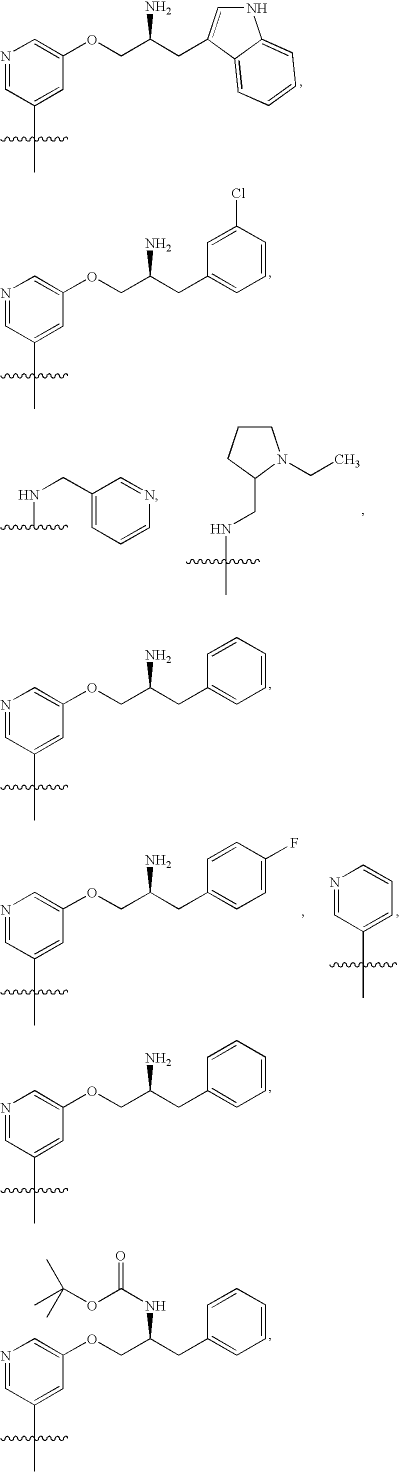

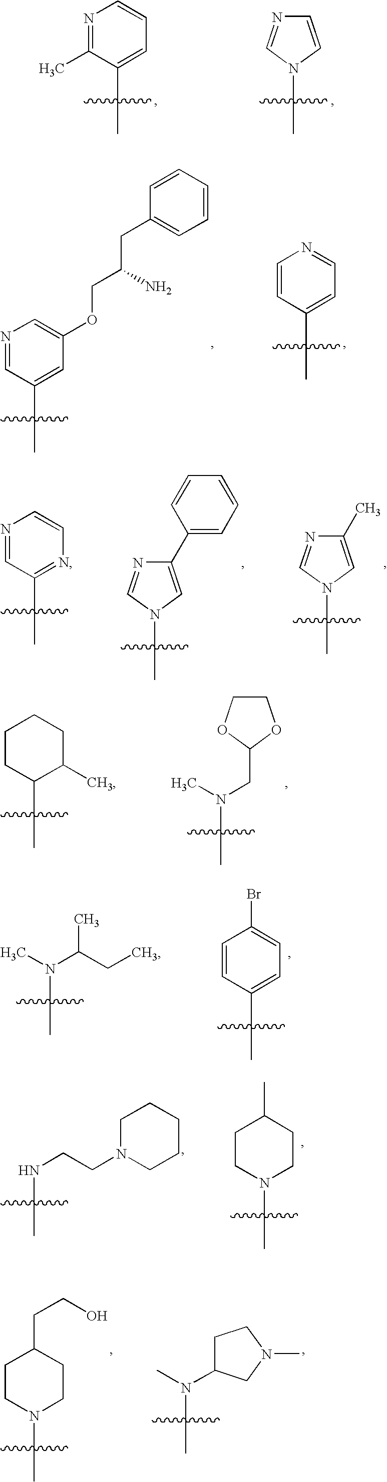

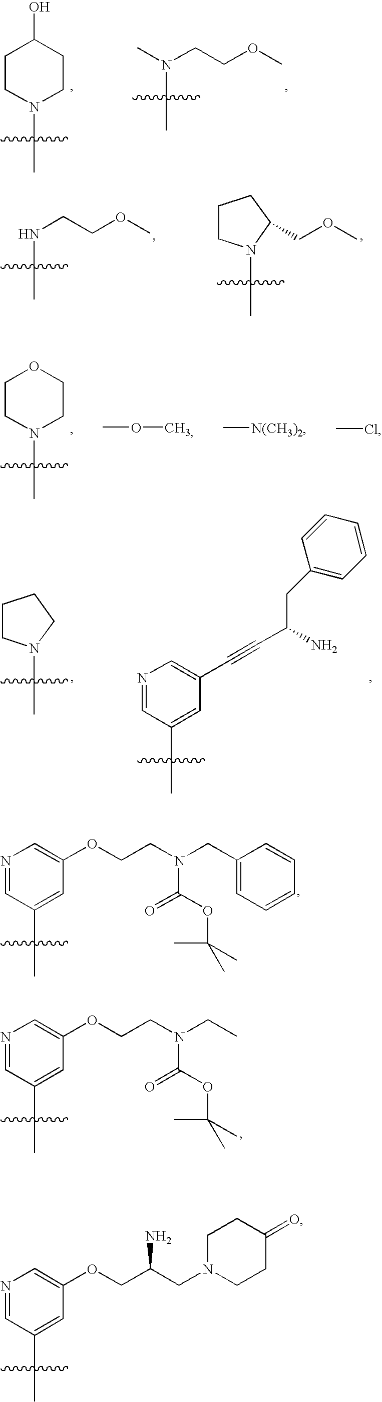

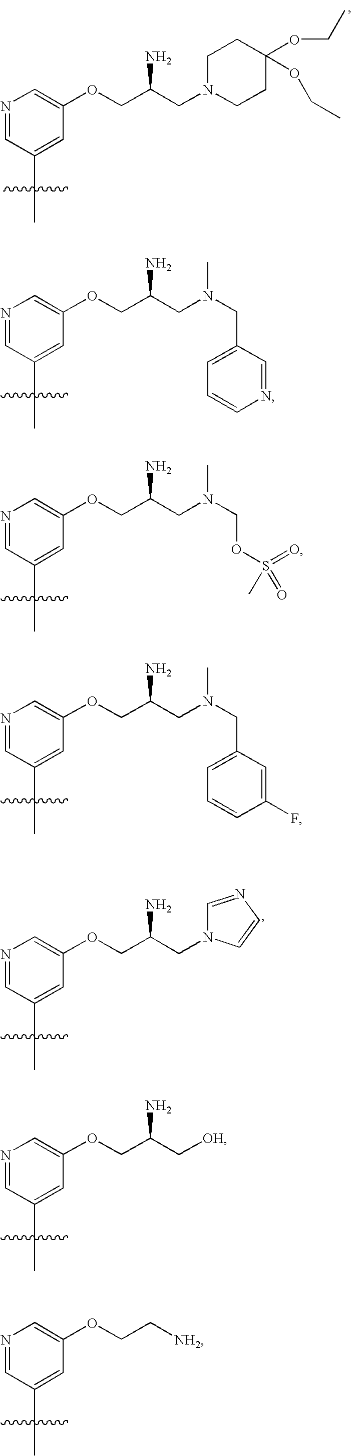

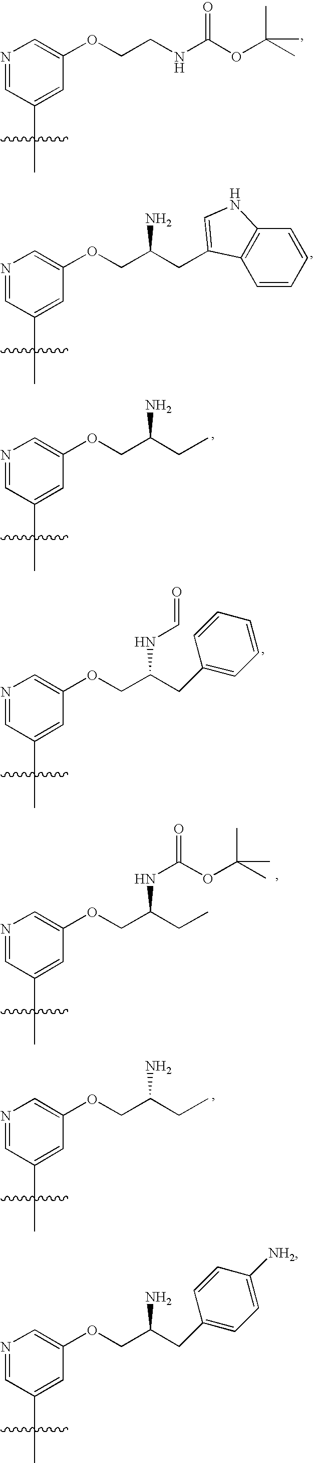

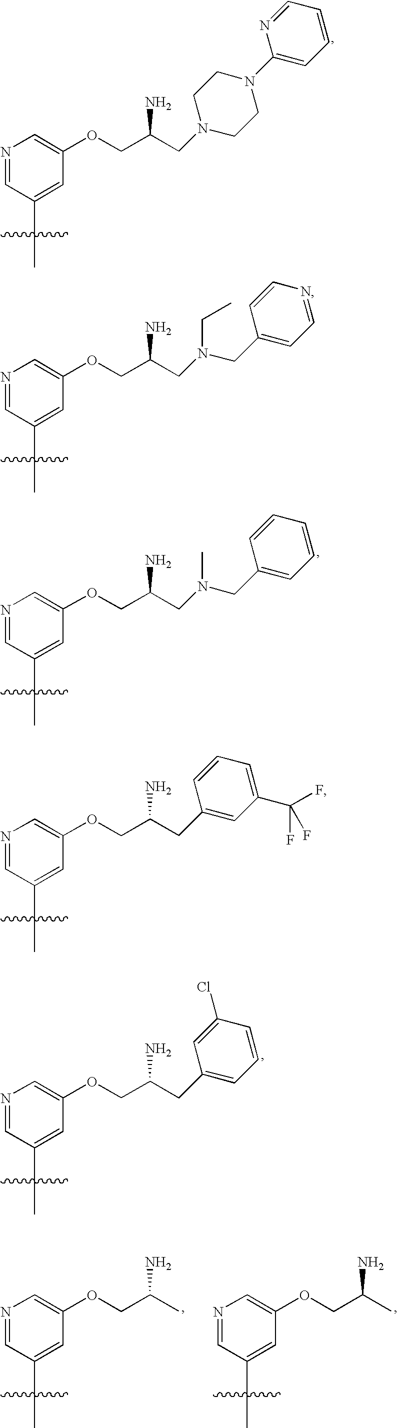

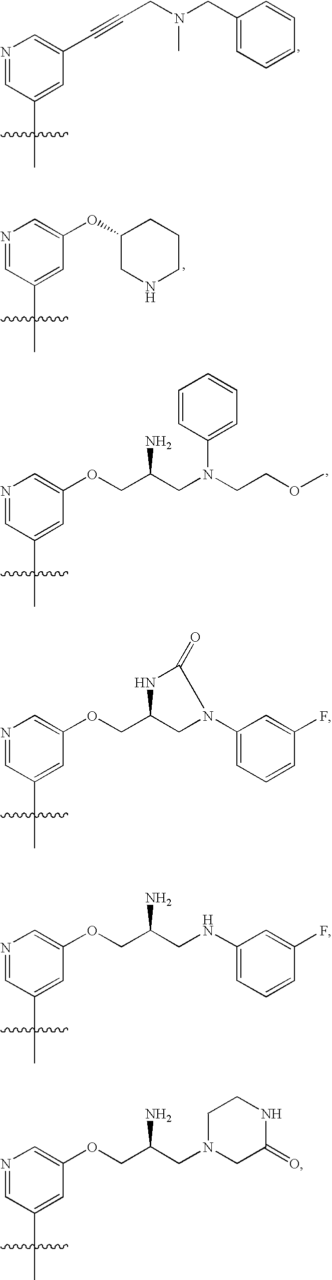

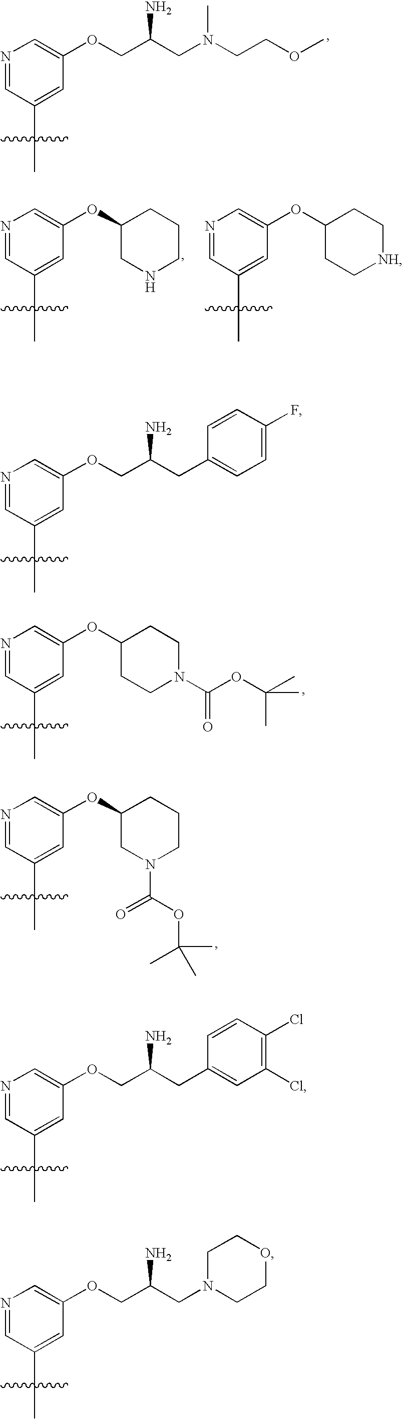

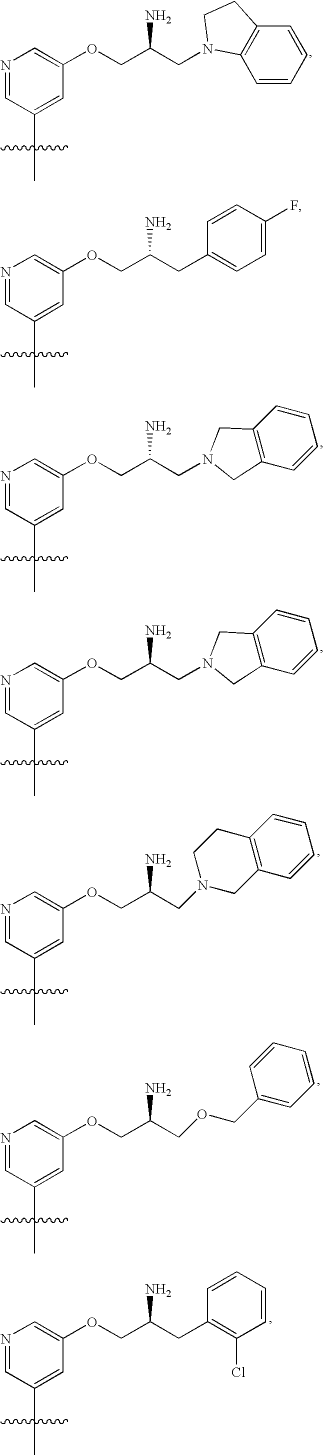

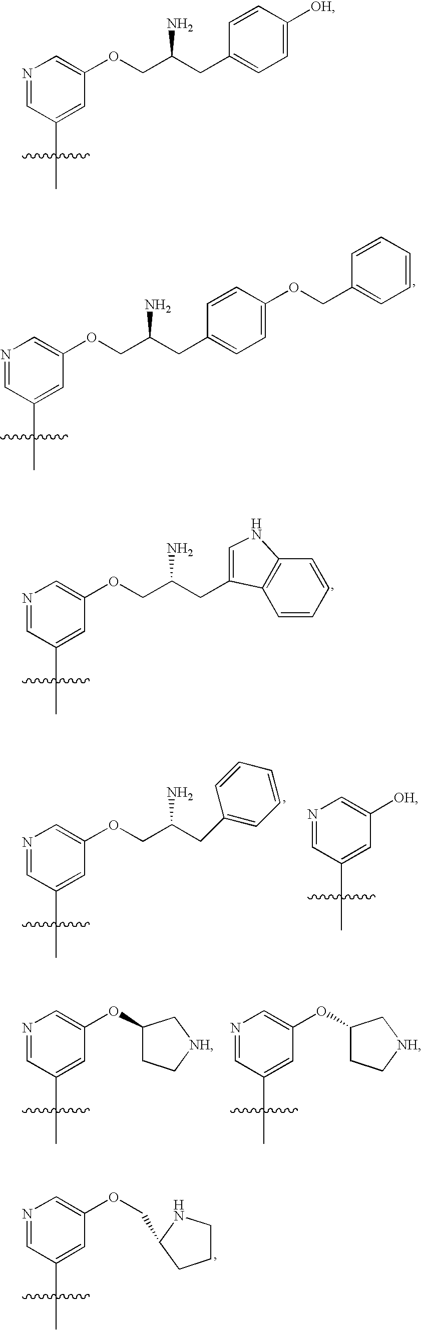

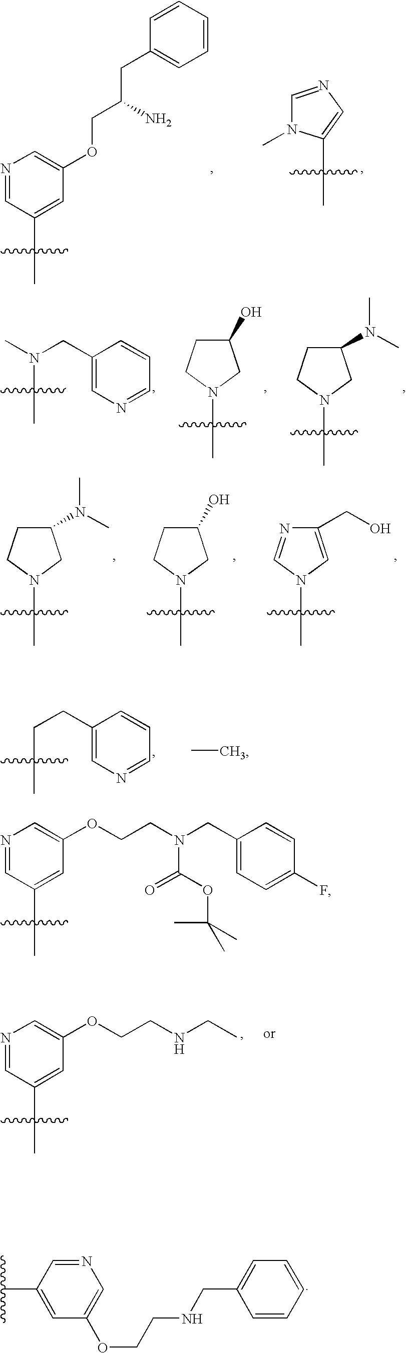

Illustrative examples of the Benzo[c][2,7]naphthyridine Derivatives include the compounds of Formula (I) as set forth below:

| Com- | | | | | | |
|---|---|---|---|---|---|---|
| pound | R¹ | R² | R³ | R⁴ | R⁵ | R⁶ |
| I-1 | 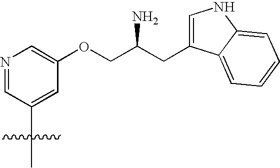 | —H | —O—CH₃ | —OCH₃ | —H | —OH |
| I-2 | 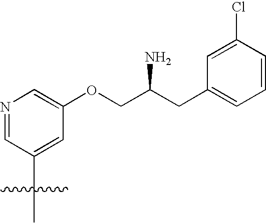 | —H | —O—CH₃ | —O—CH₃ | —H | —OH |
| I-3 | 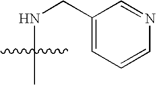 | —H | —O—CH₃ | —O—(CH₂)₂—O—CH₃ | —H | 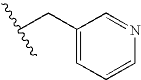 |
| I-4 | 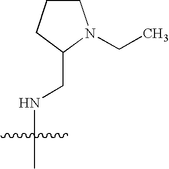 | —H | —O—CH₃ | —O—(CH₂)₂—O—CH₃ | —H | 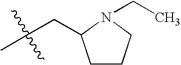 |
| I-5 | 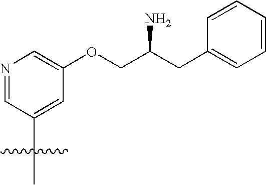 | —H | —O—CH₃ | —O—CH₃ | —H | —CH₃ |
| I-6 | —H | —H | —Br | —H | —H | 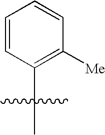 |
| I-7 | —H | —H | —Br | —H | —H | 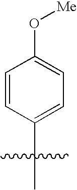 |
| I-8 | —H | —H | —H | —H | —H | 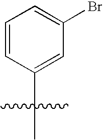 |
| I-9 | 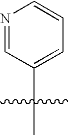 | —O—CH₃ | —H | —H | —H | —H |

and a pharmaceutically acceptable salt thereof

### 5.3 The Benzo[c][2,7]naphthyridine Derivatives of Formula (Ia)

In one embodiment, the compounds of Formula (I) are compounds of Formula (Ia):

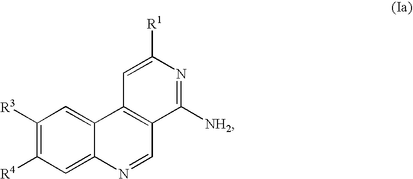

wherein R¹, R³ and R⁴ are defined as above for the compounds of Formula (I).

In one embodiment, for the compounds of Formula (Ia), R⁴ is C₁-C₆ alkylene-O—C₁-C₆ alkyl.

In another embodiment, for the compounds of Formula (Ia),

R⁴ is —Y—(C(R⁸)₂)ₖ—W′—C(R⁸)₂)_{q}—R⁷.

In yet another embodiment, for the compounds of Formula (Ia),

R⁴ is —Y—(C(R⁸)₂)ₖ—W′—C(R⁸)₂)_{q}—R⁷ and Y is —O—.

In a further embodiment, for the compounds of Formula (Ia),

R⁴ is —Y—(C(R⁸)₂)ₖ—W′—C(R⁸)₂)_{q}—R⁷ and Y is —CC—.

In one embodiment, for the compounds of Formula (Ia), R⁴ is —Y—(C(R⁸)₂)_{g}—R⁹.

In another embodiment, for the compounds of Formula (Ia), R⁴ is —Y—(C(R⁸)₂)_{g}—R⁹ and Y is —O—.

In yet another embodiment, for the compounds of Formula (Ia), R⁴ is —Y—(C(R⁸)₂)_{g}—R⁹ and g=1.

In a further embodiment, for the compounds of Formula (Ia), Y₁ is —O— and R¹⁵ is —NH₂.

In one embodiment, for the compounds of Formula (Ia), Y₁ is —O— and Y₅ is —N(CH₃)—CH₂-pyridyl.

In another embodiment, for the compounds of Formula (Ia), Y₁ is —O— and Y₅ is —OS(O)₂—C₁-C₆ alkyl.

In yet another embodiment, for the compounds of Formula (Ia), Y₁ is —O— and Y₅ is —C₁-C₆ alkyl.

In a further another embodiment, for the compounds of Formula (Ia), Y₁ is —O— and Y₅ is phenyl or halo-substituted phenyl.

In one embodiment, for the compounds of Formula (Ia), Y₁ is —O— and Y₅ is absent.

In one embodiment, for the compounds of Formula (Ia), R¹ is pyridyl-O-pyrrolidinyl.

In another embodiment, for the compounds of Formula (Ia), R¹ is imidazolyl.

In yet another embodiment, for the compounds of Formula (Ia), R² and R⁵ are —H and

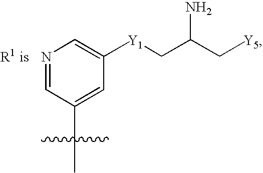

wherein Y₁ and Y₅ are as defined for the compounds of Formula (Ia).

In a further embodiment, for the compounds of Formula (Ia), R¹ is

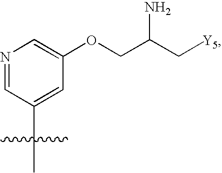

wherein Y₅ is as defined for the Benzo[c][2,7]naphthyridine Derivatives of Formula (Ia).

In a still further embodiment, for the compounds of Formula (Ia), R¹ is

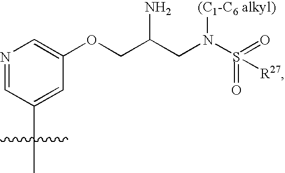

wherein C₁-C₆ alkyl and R²⁷ are as defined for the Benzo[c][2,7]naphthyridine Derivatives of Formula (Ia).

In various embodiments for the compounds of Formula (Ia), R¹ is

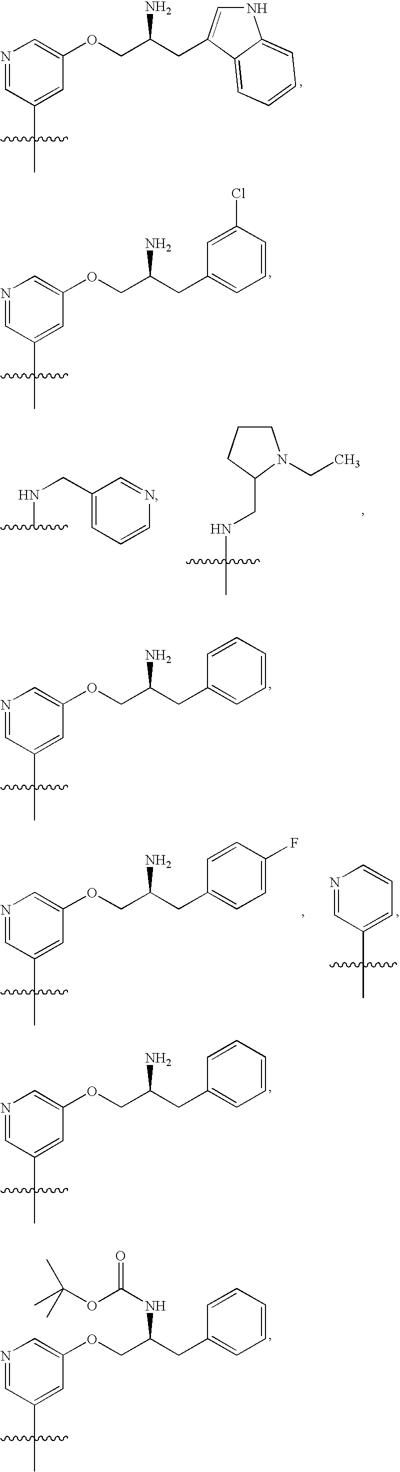

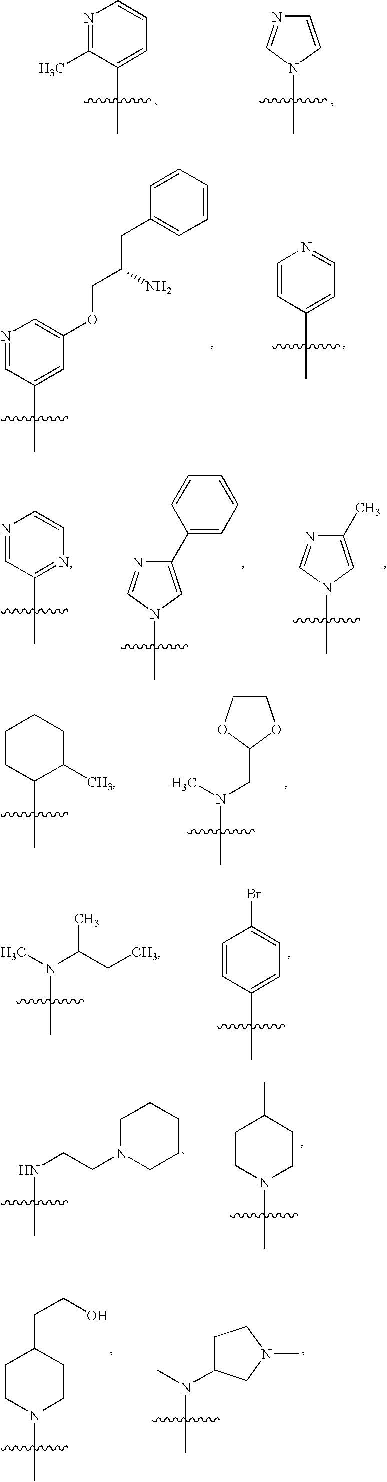

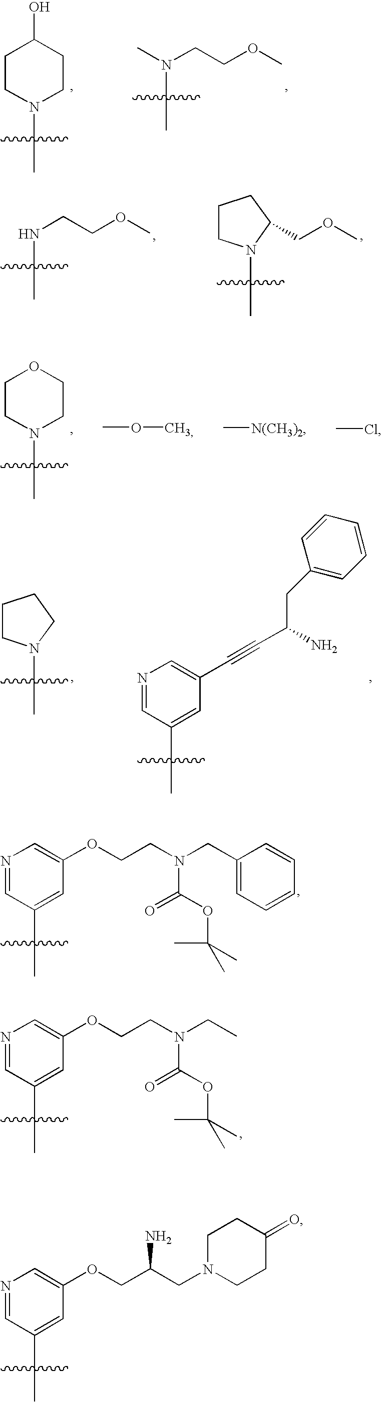

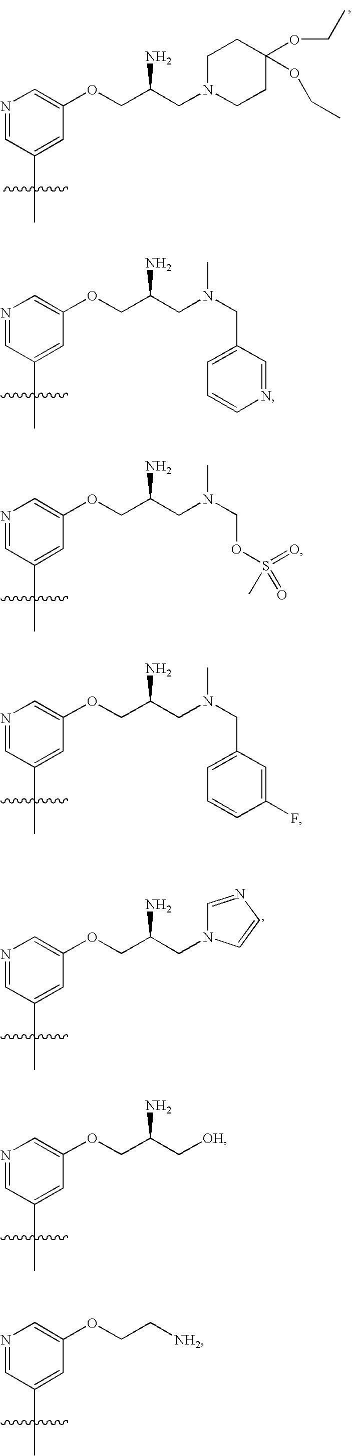

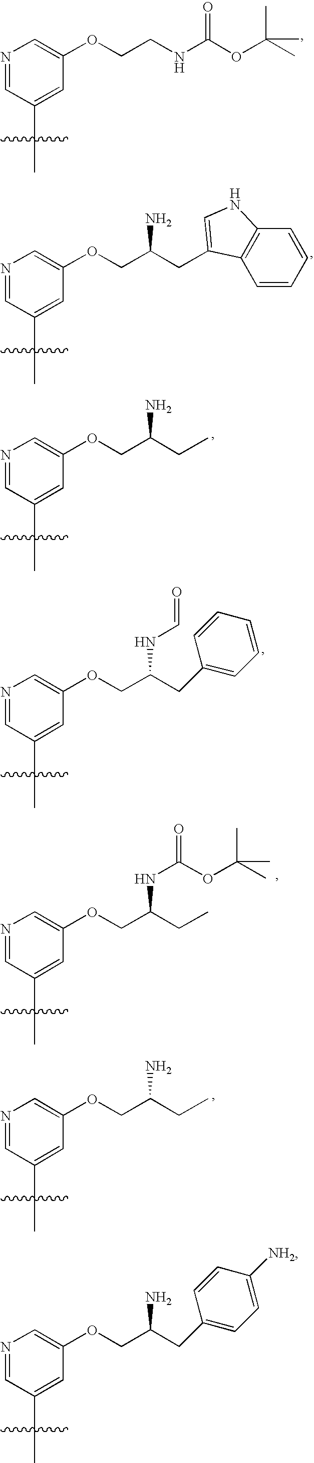

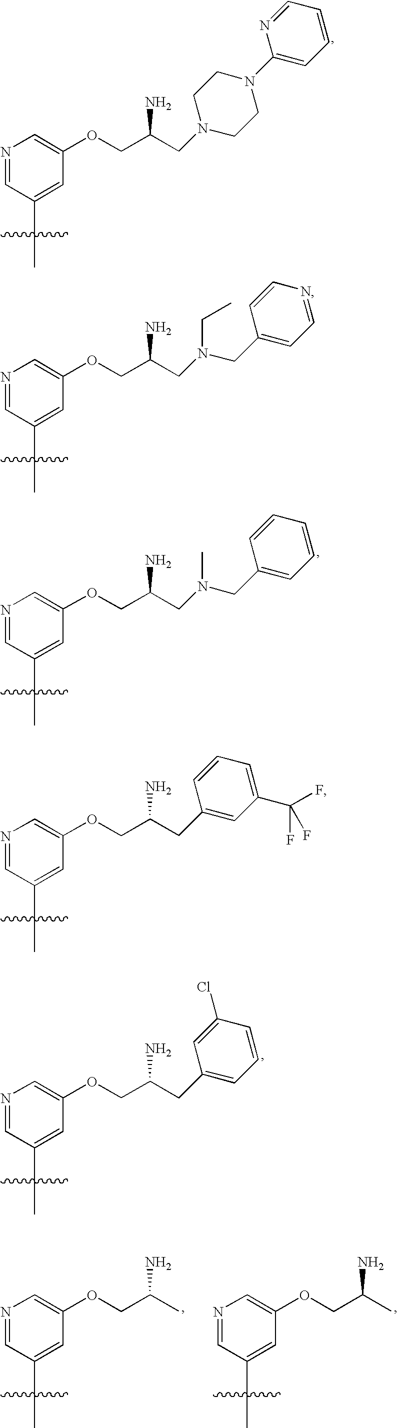

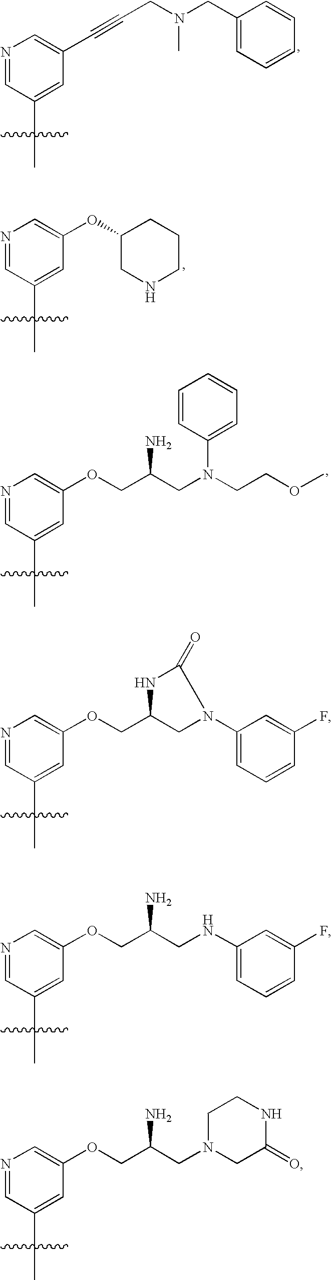

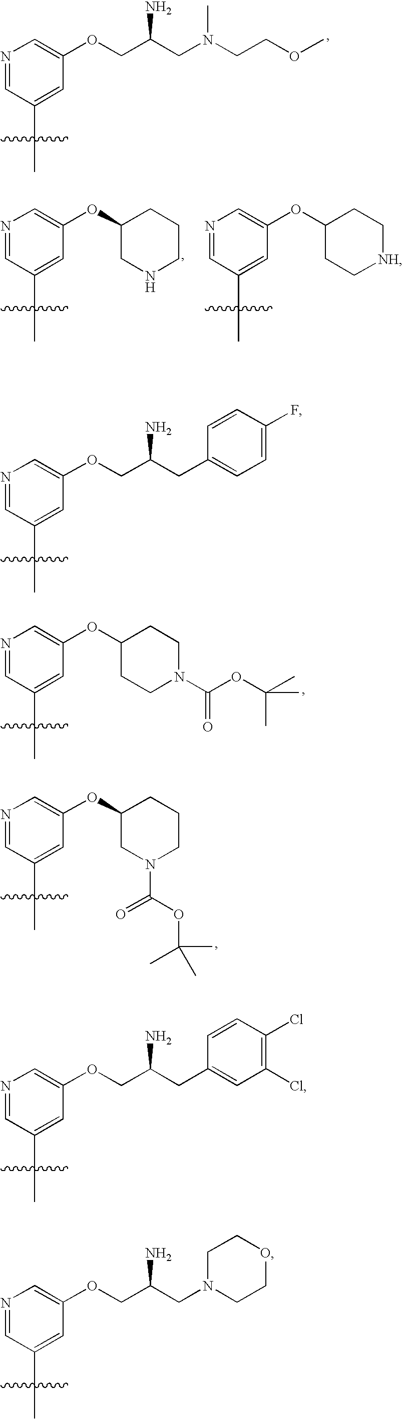

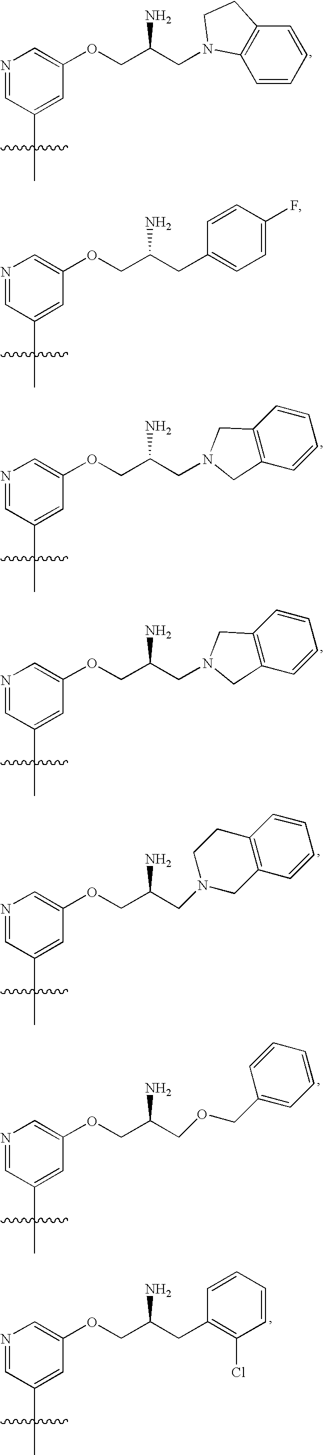

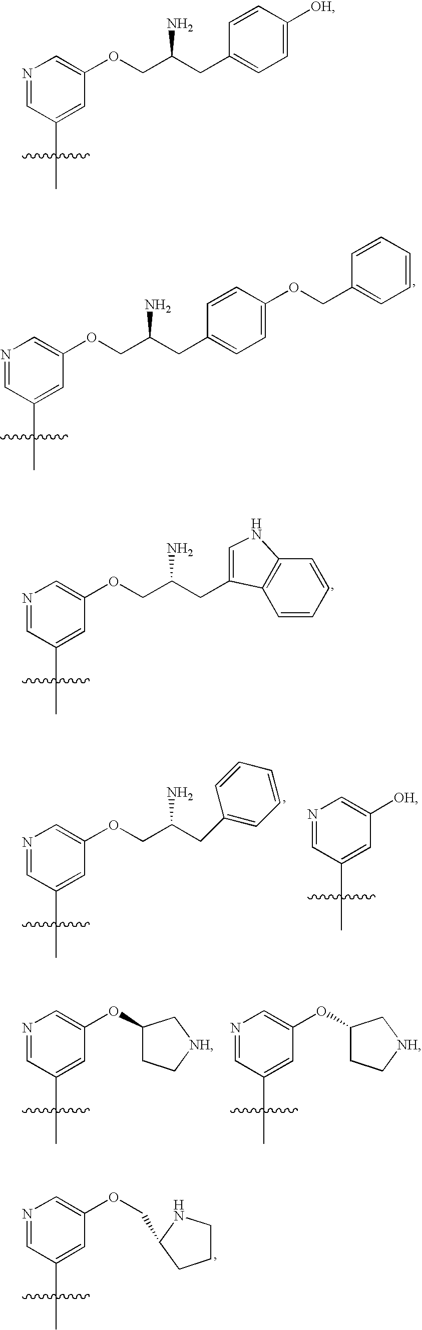

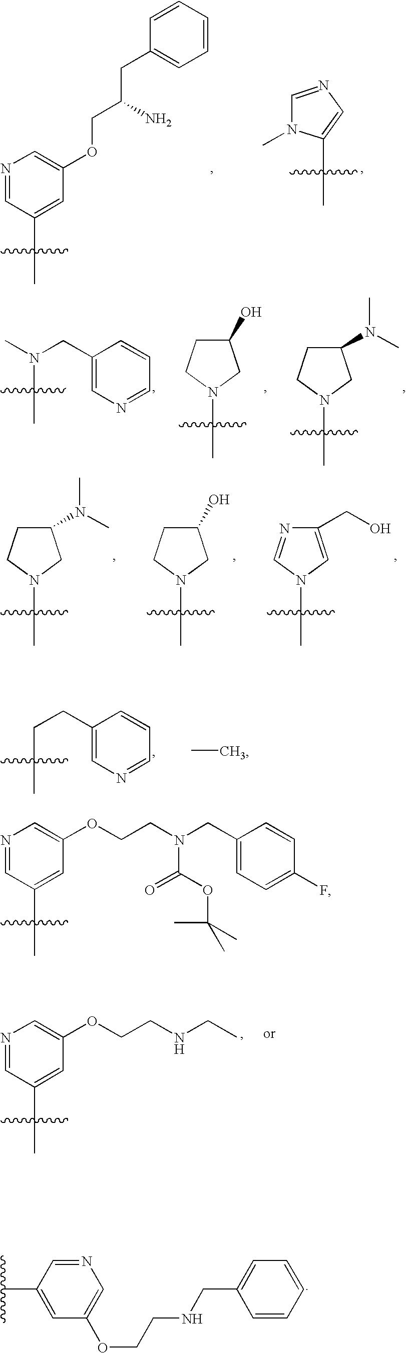

Illustrative examples of the Benzo[c][2,7]naphthyridine Derivatives include the compounds of Formula (Ia) as set forth below:

| Compound | R¹ | R³ | R⁴ |
|---|---|---|---|
| Ia-1 | 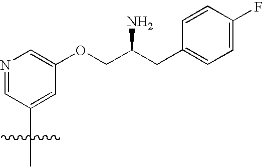 | 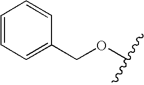 | —OCH₃ |
| Ia-2 | 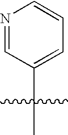 | —O—CH₃ | —O—(CH₂)₂—O—CH₃ |
| Ia-3 | 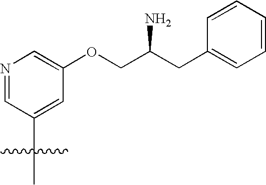 | —H | —H |
| Ia-4 | 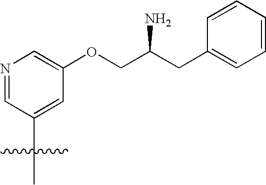 | —H | 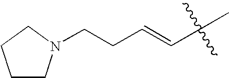 |
| Ia-5 | 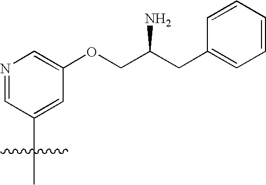 | —H | 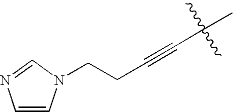 |
| Ia-6 | 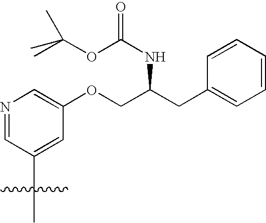 | —H | 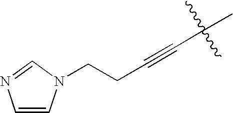 |
| Ia-7 | 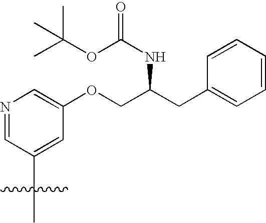 | —H | —Br |
| Ia-8 | 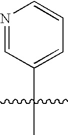 | —Br | —H |
| Ia-9 | 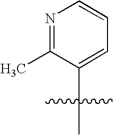 | —OCH₃ | 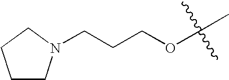 |
| Ia-10 | 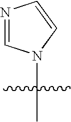 | —OCH₃ | 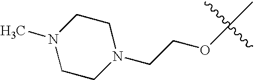 |
| Ia-11 | 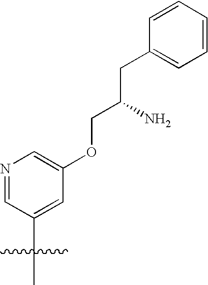 | —OCH₃ | —O—(CH₂)₂—O—CH₃ |
| Ia-12 | 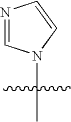 | —OCH₃ | 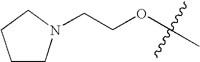 |
| Ia-13 | 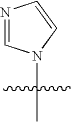 | —OCH₃ | 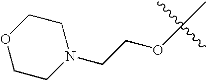 |
| Ia-14 | 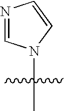 | —OCH₃ | 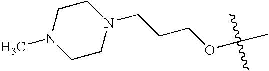 |
| Ia-15 | 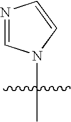 | —OCH₃ | 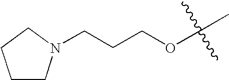 |
| Ia-16 | 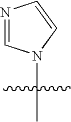 | —OCH₃ | 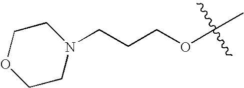 |
| Ia-17 | 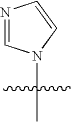 | —OCH₃ | —O—(CH₂)₃—Cl |
| Ia-18 | 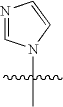 | 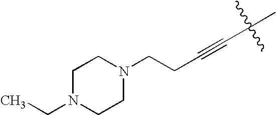 | —H |
| Ia-19 | 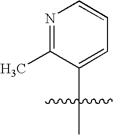 | —OCH₃ | 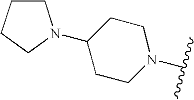 |
| Ia-20 | 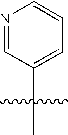 | —OCH₃ | 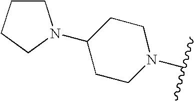 |
| Ia-21 | 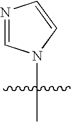 | —OCH₃ | —O—(CH₂)₂—Cl |
| Ia-22 | 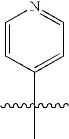 | —OCH₃ | 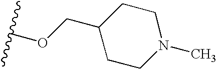 |
| Ia-23 | 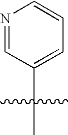 | —OCH₃ | 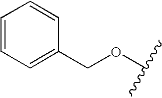 |
| Ia-24 | 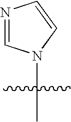 | —H | 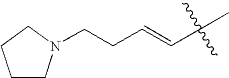 |
| Ia-25 | 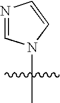 | —H | 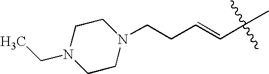 |
| Ia-26 | 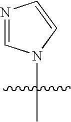 | 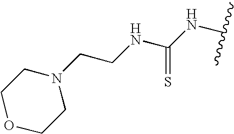 | —O—CH₂—CH₃ |
| Ia-27 | 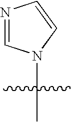 | 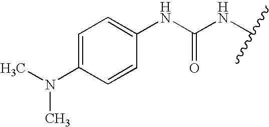 | —O—CH₂—CH₃ |
| Ia-28 | 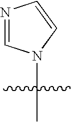 | —NH₂ | —O—CH₂—CH₃ |
| Ia-29 | 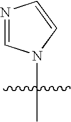 | —NH—C(O)—CH₃ | —O—CH₂—CH₃ |
| Ia-30 | 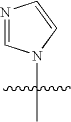 | —H | 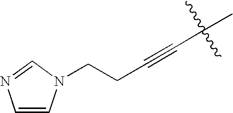 |
| Ia-31 | 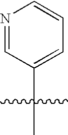 | —O—CH₃ | 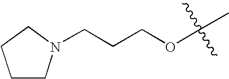 |
| Ia-32 | 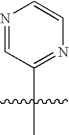 | —O—CH₃ | 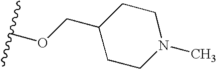 |
| Ia-33 | 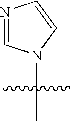 | —O—CH₃ | —OH |
| Ia-34 | 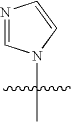 | —O—CH₃ | 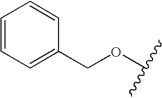 |
| Ia-35 | 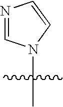 | —H | 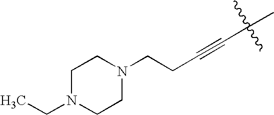 |
| Ia-36 | 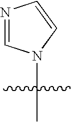 | —H | 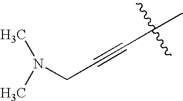 |
| Ia-37 | 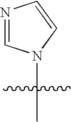 | —H | —Br |
| Ia-38 | 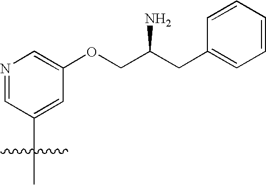 | —O—CH₃ | 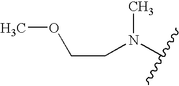 |
| Ia-39 | 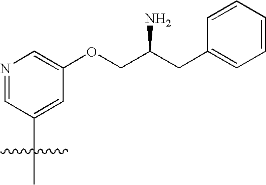 | —O—CH₃ | 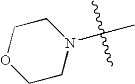 |
| Ia-40 | 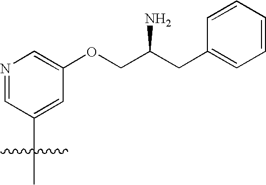 | —O—CH₃ | —S—CH₂—CH₃ |
| Ia-41 | 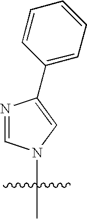 | —O—CH₃ | —O—(CH₂)₂—O—CH₃ |
| Ia-42 | 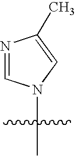 | —O—CH₃ | —O—(CH₂)₂—O—CH₃ |
| Ia-43 | 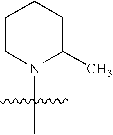 | —O—CH₃ | —O—(CH₂)₂—O—CH₃ |
| Ia-44 | 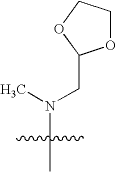 | —O—CH₃ | —O—(CH₂)₂—O—CH₃ |
| Ia-45 | 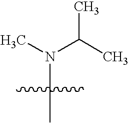 | —O—CH₃ | —O—(CH₂)₂—O—CH₃ |
| Ia-46 | 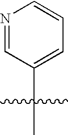 | —O—CH₂—CH₃ | —O—CH₂—CH₃ |
| Ia-47 | 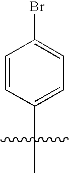 | —O—CH₂—CH₃ | —O—CH₂—CH₃ |
| Ia-48 | 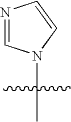 | —O—CH₃ | —O—(CH₂)₂—O—CH₃ |
| Ia-49 | 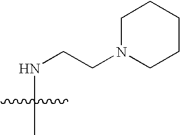 | —O—CH₃ | —O—(CH₂)₂—O—CH₃ |
| Ia-50 | 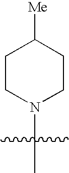 | —O—CH₃ | —O—(CH₂)₂—O—CH₃ |
| Ia-51 | 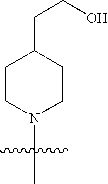 | —O—CH₃ | —O—(CH₂)₂—O—CH₃ |
| Ia-52 | 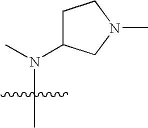 | —O—CH₃ | —O—(CH₂)₂—O—CH₃ |
| Ia-53 | 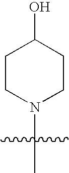 | —O—CH₃ | —O—(CH₂)₂—O—CH₃ |
| Ia-54 | 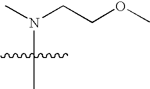 | —O—CH₃ | —O—(CH₂)₂—O—CH₃ |
| Ia-55 | 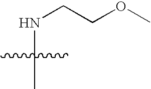 | —O—CH₃ | —O—(CH₂)₂—O—CH₃ |
| Ia-56 | 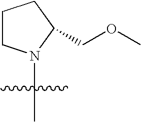 | —O—CH₃ | —O—(CH₂)₂—O—CH₃ |
| Ia-57 | 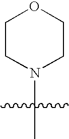 | —O—CH₃ | —O—(CH₂)₂—O—CH₃ |
| Ia-58 | —O—CH₃ | —O—CH₃ | —O—(CH₂)₂—O—CH₃ |
| Ia-60 | —N(CH₃)₂ | —O—CH₃ | —O—(CH₂)₂—O—CH₃ |
| Ia-61 | —Cl | —O—CH₃ | —O—(CH₂)₂—O—CH₃ |
| Ia-62 | 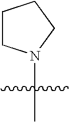 | —O—CH₃ | —O—(CH₂)₂—O—CH₃ |
| Ia-63 | 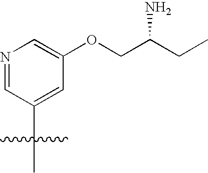 | —O—CH₃ | —O—(CH₂)₂—Cl |
| Ia-64 | 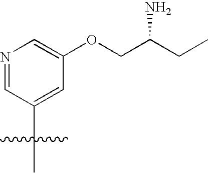 | —O—CH₃ | —O—CH₂—CH₃ |
| Ia-65 | 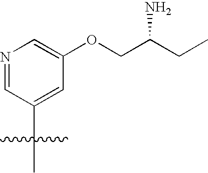 | —O—CH₃ | —O—(CH₂)₂—F |
| Ia-66 | 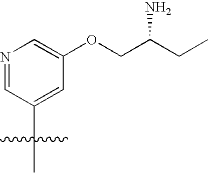 | —O—CH₃ | —O—(CH₂)₃—CF₃ |
| Ia-67 | 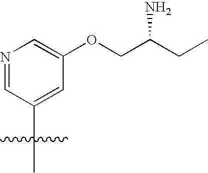 | —O—CH₃ | —O—(CH₂)₃—Cl |
| Ia-68 | 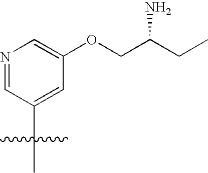 | —O—CH₃ | —O—CH₂—COOH |
| Ia-69a | 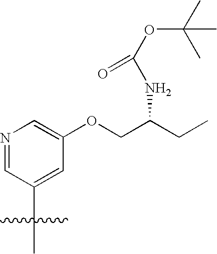 | —OH | —O—CH₃ |
| Ia-69b | 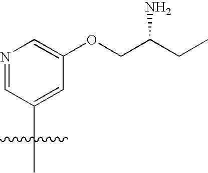 | —O—(CH₂)₂—Cl | —O—CH₃ |
| Ia-70 | 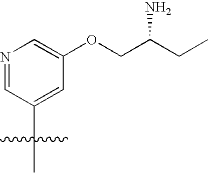 | —O—(CH₂)₂—F | —O—CH₃ |
| Ia-71a | 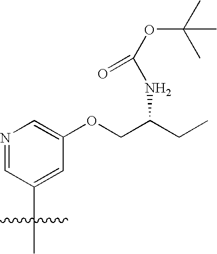 | —OH | —OH |
| Ia-71b | 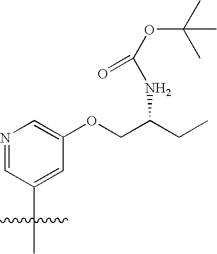 | —Br | —H |
| Ia-73 | 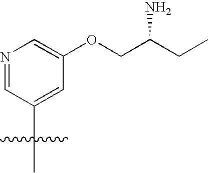 | —OH | —O—CH₃ |
| Ia-74 | 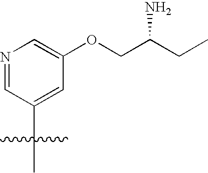 | —H | —O—CH₃ |
| Ia-75 | 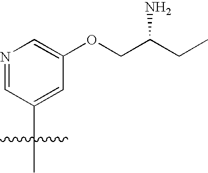 | —Br | —H |
| Ia-76 | 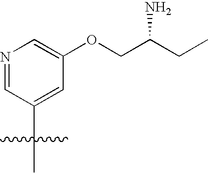 | 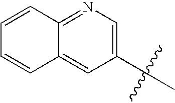 | —H |
| Ia-77 | 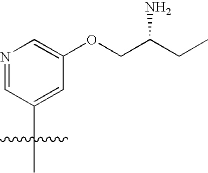 | 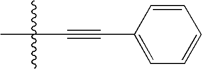 | —H |
| Ia-78 | 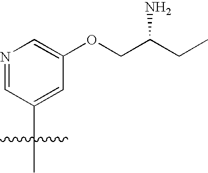 | 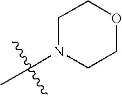 | —H |
| Ia-79 | —H | —Br | —H |
| Ia-80 | 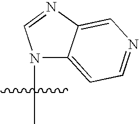 | —OH | —OH |
| Ia-81 | 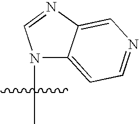 | —OH | —OMe |
| Ia-82 | 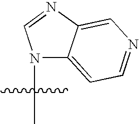 | —OMe | —OH |

and a pharmaceutically acceptable salt thereof

### 5.4 The Benzo[c][2,7]naphthyridine Derivatives of Formula (Ib)

In one embodiment, the compounds of Formula (Ia) are compounds of Formula (Ib):

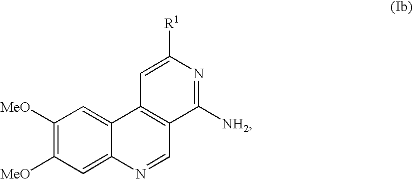

wherein R¹ is defined as above for the compounds of Formula (Ia).

In one embodiment, for the compounds of Formula (Ib), Y₁ is —O— and R¹⁵ is —NH₂.

In another embodiment, for the compounds of Formula (Ib), Y₁ is —O— and Y₅ is —N(CH₃)—CH₂-pyridyl.

In yet another embodiment, for the compounds of Formula (Ib), Y₁ is —O— and Y₅ is —OS(O)₂—C₁-C₆ alkyl.

In a further embodiment, for the compounds of Formula (Ib), Y₁ is —O— and Y₅ is —C₁-C₆ alkyl.

In one embodiment, for the compounds of Formula (Ib), Y₁ is —O— and Y₅ is phenyl or halo-substituted phenyl.

In another embodiment, for the compounds of Formula (Ib), Y₁ is —O— and Y₅ is absent.

In yet another embodiment, for the compounds of Formula (Ib), R¹ is pyridyl-O-pyrrolidinyl.

In a further embodiment, for the compounds of Formula (Ib), R¹ is imidazolyl.

In one embodiment, for the compounds of Formula (Ib), R¹ is

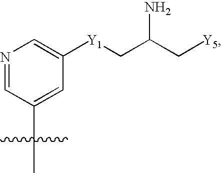

wherein Y₁ and Y₅ are as defined for the compounds of Formula (Ib).

In another embodiment, for the compounds of Formula (Ib), R¹ is

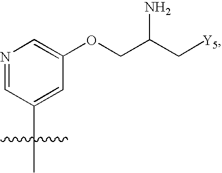

wherein Y₅ is as defined for the Benzo[c][2,7]naphthyridine Derivatives of Formula (Ib).

In a still further embodiment, for the compounds of Formula (Ib), R¹ is

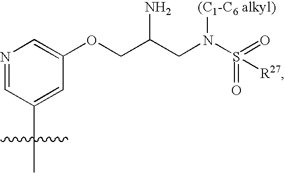

wherein C₁-C₆ alkyl and R²⁷ are as defined for the Benzo[c][2,7]naphthyridine Derivatives of Formula (Ib).

In various embodiments for the compounds of Formula (Ib), R¹ is

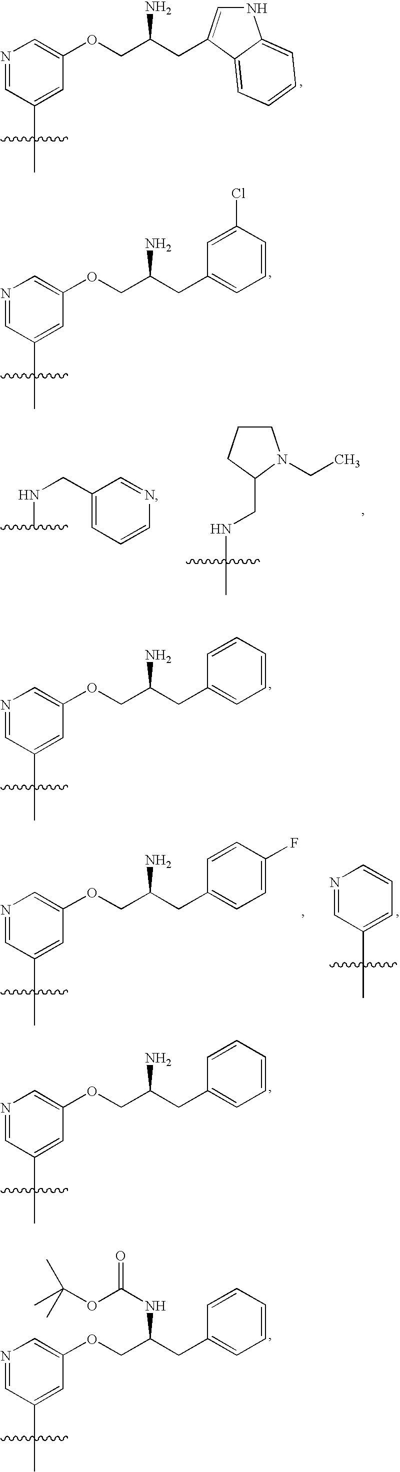

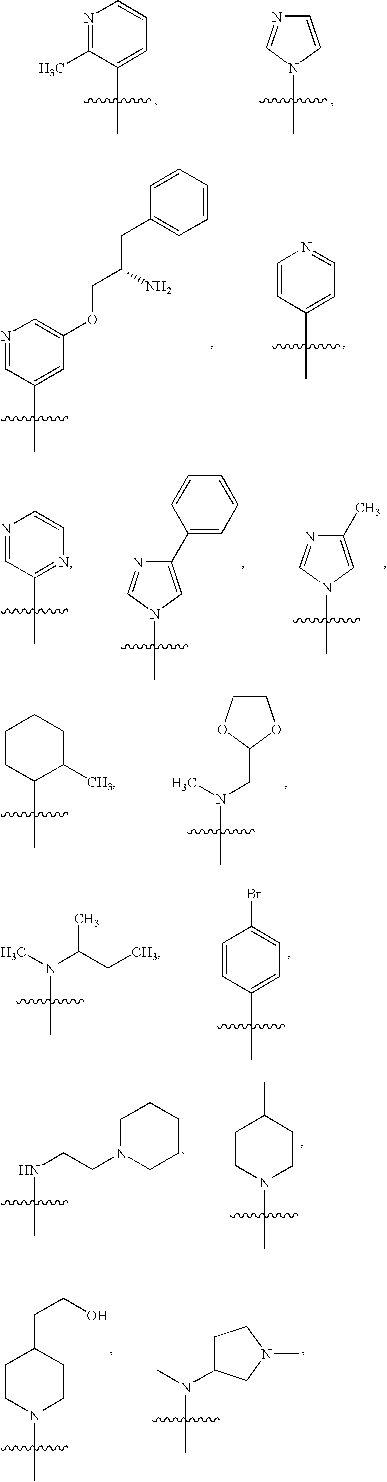

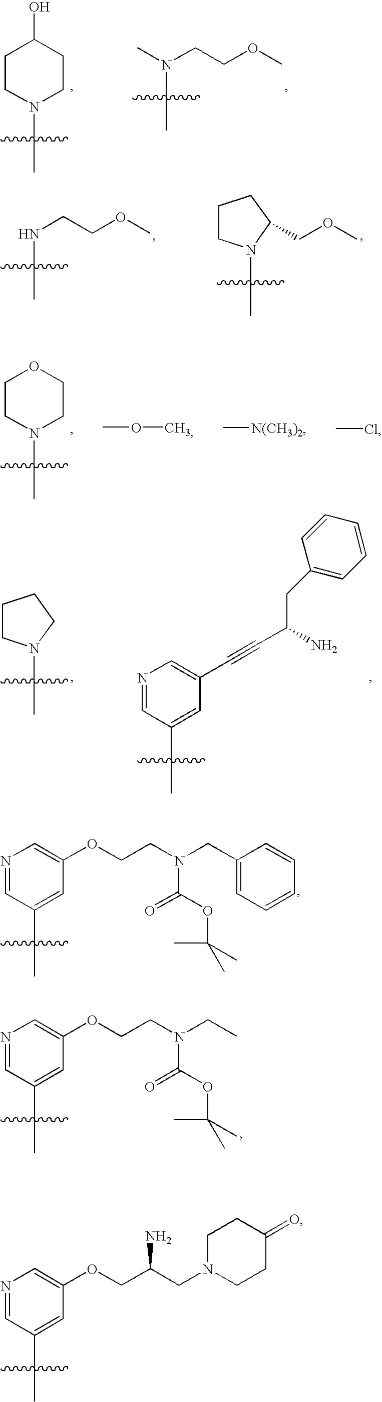

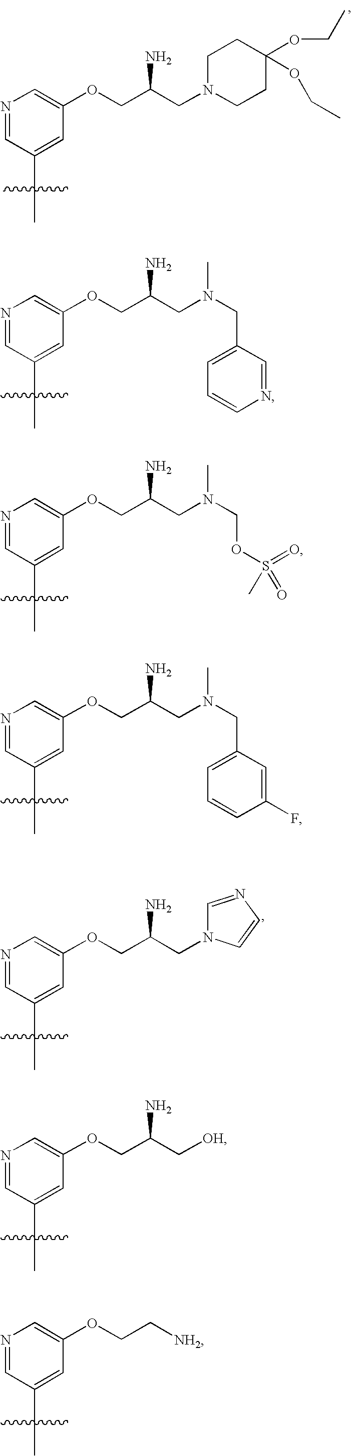

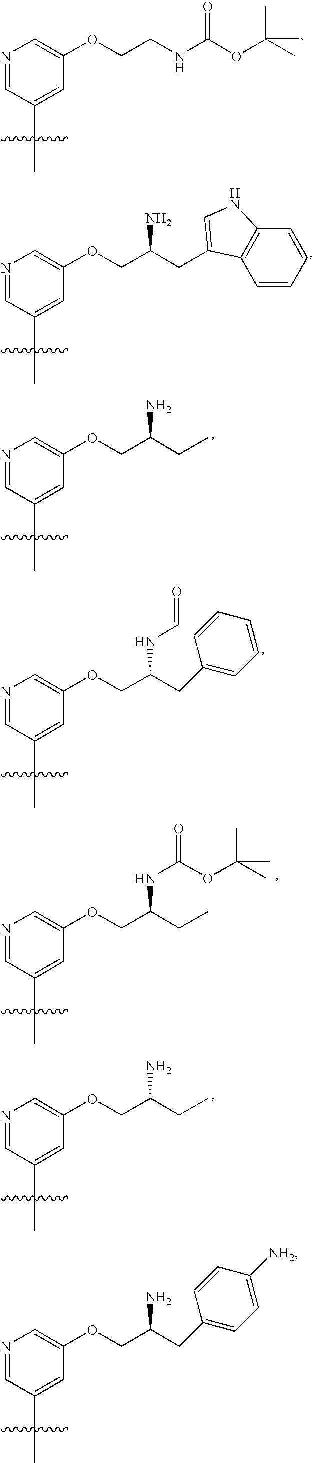

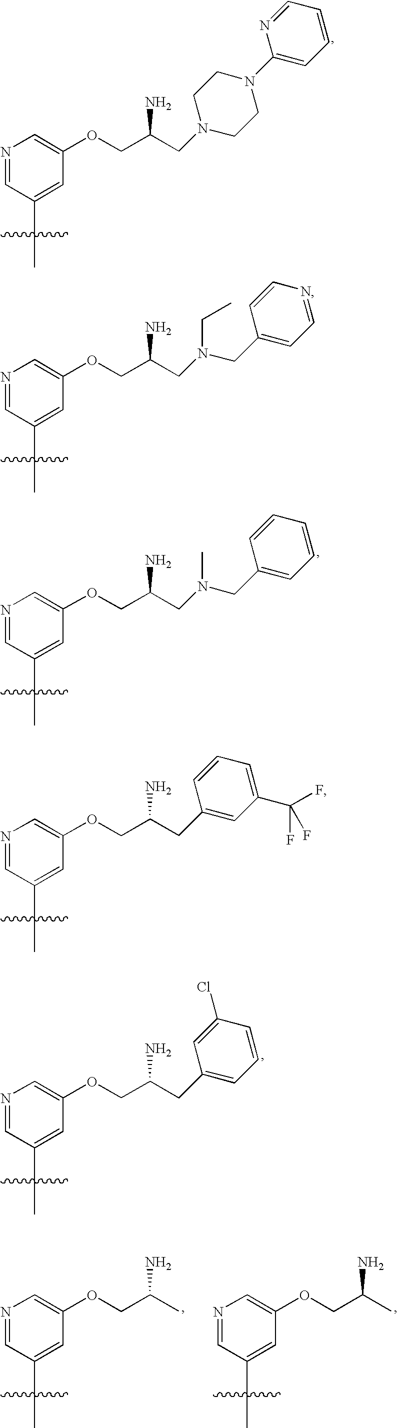

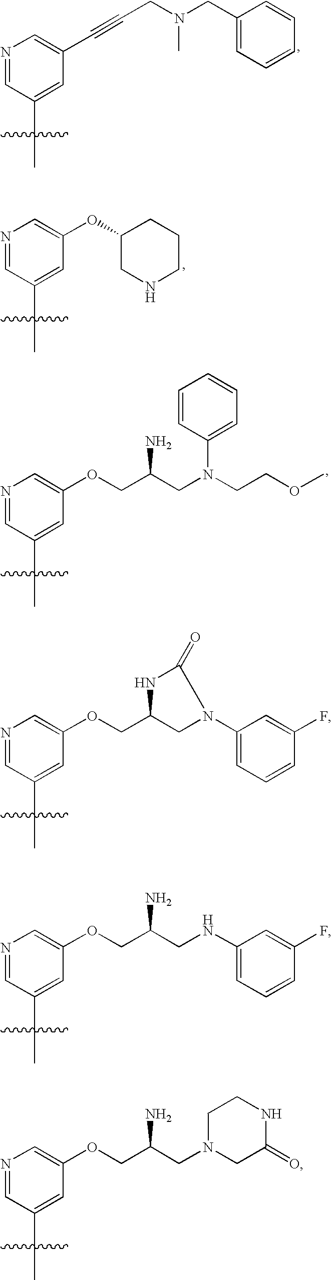

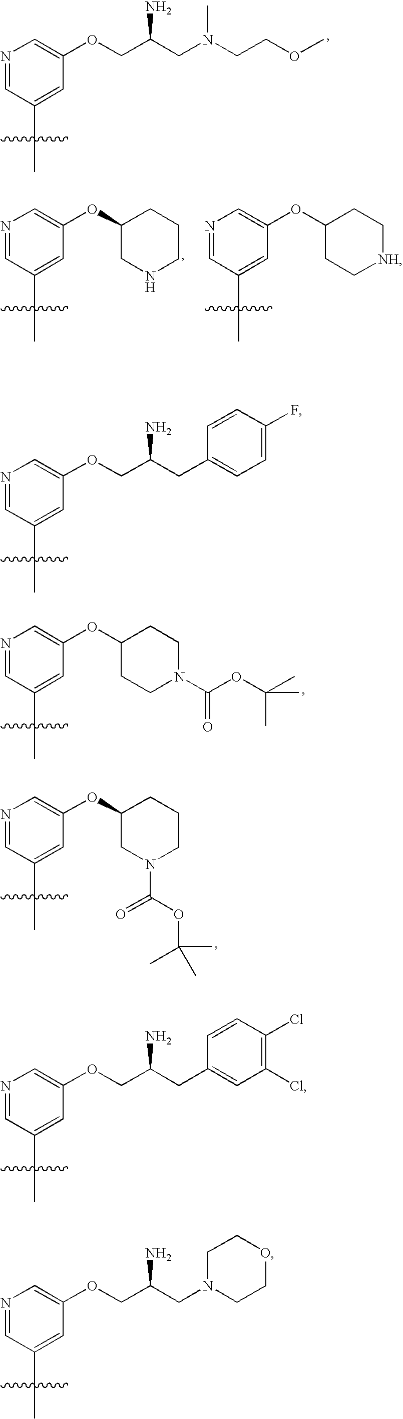

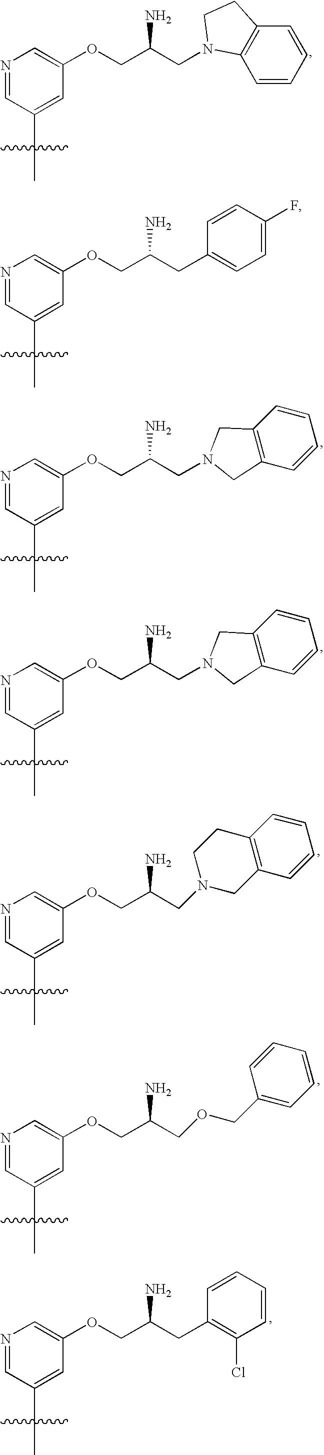

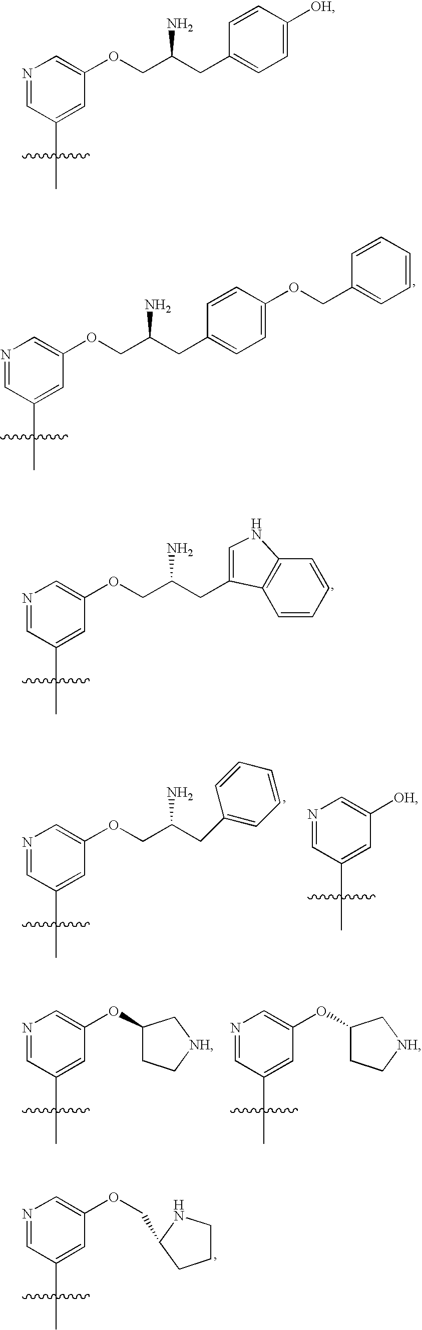

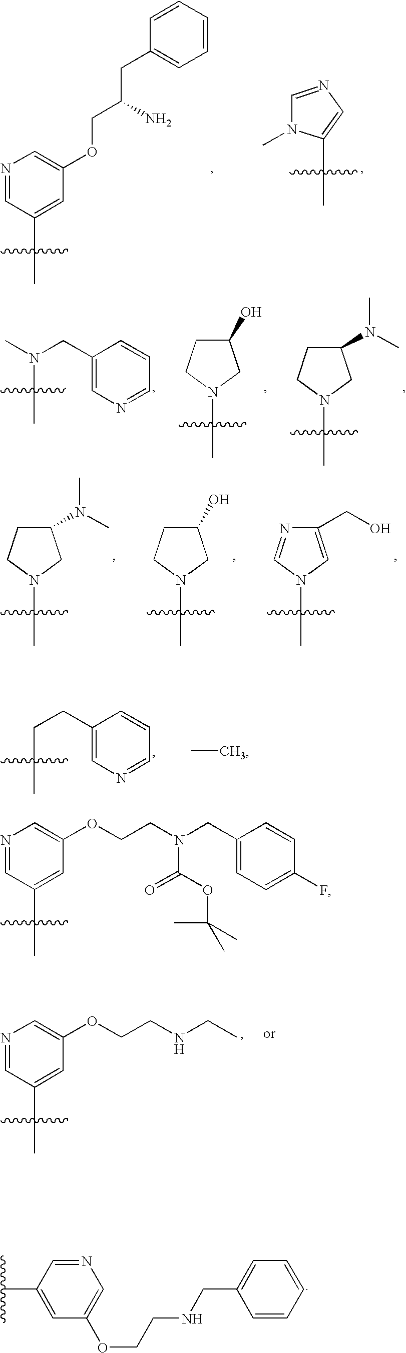

Illustrative examples of the Benzo[c][2,7]naphthyridine Derivatives include the compounds of Formula (Ib) as set forth below:

| Com- | |
|---|---|
| pound | R¹ |
| Ib-1 | 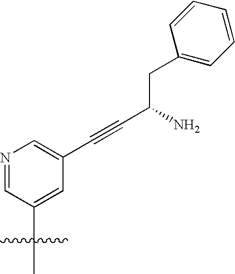 |
| Ib-2 | 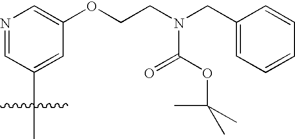 |
| Ib-3 | 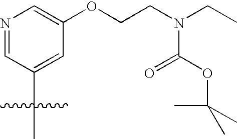 |
| Ib-4 | 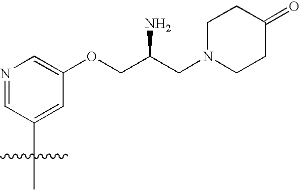 |
| Ib-5 | 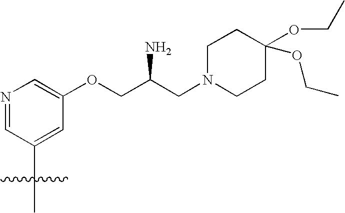 |
| Ib-6 | 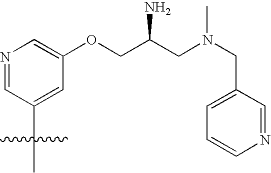 |
| Ib-7 | 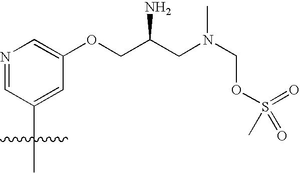 |
| Ib-8 | 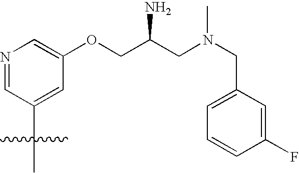 |
| Ib-9 | 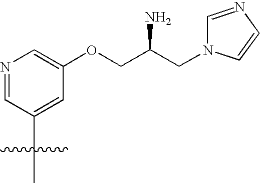 |
| Ib-10 | 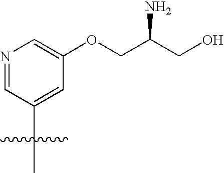 |
| Ib-11 | 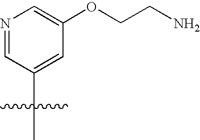 |
| Ib-12 | 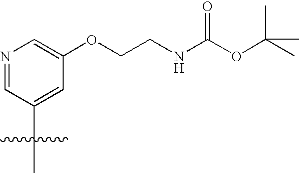 |
| Ib-13 | 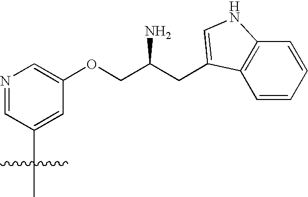 |
| Ib-14 | 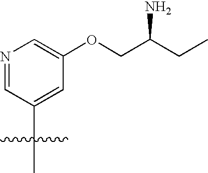 |
| Ib-15 | 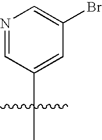 |
| Ib-16 | 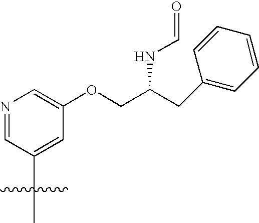 |
| Ib-17 | 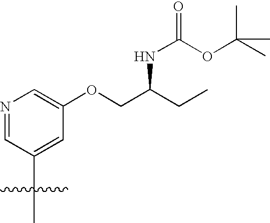 |
| Ib-18 | 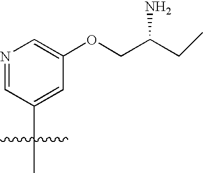 |
| Ib-19 | 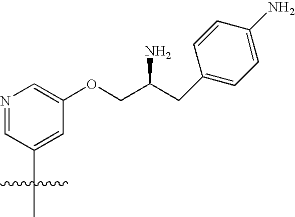 |
| Ib-20 | 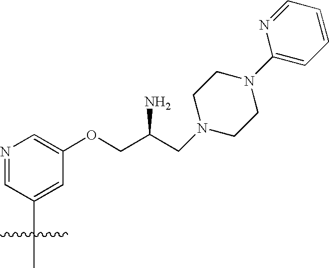 |
| Ib-21 | 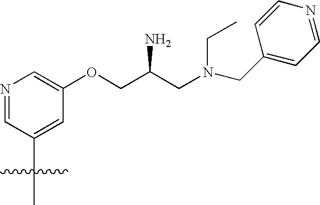 |
| Ib-22 | 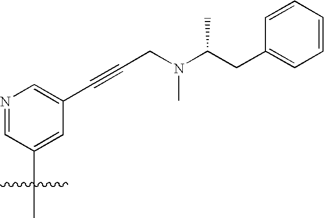 |
| Ib-23 | 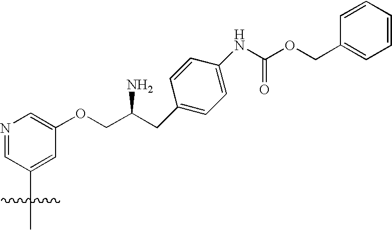 |
| Ib-24 | 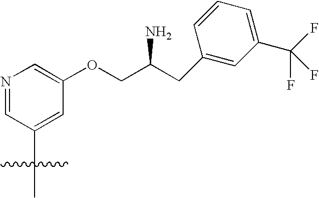 |
| Ib-25 | 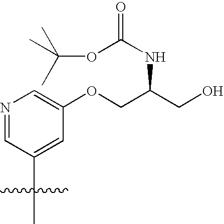 |
| Ib-26 | 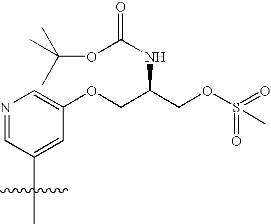 |
| Ib-27 | 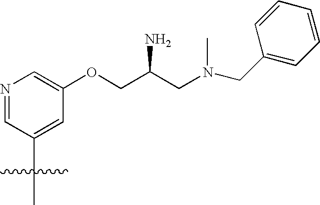 |
| Ib-28 | 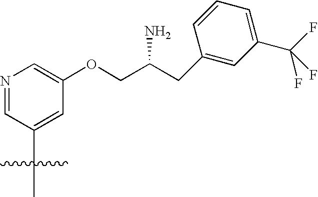 |
| Ib-29 | 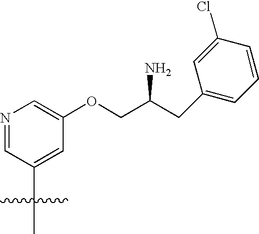 |
| Ib-30 | 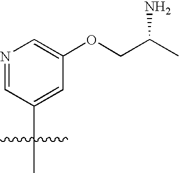 |
| Ib-31 | 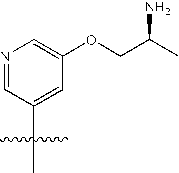 |
| Ib-32 | 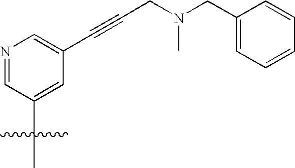 |
| Ib-33 | 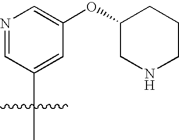 |
| Ib-34 | 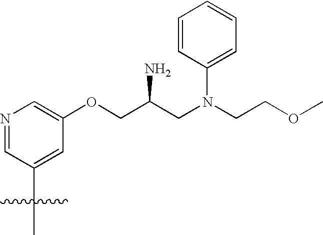 |
| Ib-35 | 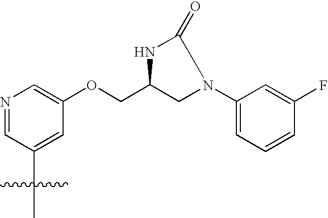 |
| Ib-36 | 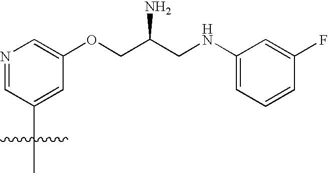 |
| Ib-37 | 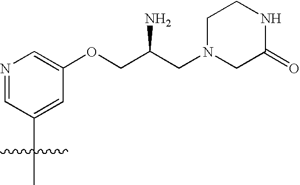 |
| Ib-38 | 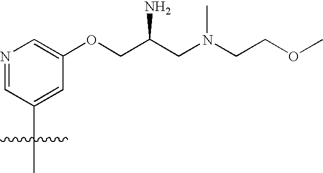 |
| Ib-39 | 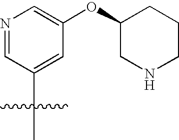 |
| Ib-40 | 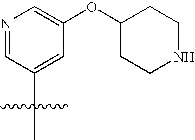 |
| Ib-41 | 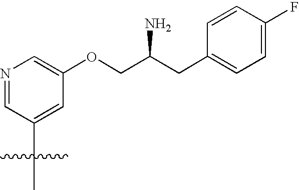 |
| Ib-42 | 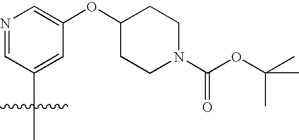 |
| Ib-43 | 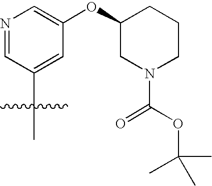 |
| Ib-44 | 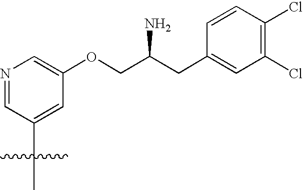 |
| Ib-45 | 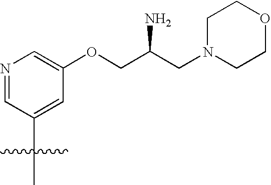 |
| Ib-46 | 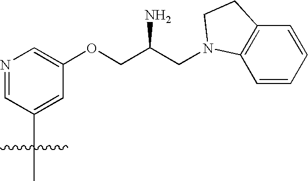 |
| Ib-47 | 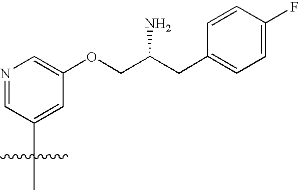 |
| Ib-48 | 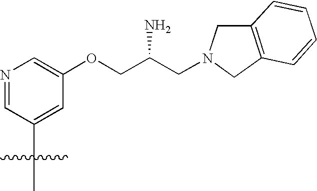 |
| Ib-49 | 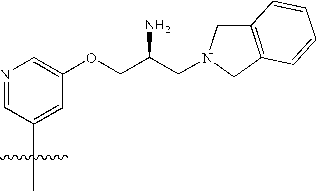 |
| Ib-50 | 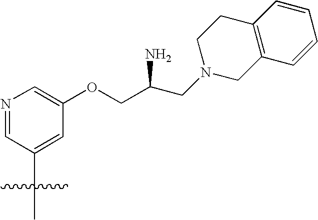 |
| Ib-51 | 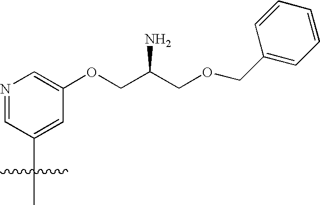 |
| Ib-52 | 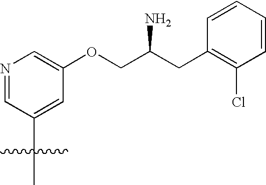 |
| Ib-53 | 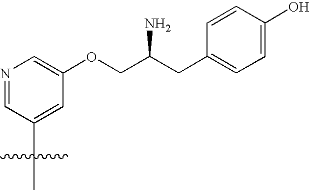 |
| Ib-54 | 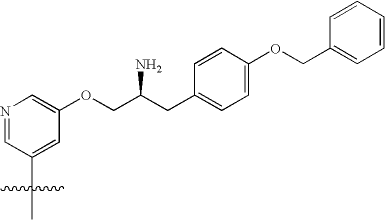 |
| Ib-55 | 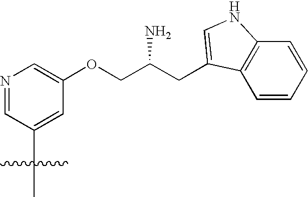 |
| Ib-56 | 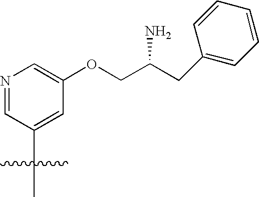 |
| Ib-57 | 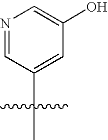 |
| Ib-58 | 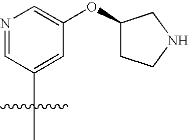 |
| Ib-59 | 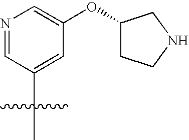 |
| Ib-60 | 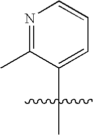 |
| Ib-61 | 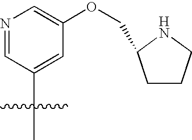 |
| Ib-62 | 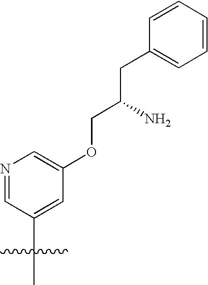 |
| Ib-63 | 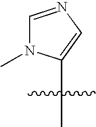 |
| Ib-64 | 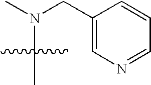 |
| Ib-65 | 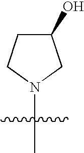 |
| Ib-66 | 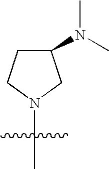 |
| Ib-67 | 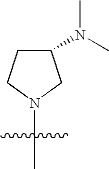 |
| Ib-68 | 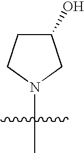 |
| Ib-69 | 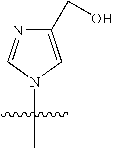 |
| Ib-70 | 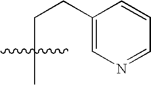 |
| Ib-71 | —CH₃ |
| Ib-72 | 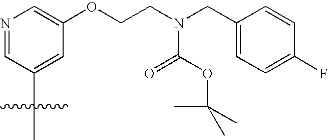 |
| Ib-73 | 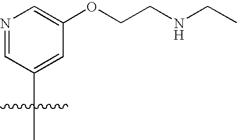 |
| Ib-74 | 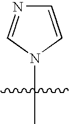 |
| Ib-75 | 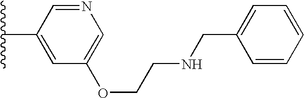 |
| Ib-76 | 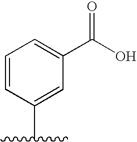 |
| Ib-77 | 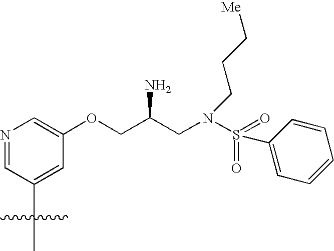 |
| Ib-78 | 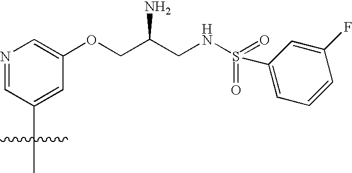 |
| Ib-79 | 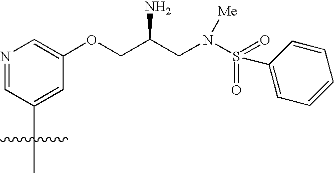 |
| Ib-80 | 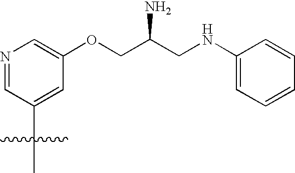 |
| Ib-81 | 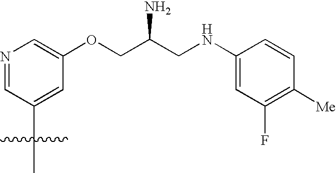 |
| Ib-82 | 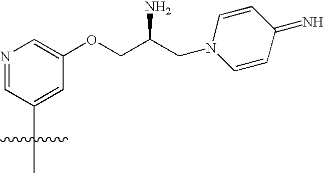 |
| Ib-83 | 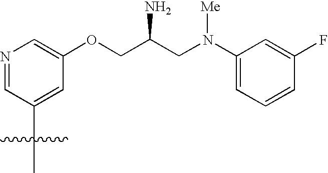 |
| Ib-84 | 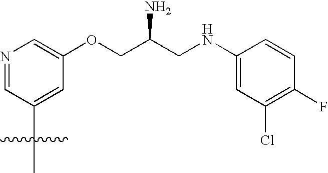 |
| Ib-85 | 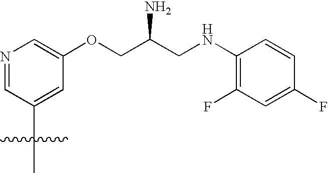 |
| Ib-86 | 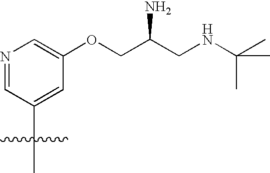 |
| Ib-87 | 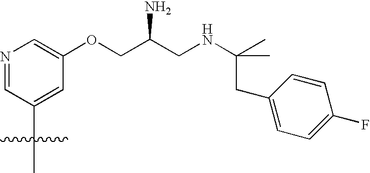 |
| Ib-89 | 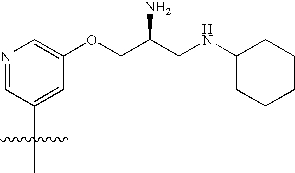 |
| Ib-90 | 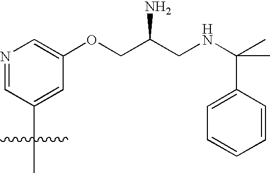 |
| Ib-91 | 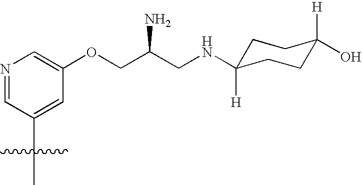 |
| Ib-92 | 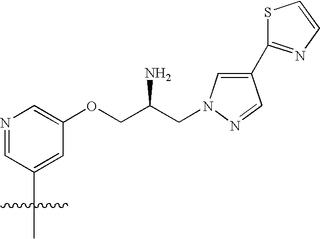 |
| Ib-93 | 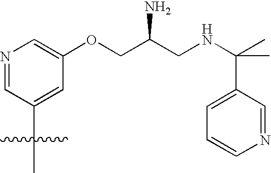 |
| Ib-94 | 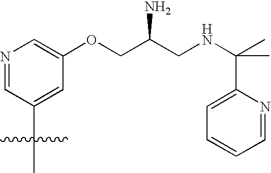 |
| Ib-95 | 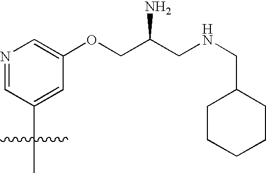 |
| Ib-96 | 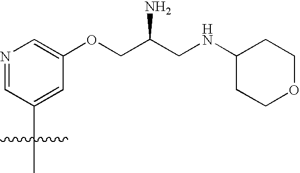 |
| Ib-97 | 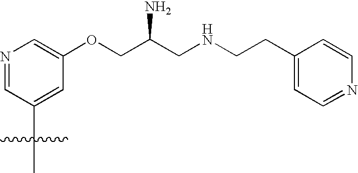 |
| Ib-98 | 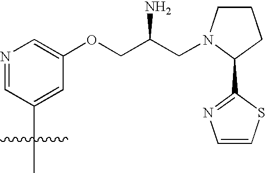 |
| Ib-100 | 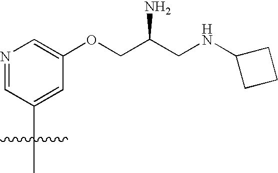 |
| Ib-101 | 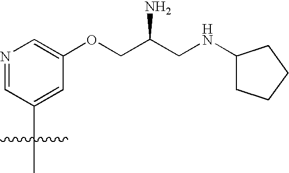 |
| Ib-102 | 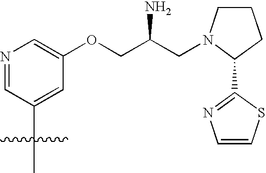 |
| Ib-103 | 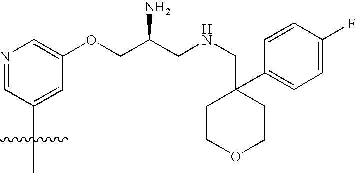 |
| Ib-104 | 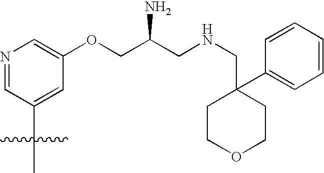 |
| Ib-105 | 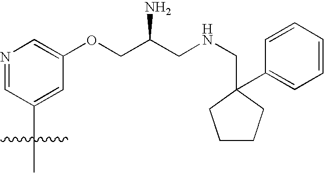 |
| Ib-106 | 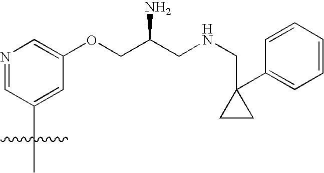 |
| Ib-107 | 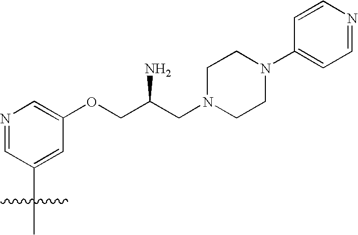 |
| Ib-108 | 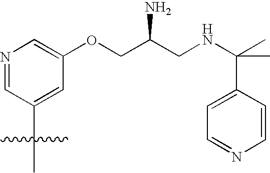 |
| Ib-109 | 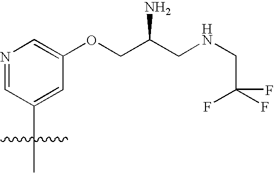 |
| Ib-110 | 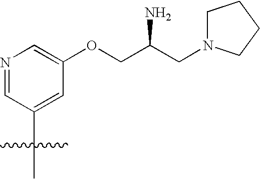 |
| Ib-111 | 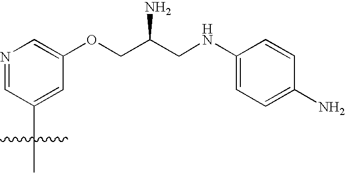 |
| Ib-112 | 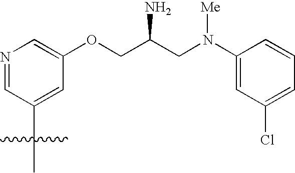 |
| Ib-113 | 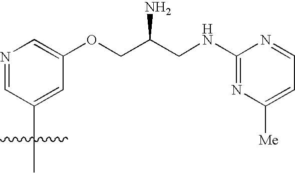 |
| Ib-114 | 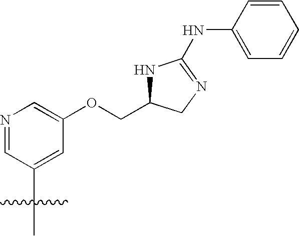 |
| Ib-115 | 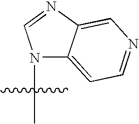 |
| Ib-116 | 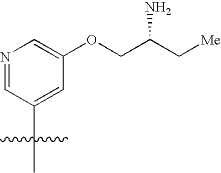 |
| Ib-117 | 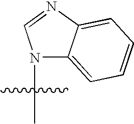 |
| Ib-118 | 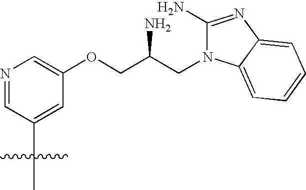 |
| Ib-119 | —OH |
| Ib-120 | 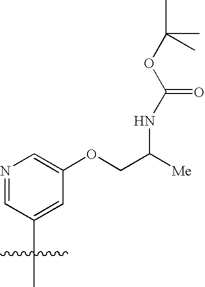 |
| Ib-121 | 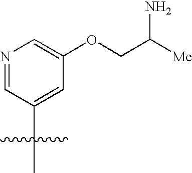 |
| Ib-122 | 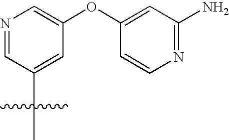 |
| Ib-141 | 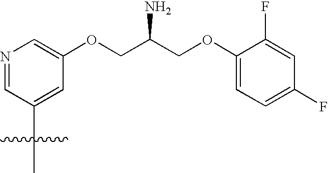 |
| Ib-142 | 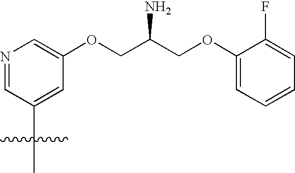 |
| Ib-143 | 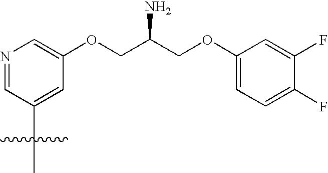 |
| Ib-144 | 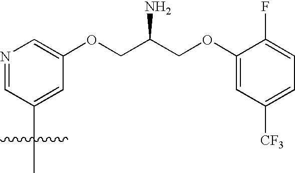 |
| Ib-145 | 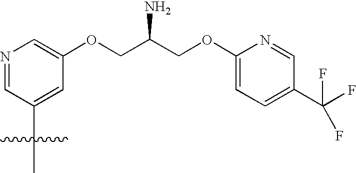 |
| Ib-146 | 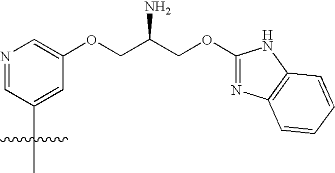 |
| Ib-147 | 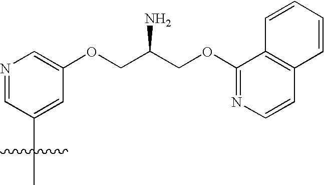 |
| Ib-148 | 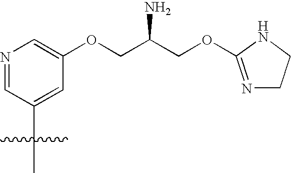 |
| Ib-149 | 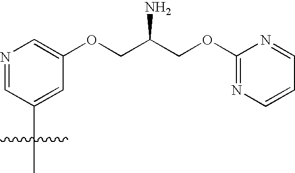 |
| Ib-150 | 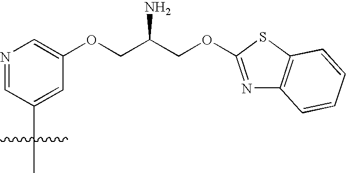 |
| Ib-151 | 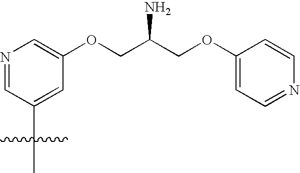 |
| Ib-152 | 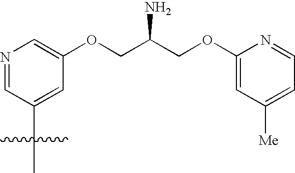 |
| Ib-153 | 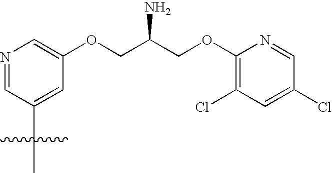 |
| Ib-154 | 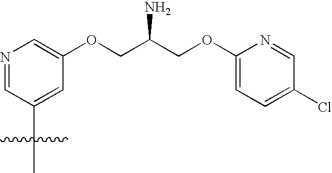 |
| Ib-155 | 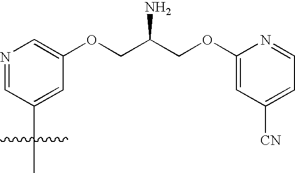 |
| Ib-156 | 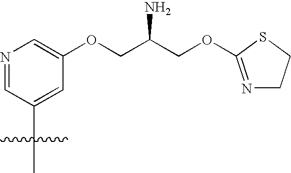 |
| Ib-157 | 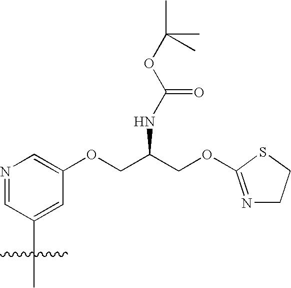 |
| Ib-158 | 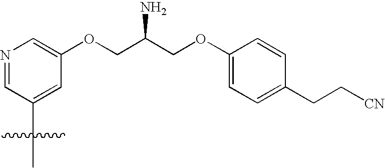 |
| Ib-159 | 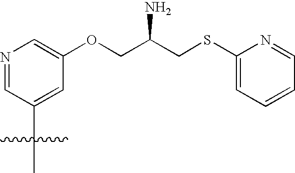 |
| Ib-160 | 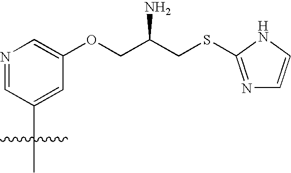 |
| Ib-161 | 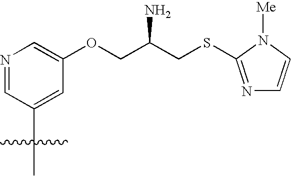 |
| Ib-162 | 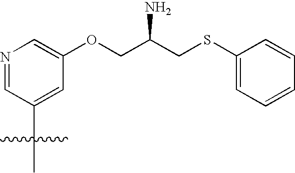 |
| Ib-163 | 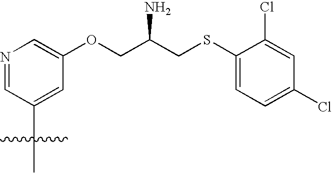 |
| Ib-164 | 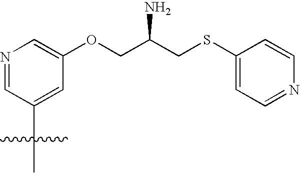 |
| Ib-165 | 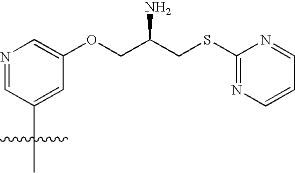 |
| Ib-166 | 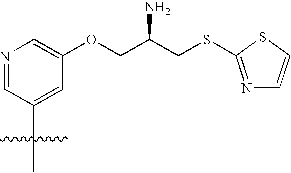 |
| Ib-167 | 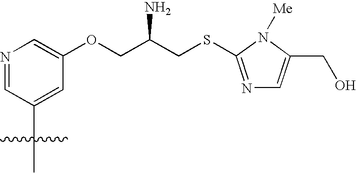 |
| Ib-168 | 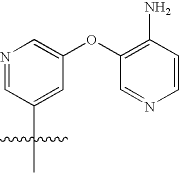 |
| Ib-169 | 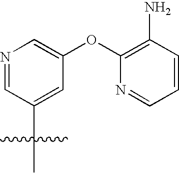 |
| Ib-170 | 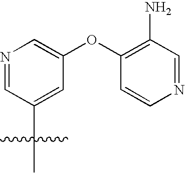 |
| Ib-171 | 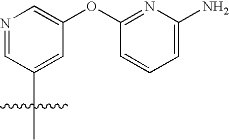 |
| Ib-172 | 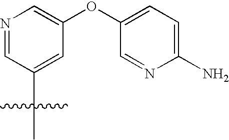 |
| Ib-173 | 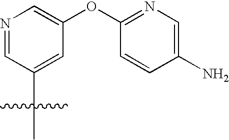 |
| Ib-174 | 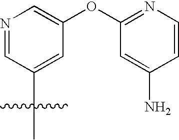 |
| Ib-175 | 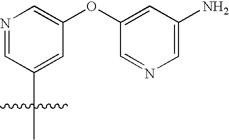 |
| Ib-176 | 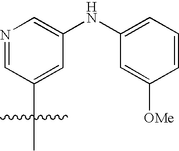 |
| Ib-177 | 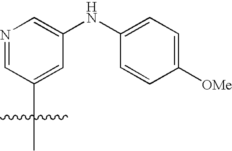 |
| Ib-178 | 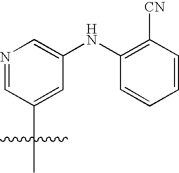 |
| Ib-179 | 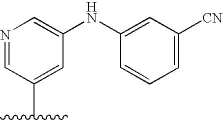 |
| Ib-180 | 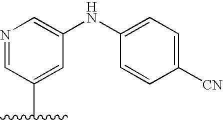 |
| Ib-181 | 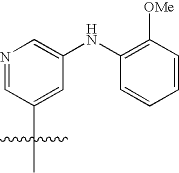 |
| Ib-182 | 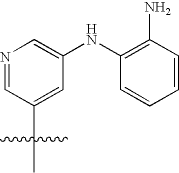 |
| Ib-183 | 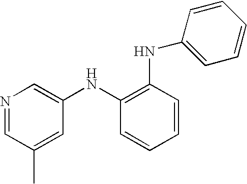 |
| Ib-184a | 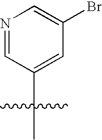 |
| Ib-184b | 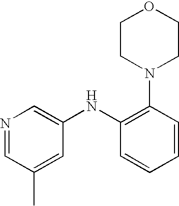 |
| Ib-185 | 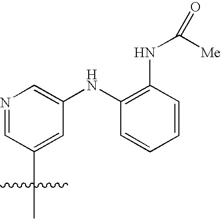 |
| Ib-186 | 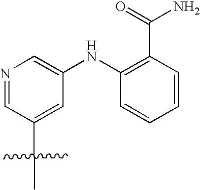 |
| Ib-187 | 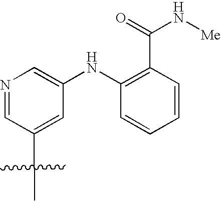 |
| Ib-188 | 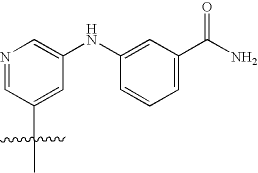 |
| Ib-189 | 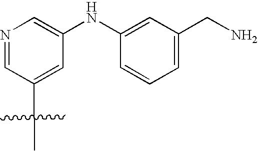 |
| Ib-190 | 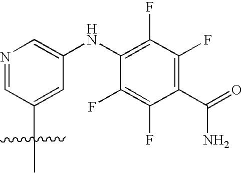 |
| Ib-191 | 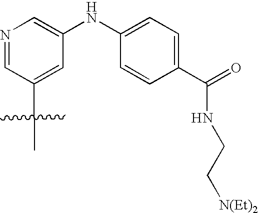 |
| Ib-192 | 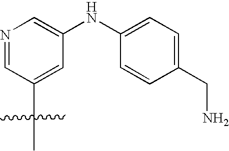 |
| Ib-193 | 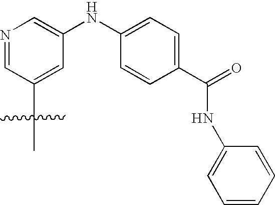 |
| Ib-194 | 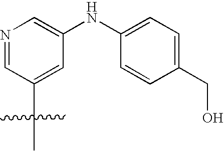 |
| Ib-195 | 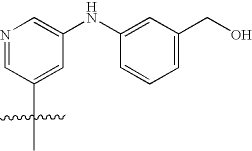 |
| Ib-196 | 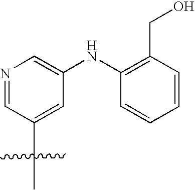 |
| Ib-197 | 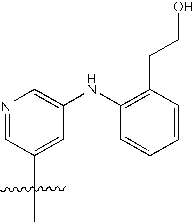 |
| Ib-198 | 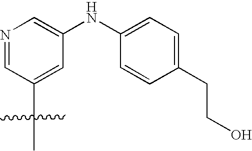 |
| Ib-199 | 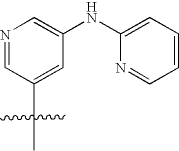 |
| Ib-200 | 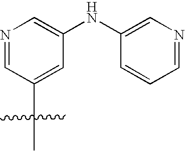 |
| Ib-201 | 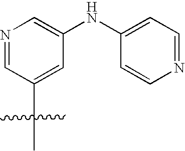 |
| Ib-202 | 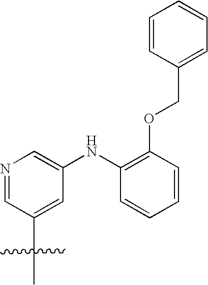 |
| Ib-203 | 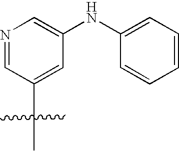 |
| Ib-204 | 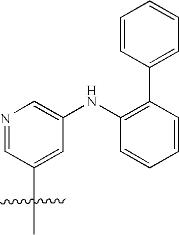 |
| Ib-205 | 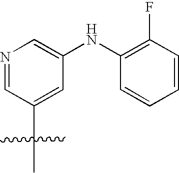 |
| Ib-206 | 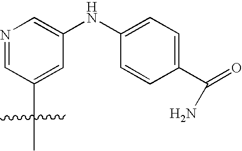 |
| Ib-207 | 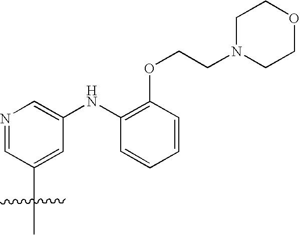 |
| Ib-208 | 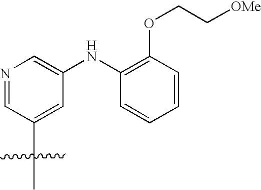 |
| Ib-209 | 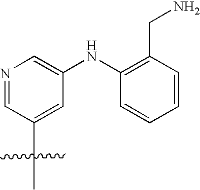 |
| Ib-210 | 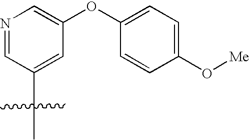 |
| Ib-211 | 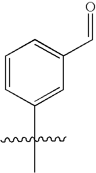 |
| Ib-212 | 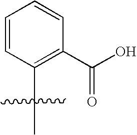 |
| Ib-213 | 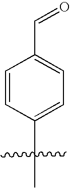 |
| Ib-214 | 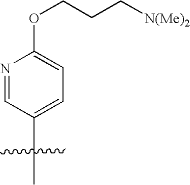 |
| Ib-215 | 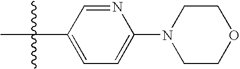 |
| Ib-216 | 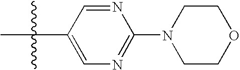 |
| Ib-217 | 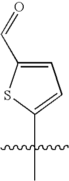 |
| Ib-218 | 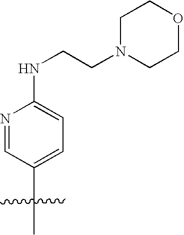 |
| Ib-219 | 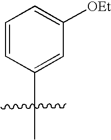 |
| Ib-220 | 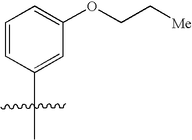 |
| Ib-221 | 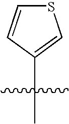 |
| Ib-222 | 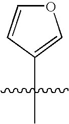 |
| Ib-223 | 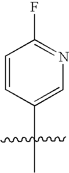 |
| Ib-224 | 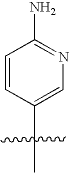 |
| Ib-226 | 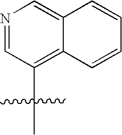 |
| Ib-227 | 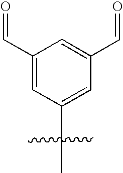 |
| Ib-228 | 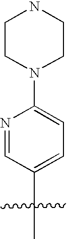 |
| Ib-229 | 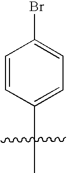 |
| Ib-230 | —H |
| Ib-231 | —Cl |
| Ib-232 | 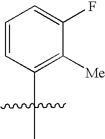 |
| Ib-233 | 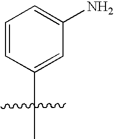 |
| Ib-234 | 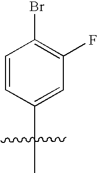 |
| Ib-235 | 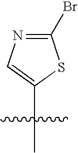 |
| Ib-236 | 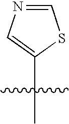 |
| Ib-237 | 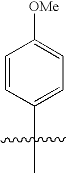 |
| Ib-238 | 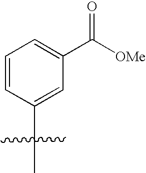 |
| Ib-239 | 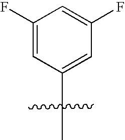 |
| Ib-240 | 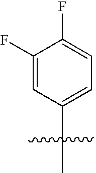 |
| Ib-241 | 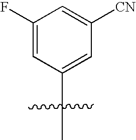 |
| Ib-242 | 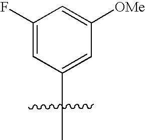 |
| Ib-243 | 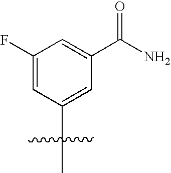 |
| Ib-244 | 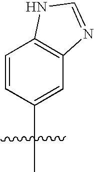 |
| Ib-245 | 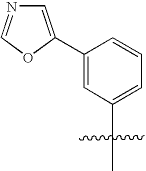 |
| Ib-246 | 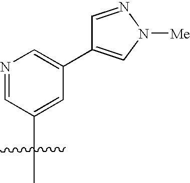 |
| Ib-247 | 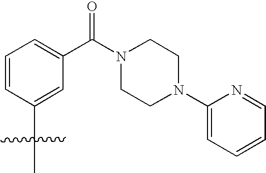 |
| Ib-248 | 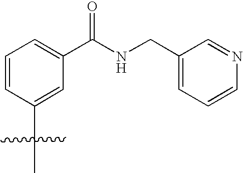 |
| Ib-249 | 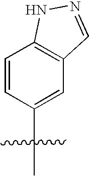 |
| Ib-250 | 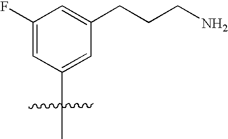 |
| Ib-251 | 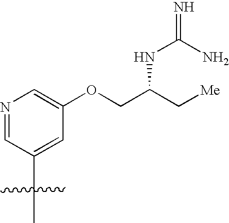 |
| Ib-252 | 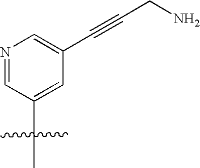 |
| Ib-253 | 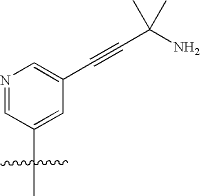 |
| Ib-254 | 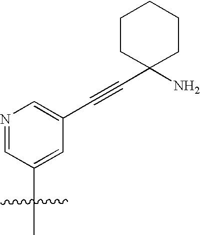 |
| Ib-255 | 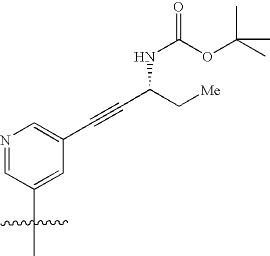 |
| Ib-256 | 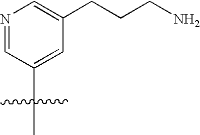 |
| Ib-257 | 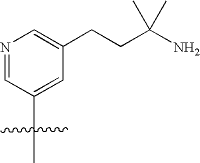 |
| Ib-258 | 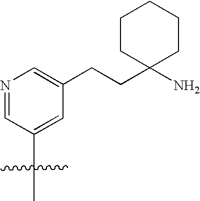 |
| Ib-259 | 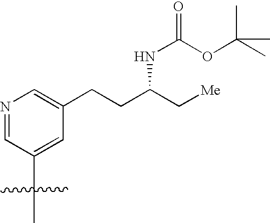 |
| Ib-260 | 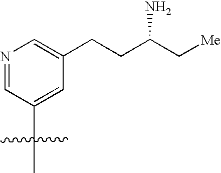 |
| Ib-261 | 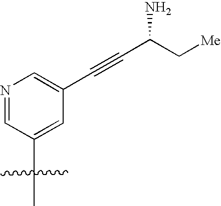 |
| Ib-262 | 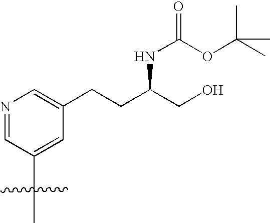 |
| Ib-263 | 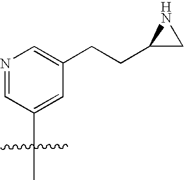 |
| Ib-264 | 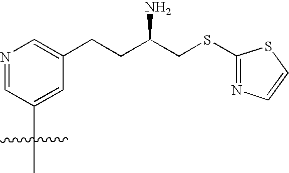 |
| Ib-265a | 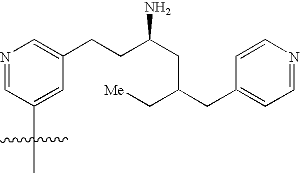 |
| Ib-265b | 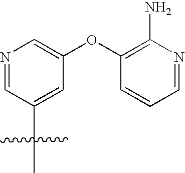 |
| Ib-266 | 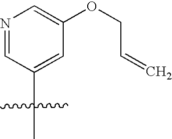 |
| Ib-267 | 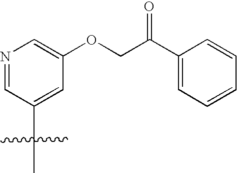 |
| Ib-268 | 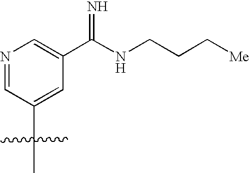 |
| Ib-269 | 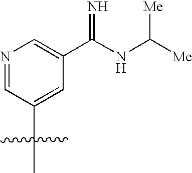 |
| Ib-270 | 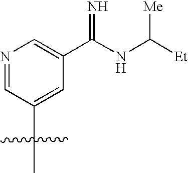 |
| Ib-271 | 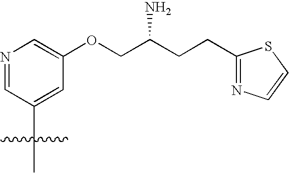 |

and a pharmaceutically acceptable salt thereof.

Illustrative examples of Benzo[c][2,7]naphthyridine Derivatives of Formula (I), (Ia) and/or (Ib) include:
9-methoxy-8-(2-methoxyethoxy)-2-pyrrolidin-1-ylbenzo[c]-2,7-naphthyridin-4-amine;
2-chloro-9-methoxy-8-(2-methoxyethoxy)benzo[c]-2,7-naphthyridin-4-amine;
9-methoxy-8-(2-methoxyethoxy)-N²,N²-dimethylbenzo[c]-2,7-naphthyridine-2,4-diamine;
2,9-dimethoxy-8-(2-methoxyethoxy)benzo[c]-2,7-naphthyridin-4-amine;
9-methoxy-8-(2-methoxyethoxy)-2-morpholin-4-ylbenzo[c]-2,7-naphthyridin-4-amine;
9-methoxy-8-(2-methoxyethoxy)-2-[(2R)-2-(methoxymethyl)pyrrolidin-1-yl]benzo[c]-2,7-naphthyridin-4-amine;
9-methoxy-8-(2-methoxyethoxy)-N²-(2-methoxyethyl)benzo[c]-2,7-naphthyridine-2,4-diamine;
9-methoxy-8-(2-methoxyethoxy)-N²-(2-methoxyethyl)-N²-methylbenzo[c]-2,7-naphthyridine-2,4-diamine;
1-[4-amino-9-methoxy-8-(2-methoxyethoxy)benzo[c]-2,7-naphthyridin-2-yl]piperidin-4-ol;
9-methoxy-8-(2-methoxyethoxy)-N²-methyl-N²-(1-methylpyrrolidin-3-yl)benzo[c]-2,7-naphthyridine-2,4-diamine;
2-{1-[4-amino-9-methoxy-8-(2-methoxyethoxy)benzo[c]-2,7-naphthyridin-2-yl]piperidin-4-yl}ethanol;
9-methoxy-8-(2-methoxyethoxy)-2-(4-methylpiperidin-1-yl)benzo[c]-2,7-naphthyridin-4-amine;
9-methoxy-8-(2-methoxyethoxy)-N²-(2-piperidin-1-ylethyl)benzo[c]-2,7-naphthyridine-2,4-diamine;
2-(1H-imidazol-1-yl)-9-methoxy-8-(2-methoxyethoxy)benzo[c]-2,7-naphthyridin-4-amine;
2-(4-bromophenyl)-8,9-diethoxybenzo[c]-2,7-naphthyridin-4-amine;
8,9-diethoxy-2-pyridin-3-ylbenzo[c]-2,7-naphthyridin-4-amine;
N²-isopropyl-9-methoxy-8-(2-methoxyethoxy)-N²-methylbenzo[c]-2,7-naphthyridine-2,4-diamine;
N²-(1,3-dioxolan-2-ylmethyl)-9-methoxy-8-(2-methoxyethoxy)-N²-methylbenzo[c]-2,7-naphthyridine-2,4-diamine;
N,N′-bis[(1-ethylpyrrolidin-2-yl)methyl]-9-methoxy-8-(2-methoxyethoxy)benzo[c]-2,7-naphthyridine-2,4-diamine;
9-methoxy-8-(2-methoxyethoxy)-2-(2-methylpiperidin-1-yl)benzo[c]-2,7-naphthyridin-4-amine;
9-methoxy-8-(2-methoxyethoxy)-2-(4-methyl-1H-imidazol-1-yl)benzo[c]-2,7-naphthyridin-4-amine;
9-methoxy-8-(2-methoxyethoxy)-N,N′-bis(pyridin-3-ylmethyl)benzo[c]-2,7-naphthyridine-2,4-diamine;
9-methoxy-8-(2-methoxyethoxy)-2-(4-phenyl-1H-imidazol-1-yl)benzo[c]-2,7-naphthyridin-4-amine;
2-(1H-imidazol-1-yl)-8,9-dimethoxybenzo[c]-2,7-naphthyridin-4-amine;
[1-(4-amino-8,9-dimethoxybenzo[c]-2,7-naphthyridin-2-yl)-1H-imidazol-4-yl]methanol;
(3S)-1-(4-amino-8,9-dimethoxybenzo[c]-2,7-naphthyridin-2-yl)pyrrolidin-3-ol;
2-[(3S)-3-(dimethylamino)pyrrolidin-1-yl]-8,9-dimethoxybenzo[c]-2,7-naphthyridin-4-amine;
2-[(3R)-3-(dimethylamino)pyrrolidin-1-yl]-8,9-dimethoxybenzo[c]-2,7-naphthyridin-4-amine;
(3R)-1-(4-amino-8,9-dimethoxybenzo[c]-2,7-naphthyridin-2-yl)pyrrolidin-3-ol;
8,9-dimethoxy-N²-methyl-N²-(pyridin-3-ylmethyl)benzo[c]-2,7-naphthyridine-2,4-diamine;
8-bromo-2-(1H-imidazol-1-yl)benzo[c]-2,7-naphthyridin-4-amine;
8-[3-(dimethylamino)prop-1-yn-1-yl]-2-(1H-imidazol-1-yl)benzo[c]-2,7-naphthyridin-4-amine;
8-[4-(4-ethylpiperazin-1-yl)but-1-yn-1-yl]-2-(1H-imidazol-1-yl)benzo[c]-2,7-naphthyridin-4-amine;
8-(benzyloxy)-2-(1H-imidazol-1-yl)-9-methoxybenzo[c]-2,7-naphthyridin-4-amine;
4-amino-2-(1H-imidazol-1-yl)-9-methoxybenzo[c]-2,7-naphthyridin-8-ol;
9-methoxy-8-[(1-methylpiperidin-4-yl)methoxy]-2-pyrazin-2-ylbenzo[c]-2,7-naphthyridin-4-amine;
9-methoxy-2-pyridin-3-yl-8-(3-pyrrolidin-1-ylpropoxy)benzo[c]-2,7-naphthyridin-4-amine;
9-Methoxy-8-[(1-methylpiperidin-4-yl)methoxy]-2-pyridin-4-ylbenzo[c]-2,7-naphthyridin-4-amine;
2-(1H-imidazol-1-yl)-8-[4-(1H-imidazol-1-yl)but-1-yn-1-yl]benzo[c]-2,7-naphthyridin-4-amine;
N-[4-amino-8-ethoxy-2-(1H-imidazol-1-yl)benzo[c]-2,7-naphthyridin-9-yl]acetamide;
8-ethoxy-2-(1H-imidazol-1-yl)benzo[c]-2,7-naphthyridine-4,9-diamine;
N-[4-amino-8-ethoxy-2-(1H-imidazol-1-yl)benzo[c]-2,7-naphthyridin-9-yl]-N′-[4-(dimethylamino)phenyl]urea;
N-[4-amino-8-ethoxy-2-(1H-imidazol-1-yl)benzo[c]-2,7-naphthyridin-9-yl]-N′-(2-morpholin-4-ylethyl)thiourea;
8-[(1E)-4-(4-ethylpiperazin-1-yl)but-1-en-1-yl]-2-(1H-imidazol-1-yl)benzo[c]-2,7-naphthyridin-4-amine;
2-(1H-imidazol-1-yl)-8-[(1E)-4-pyrrolidin-1-ylbut-1-en-1-yl]benzo[c]-2,7-naphthyridin-4-amine;
8-(benzyloxy)-9-methoxy-2-pyridin-3-ylbenzo[c]-2,7-naphthyridin-4-amine;
8-(2-chloroethoxy)-2-(1H-imidazol-1-yl)-9-methoxybenzo[c]-2,7-naphthyridin-4-amine;
8,9-dimethoxy-2-(1-methyl-1H-imidazol-5-yl)benzo[c]-2,7-naphthyridin-4-amine;
2-(5-{[(2S)-2-amino-3-phenylpropyl]oxy}pyridin-3-yl)-8,9-dimethoxybenzo[c]-2,7-naphthyridin-4-amine;
8,9-dimethoxy-2-{5-[(2R)-pyrrolidin-2-ylmethoxy]pyridin-3-yl}benzo[c]-2,7-naphthyridin-4-amine;
9-methoxy-2-pyridin-3-yl-8-(4-pyrrolidin-1-ylpiperidin-1-yl)benzo[c]-2,7-naphthyridin-4-amine;
9-methoxy-2-(2-methylpyridin-3-yl)-8-(4-pyrrolidin-1-ylpiperidin-1-yl)benzo[c]-2,7-naphthyridin-4-amine;
8,9-dimethoxy-2-(2-methylpyridin-3-yl)benzo[c]-2,7-naphthyridin-4-amine;
9-[4-(4-ethylpiperazin-1-yl)but-1-yn-1-yl]-2-(1H-imidazol-1-yl)benzo[c]-2,7-naphthyridin-4-amine;
8-(3-chloropropoxy)-2-(1H-imidazol-1-yl)-9-methoxybenzo[c]-2,7-naphthyridin-4-amine;
2-(1H-imidazol-1-yl)-9-methoxy-8-(3-morpholin-4-ylpropoxy)benzo[c]-2,7-naphthyridin-4-amine;
2-(1H-imidazol-1-yl)-9-methoxy-8-(3-pyrrolidin-1-ylpropoxy)benzo[c]-2,7-naphthyridin-4-amine;
2-(1H-imidazol-1-yl)-9-methoxy-8-[3-(4-methylpiperazin-1-yl)propoxy]benzo[c]-2,7-naphthyridin-4-amine;
2-(1H-imidazol-1-yl)-9-methoxy-8-(2-morpholin-4-ylethoxy)benzo[c]-2,7-naphthyridin-4-amine;
2-(1H-imidazol-1-yl)-9-methoxy-8-(2-pyrrolidin-1-ylethoxy)benzo[c]-2,7-naphthyridin-4-amine;
8,9-dimethoxy-2-{5-[(3S)-pyrrolidin-3-yloxy]pyridin-3-yl}benzo[c]-2,7-naphthyridin-4-amine;
8,9-dimethoxy-2-{5-[(3R)-pyrrolidin-3-yloxy]pyridin-3-yl}benzo[c]-2,7-naphthyridin-4-amine;
2-(5-{[(2S)-2-amino-3-phenylpropyl]oxy}pyridin-3-yl)-9-methoxy-8-(2-methoxyethoxy)benzo[c]-2,7-naphthyridin-4-amine;
5-(4-amino-8,9-dimethoxybenzo[c]-2,7-naphthyridin-2-yl)pyridin-3-ol;
2-(1H-imidazol-1-yl)-9-methoxy-8-[2-(4-methylpiperazin-1-yl)ethoxy]benzo[c]-2,7-naphthyridin-4-amine;
2-(5-{[(2R)-2-amino-3-phenylpropyl]oxy}pyridin-3-yl)-8,9-dimethoxybenzo[c]-2,7-naphthyridin-4-amine;
9-methoxy-2-(2-methylpyridin-3-yl)-8-(3-pyrrolidin-1-ylpropoxy)benzo[c]-2,7-naphthyridin-4-amine;
2-(5-{[(2R)-2-amino-3-(1H-indol-3-yl)propyl]oxy}pyridin-3-yl)-8,9-dimethoxybenzo[c]-2,7-naphthyridin-4-amine;
9-bromo-2-pyridin-3-ylbenzo[c]-2,7-naphthyridin-4-amine;
2-[5-({(2S)-2-amino-3-[4-(benzyloxy)phenyl]propyl}oxy)pyridin-3-yl]-8,9-dimethoxybenzo[c]-2,7-naphthyridin-4-amine;
4-((2S)-2-amino-3-{[5-(4-amino-8,9-dimethoxybenzo[c]-2,7-naphthyridin-2-yl)pyridin-3-yl]oxy}propyl)phenol;
2-(5-{[(2S)-2-amino-3-(2-chlorophenyl)propyl]oxy}pyridine-3-yl)-8,9-dimethoxybenzo[c]-2,7-naphthyridin-4-amine;
tert-butyl ((1S)-2-{[5-(4-amino-8-bromobenzo[c]-2,7-naphthyridin-2-yl)pyridin-3-yl]oxy}-1-benzylethyl)carbamate;
tert-butyl {(1S)-2-[(5-{4-amino-8-[4-(1H-imidazol-1-yl)but-1-yn-1-yl]benzo[c]-2,7-naphthyridin-2-yl}pyridine-3-yl)oxy]-1-benzylethyl}carbamate;
2-(5-{[(2S)-2-amino-3-(benzyloxy)propyl]oxy}pyridin-3-yl)-8,9-dimethoxybenzo[c]-2,7-naphthyridin-4-amine;
2-(5-{[(2S)-2-amino-3-(3,4-dihydroisoquinolin-2(1H)-yl)propyl]oxy}pyridin-3-yl)-8,9-dimethoxybenzo[c]-2,7-naphthyridin-4-amine;
2-(5-{[(2S)-2-amino-3-(1,3-dihydro-2H-isoindol-2-yl)propyl]oxy}pyridin-3-yl)-8,9-dimethoxybenzo[c]-2,7-naphthyridin-4-amine;
2-(5-{[(2R)-2-amino-3-(1,3-dihydro-2H-isoindol-2-yl)propyl]oxy}pyridin-3-yl)-8,9-dimethoxybenzo[c]-2,7-naphthyridin-4-amine;
2-(5-{[(2R)-2-amino-3-(4-fluorophenyl)propyl]oxy}pyridin-3-yl)-8,9-dimethoxybenzo[c]-2,7-naphthyridin-4-amine;
2-(5-{[(2S)-2-amino-3-phenylpropyl]oxy}pyridin-3-yl)-8-[4-(1H-imidazol-1-yl)but-1-yn-1-yl]benzo[c]-2,7-naphthyridin-4-amine;
2-(5-{[(2S)-2-amino-3-phenylpropyl]oxy}pyridin-3-yl)-8-[(1E)-4-pyrrolidin-1-ylbut-1-en-1-yl]benzo[c]-2,7-naphthyridin-4-amine;
2-(5-{[(2S)-2-amino-3-phenylpropyl]oxy}pyridin-3-yl)benzo[c]-2,7-naphthyridin-4-amine;
2-(5-{[(2S)-2-amino-3-(2,3-dihydro-1H-indol-1-yl)propyl]oxy}pyridin-3-yl)-8,9-dimethoxybenzo[c]-2,7-naphthyridin-4-amine;
2-(5-{[(2S)-2-amino-3-morpholin-4-ylpropyl]oxy}pyridin-3-yl)-8,9-dimethoxybenzo[c]-2,7-naphthyridin-4-amine;
2-(5-{[(2S)-2-amino-3-(3,4-dichlorophenyl)propyl]oxy}pyridin-3-yl)-8,9-dimethoxybenzo[c]-2,7-naphthyridin-4-amine;
tert-butyl (3S)-3-{[5-(4-amino-8,9-dimethoxybenzo[c]-2,7-naphthyridin-2-yl)pyridin-3-yl]oxy}piperidine-1-carboxylate;
tert-butyl 4-{[5-(4-amino-8,9-dimethoxybenzo[c]-2,7-naphthyridin-2-yl)pyridin-3-yl]oxy}piperidine-1-carboxylate;
2-(5-{[(2S)-2-amino-3-(4-fluorophenyl)propyl]oxy}pyridin-3-yl)-8,9-dimethoxybenzo[c]-2,7-naphthyridin-4-amine;
8,9-dimethoxy-2-[5-piperidin-4-yloxy)pyridin-3-yl]benzo[c]-2,7-naphthyridin-4-amine;
8,9-dimethoxy-2-{5-[(3S)-piperidin-3-yloxy]pyridin-3-yl}benzo[c]-2,7-naphthyridin-4-amine;
(2S)-3-{[5-(4-amino-8,9-dimethoxybenzo[c]-2,7-naphthyridin-2-yl)pyridin-3-yl]oxy}-N1-(2-methoxyethyl)-N1-methylpropane-1,2-diamine;
2-(5-{[(2S)-2-amino-3-phenylpropyl]oxy}pyridin-3-yl)-8,9-dimethoxy-N-methylbenzo[c]-2,7-naphthyridin-4-amine;
4-((2S)-2-amino-3-{[5-(4-amino-8,9-dimethoxybenzo[c]-2,7-naphthyridin-2-yl)pyridin-3-yl]oxy}propyl)piperazin-2-one;
(2S)-3-{[5-(4-amino-8,9-dimethoxybenzo[c]-2,7-naphthyridin-2-yl)pyridin-3-yl]oxy}-N1-(3-fluorophenyl)propane-1,2-diamine;
(4S)-4-({[5-(4-amino-8,9-dimethoxybenzo[c]-2,7-naphthyridin-2-yl)pyridin-3-yl]oxy}methyl)-1-(3-fluorophenyl)imidazolidin-2-one;
(2S)-3-{[5-(4-amino-8,9-dimethoxybenzo[c]-2,7-naphthyridin-2-yl)pyridin-3-yl]oxy}-N1-(2-methoxyethyl)-N1-phenylpropane-1,2-diamine;
8,9-dimethoxy-2-{5-[(3R)-piperidin-3-yloxy]pyridine-3-yl}benzo[c]-2,7-naphthyridin-4-amine;
9-methoxy-8-(2-methoxyethoxy)-2-pyridin-3-ylbenzo[c]-2,7-naphthyridin-4-amine;
2-(5-{3-[benzyl(methyl)amino]prop-1-yn-1-yl}pyridin-3-yl)-8,9-dimethoxybenzo[c]-2,7-naphthyridin-4-amine;
2-(5-{[(2S)-2-aminopropyl]oxy}pyridin-3-yl)-8,9-dimethoxybenzo[c]-2,7-naphthyridin-4-amine;
2-(5-{[(2R)-2-aminopropyl]oxy}pyridin-3-yl)-8,9-dimethoxybenzo[c]-2,7-naphthyridin-4-amine;
2-(5-{[(2S)-2-amino-3-(3-chlorophenyl)propyl]oxy}pyridin-3-yl)-8,9-dimethoxybenzo[c]-2,7-naphthyridin-4-amine;
2-[5-({(2R)-2-amino-3-[3-(trifluoromethyl)phenyl]propyl}oxy)pyridin-3-yl]-8,9-dimethoxybenzo[c]-2,7-naphthyridin-4-amine;
(2S)-3-{[5-(4-amino-8,9-dimethoxybenzo[c]-2,7-naphthyridin-2-yl)pyridin-3-yl]oxy}-N1-benzyl-N1-methylpropane-1,2-diamine;
2-(5-{[(2S)-2-amino-3-(4-fluorophenyl)propyl]oxy}pyridin-3-yl)-9-(benzyloxy)-8-methoxybenzo[c]-2,7-naphthyridin-4-amine;
(2R)-3-{[5-(4-amino-8,9-dimethoxybenzo[c]-2,7-naphthyridin-2-yl)pyridin-3-yl]oxy}-2-[(tert-butoxycarbonyl)amino]propyl methanesulfonate;
tert-butyl [(1S)-2-{[5-(4-amino-8,9-dimethoxybenzo[c]-2,7-naphthyridin-2-yl)pyridin-3-yl]oxy}-1-(hydroxymethyl)ethyl]carbamate;
2-[5-({(2S)-2-amino-3-[3-(trifluoromethyl)phenyl]propyl}oxy)pyridin-3-yl]-8,9-dimethoxybenzo[c]-2,7-naphthyridin-4-amine;
benzyl [4-((2S)-2-amino-3-{[5-(4-amino-8,9-dimethoxybenzo[c]-2,7-naphthyridin-2-yl)pyridin-3-yl]oxy}propyl)phenyl]carbamate;
2-(5-{[(2S)-2-amino-3-(3-chlorophenyl)propyl]oxy}pyridin-3-yl)-N-hydroxy-8,9-dimethoxybenzo[c]-2,7-naphthyridin-4-amine;
8,9-dimethoxy-2-[5-(3-{methyl[(1R)-1-methyl-2-phenylethyl]amino}prop-1-yn-1-yl)pyridin-3-yl]benzo[c]-2,7-naphthyridin-4-amine;
(2S)-3-{[5-(4-amino-8,9-dimethoxybenzo[c]-2,7-naphthyridin-2-yl)pyridin-3-yl]oxy}-N1-ethyl-N1-(pyridin-4-ylmethyl)propane-1,2-diamine;
2-(5-{[(2S)-2-amino-3-(4-pyridin-2-ylpiperazin-1-yl)propyl]oxy}pyridin-3-yl)-8,9-dimethoxybenzo[c]-2,7-naphthyridin-4-amine;
2-(5-{[(2S)-2-amino-3-(4-aminophenyl)propyl]oxy}pyridin-3-yl)-8,9-dimethoxybenzo[c]-2,7-naphthyridin-4-amine;
2-(5-{[(2R)-2-aminobutyl]oxy}pyridin-3-yl)-8,9-dimethoxybenzo[c]-2,7-naphthyridin-4-amine;
tert-butyl [(1S)-1-({[5-(4-amino-8,9-dimethoxybenzo[c]-2,7-naphthyridin-2-yl)pyridin-3-yl]oxy}methyl)propyl]carbamate;
N-((1R)-2-{[5-(4-amino-8,9-dimethoxybenzo[c]-2,7-naphthyridin-2-yl)pyridin-3-yl]oxy}-1-benzylethyl)formamide;
2-(5-bromopyridin-3-yl)-8,9-dimethoxybenzo[c]-2,7-naphthyridin-4-amine;
2-(5-{[(2S)-2-aminobutyl]oxy}pyridin-3-yl)-8,9-dimethoxybenzo[c]-2,7-naphthyridin-4-amine;
2-(5-{[(2S)-2-amino-3-(1H-indol-3-yl)propyl]oxy}pyridin-3-yl)-8,9-dimethoxybenzo[c]-2,7-naphthyridin-4-amine;
2-(5-{[(2S)-2-amino-3-(1H-indol-3-yl)propyl]oxy}pyridin-3-yl)-N-hydroxy-8,9-dimethoxybenzo[c]-2,7-naphthyridin-4-amine;
(2S)-2-amino-3-{[5-(4-amino-8,9-dimethoxybenzo[c]-2,7-naphthyridin-2-yl)pyridin-3-yl]oxy}propan-1-ol;
2-(5-{[(2S)-2-amino-3-(1H-imidazol-1-yl)propyl]oxy}pyridin-3-yl)-8,9-dimethoxybenzo[c]-2,7-naphthyridin-4-amine;
(2S)-3-{[5-(4-amino-8,9-dimethoxybenzo[c]-2,7-naphthyridin-2-yl)pyridin-3-yl]oxy}-N1-(3-fluorobenzyl)-N1-methylpropane-1,2-diamine;
(2R)-2-amino-3-{[5-(4-amino-8,9-dimethoxybenzo[c]-2,7-naphthyridin-2-yl)pyridin-3-yl]oxy}propyl methanesulfonate;
(2S)-3-{[5-(4-amino-8,9-dimethoxybenzo[c]-2,7-naphthyridin-2-yl)pyridin-3-yl]oxy}-N1-methyl-N1-(pyridin-3-ylmethyl)propane-1,2-diamine;
2-(5-{[(2S)-2-amino-3-(4,4-diethoxypiperidin-1-yl)propyl]oxy}pyridin-3-yl)-8,9-dimethoxybenzo[c]-2,7-naphthyridin-4-amine;
1-[(2S)-2-amino-3-{[5-(4-amino-8,9-dimethoxybenzo[c]-2,7-naphthyridin-2-yl)pyridin-3-yl]oxy}propyl]piperidin-4-one;
2-{5-[2-(ethylamino)ethoxy]pyridin-3-yl}-8,9-dimethoxybenzo[c]-2,7-naphthyridin-4-amine;
tert-butyl-2-(5-(4-amino-8,9-dimethoxybenzo[c][2,7]naphthyridin-2-yl)pyridin-3-yloxy)ethyl(ethyl)carbamate;
tert-butyl-2-(5-(4-amino-8,9-dimethoxybenzo[c][2,7]naphthyridin-2-yl)pyridin-3-yloxy)ethylcarbamate;
tert-butyl-2-(5-(4-amino-8,9-dimethoxybenzo[c][2,7]naphthyridin-2-yl)pyridin-3-yloxy)ethyl (benzyl)carbamate;
2-(5-(2-aminoethoxy)pyridin-3-yl)-8,9-dimethoxybenzo[c][2,7]naphthyridin-4-amine;
tert-Butyl-2-(5-(4-amino-8,9-dimethoxybenzo[c][2,7]naphthyridin-2-yl)pyridin-3-yloxy)ethyl(4-fluorobenzyl)carbamate;
2-{5-[(3S)-3-amino-4-phenylbut-1-yn-1-yl]pyridin-3-yl}-8,9-dimethoxybenzo[c]-2,7-naphthyridin-4-amine;
N1-[5-(4-Amino-8,9-dimethoxy-benzo[c][2,7]naphthyridin-2-yl)-pyridin-3-yl]-propane-1,2-diamine;
N2-[5-(4-Amino-8,9-dimethoxy-benzo[c][2,7]naphthyridin-2-yl)-pyridin-3-yl]-propane-1,2-diamine;
2-(5-{[(2S)-2-amino-3-phenylpropyl]oxy}pyridin-3-yl)-9-methoxy-N8-(2-methoxyethyl)-N8-methylbenzo[c]-2,7-naphthyridine-4,8-diamine;
2-(5-{[(2S)-2-amino-3-phenylpropyl]oxy}pyridin-3-yl)-8-(ethylthio)-9-methoxybenzo[c]-2,7-naphthyridin-4-amine;
2-(5-{[(2S)-2-amino-3-phenylpropyl]oxy}pyridin-3-yl)-9-methoxy-8-morpholin-4-ylbenzo[c]-2,7-naphthyridin-4-amine;
8,9-dimethoxy-2-(2-pyridin-3-ylethyl)benzo[c]-2,7-naphthyridin-4-amine;
8,9-dimethoxy-2-methylbenzo[c]-2,7-naphthyridin-4-amine,
   and a pharmaceutically acceptable salt thereof.

Illustrative examples of Benzo[c][2,7]naphthyridine Derivatives of Formula (I), (Ia) and/or (Ib) include:
8,9-dimethoxy-2-pyridin-3-ylbenzo[c][2,7]naphthyridin-4-amine;
tert-butyl [(1R)-1-({[5-(4-amino-8,9-dimethoxybenzo[c][2,7]naphthyridin-2-yl)pyridin-3-yl]oxy}methyl)propyl]carbamate;
2-(5-{[(2S)-2-amino-3-pyridin-4-ylpropyl]oxy}pyridin-3-yl)-8,9-dimethoxybenzo[c]-2,7-naphthyridin-4-amine;
2-(5-{[(2S)-2-amino-3-phenylpropyl]oxy}pyridin-3-yl)-9-(benzyloxy)-8-methoxybenzo[c]-2,7-naphthyridin-4-amine;
2-(5-{[(2S)-2-amino-3-phenylpropyl]oxy}pyridin-3-yl)-9-methoxy-8-(pyridin-3-ylmethoxy)benzo[c]-2,7-naphthyridin-4-amine;
(2S)-3-{[5-(4-amino-8,9-dimethoxybenzo[c]-2,7-naphthyridin-2-yl)pyridin-3-yl]oxy}-N1,N1-diethylpropane-1,2-diamine;
(2S)-3-{[5-(4-amino-8,9-dimethoxybenzo[c]-2,7-naphthyridin-2-yl)pyridin-3-yl]oxy}-N1-(2-furylmethyl)-N1-methylpropane-1,2-diamine;
tert-butyl {(1R)-3-[5-(4-amino-8,9-dimethoxybenzo[c]-2,7-naphthyridin-2-yl)pyridin-3-yl]-1-benzylpropyl}carbamate;
2-{5-[(3R)-3-amino-4-phenylbutyl]pyridin-3-yl}-8,9-dimethoxybenzo[c]-2,7-naphthyridin-4-amine;
2-{5-[(2S)-aziridin-2-ylmethoxy]pyridin-3-yl}-8,9-dimethoxybenzo[c]-2,7-naphthyridin-4-amine;
(2S)-3-{[5-(4-amino-8,9-dimethoxybenzo[c]-2,7-naphthyridin-2-yl)pyridin-3-yl]oxy}-N1-methyl-N1-(2-pyridin-2-ylethyl)propane-1,2-diamine;
(2S)-3-{[5-(4-amino-8,9-dimethoxybenzo[c]-2,7-naphthyridin-2-yl)pyridin-3-yl]oxy}-N1-(4-methoxybenzyl)-N1-methylpropane-1,2-diamine;
(2S)-3-{[5-(4-amino-8,9-dimethoxybenzo[c]-2,7-naphthyridin-2-yl)pyridin-3-yl]oxy}-N1-(4-fluorobenzyl)-N1-methylpropane-1,2-diamine;
(2S)-3-{[5-(4-amino-8,9-dimethoxybenzo[c]-2,7-naphthyridin-2-yl)pyridin-3-yl]oxy}-N1-(2-chloro-6-fluorobenzyl)-N1-ethylpropane-1,2-diamine;
(2S)-3-{[5-(4-amino-8,9-dimethoxybenzo[c]-2,7-naphthyridin-2-yl)pyridin-3-yl]oxy}-N1-(2-chlorobenzyl)-N1-methylpropane-1,2-diamine;
(2S)-3-{[5-(4-amino-8,9-dimethoxybenzo[c]-2,7-naphthyridin-2-yl)pyridin-3-yl]oxy}-N1-methyl-N1-(pyridin-2-ylmethyl)propane-1,2-diamine;
(2S)-3-{[5-(4-amino-8,9-dimethoxybenzo[c]-2,7-naphthyridin-2-yl)pyridin-3-yl]oxy}-N1-(3-methoxypropyl)-N1-(pyridin-2-ylmethyl)propane-1,2-diamine;
tert-butyl [(1R)-2-{[5-(4-amino-8,9-dimethoxybenzo[c]-2,7-naphthyridin-2-yl)pyridin-3-yl]oxy}-1-(hydroxymethyl)ethyl]carbamate;
3-{[(2S)-2-amino-3-{[5-(4-amino-8,9-dimethoxybenzo[c]-2,7-naphthyridin-2-yl)pyridin-3-yl]oxy}propyl](benzyl)amino}propan-1-ol;
2-{[5-(4-amino-8,9-dimethoxybenzo[c]-2,7-naphthyridin-2-yl)pyridin-3-yl]oxy}propane-1,3-diamine;
(2S)-3-{[5-(4-amino-8,9-dimethoxybenzo[c]-2,7-naphthyridin-2-yl)pyridin-3-yl]oxy}-2-[(tert-butoxycarbonyl)amino]propyl methanesulfonate;
2-(5-{2-[(4-fluorobenzyl)amino]ethoxy}pyridin-3-yl)-8,9-dimethoxybenzo[c]-2,7-naphthyridin-4-amine;
(2S)-2-amino-3-{[5-(4-amino-8,9-dimethoxybenzo[c]-2,7-naphthyridin-2-yl)pyridin-3-yl]oxy}propyl methanesulfonate;
2-(5-{[(2R)-2-amino-3-(benzylthio)propyl]oxy}pyridin-3-yl)-8,9-dimethoxybenzo[c]-2,7-naphthyridin-4-amine;
(2R)-3-{[5-(4-amino-8,9-dimethoxybenzo[c][2,7]naphthyridin-2-yl)pyridin-3-yl]oxy}-N1-ethyl-N1-(pyridin-4-ylmethyl)propane-1,2-diamine;
2-(5-{2-[benzyl(methyl)amino]ethoxy}pyridin-3-yl)-8,9-dimethoxybenzo[c]-2,7-naphthyridin-4-amine;
2-[5-({(2R)-2-amino-3-[(4-methoxybenzyl)thio]propyl}oxy)pyridin-3-yl]-8,9-dimethoxybenzo[c]-2,7-naphthyridin-4-amine;
2-[5-({(2R)-2-amino-3-[(4-chlorobenzyl)thio]propyl}oxy)pyridin-3-yl]-8,9-dimethoxybenzo[c]-2,7-naphthyridin-4-amine;
2-(5-{[(2S)-2-amino-3-phenylpropyl]oxy}pyridin-3-yl)-8-methoxy-9-(2-methoxyethoxy)benzo[c]-2,7-naphthyridin-4-amine;
2-[5-({(2R)-2-amino-3-[(2-phenylethyl)thio]propyl}oxy)pyridin-3-yl]-8,9-dimethoxybenzo[c]-2,7-naphthyridin-4-amine;
(2R)-3-{[5-(4-amino-8,9-dimethoxybenzo[c]-2,7-naphthyridin-2-yl)pyridin-3-yl]oxy}-N1-(3-fluorobenzyl)-N1-methylpropane-1,2-diamine;
(2R)-3-{[5-(4-amino-8,9-dimethoxybenzo[c][2,7]naphthyridin-2-yl)pyridin-3-yl]oxy}-N1-methyl-N1-(pyridin-3-ylmethyl)propane-1,2-diamine;
1-ylpropoxy)benzo[c]-2,7-naphthyridin-4-amine
2-(5-{[(2S)-2-amino-3-phenylpropyl]oxy}pyridin-3-yl)-9-methoxy-8-(3-pyrrolidin-;
2-[5-({(2R)-2-amino-3-[(4-fluorobenzyl)thio]propyl}oxy)pyridin-3-yl]-8,9-dimethoxybenzo[c]-2,7-naphthyridin-4-amine;
2-(5-{[(2R)-2-amino-3-{[3-(trifluoromethyl)benzyl]thio}propyl]oxy}pyridin-3-yl)-8,9-dimethoxybenzo[c]-2,7-naphthyridin-4-amine;
2-{5-[(2R)-aziridin-2-ylmethoxy]pyridin-3-yl}-8,9-dimethoxybenzo[c]-2,7-naphthyridin-4-amine;
2-(5-{[(2S)-2-amino-3-phenylpropyl]oxy}pyridin-3-yl)-8-methoxy-9-(2-morpholin-4-ylethoxy)benzo[c]-2,7-naphthyridin-4-amine;
(2R)-3-{[5-(4-amino-8,9-dimethoxybenzo[c]-2,7-naphthyridin-2-yl)pyridin-3-yl]oxy}-N1-(4-fluorobenzyl)-N1-methylpropane-1,2-diamine;
2-{[(2S)-2-amino-3-{[5-(4-amino-8,9-dimethoxybenzo[c]-2,7-naphthyridin-2-yl)pyridin-3-yl]oxy}propyl](pyridin-3-ylmethyl)amino}ethanol;
2-(5-{[(2S)-2-amino-3-(2-pyridin-4-ylpyrrolidin-1-yl)propyl]oxy}pyridin-3-yl)-8,9-dimethoxybenzo[c]-2,7-naphthyridin-4-amine;
(2S)-3-{[5-(4-amino-8,9-dimethoxybenzo[c]-2,7-naphthyridin-2-yl)pyridin-3-yl]oxy}-N1-(3-methoxypropyl)-N1-(pyridin-4-ylmethyl)propane-1,2-diamine;
(2S)-3-{[5-(4-amino-8,9-dimethoxybenzo[c]-2,7-naphthyridin-2-yl)pyridin-3-yl]oxy}-N1-(3-chlorobenzyl)-N1-methylpropane-1,2-diamine;
(2S)-3-{[5-(4-amino-8,9-dimethoxybenzo[c]-2,7-naphthyridin-2-yl)pyridin-3-yl]oxy}-N1-(2,5-difluorobenzyl)-N1-methylpropane-1,2-diamine;
(2S)-3-{[5-(4-amino-8,9-dimethoxybenzo[c]-2,7-naphthyridin-2-yl)pyridin-3-yl]oxy}-N1-(2,4-difluorobenzyl)-N1-methylpropane-1,2-diamine;
(2S)-3-{[5-(4-amino-8,9-dimethoxybenzo[c]-2,7-naphthyridin-2-yl)pyridin-3-yl]oxy}-N1-methyl-N1-(pyridin-4-ylmethyl)propane-1,2-diamine;
(2R)-3-{[5-(4-amino-8,9-dimethoxybenzo[c]-2,7-naphthyridin-2-yl)pyridin-3-yl]oxy}-N1-benzyl-N1-ethylpropane-1,2-diamine;
(2R)-3-{[5-(4-amino-8,9-dimethoxybenzo[c]-2,7-naphthyridin-2-yl)pyridin-3-yl]oxy}-N1-benzyl-N1-methylpropane-1,2-diamine;
8,9-dimethoxy-2-{5-[2-(methylamino)ethoxy]pyridin-3-yl}benzo[c]-2,7-naphthyridin-4-amine;
(2R)-3-{[5-(4-amino-8,9-dimethoxybenzo[c]-2,7-naphthyridin-2-yl)pyridin-3-yl]oxy}-N1-(2-chlorobenzyl)-N1-methylpropane-1,2-diamine;
(2S)-3-{[5-(4-amino-8,9-dimethoxybenzo[c]-2,7-naphthyridin-2-yl)pyridin-3-yl]oxy}-N1-(4-fluorobenzyl)propane-1,2-diamine;
(2S)-3-{[5-(4-amino-8,9-dimethoxybenzo[c]-2,7-naphthyridin-2-yl)pyridin-3-yl]oxy}-N1-(pyridin-4-ylmethyl)propane-1,2-diamine;
(2S)-3-{[5-(4-amino-8,9-dimethoxybenzo[c]-2,7-naphthyridin-2-yl)pyridin-3-yl]oxy}-N1-(4-chloro-3-fluorobenzyl)propane-1,2-diamine;
(2S)-3-{[5-(4-amino-8,9-dimethoxybenzo[c]-2,7-naphthyridin-2-yl)pyridin-3-yl]oxy}-N1-[(2S)-pyrrolidin-2-ylmethyl]propane-1,2-diamine;
2-[5-({(2R)-2-amino-3-[(2-furylmethyl)thio]propyl}oxy)pyridin-3-yl]-8,9-dimethoxybenzo[c]-2,7-naphthyridin-4-amine;
(2R)-3-{[5-(4-amino-8,9-dimethoxybenzo[c]-2,7-naphthyridin-2-yl)pyridin-3-yl]oxy}-N1-(2,5-difluorobenzyl)-N1-methylpropane-1,2-diamine;
2-[5-({(2R)-2-amino-3-[(2-pyridin-4-ylethyl)thio]propyl}oxy)pyridin-3-yl]-8,9-dimethoxybenzo[c]-2,7-naphthyridin-4-amine;
(2R)-3-{[5-(4-amino-8,9-dimethoxybenzo[c]-2,7-naphthyridin-2-yl)pyridin-3-yl]oxy}-N1-(3,5-difluorobenzyl)propane-1,2-diamine;
(2R)-3-{[5-(4-amino-8,9-dimethoxybenzo[c]-2,7-naphthyridin-2-yl)pyridin-3-yl]oxy}-N1-(2-chloro-6-fluorobenzyl)-N1-ethylpropane-1,2-diamine;
tert-butyl [(1R)-1-({[5-(4-amino-8,9-dimethoxybenzo[c]-2,7-naphthyridin-2-yl)pyridin-3-yl]oxy}methyl)-3-methylbutyl]carbamate;
tert-butyl [(1S)-1-({[5-(4-amino-8,9-dimethoxybenzo[c]-2,7-naphthyridin-2-yl)pyridin-3-yl]oxy}methyl)-3-methylbutyl]carbamate;
2-(5-{[(2S)-2-amino-4-methylpentyl]oxy}pyridin-3-yl)-8,9-dimethoxybenzo[c]-2,7-naphthyridin-4-amine;
2-(5-{[(2R)-2-amino-4-methylpentyl]oxy}pyridin-3-yl)-8,9-dimethoxybenzo[c]-2,7-naphthyridin-4-amine;
(2R)-3-{[5-(4-amino-8,9-dimethoxybenzo[c]-2,7-naphthyridin-2-yl)pyridin-3-yl]oxy}-N1-(2-methoxyethyl)-N1-methylpropane-1,2-diamine;
(2R)-3-{[5-(4-amino-8,9-dimethoxybenzo[c]-2,7-naphthyridin-2-yl)pyridin-3-yl]oxy}-N1-(2-furylmethyl)-N1-methylpropane-1,2-diamine;
(2R)-3-{[5-(4-amino-8,9-dimethoxybenzo[c]-2,7-naphthyridin-2-yl)pyridin-3-yl]oxy}-N1-ethyl-N1-[1-(2-furyl)-2-phenylethyl]propane-1,2-diamine;
tert-butyl [(1S)-2-{[5-(4-amino-8,9-dimethoxybenzo[c]-2,7-naphthyridin-2-yl)pyridin-3-yl]oxy}-1-(cyclohexylmethyl)ethyl]carbamate;
tert-butyl [(1S,2S)-1-({[5-(4-amino-8,9-dimethoxybenzo[c]-2,7-naphthyridin-2-yl)pyridin-3-yl]oxy}methyl)-2-methylbutyl]carbamate;
2-(5-{2-[(1-ethylpropyl)amino]ethoxy}pyridin-3-yl)-8,9-dimethoxybenzo[c]-2,7-naphthyridin-4-amine;
2-(5-{[(2S)-2-amino-3-cyclohexylpropyl]oxy}pyridin-3-yl)-8,9-dimethoxybenzo[c]-2,7-naphthyridin-4-amine;
2-(5-{[(2S,3S)-2-amino-3-methylpentyl]oxy}pyridin-3-yl)-8,9-dimethoxybenzo[c]-2,7-naphthyridin-4-amine;
tert-butyl (3-{[5-(4-amino-8,9-dimethoxybenzo[c]-2,7-naphthyridin-2-yl)pyridin-3-yl]oxy}propyl)carbamate;
2-[5-(3-aminopropoxy)pyridin-3-yl]-8,9-dimethoxybenzo[c]-2,7-naphthyridin-4-amine;
2-{4-[(2R)-2-amino-3-{[5-(4-amino-8,9-dimethoxybenzo[c]-2,7-naphthyridin-2-yl)pyridin-3-yl]oxy}propyl]piperazin-1-yl}-N-isopropylacetamide;
tert-butyl [(1R)-1-({[5-(4-amino-8,9-dimethoxybenzo[c]-2,7-naphthyridin-2-yl)pyridin-3-yl]oxy}methyl)-2-methylpropyl]carbamate;
tert-butyl [(1S)-1-({[5-(4-amino-8,9-dimethoxybenzo[c]-2,7-naphthyridin-2-yl)pyridin-3-yl]oxy}methyl)-2-methylpropyl]carbamate;
2-(5-{[(2R)-2-amino-3-methylbutyl]oxy}pyridin-3-yl)-8,9-dimethoxybenzo[c]-2,7-naphthyridin-4-amine;
2-(5-{[(2S)-2-amino-3-methylbutyl]oxy}pyridin-3-yl)-8,9-dimethoxybenzo[c]-2,7-naphthyridin-4-amine;
2-(5-{[(2R)-2-amino-3-(4-methylpiperazin-1-yl)propyl]oxy}pyridin-3-yl)-8,9-dimethoxybenzo[c][2,7]naphthyridin-4-amine;
2-(5-ethoxypyridin-3-yl)-8,9-dimethoxybenzo[c][2,7]naphthyridin-4-amine;
2-(5-fluoropyridin-3-yl)-8,9-dimethoxybenzo[c][2,7]naphthyridin-4-amine;
2-[5-(azetidin-3-yloxy)pyridin-3-yl]-8,9-dimethoxybenzo[c][2,7]naphthyridin-4-amine;
tert-butyl (3R)-3-{[5-(4-amino-8,9-dimethoxybenzo[c][2,7]naphthyridin-2-yl)pyridin-3-yl]oxy}piperidine-1-carboxylate;
tert-butyl 4-(4-amino-8,9-dimethoxybenzo[c][2,7]naphthyridin-2-yl)piperidine-1-carboxylate;
8,9-dimethoxy-2-piperidin-4-ylbenzo[c][2,7]naphthyridin-4-amine;
3-{[5-(4-amino-8,9-dimethoxybenzo[c][2,7]naphthyridin-2-yl)pyridin-3-yl]oxy}propane-1,2-diol;
8,9-dimethoxy-2-pyridin-2-ylbenzo[c][2,7]naphthyridin-4-amine;
tert-butyl 2-(4-amino-8,9-dimethoxybenzo[c][2,7]naphthyridin-2-yl)pyrrolidine-1-carboxylate;
tert-butyl 3-(4-amino-8,9-dimethoxybenzo[c][2,7]naphthyridin-2-yl)pyrrolidine-1-carboxylate;
tert-butyl 2-(4-amino-8,9-dimethoxybenzo[c][2,7]naphthyridin-2-yl)piperidine-1-carboxylate;
tert-butyl 3-(4-amino-8,9-dimethoxybenzo[c][2,7]naphthyridin-2-yl)piperidine-1-carboxylate;
tert-butyl [(1S,2R)-2-{[5-(4-amino-8,9-dimethoxybenzo[c][2,7]naphthyridin-2-yl)pyridin-3-yl]oxy}cyclohexyl]carbamate;
2-[(3-{[5-(4-amino-8,9-dimethoxybenzo[c][2,7]naphthyridin-2-yl)pyridin-3-yl]oxy}propyl)amino]propane-1,3-diol;
8,9-dimethoxy-2-pyrrolidin-2-ylbenzo[c][2,7]naphthyridin-4-amine;
8,9-dimethoxy-2-pyrrolidin-3-ylbenzo[c][2,7]naphthyridin-4-amine;
8,9-dimethoxy-2-piperidin-2-ylbenzo[c][2,7]naphthyridin-4-amine;
8,9-dimethoxy-2-piperidin-3-ylbenzo[c][2,7]naphthyridin-4-amine;
2-(5-{[(1R,2S)-2-aminocyclohexyl]oxy}pyridin-3-yl)-8,9-dimethoxybenzo[c][2,7]naphthyridin-4-amine;
tert-butyl [(1S,2S)-2-{[5-(4-amino-8,9-dimethoxybenzo[c][2,7]naphthyridin-2-yl)pyridin-3-yl]oxy}cyclopentyl]carbamate;
2-(5-{[(1S,2S)-2-aminocyclopentyl]oxy}pyridin-3-yl)-8,9-dimethoxybenzo[c][2,7]naphthyridin-4-amine;
(2S)-3-{[5-(4-amino-8,9-dimethoxybenzo[c][2,7]naphthyridin-2-yl)pyridin-3-yl]oxy}-N1-methyl-N1-(1-methylpiperidin-4-yl)propane-1,2-diamine;
(2S)-3-{[5-(4-amino-8,9-dimethoxybenzo[c][2,7]naphthyridin-2-yl)pyridin-3-yl]oxy}-N1-[3-(dimethylamino)propyl]-N1-methylpropane-1,2-diamine;
(2S)-3-{[5-(4-amino-8,9-dimethoxybenzo[c][2,7]naphthyridin-2-yl)pyridin-3-yl]oxy}-N1-[2-(dimethylamino)ethyl]-N1-methylpropane-1,2-diamine;
(2S)-3-{[5-(4-amino-8,9-dimethoxybenzo[c][2,7]naphthyridin-2-yl)pyridin-3-yl]oxy}-N1,N1-bis(2-methoxyethyl)propane-1,2-diamine;
2-[5-(1H-imidazol-5-ylmethoxy)pyridin-3-yl]-8,9-dimethoxybenzo[c][2,7]naphthyridin-4-amine;
8,9-dimethoxy-2-(2,2,8-trimethyl-4H-[1,3]dioxino[4,5-c]pyridin-5-yl)benzo[c][2,7]naphthyridin-4-amine;
8,9-dimethoxy-2-(6-methylpyridin-3-yl)benzo[c][2,7]naphthyridin-4-amine;
8,9-dimethoxy-2-(1-methyl-1H-imidazol-2-yl)benzo[c][2,7]naphthyridin-4-amine;
8,9-dimethoxy-2-pyrimidin-2-ylbenzo[c][2,7]naphthyridin-4-amine;
2-(1H-imidazol-2-yl)-8,9-dimethoxybenzo[c][2,7]naphthyridin-4-amine;
8,9-dimethoxy-2-pyrazin-2-ylbenzo[c][2,7]naphthyridin-4-amine;
(2S)-3-({5-[4-amino-9-methoxy-8-(2-methoxyethoxy)benzo[c][2,7]naphthyridin-2-yl]pyridin-3-yl}oxy)-N1-ethyl-N1-(pyridin-4-ylmethyl)propane-1,2-diamine;
2-{[5-(4-amino-8,9-dimethoxybenzo[c][2,7]naphthyridin-2-yl)pyridin-3-yl]oxy}ethanol;
2-(5-{[(2R)-2-aminobutyl]oxy}pyridin-3-yl)-9-methoxy-8-(2-methoxyethoxy)benzo[c][2,7]naphthyridin-4-amine;
8,9-dimethoxy-2-(4H-1,2,4-triazol-3-yl)benzo[c][2,7]naphthyridin-4-amine;
2-(5-{[(2S)-2-amino-3-(pyridin-3-yloxy)propyl]oxy}pyridin-3-yl)-8,9-dimethoxybenzo[c][2,7]naphthyridin-4-amine;
2-(5-{[(2S)-2-amino-3-(3-fluorophenoxy)propyl]oxy}pyridin-3-yl)-8,9-dimethoxybenzo[c][2,7]naphthyridin-4-amine;
2-(5-{[(2R)-2-amino-3-(2-chloro-5-fluorophenoxy)propyl]oxy}pyridin-3-yl)-8,9-dimethoxybenzo[c][2,7]naphthyridin-4-amine;
2-(5-{[(2R)-2-amino-3-(2-chloro-4-fluorophenoxy)propyl]oxy}pyridin-3-yl)-8,9-dimethoxybenzo[c][2,7]naphthyridin-4-amine;
2-[5-({(2R)-2-amino-3-[(4-chloropyridin-2-yl)oxy]propyl}oxy)pyridin-3-yl]-8,9-dimethoxybenzo[c][2,7]naphthyridin-4-amine;
2-(5-{[(2S)-2-amino-3-(3-methoxyphenoxy)propyl]oxy}pyridin-3-yl)-8,9-dimethoxybenzo[c][2,7]naphthyridin-4-amine;
3-(4-amino-8,9-dimethoxybenzo[c][2,7]naphthyridin-2-yl)benzonitrile;
8,9-dimethoxy-2-[5-(methoxymethoxy)pyridin-3-yl]benzo[c][2,7]naphthyridin-4-amine;
2-(3-iodophenyl)-8,9-dimethoxybenzo[c][2,7]naphthyridin-4-amine;
2-(3-fluorophenyl)-8,9-dimethoxybenzo[c][2,7]naphthyridin-4-amine;
8-(2-chloroethoxy)-9-methoxy-2-pyridin-3-ylbenzo[c][2,7]naphthyridin-4-amine;
tert-butyl {(1R)-2-{[5-(4-amino-8,9-dimethoxybenzo[c][2,7]naphthyridin-2-yl)pyridin-3-yl]oxy}-1-[(3-fluorophenoxy)methyl]ethyl}carbamate;
2-(5-{[(2R)-2-(diethylamino)butyl]oxy}pyridin-3-yl)-8,9-dimethoxybenzo[c][2,7]naphthyridin-4-amine;
tert-butyl {(1R)-2-{[5-(4-amino-8,9-dimethoxybenzo[c][2,7]naphthyridin-2-yl)pyridin-3-yl]oxy}-1-[(3-methoxyphenoxy)methyl]ethyl}carbamate;
2-(5-{[(2R)-2-amino-3-(3-fluorophenoxy)propyl]oxy}pyridin-3-yl)-8,9-dimethoxybenzo[c][2,7]naphthyridin-4-amine;
2-(5-{[(2R)-2-amino-3-(3-methoxyphenoxy)propyl]oxy}pyridin-3-yl)-8,9-dimethoxybenzo[c][2,7]naphthyridin-4-amine;
8,9-dimethoxy-2-[3-(1H-pyrazol-1-yl)phenyl]benzo[c][2,7]naphthyridin-4-amine;
9-methoxy-8-(3-piperidin-1-ylpropoxy)-2-pyridin-3-ylbenzo[c][2,7]naphthyridin-4-amine;
2-(5-{[(2R)-2-aminobutyl]oxy}pyridin-3-yl)-8-(2-chloroethoxy)-9-methoxybenzo[c][2,7]naphthyridin-4-amine;
4-{[(2S)-2-amino-3-{[5-(4-amino-8,9-dimethoxybenzo[c][2,7]naphthyridin-2-yl)pyridin-3-yl]oxy}propyl]oxy}benzonitrile;
2-(5-{[(2R)-2-amino-3-(2-methoxyphenoxy)propyl]oxy}pyridin-3-yl)-8,9-dimethoxybenzo[c][2,7]naphthyridin-4-amine;
2-(5-{[(2S)-2-amino-3-(1H-pyrazol-1-yl)propyl]oxy}pyridin-3-yl)-8,9-dimethoxybenzo[c][2,7]naphthyridin-4-amine;
2-[(2S)-2-amino-3-{[5-(4-amino-8,9-dimethoxybenzo[c][2,7]naphthyridin-2-yl)pyridin-3-yl]oxy}propyl]-1H-isoindole-1,3(2H)-dione;
4-{[(2S)-2-amino-3-{[5-(4-amino-8,9-dimethoxybenzo[c][2,7]naphthyridin-2-yl)pyridin-3-yl]oxy}propyl]oxy}-6-methyl-2H-pyran-2-one;
2-(5-{[(2R)-2-amino-3-(quinolin-2-yloxy)propyl]oxy}pyridin-3-yl)-8,9-dimethoxybenzo[c][2,7]naphthyridin-4-amine;
8,9-dimethoxy-2-[3-(1H-pyrazol-4-yl)phenyl]benzo[c][2,7]naphthyridin-4-amine;
2-(5-{[(2R)-2-aminobutyl]oxy}pyridin-3-yl)-9-methoxy-8-(2,2,2-trifluoroethoxy)benzo[c][2,7]naphthyridin-4-amine;
tert-butyl [(1S)-2-{[5-(4-amino-8,9-dimethoxybenzo[c][2,7]naphthyridin-2-yl)pyridin-3-yl]oxy}-1-{[ethyl(pyridin-4-ylmethyl)amino]methyl}ethyl]carbamate;
2-[5-({(2R)-2-amino-3-[(6-chloropyridin-2-yl)oxy]propyl}oxy)pyridin-3-yl]-8,9-dimethoxybenzo[c][2,7]naphthyridin-4-amine;
2-(5-{[(2R)-2-amino-3-(pyridin-2-yloxy)propyl]oxy}pyridin-3-yl)-8,9-dimethoxybenzo[c][2,7]naphthyridin-4-amine;
2-(5-{[(2S)-2-amino-3-(3-chloro-4-fluorophenoxy)propyl]oxy}pyridin-3-yl)-8,9-dimethoxybenzo[c][2,7]naphthyridin-4-amine;
2-(5-{[(2S)-2-amino-3-(3-chlorophenoxy)propyl]oxy}pyridin-3-yl)-8,9-dimethoxybenzo[c][2,7]naphthyridin-4-amine;
2-(5-{[(2R)-2-(benzylamino)butyl]oxy}pyridin-3-yl)-8,9-dimethoxybenzo[c][2,7]naphthyridin-4-amine;
2-({[(1R)-1-({[5-(4-amino-8,9-dimethoxybenzo[c][2,7]naphthyridin-2-yl)pyridin-3-yl]oxy}methyl)propyl]amino}methyl)benzonitrile;
3-({[(1R)-1-({[5-(4-amino-8,9-dimethoxybenzo[c][2,7]naphthyridin-2-yl)pyridin-3-yl]oxy}methyl)propyl]amino}methyl)benzonitrile;
4-({[(1R)-1-({[5-(4-amino-8,9-dimethoxybenzo[c][2,7]naphthyridin-2-yl)pyridin-3-yl]oxy}methyl)propyl]amino}methyl)benzonitrile;
2-[5-({(2R)-2-[(3-aminobenzyl)amino]butyl}oxy)pyridin-3-yl]-8,9-dimethoxybenzo[c][2,7]naphthyridin-4-amine;
3-({[(1R)-1-({[5-(4-amino-8,9-dimethoxybenzo[c][2,7]naphthyridin-2-yl)pyridin-3-yl]oxy}methyl)propyl]amino}methyl)phenol;
8,9-dimethoxy-2-[5-({(2R)-2-[(4-methoxybenzyl)amino]butyl}oxy)pyridin-3-yl]benzo[c][2,7]naphthyridin-4-amine;
2-(5-{[(2R)-2-aminobutyl]oxy}pyridin-3-yl)-9-methoxy-8-(tetrahydro-2H-pyran-4-yloxy)benzo[c][2,7]naphthyridin-4-amine;
2-(5-{[(2R)-2-aminobutyl]oxy}pyridin-3-yl)-8-butoxy-9-methoxybenzo[c][2,7]naphthyridin-4-amine;
   and a pharmaceutically acceptable salt thereof.

Additional illustrative examples of Benzo[c][2,7]naphthyridine Derivatives of Formula (I), (Ia) and/or (Ib) include:
2-(5-bromopyridin-3-yl)-8,9-dimethoxybenzo[c]-2,7-naphthyridin-4-amine;
3-(4-amino-8,9-dimethoxybenzo[c][2,7]naphthyridin-2-yl)benzoic acid;
2-(5-{[(2R)-2-aminobutyl]oxy}pyridin-3-yl)-8-(2-chloroethoxy)-9-methoxybenzo[c][2,7]naphthyridin-4-amine;
2-(1H-imidazo[4,5-c]pyridin-1-yl)-8,9-dimethoxybenzo[c][2,7]naphthyridin-4-amine;
2-(1H-imidazo[4,5-c]pyridin-1-yl)-8,9-dimethoxybenzo[c][2,7]naphthyridin-4-amine;
8,9-dimethoxy-2-[3-(1,3-oxazol-5-yl)phenyl]benzo[c][2,7]naphthyridin-4-amine;
2-[5-(3-aminopropyl)pyridin-3-yl]-8,9-dimethoxybenzo[c][2,7]naphthyridin-4-amine;
10-methoxy-2-pyridin-3-ylbenzo[c][2,7]naphthyridin-4-amine;
2-(3-aminophenyl)-8,9-dimethoxybenzo[c][2,7]naphthyridin-4-amine;
2-(5-{[(2R)-2-amino-3-(2-fluorophenoxy)propyl]oxy}pyridin-3-yl)-8,9-dimethoxybenzo[c][2,7]naphthyridin-4-amine;
2-(5-{[(2R)-2-amino-3-(2,4-difluorophenoxy)propyl]oxy}pyridin-3-yl)-8,9-dimethoxybenzo[c][2,7]naphthyridin-4-amine;
2-(5-{[(2S)-2-amino-3-(3,4-difluorophenoxy)propyl]oxy}pyridin-3-yl)-8,9-dimethoxybenzo[c][2,7]naphthyridin-4-amine;
N-[(2S)-2-amino-3-{[5-(4-amino-8,9-dimethoxybenzo[c][2,7]naphthyridin-2-yl)pyridin-3-yl]oxy}propyl]-N-butylbenzenesulfonamide;
2-(5-{[(2R)-2-amino-3-(pyridin-2-ylthio)propyl]oxy}pyridin-3-yl)-8,9-dimethoxybenzo[c][2,7]naphthyridin-4-amine;
2-(5-{[(2R)-2-amino-3-(1H-imidazol-2-ylthio)propyl]oxy}pyridin-3-yl)-8,9-dimethoxybenzo[c][2,7]naphthyridin-4-amine;
2-[5-({(2R)-2-amino-3-[(1-methyl-1H-imidazol-2-yl)thio]propyl}oxy)pyridin-3-yl]-8,9-dimethoxybenzo[c][2,7]naphthyridin-4-amine;
2-(5-{[(2R)-2-aminobutyl]oxy}pyridin-3-yl)-8-(2-fluoroethoxy)-9-methoxybenzo[c][2,7]naphthyridin-4-amine;
2-(5-{[(2R)-2-aminobutyl]oxy}pyridin-3-yl)-9-(2-chloroethoxy)-8-methoxybenzo[c][2,7]naphthyridin-4-amine;
2-(5-{[(2R)-2-aminobutyl]oxy}pyridin-3-yl)-9-methoxy-8-(4,4,4-trifluorobutoxy)benzo[c][2,7]naphthyridin-4-amine;
2-(5-{[(2R)-2-aminobutyl]oxy}pyridin-3-yl)-8-(3-chloropropoxy)-9-methoxybenzo[c][2,7]naphthyridin-4-amine;
N-[(2S)-2-amino-3-{[5-(4-amino-8,9-dimethoxybenzo[c][2,7]naphthyridin-2-yl)pyridin-3-yl]oxy}propyl]-3-fluorobenzenesulfonamide;
2-[5-({(2R)-2-amino-3-[2-fluoro-5-(trifluoromethyl)phenoxy]propyl}oxy)pyridin-3-yl]-8,9-dimethoxybenzo[c][2,7]naphthyridin-4-amine;
2-(5-{[(2R)-2-amino-3-(phenylthio)propyl]oxy}pyridin-3-yl)-8,9-dimethoxybenzo[c][2,7]naphthyridin-4-amine;
2-[5-({(2R)-2-amino-3-[(2,4-dichlorophenyl)thio]propyl}oxy)pyridin-3-yl]-8,9-dimethoxybenzo[c][2,7]naphthyridin-4-amine;
N-[(2S)-2-amino-3-{[5-(4-amino-8,9-dimethoxybenzo[c][2,7]naphthyridin-2-yl)pyridin-3-yl]oxy}propyl]-N-methylbenzenesulfonamide;
2-(5-{[(2R)-2-amino-3-(pyridin-4-ylthio)propyl]oxy}pyridin-3-yl)-8,9-dimethoxybenzo[c][2,7]naphthyridin-4-amine;
tert-butyl [(1R)-1-({[5-(4-amino-9-hydroxy-8-methoxybenzo[c][2,7]naphthyridin-2-yl)pyridin-3-yl]oxy}methyl)propyl]carbamate;
4-amino-2-(5-{[(2R)-2-aminobutyl]oxy}pyridin-3-yl)-8-methoxybenzo[c][2,7]naphthyridin-9-ol
2-(4-bromo-3-fluorophenyl)-8,9-dimethoxybenzo[c][2,7]naphthyridin-4-amine;
2-(4-bromophenyl)-8,9-dimethoxybenzo[c][2,7]naphthyridin-4-amine;
2-(5-{[(2R)-2-amino-3-(pyrimidin-2-ylthio)propyl]oxy}pyridin-3-yl)-8,9-dimethoxybenzo[c][2,7]naphthyridin-4-amine;
2-(5-{[(2R)-2-amino-3-{[5-(trifluoromethyl)pyridin-2-yl]oxy}propyl]oxy}pyridin-3-yl)-8,9-dimethoxybenzo[c][2,7]naphthyridin-4-amine;
2-(5-{[(2R)-2-amino-3-(1,3-thiazol-2-ylthio)propyl]oxy}pyridin-3-yl)-8,9-dimethoxybenzo[c][2,7]naphthyridin-4-amine;
2-{[4-amino-2-(5-{[(2R)-2-aminobutyl]oxy}pyridin-3-yl)-9-methoxybenzo[c][2,7]naphthyridin-8-yl]oxy}ethanol;
{[4-amino-2-(5-{[(2R)-2-aminobutyl]oxy}pyridin-3-yl)-9-methoxybenzo[c][2,7]naphthyridin-8-yl]oxy}acetic acid;
2-(5-{[(2R)-2-aminobutyl]oxy}pyridin-3-yl)-8-methoxybenzo[c][2,7]naphthyridin-4-amine;
tert-butyl [(1R)-1-({[5-(4-amino-8,9-dihydroxybenzo[c][2,7]naphthyridin-2-yl)pyridin-3-yl]oxy}methyl)propyl]carbamate;
4-amino-2-(1H-imidazo[4,5-c]pyridin-1-yl)benzo[c][2,7]naphthyridine-8,9-diol;
8,9-dimethoxy-2-[5-(1-methyl-1H-pyrazol-4-yl)pyridin-3-yl]benzo[c][2,7]naphthyridin-4-amine;
2-(3-fluoro-2-methylphenyl)-8,9-dimethoxybenzo[c][2,7]naphthyridin-4-amine;
2-[5-(3-aminoprop-1-yn-1-yl)pyridin-3-yl]-8,9-dimethoxybenzo[c][2,7]naphthyridin-4-amine;
2-[5-(3-amino-3-methylbut-1-yn-1-yl)pyridin-3-yl]-8,9-dimethoxybenzo[c][2,7]naphthyridin-4-amine;
2-{5-[(3R)-3-aminopent-1-yn-1-yl]pyridin-3-yl}-8,9-dimethoxybenzo[c][2,7]naphthyridin-4-amine;
tert-butyl {(1R)-3-[5-(4-amino-8,9-dimethoxybenzo[c][2,7]naphthyridin-2-yl)pyridin-3-yl]-1-ethylprop-2-yn-1-yl}carbamate;
4-amino-2-(1H-imidazo[4,5-c]pyridin-1-yl)-8-methoxybenzo[c][2,7]naphthyridin-9-ol;
4-amino-2-(1H-imidazo[4,5-c]pyridin-1-yl)-9-methoxybenzo[c][2,7]naphthyridin-8-ol;
2-(1H-benzimidazol-1-yl)-8,9-dimethoxybenzo[c][2,7]naphthyridin-4-amine;
tert-butyl {(1R)-3-[5-(4-amino-8,9-dimethoxybenzo[c][2,7]naphthyridin-2-yl)pyridin-3-yl]-1-ethylpropyl}carbamate;
2-{5-[(3R)-3-aminopentyl]pyridin-3-yl}-8,9-dimethoxybenzo[c][2,7]naphthyridin-4-amine;
2-[5-(3-amino-3-methylbutyl)pyridin-3-yl]-8,9-dimethoxybenzo[c][2,7]naphthyridin-4-amine;
2-(5-{[(2R)-2-amino-3-(1H-benzimidazol-2-yloxy)propyl]oxy}pyridin-3-yl)-8,9-dimethoxybenzo[c][2,7]naphthyridin-4-amine;
2-(5-{[(2R)-2-amino-3-(isoquinolin-1-yloxy)propyl]oxy}pyridin-3-yl)-8,9-dimethoxybenzo[c][2,7]naphthyridin-4-amine;
(2S)-3-{[5-(4-amino-8,9-dimethoxybenzo[c][2,7]naphthyridin-2-yl)pyridin-3-yl]oxy}-N1-phenylpropane-1,2-diamine;
2-(5-{[(2R)-2-amino-3-(4,5-dihydro-1H-imidazol-2-yloxy)propyl]oxy}pyridin-3-yl)-8,9-dimethoxybenzo[c][2,7]naphthyridin-4-amine;
2-(5-{[(2R)-2-amino-3-(pyrimidin-2-yloxy)propyl]oxy}pyridin-3-yl)-8,9-dimethoxybenzo[c][2,7]naphthyridin-4-amine
(2S)-3-{[5-(4-amino-8,9-dimethoxybenzo[c][2,7]naphthyridin-2-yl)pyridin-3-yl]oxy}-N1-(3-fluoro-4-methylphenyl)propane-1,2-diamine;
2-{5-[2-(1-aminocyclohexyl)ethyl]pyridin-3-yl}-8,9-dimethoxybenzo[c][2,7]naphthyridin-4-amine;
2-{5-[(1-aminocyclohexyl)ethynyl]pyridin-3-yl}-8,9-dimethoxybenzo[c][2,7]naphthyridin-4-amine;
2-(2-bromo-1,3-thiazol-5-yl)-8,9-dimethoxybenzo[c][2,7]naphthyridin-4-amine;
8,9-dimethoxy-2-(1,3-thiazol-5-yl)benzo[c][2,7]naphthyridin-4-amine;
8,9-dimethoxy-2-(6-methoxypyridin-3-yl)benzo[c][2,7]naphthyridin-4-amine;
8,9-dimethoxy-2-(6-methoxypyridin-3-yl)benzo[c][2,7]naphthyridin-4-amine
2-chloro-8,9-dimethoxybenzo[c][2,7]naphthyridin-4-amine;
3-(4-amino-8,9-dimethoxybenzo[c][2,7]naphthyridin-2-yl)benzaldehyde;
2-(4-amino-8,9-dimethoxybenzo[c][2,7]naphthyridin-2-yl)benzoic acid;
4-(4-amino-8,9-dimethoxybenzo[c][2,7]naphthyridin-2-yl)benzaldehyde;
2-{6-[3-(dimethylamino)propoxy]pyridin-3-yl}-8,9-dimethoxybenzo[c][2,7]naphthyridin-4-amine;
8,9-dimethoxy-2-(6-morpholin-4-ylpyridin-3-yl)benzo[c][2,7]naphthyridin-4-amine;
(2S)-3-{[5-(4-amino-8,9-dimethoxybenzo[c][2,7]naphthyridin-2-yl)pyridin-3-yl]oxy}-N1-(3-fluorophenyl)-N1-methylpropane-1,2-diamine;
(2S)-3-{[5-(4-amino-8,9-dimethoxybenzo[c][2,7]naphthyridin-2-yl)pyridin-3-yl]oxy}-N1-(3-chloro-4-fluorophenyl)propane-1,2-diamine;
(2S)-3-{[5-(4-amino-8,9-dimethoxybenzo[c][2,7]naphthyridin-2-yl)pyridin-3-yl]oxy}-N1-(2,4-difluorophenyl)propane-1,2-diamine;
(2S)-3-{[5-(4-amino-8,9-dimethoxybenzo[c][2,7]naphthyridin-2-yl)pyridin-3-yl]oxy}-N1-tert-butylpropane-1,2-diamine;
(2S)-3-{[5-(4-amino-8,9-dimethoxybenzo[c][2,7]naphthyridin-2-yl)pyridin-3-yl]oxy}-N1-[2-(4-fluorophenyl)-1,1-dimethylethyl]propane-1,2-diamine;
2-(5-{[(2S)-2-amino-3-(2-amino-1H-benzimidazol-1-yl)propyl]oxy}pyridin-3-yl)-8,9-dimethoxybenzo[c][2,7]naphthyridin-4-amine;
methyl 3-(4-amino-8,9-dimethoxybenzo[c][2,7]naphthyridin-2-yl)benzoate;
8,9-dimethoxy-2-(2-morpholin-4-ylpyrimidin-5-yl)benzo[c][2,7]naphthyridin-4-amine;
5-(4-amino-8,9-dimethoxybenzo[c][2,7]naphthyridin-2-yl)thiophene-2-carbaldehyde;
8,9-dimethoxy-2-{6-[(2-morpholin-4-ylethyl)amino]pyridin-3-yl}benzo[c][2,7]naphthyridin-4-amine;
2-(3-furyl)-8,9-dimethoxybenzo[c][2,7]naphthyridin-4-amine;
2-(6-fluoropyridin-3-yl)-8,9-dimethoxybenzo[c][2,7]naphthyridin-4-amine;
2-(6-aminopyridin-3-yl)-8,9-dimethoxybenzo[c][2,7]naphthyridin-4-amine;
(2S)-3-{[5-(4-amino-8,9-dimethoxybenzo[c][2,7]naphthyridin-2-yl)pyridin-3-yl]oxy}-N1-cyclohexylpropane-1,2-diamine;
(2S)-3-{[5-(4-amino-8,9-dimethoxybenzo[c][2,7]naphthyridin-2-yl)pyridin-3-yl]oxy}-N1-(1-methyl-1-phenylethyl)propane-1,2-diamine;
2-[5-({(2S)-2-amino-3-[4-(1,3-thiazol-2-yl)-1H-pyrazol-1-yl]propyl}oxy)pyridin-3-yl]-8,9-dimethoxybenzo[c][2,7]naphthyridin-4-amine;
(2S)-3-{[5-(4-amino-8,9-dimethoxybenzo[c][2,7]naphthyridin-2-yl)pyridin-3-yl]oxy}-N1-(1-methyl-1-pyridin-3-ylethyl)propane-1,2-diamine
(2S)-3-{[5-(4-amino-8,9-dimethoxybenzo[c][2,7]naphthyridin-2-yl)pyridin-3-yl]oxy}-N1-(1-methyl-1-pyridin-2-ylethyl)propane-1,2-diamine;
(2S)-3-{[5-(4-amino-8,9-dimethoxybenzo[c][2,7]naphthyridin-2-yl)pyridin-3-yl]oxy}-N1-(tetrahydro-2H-pyran-4-yl)propane-1,2-diamine;
(2S)-3-{[5-(4-amino-8,9-dimethoxybenzo[c][2,7]naphthyridin-2-yl)pyridin-3-yl]oxy}-N1-(2-pyridin-4-ylethyl)propane-1,2-diamine;
tert-butyl [(1R)-3-[5-(4-amino-8,9-dimethoxybenzo[c][2,7]naphthyridin-2-yl)pyridin-3-yl]-1-(hydroxymethyl)propyl]carbamate;
2-[5-({(2S)-2-amino-3-[(2S)-2-(1,3-thiazol-2-yl)pyrrolidin-1-yl]propyl}oxy)pyridin-3-yl]-8,9-dimethoxybenzo[c][2,7]naphthyridin-4-amine;
2-(3,5-difluorophenyl)-8,9-dimethoxybenzo[c][2,7]naphthyridin-4-amine;
2-(3,4-difluorophenyl)-8,9-dimethoxybenzo[c][2,7]naphthyridin-4-amine;
3-(4-amino-8,9-dimethoxybenzo[c][2,7]naphthyridin-2-yl)-N-(pyridin-3-ylmethyl)benzamide;
2-(5-{2-[(2R)-aziridin-2-yl]ethyl}pyridin-3-yl)-8,9-dimethoxybenzo[c][2,7]naphthyridin-4-amine;
2-[5-({(2S)-2-amino-3-[(2R)-2-(1,3-thiazol-2-yl)pyrrolidin-1-yl]propyl}oxy)pyridin-3-yl]-8,9-dimethoxybenzo[c][2,7]naphthyridin-4-amine;
2-[5-({(2S)-2-amino-3-[(2S)-2-(1,3-thiazol-2-yl)pyrrolidin-1-yl]propyl}oxy)pyridin-3-yl]-8,9-dimethoxybenzo[c][2,7]naphthyridin-4-amine;
3-(4-amino-8,9-dimethoxybenzo[c][2,7]naphthyridin-2-yl)-5-fluorobenzonitrile;
(2R)-4-[5-(4-amino-8,9-dimethoxybenzo[c][2,7]naphthyridin-2-yl)pyridin-3-yl]-N1-ethyl-N1-(pyridin-4-ylmethyl)butane-1,2-diamine;
2-[5-({(2S)-2-amino-3-[(2R)-2-(1,3-thiazol-2-yl)pyrrolidin-1-yl]propyl}oxy)pyridin-3-yl]-8,9-dimethoxybenzo[c][2,7]naphthyridin-4-amine;
(2S)-3-{[5-(4-amino-8,9-dimethoxybenzo[c][2,7]naphthyridin-2-yl)pyridin-3-yl]oxy}-N1-[(4-phenyltetrahydro-2H-pyran-4-yl)methyl]propane-1,2-diamine;
2-(5-{[(2S)-2-amino-3-(4-pyridin-4-ylpiperazin-1-yl)propyl]oxy}pyridin-3-yl)-8,9-dimethoxybenzo[c][2,7]naphthyridin-4-amine;
(2S)-3-{[5-(4-amino-8,9-dimethoxybenzo[c][2,7]naphthyridin-2-yl)pyridin-3-yl]oxy}-N1-(1-methyl-1-pyridin-4-ylethyl)propane-1,2-diamine;
2-(5-{[(2R)-2-amino-3-(1,3-benzothiazol-2-yloxy)propyl]oxy}pyridin-3-yl)-8,9-dimethoxybenzo[c][2,7]naphthyridin-4-amine;
2-{5-[(3R)-3-amino-4-(1,3-thiazol-2-ylthio)butyl]pyridin-3-yl}-8,9-dimethoxybenzo[c][2,7]naphthyridin-4-amine;
2-(3-fluoro-5-methoxyphenyl)-8,9-dimethoxybenzo[c][2,7]naphthyridin-4-amine;
2-(5-{[(2S)-2-amino-3-pyrrolidin-1-ylpropyl]oxy}pyridin-3-yl)-8,9-dimethoxybenzo[c][2,7]naphthyridin-4-amine;
2-(5-{[(2S)-2-amino-3-(pyridin-4-yloxy)propyl]oxy}pyridin-3-yl)-8,9-dimethoxybenzo[c][2,7]naphthyridin-4-amine;
2-[5-({(2R)-2-amino-3-[(4-methylpyridin-2-yl)oxy]propyl}oxy)pyridin-3-yl]-8,9-dimethoxybenzo[c][2,7]naphthyridin-4-amine;
2-[5-({(2R)-2-amino-3-[(3,5-dichloropyridin-2-yl)oxy]propyl}oxy)pyridin-3-yl]-8,9-dimethoxybenzo[c][2,7]naphthyridin-4-amine;
2-[5-({(2R)-2-amino-3-[(5-chloropyridin-2-yl)oxy]propyl}oxy)pyridin-3-yl]-8,9-dimethoxybenzo[c][2,7]naphthyridin-4-amine;
2-{5-[(4-aminopyridin-3-yl)oxy]pyridin-3-yl}-8,9-dimethoxybenzo[c][2,7]naphthyridin-4-amine;
3-{[(2S)-2-amino-3-{[5-(4-amino-8,9-dimethoxybenzo[c][2,7]naphthyridin-2-yl)pyridin-3-yl]oxy}propyl]oxy}benzonitrile;
2-(5-{[(2R)-2-amino-3-(1,3-thiazol-2-yloxy)propyl]oxy}pyridin-3-yl)-8,9-dimethoxybenzo[c][2,7]naphthyridin-4-amine;
2-(5-anilinopyridin-3-yl)-8,9-dimethoxybenzo[c][2,7]naphthyridin-4-amine;
tert-butyl {(1R)-2-{[5-(4-amino-8,9-dimethoxybenzo[c][2,7]naphthyridin-2-yl)pyridin-3-yl]oxy}-1-[(1,3-thiazol-2-yloxy)methyl]ethyl}carbamate;
N-[(2S)-2-amino-3-{[5-(4-amino-8,9-dimethoxybenzo[c][2,7]naphthyridin-2-yl)pyridin-3-yl]oxy}propyl]benzene-1,4-diamine;
(2S)-3-{[5-(4-amino-8,9-dimethoxybenzo[c][2,7]naphthyridin-2-yl)pyridin-3-yl]oxy}-N1-(3-chlorophenyl)-N1-methylpropane-1,2-diamine;
(2S)-3-{[5-(4-amino-8,9-dimethoxybenzo[c][2,7]naphthyridin-2-yl)pyridin-3-yl]oxy}-N1-(4-methylpyrimidin-2-yl)propane-1,2-diamine;
2-{5-[(5-aminopyridin-2-yl)oxy]pyridin-3-yl}-8,9-dimethoxybenzo[c][2,7]naphthyridin-4-amine;
2-{5-[(3-aminopyridin-2-yl)oxy]pyridin-3-yl}-8,9-dimethoxybenzo[c][2,7]naphthyridin-4-amine;
2-{5-[(3-aminopyridin-4-yl)oxy]pyridin-3-yl}-8,9-dimethoxybenzo[c][2,7]naphthyridin-4-amine;
2-{5-[(6-aminopyridin-2-yl)oxy]pyridin-3-yl}-8,9-dimethoxybenzo[c][2,7]naphthyridin-4-amine;
2-{5-[(6-aminopyridin-3-yl)oxy]pyridin-3-yl}-8,9-dimethoxybenzo[c][2,7]naphthyridin-4-amine;
2-{5-[(4-aminopyridin-2-yl)oxy]pyridin-3-yl}-8,9-dimethoxybenzo[c][2,7]naphthyridin-4-amine;
2-isoquinolin-4-yl-8,9-dimethoxybenzo[c][2,7]naphthyridin-4-amine;
5-(4-amino-8,9-dimethoxybenzo[c][2,7]naphthyridin-2-yl)isophthalaldehyde;
8,9-dimethoxy-2-(6-piperazin-1-ylpyridin-3-yl)benzo[c][2,7]naphthyridin-4-amine;
2-{5-[(2-aminopyridin-3-yl)oxy]pyridin-3-yl}-8,9-dimethoxybenzo[c][2,7]naphthyridin-4-amine;
tert-butyl [(1R)-1-({[5-(4-amino-9-bromobenzo[c][2,7]naphthyridin-2-yl)pyridin-3-yl]oxy}methyl)propyl]carbamate;
2-(5-{[(2R)-2-aminobutyl]oxy}pyridin-3-yl)-9-bromobenzo[c][2,7]naphthyridin-4-amine
2-(5-{[(2R)-2-aminobutyl]oxy}pyridin-3-yl)-9-(phenylethynyl)benzo[c][2,7]naphthyridin-4-amine;
tert-butyl (2-{[5-(4-amino-8,9-dimethoxybenzo[c][2,7]naphthyridin-2-yl)pyridin-3-yl]oxy}-1-methylethyl)carbamate;
2-[5-(2-aminopropoxy)pyridin-3-yl]-8,9-dimethoxybenzo[c][2,7]naphthyridin-4-amine;
9-bromobenzo[c][2,7]naphthyridin-4-amine;
2-{5-[(5-aminopyridin-3-yl)oxy]pyridin-3-yl}-8,9-dimethoxybenzo[c][2,7]naphthyridin-4-amine;
2-(5-{[(2R)-2-aminobutyl]oxy}pyridin-3-yl)-9-morpholin-4-ylbenzo[c][2,7]naphthyridin-4-amine;
9-bromo-N-(2-methylphenyl)benzo[c][2,7]naphthyridin-4-amine;
2-(5-(2-aminopyridin-4-yloxy)pyridin-3-yl)-8,9-dimethoxybenzo[c][2,7]naphthyridin-4-amine;
   and a pharmaceutically acceptable salt thereof.

Still additional illustrative examples of Benzo[c][2,7]naphthyridine Derivatives of Formula (I), (Ia) and/or (Ib) include:
2-(5-{[(5S)-2-anilino-4,5-dihydro-1H-imidazol-5-yl]methoxy}pyridin-3-yl)-8,9-dimethoxybenzo[c][2,7]naphthyridin-4-amine;
8,9-dimethoxy-2-{5-[(3-methoxyphenyl)amino]pyridin-3-yl}benzo[c][2,7]naphthyridin-4-amine;
8,9-dimethoxy-2-{5-[(4-methoxyphenyl)amino]pyridin-3-yl}benzo[c][2,7]naphthyridin-4-amine;
2-{[5-(4-amino-8,9-dimethoxybenzo[c][2,7]naphthyridin-2-yl)pyridin-3-yl]amino}benzonitrile;
3-{[5-(4-amino-8,9-dimethoxybenzo[c][2,7]naphthyridin-2-yl)pyridin-3-yl]amino}benzonitrile;
4-{[5-(4-amino-8,9-dimethoxybenzo[c][2,7]naphthyridin-2-yl)pyridin-3-yl]amino}benzonitrile;
8,9-dimethoxy-2-{5-[(2-methoxyphenyl)amino]pyridin-3-yl}benzo[c][2,7]naphthyridin-4-amine;
1-[(1R)-1-({[5-(4-amino-8,9-dimethoxybenzo[c][2,7]naphthyridin-2-yl)pyridin-3-yl]oxy}methyl)propyl]guanidine;
8,9-dimethoxy-2-[5-(4-methoxyphenoxy)pyridin-3-yl]benzo[c][2,7]naphthyridin-4-amine;
(2-{[(2R)-2-amino-3-{[5-(4-amino-8,9-dimethoxybenzo[c][2,7]naphthyridin-2-yl)pyridin-3-yl]oxy}propyl]thio}-1-methyl-1H-imidazol-5-yl)methanol;
N-[5-(4-amino-8,9-dimethoxybenzo[c][2,7]naphthyridin-2-yl)pyridin-3-yl]benzene-1,2-diamine;
N-[5-(4-amino-8,9-dimethoxybenzo[c][2,7]naphthyridin-2-yl)pyridin-3-yl]-N′-phenylbenzene-1,2-diamine;
8,9-dimethoxy-2-{5-[(2-morpholin-4-ylphenyl)amino]pyridin-3-yl}benzo[c][2,7]naphthyridin-4-amine;
N-(2-{[5-(4-amino-8,9-dimethoxybenzo[c][2,7]naphthyridin-2-yl)pyridin-3-yl]amino}phenyl)acetamide;
2-[3-(3-aminopropyl)-5-fluorophenyl]-8,9-dimethoxybenzo[c][2,7]naphthyridin-4-amine;
2-(5-{[(2S)-2-amino-3-(4-iminopyridin-1(4H)-yl)propyl]oxy}pyridin-3-yl)-8,9-dimethoxybenzo[c][2,7]naphthyridin-4-amine;
2-{[5-(4-amino-8,9-dimethoxybenzo[c][2,7]naphthyridin-2-yl)pyridin-3-yl]amino}benzamide;
2-{[5-(4-amino-8,9-dimethoxybenzo[c][2,7]naphthyridin-2-yl)pyridin-3-yl]amino}-N-methylbenzamide;
3-{[5-(4-amino-8,9-dimethoxybenzo[c][2,7]naphthyridin-2-yl)pyridin-3-yl]amino}benzamide;
2-(5-{[3-(aminomethyl)phenyl]amino}pyridin-3-yl)-8,9-dimethoxybenzo[c][2,7]naphthyridin-4-amine;
4-{[5-(4-amino-8,9-dimethoxybenzo[c][2,7]naphthyridin-2-yl)pyridin-3-yl]amino}-2,3,5,6-tetrafluorobenzamide;
4-{[5-(4-amino-8,9-dimethoxybenzo[c][2,7]naphthyridin-2-yl)pyridin-3-yl]amino}-N-[2-(diethylamino)ethyl]benzamide;
2-(5-{[4-(aminomethyl)phenyl]amino}pyridin-3-yl)-8,9-dimethoxybenzo[c][2,7]naphthyridin-4-amine;
4-{[5-(4-amino-8,9-dimethoxybenzo[c][2,7]naphthyridin-2-yl)pyridin-3-yl]amino}-N-phenylbenzamide;
(4-{[5-(4-amino-8,9-dimethoxybenzo[c][2,7]naphthyridin-2-yl)pyridin-3-yl]amino}phenyl)methanol;
(3-{[5-(4-amino-8,9-dimethoxybenzo[c][2,7]naphthyridin-2-yl)pyridin-3-yl]amino}phenyl)methanol;
(2-{[5-(4-amino-8,9-dimethoxybenzo[c][2,7]naphthyridin-2-yl)pyridin-3-yl]amino}phenyl)methanol;
2-(2-{[5-(4-amino-8,9-dimethoxybenzo[c][2,7]naphthyridin-2-yl)pyridin-3-yl]amino}phenyl)ethanol;
2-(4-{[5-(4-amino-8,9-dimethoxybenzo[c][2,7]naphthyridin-2-yl)pyridin-3-yl]amino}phenyl)ethanol;
8,9-dimethoxy-2-[5-(pyridin-2-ylamino)pyridin-3-yl]benzo[c][2,7]naphthyridin-4-amine;
8,9-dimethoxy-2-[5-(pyridin-3-ylamino)pyridin-3-yl]benzo[c][2,7]naphthyridin-4-amine;
8,9-dimethoxy-2-[5-(pyridin-4-ylamino)pyridin-3-yl]benzo[c][2,7]naphthyridin-4-amine;
2-(3-ethoxyphenyl)-8,9-dimethoxybenzo[c][2,7]naphthyridin-4-amine;
8,9-dimethoxy-2-(3-propoxyphenyl)benzo[c][2,7]naphthyridin-4-amine;
4-amino-8,9-dimethoxybenzo[c][2,7]naphthyridin-2-ol;
8,9-dimethoxy-2-(3-thienyl)benzo[c][2,7]naphthyridin-4-amine;
4-{[(2S)-2-amino-3-{[5-(4-amino-8,9-dimethoxybenzo[c][2,7]naphthyridin-2-yl)pyridin-3-yl]oxy}propyl]amino}cyclohexanol;
(2S)-3-{[5-(4-amino-8,9-dimethoxybenzo[c][2,7]naphthyridin-2-yl)pyridin-3-yl]oxy}-N1-(cyclohexylmethyl)propane-1,2-diamine;
2-(5-{[2-(benzyloxy)phenyl]amino}pyridin-3-yl)-8,9-dimethoxybenzo[c][2,7]naphthyridin-4-amine;
8,9-dimethoxy-2-{3-[(4-pyridin-2-ylpiperazin-1-yl)carbonyl]phenyl}benzo[c][2,7]naphthyridin-4-amine;
(2S)-3-{[5-(4-amino-8,9-dimethoxybenzo[c][2,7]naphthyridin-2-yl)pyridin-3-yl]oxy}-N1-cyclobutylpropane-1,2-diamine;
(2S)-3-{[5-(4-amino-8,9-dimethoxybenzo[c][2,7]naphthyridin-2-yl)pyridin-3-yl]oxy}-N1-cyclopentylpropane-1,2-diamine;
5-(4-amino-8,9-dimethoxybenzo[c][2,7]naphthyridin-2-yl)-N-butylpyridine-3-carboximidamide;
5-(4-amino-8,9-dimethoxybenzo[c][2,7]naphthyridin-2-yl)-N-isopropylpyridine-3-carboximidamide;
5-(4-amino-8,9-dimethoxybenzo[c][2,7]naphthyridin-2-yl)-N-sec-butylpyridine-3-carboximidamide;
2-(1H-indazol-5-yl)-8,9-dimethoxybenzo[c][2,7]naphthyridin-4-amine;
(2S)-3-{[5-(4-amino-8,9-dimethoxybenzo[c][2,7]naphthyridin-2-yl)pyridin-3-yl]oxy}-N1-{[4-(4-fluorophenyl)tetrahydro-2H-pyran-4-yl]methyl}propane-1,2-diamine;
(2S)-3-{[5-(4-amino-8,9-dimethoxybenzo[c][2,7]naphthyridin-2-yl)pyridin-3-yl]oxy}-N1-[(1-phenylcyclopentyl)methyl]propane-1,2-diamine;
(2S)-3-{[5-(4-amino-8,9-dimethoxybenzo[c][2,7]naphthyridin-2-yl)pyridin-3-yl]oxy}-N1-[(1-phenylcyclopropyl)methyl]propane-1,2-diamine;
(2S)-3-{[5-(4-amino-8,9-dimethoxybenzo[c][2,7]naphthyridin-2-yl)pyridin-3-yl]oxy}-N1-(2,2,2-trifluoroethyl)propane-1,2-diamine;
3-(4-amino-8,9-dimethoxybenzo[c][2,7]naphthyridin-2-yl)-5-fluorobenzamide;
2-[5-(allyloxy)pyridin-3-yl]-8,9-dimethoxybenzo[c][2,7]naphthyridin-4-amine;
2-{[5-(4-amino-8,9-dimethoxybenzo[c][2,7]naphthyridin-2-yl)pyridin-3-yl]oxy}-1-phenylethanone;
3-(4-{[(2S)-2-amino-3-{[5-(4-amino-8,9-dimethoxybenzo[c][2,7]naphthyridin-2-yl)pyridin-3-yl]oxy}propyl]oxy}phenyl)propanenitrile;
2-[5-(biphenyl-2-ylamino)pyridin-3-yl]-8,9-dimethoxybenzo[c][2,7]naphthyridin-4-amine;
2-{5-[(2-fluorophenyl)amino]pyridin-3-yl}-8,9-dimethoxybenzo[c][2,7]naphthyridin-4-amine;
4-{[5-(4-amino-8,9-dimethoxybenzo[c][2,7]naphthyridin-2-yl)pyridin-3-yl]amino}benzamide;
2-(5-{[(2R)-2-aminobutyl]oxy}pyridin-3-yl)-9-quinolin-3-ylbenzo[c][2,7]naphthyridin-4-amine;
9-bromo-N-(4-methoxyphenyl)benzo[c][2,7]naphthyridin-4-amine;
2-(5-{[2-(aminomethyl)phenyl]amino}pyridin-3-yl)-8,9-dimethoxybenzo[c][2,7]naphthyridin-4-amine;
N-(3-bromophenyl)-8,9-dimethoxybenzo[c][2,7]naphthyridin-4-amine;
8,9-dimethoxy-2-(5-{[2-(2-morpholin-4-ylethoxy)phenyl]amino}pyridin-3-yl)benzo[c][2,7]naphthyridin-4-amine;
8,9-dimethoxy-2-(5-{[2-(2-methoxyethoxy)phenyl]amino}pyridin-3-yl)benzo[c][2,7]naphthyridin-4-amine;
   and a pharmaceutically acceptable salt thereof

### 5.5 Methods For Making The Benzo[c][2,7]naphthyridine Derivatives

Examples of synthetic pathways that are useful for making Benzo[c][2,7]naphthyridine Derivatives are set forth in the Examples below and generalized in Schemes 1a-9.

A Benzo[c][2,7]naphthyridine Derivative represented by formula 5a can be prepared as illustrated in Scheme 1a.

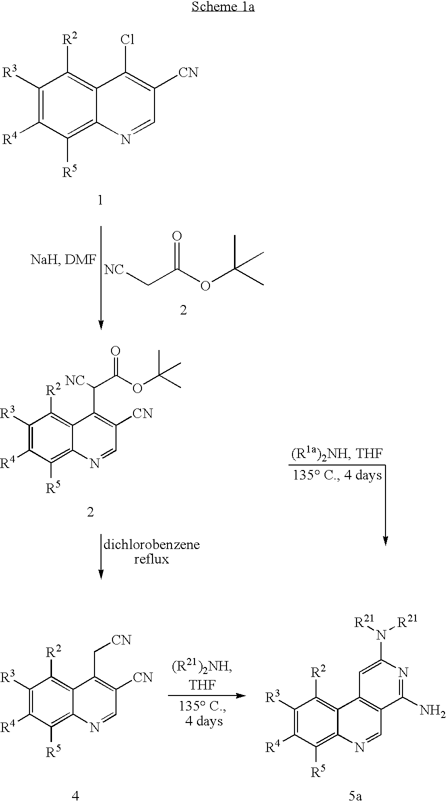

wherein R²¹₂NH represents H—NR¹²R¹³, H—NR¹²(C(R¹²)₂)ₙ—R¹³ or R²¹₂NH represents H—R^{13″}, wherein R², R³, R⁴, R⁵, R¹², R¹³, and n are as defined above for the Benzo[c][2,7]naphthyridine Derivates of Formula (I). H—R^{13″} represents a heterocycle that comprises a nucleophilic nitrogen atoms that can be easily alkylated or acylated (a subset of R¹³).

According to Scheme 1a, reaction of a 4-chloro-3-cyanoquinoline compound of formula 1 with an anion derived from a cyanoacetate, e.g. a tri-(C₁-C₆ alkyl)methyl cyanoacetate such as t-butyl cyanoacetate, (formula 2) provides a cyanoquinoline compound of formula 3. This compound can be thermally decarboxylated by heating in an inert solvent, such as dichlorobenzene to provide a dicyano compound of formula 4. Reaction of a dicyano compound of formula 4 with a primary or a secondary amine (R²¹)₂NH at elevated temperature for an extended period of time then provides a Benzo[c][2,7]naphthyridine Derivative represented by formula 5a. Alternatively, the reaction of the compound of formula 3 under similar reaction conditions can directly provide a Benzo[c][2,7]naphthyridine Derivative represented by formula 5a. If necessary, substituents on the compound of formula 1, as well as other compounds described herein, can be protected with one or more suitable protecting groups compatible with subsequent reactions and de-protected at a later stage. (see, e.g. T. W. Green and P. G. M. Wuts, Protective Groups in Organic Synthesis, Wiley-Interscience, New York, 1999, incorporated herein by reference in its entirety).

Scheme 1b illustrates an alternative method of synthesis of a Benzo[c][2,7]naphthyridine Derivative.

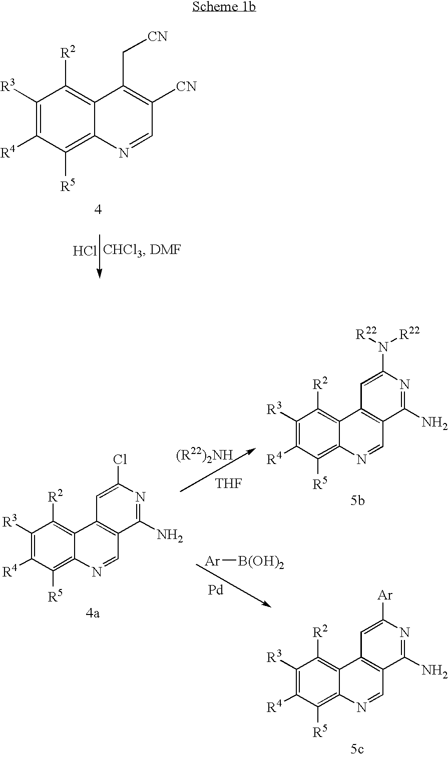

wherein R², R³, R⁴ and R⁵ are as defined above for Benzo[c][2,7]naphthyridine Derivatives of Formula (I), the amine (R²²)₂NH is H—NR¹²R¹³, H—NR¹³(C(R¹²)₂)ₙ—R¹³ or R²²₂NH is H—R^{13″}, wherein R¹², R¹³, and n are as defined above for a Benzo[c][2,7]naphthyridine Derivatives of Formula (I), (Ia), or (Ib). H—R^{13″} represents a non-aromatic, non-lactam nitrogen-containing heterocycle, and Ar is an aryl or heteroaryl group.

Scheme 1b also illustrates preparation of a compound where an aryl or heteroaryl group is attached to the core structure via a carbon-carbon bond. The reagent Ar—B(OH)₂ represents an aryl or heteroaryl compound substituted on a carbon with a boronic acid functional group. The reaction of the compound of formula 4 (e.g., prepared as described in Scheme 1a) with hydrogen chloride gas in a mixture of alcohol-free chloroform and DMF provides a chloro compound of formula 4a, which can be reacted with an amine, or a heterocycle containing a nucleophic amine to provide a Benzo[c][2,7]naphthyridine Derivative represented by formula 5b. Additionally, the chloro compound of formula 4a can be coupled with an aryl or heteroaryl boronic acid derivative Ar—B(OH)₂ using palladium catalysis to provide a Benzo[c][2,7]naphthyridine Derivative represented by formula 5c.

The primary amine of Benzo[c][2,7]naphthyridine derivatives of formula 5a, 5b and 5c, can be alkylated or oxidized using methods known in the art (see, e.g. March, Advanced Organic Chemistry Reactions, Mechanisms and Structure, Fourth Edition, John Wiley and Sons, 1992, incorporated by reference herein in its entirety) to attach an R⁶ other than hydrogen, wherein R⁶ is as defined above for the compounds of Formula (I), (Ia), or (Ib).

Methods used to prepare the 4-chloro-3-cyanoquinoline compounds of formula 1 are described in the following patents and patent applications: WO 98/43960, U.S. Pat. No. 6,288,082, U.S. Pat. No. 6,002,008, WO 00/18761 and WO 00/18740, all of which are incorporated be reference herein in their entirety. A typical method is shown in Scheme 2.

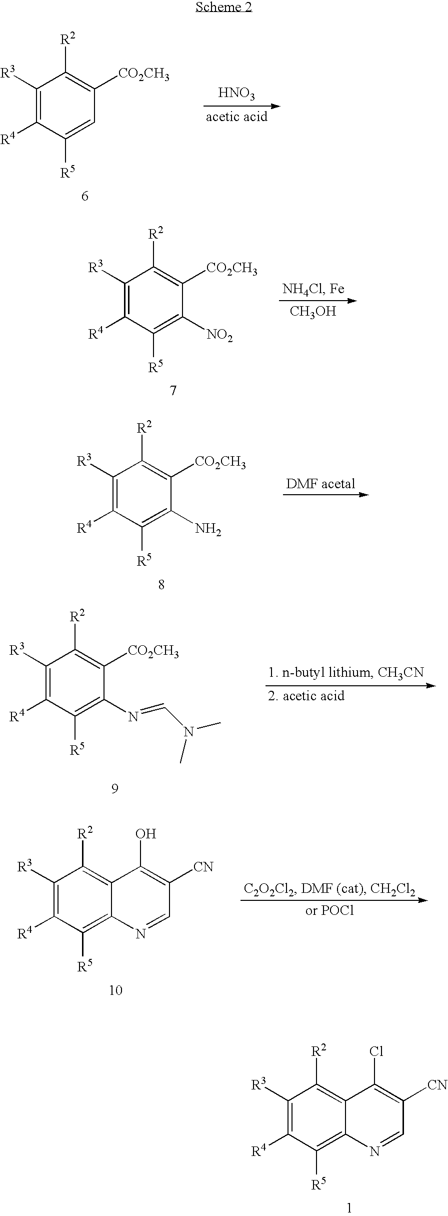

wherein R², R³, R⁴ and R⁵ are as defined above for the Benzo[c][2,7]naphthyridine derivatives of Formula (I).

A benzoic acid ester of formula 6 can be nitrated to provide a nitro-derivative of formula 7. Where necessary, substituents on the compound of formula 6 can be protected with one or more suitable protecting group compatible with the subsequent reactions and de-protected at a later stage (see, e.g. T. W. Green and P. G. M. Wuts, Protective Groups in Organic Synthesis, Wiley-Interscience, New York, 1999, incorporated herein by reference in its entirety). If regioisomers are obtained in the nitration step, they can be separated by various methods, such as chromatography and fractional crystallization. The nitrate group of formula 7 can be reduced, for example, using a refluxing mixture of ammonium chloride and iron in methanol or a similar solvent, to provide an amine of formula 8. The amino group of the compound of formula 8 can be converted to an amidine derivative of formula 9, e.g., with a DMF-acetal. The reaction of the amidine compound of formula 9 with an excess of the lithium anion of acetonitrile at temperature below 40° C., which can be produced by the reaction of acetonitrile with n-butyl lithium at low temperature, followed by warming and treatment with acetic acid, provides a 3-cyano-4-hydroxy-quinoline compound of formula 10. The 4-chloro-3-cyanoquinoline compound of formula 1, useful in preparation of a Benzo[c][2,7]naphthyridine Derivative, can obtained, for example, by reaction of a cyanoquinoline compound of formula 10 with oxalyl chloride in refluxing methylene chloride in the presence of a catalytic amount of DMF, or alternatively, by heating the cyanoquinoline compound 10 with phosphorous oxychloride.

The methods outlined in Scheme 1a are useful for the preparation of a Benzo[c][2,7]naphthyridine Derivative where R¹ is attached to the benzo[c][2,7]naphthyridine core structure by a carbon-nitrogen bond. One mode of preparation of a Benzo[c][2,7]naphthyridine Derivative, where R¹ is attached to the core by a carbon-carbon bond is outlined in Scheme 3a.

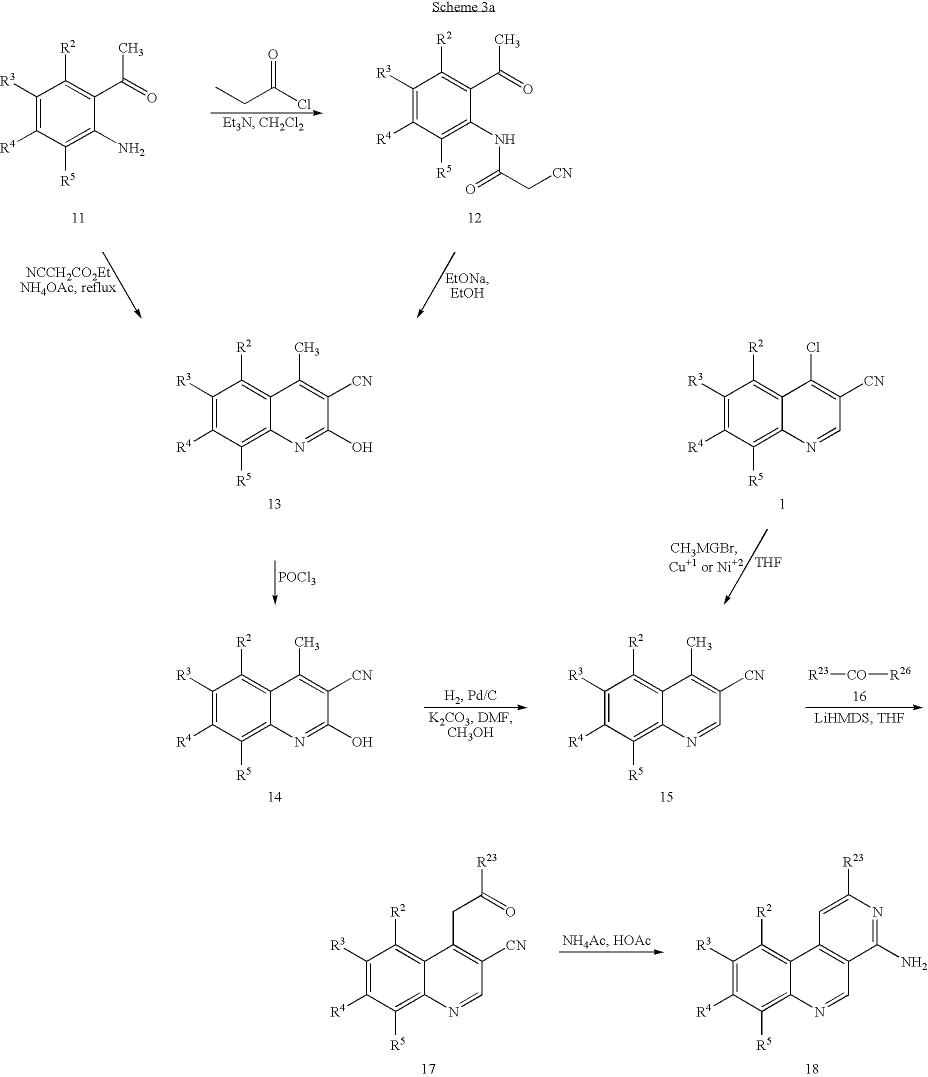

wherein R², R³, R⁴ and R⁵ are defined as for Benzo[c][2,7]naphthyridine Derivatives of Formula (I), R²—CO—R²⁶ represents an ester, acid chloride, or imidazolide where R²⁶ is a —O—C₁-C₆ alkyl, an ester, an acyl halide, or an imidazolide, or an imidazole bonded via its nitrogen atom, R′ is a subset of R¹ which is attached to the core structure by a carbon-carbon bond.

The reaction of a 2-amino-acetophenone compound of formula 11 with the acid chloride derived from cyanoacetic acid provides an amide of formula 12, which can be cyclized using a strong base, e.g. sodium ethoxide in refluxing ethanol to provide a cyanoquinoline compound of formula 13. Alternatively, the cyanoquinolone compound of formula 13 can be prepared directly from the compound of formula 11, by refluxing it with a mixture of ethyl cyanoacetate and ammonium acetate. The hydroxyl group of the cyanoquinoline compound of formula 13 can be chlorinated using a chlorinating reagent such as phosphorous oxychloride or SOCl₂ to provide a 2-chloro-3 cyano-4-methyl-quinoline compound of formula 14. The chlorine atom of the compound of formula 14 can be removed by hydrogenolysis using a palladium catalyst and a strong base such as potassium carbonate or triethylamine in a solvent such as DMF or a DMF/methanol mixture, resulting in formation of a 4-methyl-3-cyanoquinoline compound of formula 15. An alternate preparation of the 4-methyl-3-cyanoquinoline compound of formula 15 can be accomplished by reaction of the 4-chloro-3-cyanoquinoline compound of formula 1 (from Schemes 1a and 2 above), with an excess of methyl magnesium bromide in the presence of a copper or nickel catalyst in THF. At low temperature, the anion derived from the compound of formula 15 can be prepared by reaction with a strong base, e.g., lithium hexamethyldisilazane, in an inert solvent such as THF. The anion of the compound of formula 15 can be condensed with an ester, an acid halide, or an imidazolide, e.g., an imidazolide of formula 16, to provide a ketone of formula 17. Reacting the compound of formula 17 with ammonium acetate in a solvent such as acetic acid, DMF, or molten phenol, provides a Benzo[c][2,7]naphthyridine Derivative represented by formula 18. Where necessary, the substituents on 1, 11, and 16 can be protected with a protecting group compatible with the subsequent reactions.

Another way to prepare the 4-methyl-3-cyanoquinoline compounds is illustrated in Scheme 3b. Where necessary, the substituents R², R³, R⁴ or R⁵ can be protected with a protecting group compatible with the subsequent reactions.

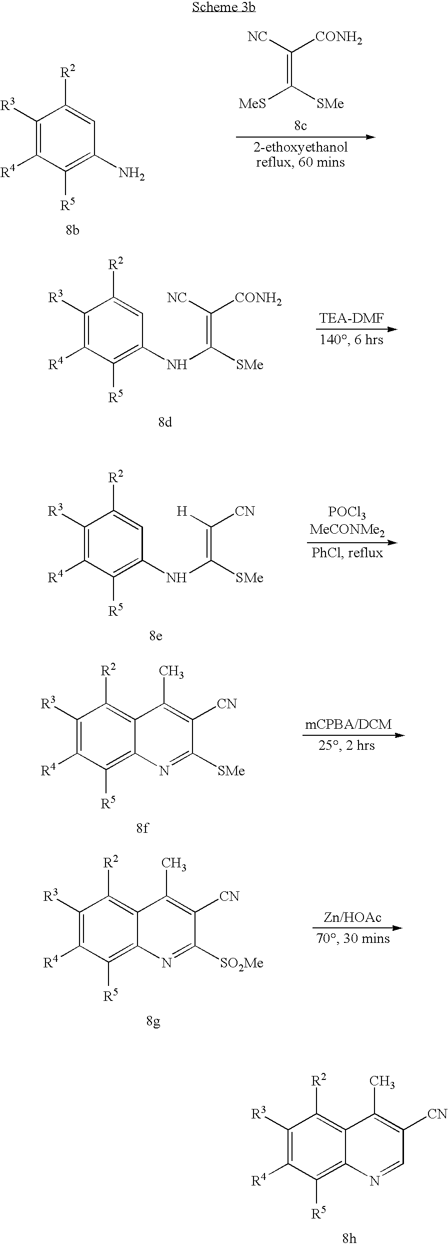

wherein R², R³, R⁴ and R⁵ are as defined above for Benzo[c][2,7]naphthyridine Derivatives of Formula (I).

The reaction of the amine of formula 8b with the ethylene reagent formula 8c in refluxing 2-ethoxyethanol provides a secondary amine of formula 8d. Heating a solution of the compound of formula 8d in DMF with a base, such as triethylamine, provides the compound of formula 8e, which on refluxing with phosphorous oxychloride and dimethylacetamide in chlorobenzenze results in the formation of the thiomethyl compound of formula 8f. The thiomethyl substituent can then be removed, e.g., by oxidation to the sulfone compound of formula 8g using m-chloroperbenzoic acid followed by reductive removal of the sulfone group, e.g., using zinc and acetic acid, to provide the 4-methyl-3-cyanoquinoline compound of formula 15.

Some of the compounds useful in preparation of some of the Benzo[c][2,7]naphthyridine Derivatives where R¹ is represented by the formula:

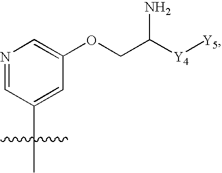

wherein Y₄ and Y₅ are as defined for the Benzo[c][2,7]naphthyridine Derivatives of Formula (I), can be prepared as illustrated in Scheme 4.

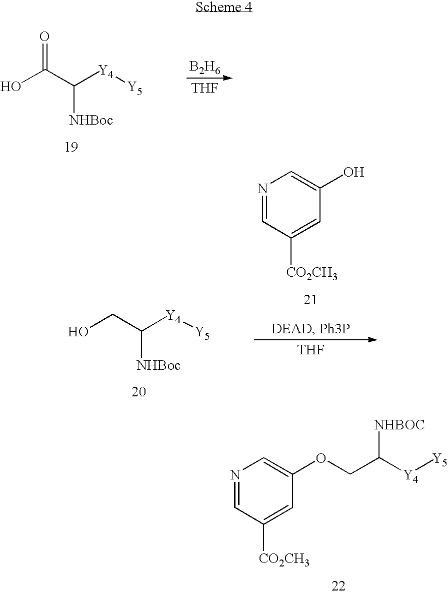

wherein Y₄ and Y₅ are as defined above for the Benzo[c][2,7]naphthyridine Derivatives of Formula (I), (Ia), or (Ib).

The carboxylic acid compound of formula 19 can be reduced with a reducing agent such as borane in a solvent such as THF. The derivative of formula 19 can be used as either enantiomer or as the racemic mixture. The resulting alcohol compound of formula 20 can be coupled to the 3-hydroxy nicotinate methyl ester of formula 21 via a Mitsumobu reaction to provide the ester compound of formula 22. Finally, the compound of formula 22, as described, e.g., in Scheme 5a, can be used to prepare a Benzo[c][2,7]naphthyridine Derivative. In some cases, it might be preferable to hydrolyze the ester compound of formula 22 and convert it to its imidazolide and use it as described in Scheme 3a to prepare a Benzo[c][2,7]naphthyridine Derivative.

Benzo[c][2,7]naphthyridine Derivatives where R¹ is a pyridine ring of the form:

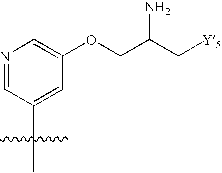

and Y′₅ is a subset of Y₅, defined above for the Benzo[c][2,7]naphthyridine Derivatives of Formula (I), (Ia), or (Ib), wherein Y′₅ comprises is an amino group or heterocycle that is attached via its nitrogen atom, can be prepared according to Scheme 5a.

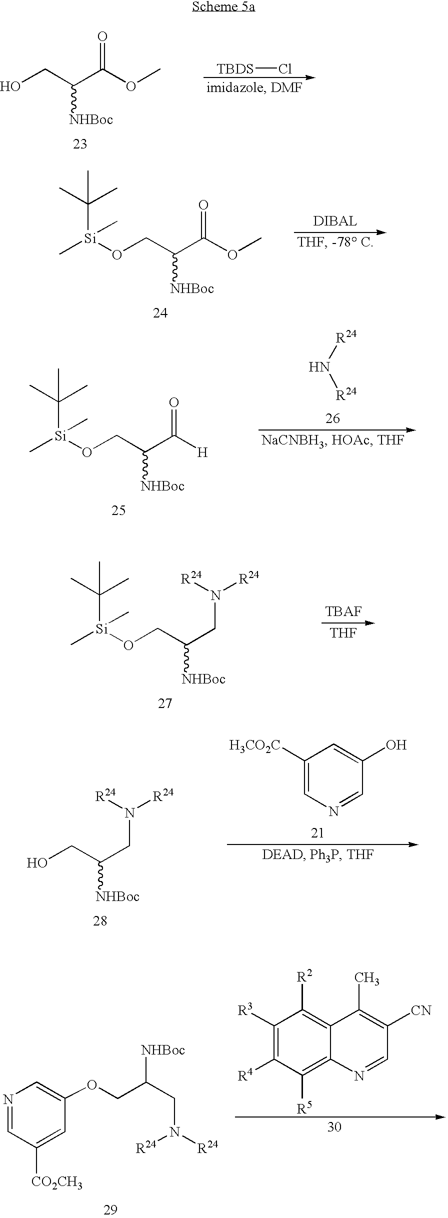

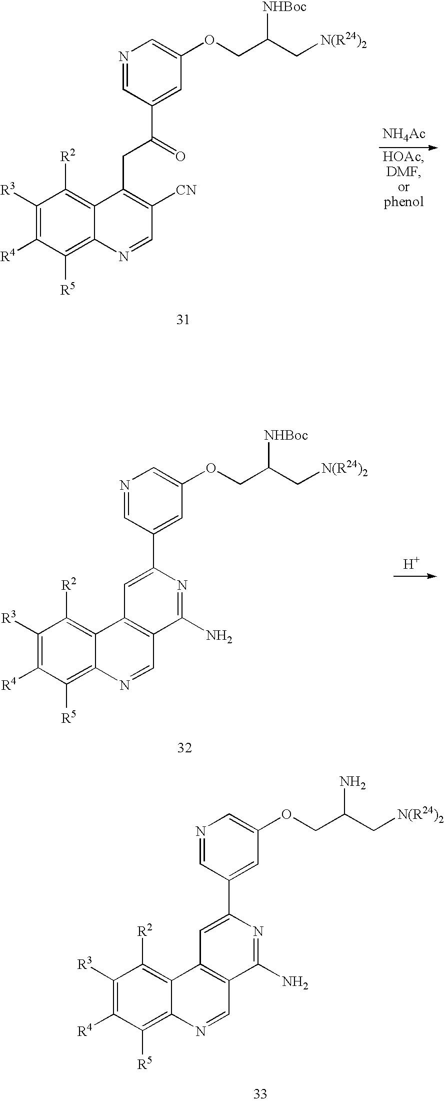

wherein R², R³, R⁴ and R⁵ are as defined above for the Benzo[c][2,7]naphthyridine Derivatives of Formula (I), N(R²⁴)₂ represents a subset of Y₅ comprising an amine that is sufficiently nucleophilic to participate in reductive animation reaction with an aldehyde.

Either enantiomer or a racemic mixture of the hydroxyl compound of formula 23 can be used as the starting material. The hydroxyl group is first protected, for example as a t-butyl dimethylsilyl derivative of formula 24. Other silyl protecting groups, for example trimethylsilyl (TMS), triethylsilyl (TES), tert-butyldimethylsilyl (TBDS), tri-isopropylsilyl (TIPS) and tert-butyldiphenylsilyl (TBDPS) can also be used. The ester group can then be reduced to the aldehyde of formula 25, e.g., by using a reducing agent such as DIBAL at low temperatures. The resulting aldehyde of formula 25 can then be coupled with an amine of formula 26 by a reductive amination reaction to provide the compound of formula 27. The silyl protecting group can then be removed using a source of fluoride ion, such as ammonium fluoride or a tri-(C₁-C₆ alkyl) ammonium fluoride, and the resulting alcohol compound of formula 28 can be coupled to 3-hydroxy nicotinate methyl ester of formula 21, via a Mitsumobu reaction to provide the ester compound of formula 29. The compound of formula 29, as described in Scheme 5, is useful for preparation of a Benzo[c][2,7]naphthyridine Derivative. In some cases, it is preferable to hydrolyze the ester compound of formula 29, convert it to its imidazolide and used this as described in Scheme 3a to prepare a Benzo[c][2,7]naphthyridine Derivative. At temperature below −40° C., the compound of formula 29 can react with the anion derived from the methylcyanoquinoline compound of formula 30 using lithium hexamethyldisilazine. The resulting ketone derivative of formula 31 can be reacted with ammonium acetate by heating in a solvent such as acetic acid, DMF, or phenol to provide the protected benzo[c][2,7]naphthyridine compound of formula 32. In the final synthetic step, the amine protecting group (e.g., Boc) is removed with an acid to provide a Benzo[c][2,7]naphthyridine Derivative represented by formula 33.

Certain Benzo[c][2,7]naphthyridine Derivatives of Formula (I) can be prepared according to Scheme 5b.

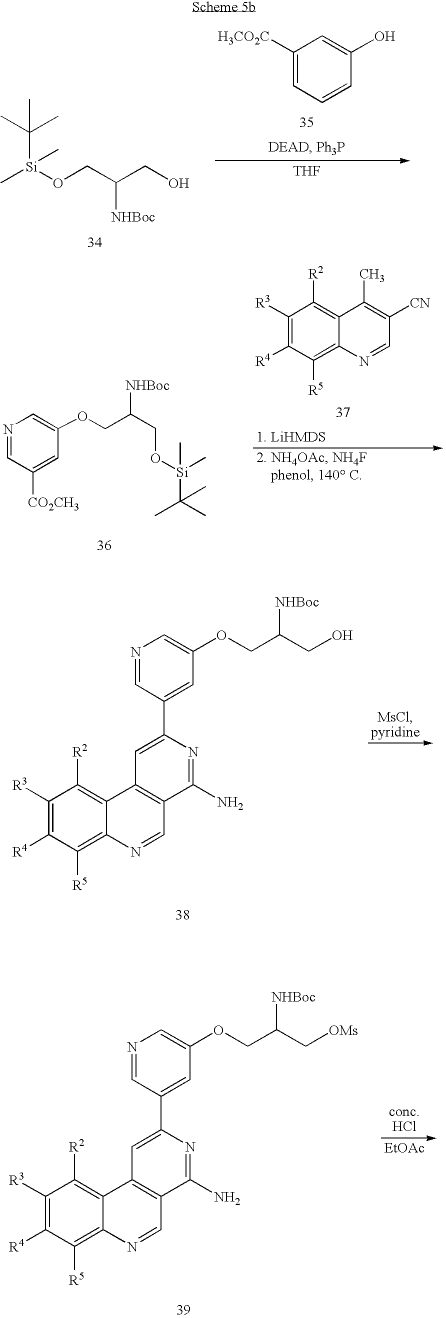

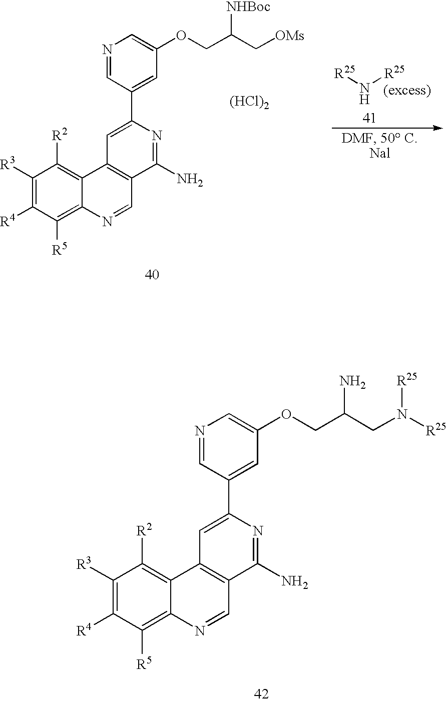

wherein R², R³, R⁴, R⁵ are as defined above for Benzo[c][2,7]naphthyridine Derivatives of Formula (I) and N(R²⁵)₂ is a subset of Y₅ comprising an amino group or heterocycle that is attached via its nitrogen atom.

Either enantiomer or a racemic mixture of the hydroxy compound of formula 34 can be coupled to the 3-hydroxy nicotinate methyl ester of formula 35 via a Mitsumobu reaction, to provide the ester of formula 36, which can be reacted with the methylcyanoquinoline of formula 37 to provide the alcohol of formula 38. Ammonium fluoride is added to the reaction mixture to remove the silyl protecting group. The reaction of the compound of formula 38 with methane sulfonyl chloride (MsCl) provides the mesylate compound of formula 39, which after removal of the Boc protecting group with hydrochloric acid in ethyl acetate, provides the mesylate compound of formula 40 as a hydrogen chloride salt. The amine of formula 41 in which presents an amino containing compound or heterocycle that is sufficiently nucleophilic to participate in a displacement reaction with the mesylate intermediate 40. The reaction of the mesylate compound of formula 40 with the amine of formula 41 in the presence of sodium iodide then provides the Benzo[c][2,7]naphthyridine Derivatives represented by formula 42.

Certain Benzo[c][2,7]naphthyridine Derivatives of Formula (I) can be prepared according to Scheme 6.

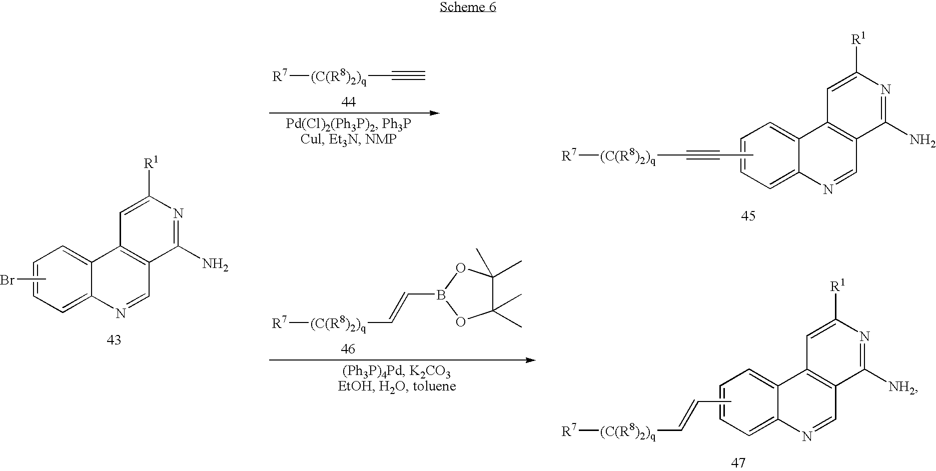

wherein Y is —(CH₂)ₐ—, a=0, k=0, W′ is —CC—, or —CH═CH— and R¹, R³, R⁴ R⁷, R⁸, and q are as defined above for Benzo[c][2,7]naphthyridine Derivatives of Formula (I) or (Ia). The bromo-substituted compound of formula 43, which is a Benzo[c][2,7]naphthyridine Derivative, can be reacted with an acetylene derivative 44 using a palladium and copper catalyst system in the presence of a base, e.g., triethylamine, in a solvent such as N-methylpyrrolidinone, to provide the compound of formula 45. Additionally, the bromo-substituted compound of formula 43 can be reacted with an alkene of formula 46 in the presence of a palladium catalyst and base, such as potassium carbonate, in a mixture of ethanol, water, and toluene, to provide the Benzo[c][2,7]naphthyridine Derivative represented by formula 47.

Additional compounds useful in preparation of the Benzo[c][2,7]naphthyridine Derivatives can be prepared as outlined in Scheme 7 below.

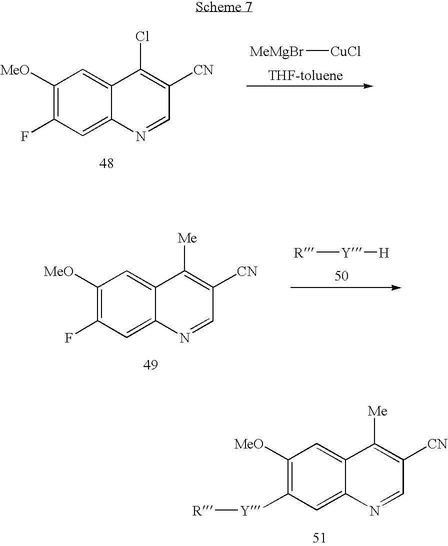

wherein the moiety R′″—Y′″— in the compound of formula 50, is

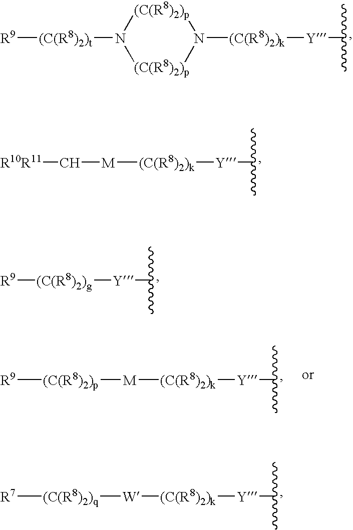

wherein g, k, p, t, R⁷, R⁸, R⁹, R¹⁰, R¹¹, M and W′ are defined above for the Benzo[c][2,7]naphthyridine Derivatives of Formula (I) or (Ia), and Y′″—S—, —O—, and —N(R⁸)—. Thus, the 4-chloro-3-cyano-quinoline compound of formula 50 can be a mercaptan, an alcohol, a primary amine or a secondary amine. The 4-chloro-3-cyano-quinoline compound of formula 48 can be converted to the 4-methyl-3-cyanoquinoline compound of formula 49, for example, by using the copper catalyzed substitution with methyl magnesium bromide as illustrated in Scheme 7. When R′″—Y′″—H is an alcohol or mercaptan (e.g., Y′″ is —S— or —O—), the anion is first formed using a base, such as lithium hexamethyldisilazane or sodium hydride, in a solvent such as THF. This anion is reacted at temperatures from 0-80° C. with the compound of formula 49 to provide a 4-methyl-3-cyanoquinoline compound of formula 51. When the compound of formula 50 is a primary or secondary amine (i.e., Y′″ is —N(R⁸)—), it can be heated with the compound of formula 49 in a solvent such as N-methylpyrrolidinone at about 100° C. to provide the 4-methyl-3-cyanoquinoline compound of formula 51. A compound of formula 51 is useful in preparation of a Benzo[c][2,7]naphthyridine Derivative.

Scheme 8 illustrates preparation of additional compounds useful in preparation of a Benzo[c][2,7]naphthyridine Derivative.

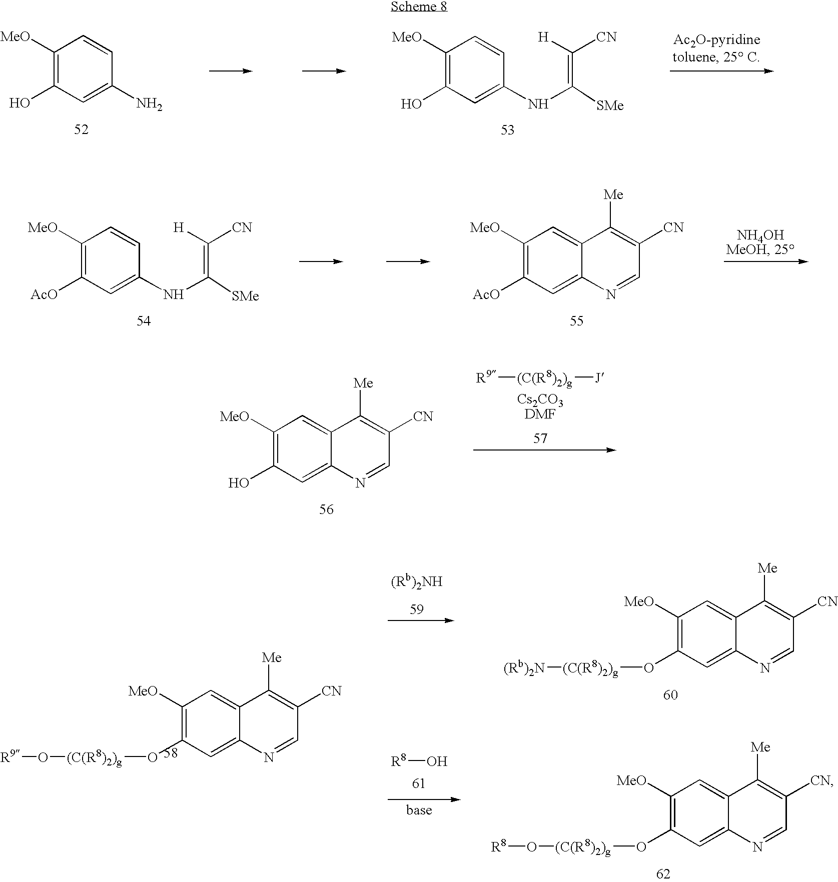

wherein R⁸ and g are as defined above for the Benzo[c][2,7]naphthyridine Derivatives of Formula (I), R^{9″} is —F, —Cl, —Br, —I or —OR⁸, J′ is a —Br, —I, -mesylate, or -tosylate group and (R^{b})₂N— in the represents an amine or a heterocycle that contains a nucleophilic nitrogen atom that can displace the chlorine atom when R^{9″} is —Cl.

The aniline compound of formula 52 can be converted to the secondary amine of formula 53, for example as described in Scheme 3b. The hydroxyl group of the compound of formula 53 can be protected, for example, as an acetate by reaction with an acetylating agent, such as acetic anhydride and pyridine in toluene, to provide a secondary amine of formula 54, which can be converted to the 4-methyl-3-cyanoquinoline compound of formula 55, for example by using methods outlined in Scheme 3b. The acetate group can be removed, for example with ammonium hydroxide, to provide the 4-methyl-3-cyanoquinoline compound of formula 56. The reaction of the compound of formula 56 with the compound of formula 57 using cesium carbonate in DMF, provides the 4-methyl-3-cyanoquinoline compound of formula 58. R^{b}₂N— in the compound of formula 59 represents an amine or a heterocycle that contains a nucleophilic nitrogen atom that can displace the chlorine atom in the compound of formula 58 when R⁹ is —Cl. The displacement can be carried out in an inert solvent, e.g., THF, toluene or DMF, at elevated temperature, for example 60°-100° C., to provide a 4-methyl-3-cyanoquinoline compound of formula 60, useful for preparation of a Benzo[c][2,7]naphthyridine Derivative. Reaction of the compound of formula 58 with an alkoxide anion derived from an alcohol of formula 61 using a base, such as sodium hydride in a solvent such as DMF or THF, provides the 4-methyl-3-cyanoquinoline compound of formula 62, useful in preparation of a Benzo[c][2,7]naphthyridine Derivative.

Certain Benzo[c][2,7]naphthyridine Derivatives of Formula (I) can be prepared according to Scheme 9.

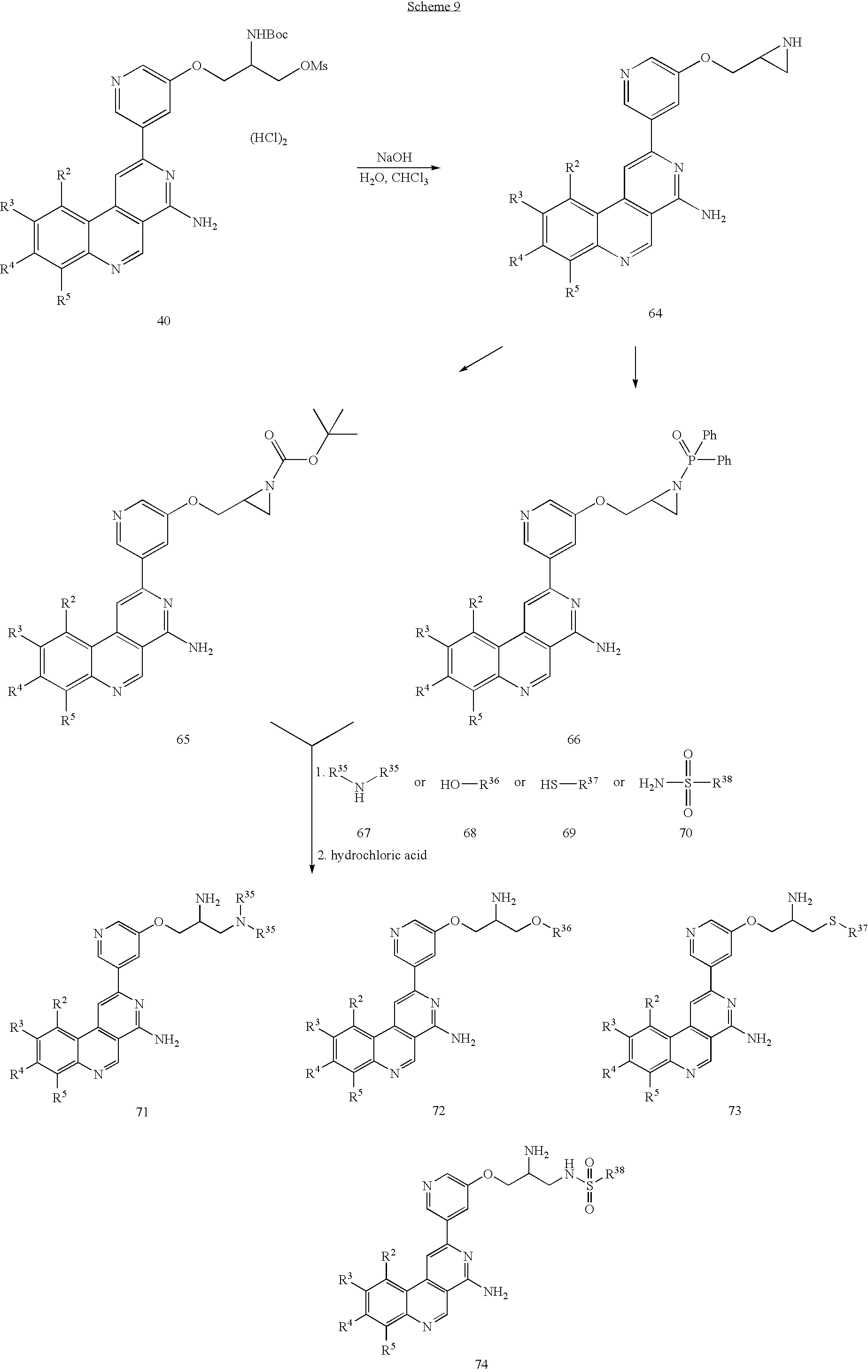

wherein R², R³, R⁴, R⁵ are as defined above for Benzo[c][2,7]naphthyridine Derivatives of Formula (I). N(R³⁵)₂ is a subset of Y₅ comprising an amino group or a heterocycle that is attached via its nitrogen atom. HO—R³⁶ can be a phenol derivative or a hydroxyl-substituted mono or bicyclic heterocycle and HS—R³⁷ can be a mercaptan, a thiophenol derivative, or a sulfhydryl substituted mono or bicyclic heterocycle. NH₂—SO₂—R³⁸ is a sulfonamide, wherein R³⁸ can be an alkyl derivative, phenyl derivative, or a mono or bicyclic heterocycle. Either enantiomer of 40, prepared as shown in Scheme 5b can be converted to either enantiomer of the aziridine derivative 64 by the reaction with a base (e.g., aqueous sodium hydroxide) in a two phase system using chloroform. The aziridine derivative 64 can be protected and activated for subsequent reactions by making the protected aziridine derivatives 65 or 66. Heating the protected aziridine derivatives 65 or 66 with amine 67 in an inert solvent, such as methoxyethanol or dimethylforamide, using a catalyst such as lithium iodide, followed by removal of the protecting group using an aqueous acid, such as hydrochloric acid, provides either entantiomer of the Benzo[c][2,7]naphthyridine Derivatives of this invention represented by formula 71. Heating the protected aziridine derivatives 65 or 66 with alcohol 68 in an inert solvent such as dimethylformamide, using a base such as cesium carbonate, followed by removal of the protecting group using an aqueous acid such as hydrochloric acid, provides either entantiomer of the Benzo[c][2,7]naphthyridine Derivatives represented by formula 72. Heating the protected aziridine derivatives 65 or 66 with 69 in an inert solvent such as dimethylformamide, using a base such as cesium carbonate, followed by removal of the protecting group using an aqueous acid such as hydrochloric acid, provides either entantiomer of the Benzo[c][2,7]naphthyridine Derivatives represented by formula 73. Heating the protected aziridine derivatives 65 or 66 with sulfonamide 70 in an inert solvent such as dimethylformamide, using a base such as cesium carbonate, followed by removal of the protecting group using an aqueous acid, such as hydrochloric acid, provides either entantiomer of the compounds described herein represented by formula 74.

In addition to the methods described in Schemes 1a-9, the Benzo[c][2,7]naphthyridine Derivatives can be prepared by using the methods described in the specific examples given below.

The compounds that can be made using the methods provided above in Schemes 1a-8 and the methods described in the specific examples herein, can be derivatized using methods known to one skilled in the art of organic synthesis in order to provide the entire scope of the Benzo[c][2,7]naphthyridine Derivatives of Formula (I).

### 5.6 Methods For Using The Benzo[c][2,7]naphthyridine Derivatives

In accordance with the invention, the Benzo[c][2,7]naphthyridine Derivatives are administered to a subject in need of treatment or prevention of a proliferative disorder.

### 5.6.1 Methods for Treating or Preventing a Proliferative Disorder

A proliferative disorder can be treated or prevented by administration of an effective amount of a Benzo[c][2,7]naphthyridine Derivative.

Proliferative disorders that can be treated or prevented by administering an effective amount of a Benzo[c][2,7]naphthyridine Derivative include, but are not limited to, cancer, uterine fibroids, benign prostatic hyperplasia, familial adenomatosis polyposis, neuro-fibromatosis, atherosclerosis, pulmonary fibrosis, arthritis, psoriasis, glomerulonephritis, restenosis following angioplasty or vascular surgery, hypertrophic scar formation, an inflammatory bowel disease, transplantation rejection, endotoxic shock, a fungal infection, a defective apoptosis-associated condition, or a proliferative disease that is dependent on PDK-1 activity.

In one embodiment, the proliferative disorder is cancer.

In another embodiment, the proliferative disorder is a proliferative disorder that is dependent on PDK-1 activity.

### 5.6.2 Methods for Treating or Preventing Cancer

The Benzo[c][2,7]naphthyridine Derivatives can be used to treat or prevent cancer.

The invention provides methods for treating or preventing cancer, comprising administering to a subject in need of such treatment or prevention an effective amount of a Benzo[c][2,7]naphthyridine Derivative.

Examples of cancers treatable or preventable using the Benzo[c][2,7]naphthyridine Derivatives include, but are not limited to, a cancer which expresses PDK-1, the cancers disclosed below in Table 1 and metastases thereof.

TABLE 1

Solid tumors, including but not limited to:
fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon cancer
colorectal cancer
kidney cancer
pancreatic cancer
bone cancer
breast cancer
ovarian cancer
prostate cancer
esophageal cancer
stomach cancer
oral cancer
nasal cancer
throat cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
uterine cancer
testicular cancer
small cell lung carcinoma
bladder carcinoma
lung cancer
epithelial carcinoma
skin cancer
melanoma
neuroblastoma
retinoblastoma
Blood-borne cancers, including but not limited to:
acute lymphoblastic leukemia
acute lymphoblastic B-cell leukemia
acute lymphoblastic T-cell leukemia
acute myeloblastic leukemia
acute promyelocytic leukemia
acute monoblastic leukemia
acute erythroleukemic leukemia
acute megakaryoblastic leukemia
acute myelomonocytic leukemia
acute nonlymphocyctic leukemia
acute undifferentiated leukemia
chronic myelocytic leukemia (“CML”)
chronic lymphocytic leukemia (“CLL”)
hairy cell leukemia
multiple myeloma
Lymphomas, including but not limited to:
Hodgkin's disease
non-Hodgkin's Lymphomas:
Multiple myeloma
Waldenström's macroglobulinemia
Heavy chain disease
Polycythemia vera
CNS and brain cancers, including but not limited to:
glioma
pilocytic astrocytoma
astrocytoma
anaplastic astrocytoma
glioblastoma multiforme
medulloblastoma
craniopharyngioma
ependymoma
pinealoma
hemangioblastoma
acoustic neuroma
oligodendroglioma
meningioma
vestibular schwannoma
adenoma
metastatic brain tumor
meningioma
spinal tumor
medulloblastoma

In one embodiment, the cancer is lung cancer, breast cancer, colorectal cancer, prostate cancer, a leukemia, a lymphoma, a skin cancer, a brain cancer, a cancer of the central nervous system, ovarian cancer, uterine cancer, stomach cancer, pancreatic cancer, esophageal cancer, kidney cancer, liver cancer, or a head and neck cancer.

In another embodiment, the cancer is metastatic cancer.

In yet another embodiment, the cancer is a cancer which expresses PDK-1.

In still another embodiment, the subject has previously undergone or is presently undergoing treatment for cancer. Such previous treatments include, but are not limited to, prior chemotherapy, radiation therapy, surgery or immunotherapy, such as cancer vaccines.

The Benzo[c][2,7]naphthyridine Derivatives are also useful for the treatment or prevention of a cancer caused by a virus. Such viruses include human papilloma virus, which can lead to cervical cancer (see, e.g., Hernandez-Avila et al., Archives of Medical Research (1997) 28:265-271); Epstein-Barr virus (EBV), which can lead to lymphoma (see, e.g., Herrmann et al., J Pathol (2003) 199(2):140-5); hepatitis B or C virus, which can lead to liver carcinoma (see, e.g., El-Serag, J Clin Gastroenterol (2002) 35(5 Suppl 2):S72-8); human T cell leukemia virus (HTLV)-I, which can lead to T-cell leukemia (see e.g., Mortreux et al., Leukemia (2003) 17(1):26-38); human herpesvirus-8 infection, which can lead to Kaposi's sarcoma (see, e.g., Kadow et al., Curr Opin Investig Drugs (2002) 3(11): 1574-9); and Human Immune deficiency Virus (HIV) infection, which can lead to cancer as a consequence of immunodeficiency (see, e.g., Dal Maso et al., Lancet Oncol (2003) 4(2):110-9).

The Benzo[c][2,7]naphthyridine Derivatives can also be administered to prevent the progression of a cancer, including but not limited to the cancers listed in Table 1. Such prophylactic use includes that in which non-neoplastic cell growth consisting of hyperplasia, metaplasia, or most particularly, dysplasia has occurred.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a subject, can indicate the desirability of prophylactic/therapeutic administration of the Benzo[c][2,7]naphthyridine Derivatives. Such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 dalton cell surface protein, etc. (see also id., at pp. 84-90 for characteristics associated with a transformed or malignant phenotype).

In a specific embodiment, leukoplakia, a benign-appearing hyperplastic or dysplastic lesion of the epithelium, or Bowen's disease, a carcinoma in situ, are treatable or preventable according to the present methods.

In another embodiment, fibrocystic disease (cystic hyperplasia, mammary dysplasia, particularly adenosis (benign epithelial hyperplasia)) are treatable or preventable according to the present methods.

In other embodiments, a subject that exhibits one or more of the following predisposing factors for malignancy can be administered an amount of a Benzo[c][2,7]naphthyridine Derivative which is effective to treat or prevent cancer: a chromosomal translocation associated with a malignancy (e.g., the Philadelphia chromosome for chronic myelogenous leukemia, t(14;18) for follicular lymphoma); familial polyposis or Gardner's syndrome; benign monoclonal gammopathy; a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; and exposure to carcinogens (e.g., smoking, second-hand smoke exposure, and inhalation of or contacting with certain chemicals).

### 5.6.3 Combination Chemotherapy for the Treatment of Cancer

In one embodiment, the present methods for treating cancer or preventing cancer further comprise administering another anticancer agent.

In one embodiment, the present invention provides methods for treating or preventing cancer in a subject, the method comprising the administration of an effective amount of: (i) a Benzo[c][2,7]naphthyridine Derivative and (ii) another anticancer agent.

In one embodiment, (i) a Benzo[c][2,7]naphthyridine Derivative and (ii) another anticancer agent are administered in doses commonly employed when such agents are used as monotherapy for the treatment of cancer.

In another embodiment, (i) a Benzo[c][2,7]naphthyridine Derivative and (ii) another anticancer agent act synergistically and are administered in doses that are less than the doses commonly employed when such agents are used as monotherapy for the treatment of cancer.

The dosage of the (i) a Benzo[c][2,7]naphthyridine Derivative, and (ii) another anticancer agent administered as well as the dosing schedule can depend on various parameters, including, but not limited to, the cancer being treated, the subject's general health, and the administering physician's discretion.

A Benzo[c][2,7]naphthyridine Derivative can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the other anticancer agent to a subject in need thereof. In various embodiments, i) a Benzo[c][2,7]naphthyridine Derivative, and (ii) another anticancer agent are administered 1 minute apart, 10 minutes apart, 30 minutes apart, less than 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart, or no more than 48 hours apart. In one embodiment, i) a Benzo[c][2,7]naphthyridine Derivative, and (ii) another anticancer agent are administered within 3 hours of each other. In another embodiment, i) a Benzo[c][2,7]naphthyridine Derivative, and (ii) another anticancer agent are administered 1 minute to 24 hours apart.

In one embodiment, an effective amount of a Benzo[c][2,7]naphthyridine Derivative and an effective amount of another anticancer agent are present in the same composition. In one embodiment, this composition is useful for oral administration. In another embodiment, this composition is useful for intravenous administration.

Cancers that can be treated or prevented by administering a Benzo[c][2,7]naphthyridine Derivative and another anticancer agent include, but are not limited to, the list of cancers set forth in Table 1.

In one embodiment the cancer is lung cancer, breast cancer, colorectal cancer, prostate cancer, a leukemia, a lymphoma, a skin cancer, a brain cancer, a cancer of the central nervous system, ovarian cancer, uterine cancer, stomach cancer, pancreatic cancer, esophageal cancer, kidney cancer, liver cancer, or a head and neck cancer.

The Benzo[c][2,7]naphthyridine Derivative and the other anticancer agent can act additively or synergistically. A synergistic combination of a Benzo[c][2,7]naphthyridine Derivative and another anticancer agent might allow the use of lower dosages of one or both of these agents and/or less frequent dosages of one or both of the Benzo[c][2,7]naphthyridine Derivatives and other anticancer agents and/or to administer the agents less frequently can reduce any toxicity associated with the administration of the agents to a subject without reducing the efficacy of the agents in the treatment of cancer. In addition, a synergistic effect might result in the improved efficacy of these agents in the treatment of cancer and/or the reduction of any adverse or unwanted side effects associated with the use of either agent alone.

In one embodiment, a Benzo[c][2,7]naphthyridine Derivative and another anticancer agent may act synergistically when administered in doses typically employed when such agents are used as monotherapy for the treatment of cancer. In another embodiment, a Benzo[c][2,7]naphthyridine Derivative and another anticancer agent may act synergistically when administered in doses that are less than doses typically employed when such agents are used as monotherapy for the treatment of cancer.

In one embodiment, the administration of an effective amount of a Benzo[c][2,7]naphthyridine Derivative and an effective amount of another anticancer agent inhibits the resistance of a cancer to the Benzo[c][2,7]naphthyridine Derivative and/or the other anticancer agent. In one embodiment, the cancer is a solid tumor.

In one embodiment, other anticancer agents useful in the methods and compositions of the present invention include, but are not limited to, a drug listed in Table 2 or a pharmaceutically acceptable salt thereof.

TABLE 2

| | |
|---|---|
| Alkylating agents | |
| Nitrogen mustards: | Cyclophosphamide |
| | Ifosfamide |
| | Trofosfamide |
| | Chlorambucil |
| Nitrosoureas: | Carmustine (BCNU) |
| | Lomustine (CCNU) |
| Alkylsulphonates: | Busulfan |
| | Treosulfan |
| Triazenes: | Dacarbazine |
| | Procarbazine |
| | Temozolomide |
| Platinum complexes: | Cisplatin |
| | Carboplatin |
| | Aroplatin |
| | Oxaliplatin |
| Plant Alkaloids | |
| Vinca alkaloids: | Vincristine |
| | Vinblastine |
| | Vindesine |
| | Vinorelbine |
| Taxanes: | Paclitaxel |
| | Docetaxel |
| DNA Topoisomerase Inhibitors | |
| Epipodophyllins: | Etoposide |
| | Teniposide |
| | Topotecan |
| | Irinotecan |
| | 9-aminocamptothecin |
| | Camptothecin |
| | Crisnatol |
| Mitomycins: | Mitomycin C |
| Anti-folates: | |
| DHFR inhibitors: | Methotrexate |
| | Trimetrexate |
| IMP dehydrogenase Inhibitors: | Mycophenolic acid |
| | Tiazofurin |
| | Ribavirin |
| | EICAR |
| Ribonuclotide reductase | Hydroxyurea |
| Inhibitors: | Deferoxamine |
| Pyrimidine analogs: | |
| Uracil analogs: | 5-Fluorouracil |
| | Fluoxuridine |
| | Doxifluridine |
| | Ralitrexed |
| Cytosine analogs: | Cytarabine |
| | Cytosine arabinoside |
| | Fludarabine |
| | Gemcitabine |
| | Capecitabine |
| Purine analogs: | Mercaptopurine |
| | Thioguanine |
| | O-6-benzylguanine |
| DNA Antimetabolites: | 3-HP |
| | 2′-deoxy-5-fluorouridine |
| | 5-HP |
| | alpha-TGDR |
| DNA Antimetabolites: | |
| | aphidicolin glycinate |
| | ara-C |
| | 5-aza-2′-deoxycytidine |
| | beta-TGDR |
| | cyclocytidine |
| | guanazole |
| | inosine glycodialdehyde |
| | macebecin II |
| | Pyrazoloimidazole |
| Hormonal therapies: | |
| Receptor antagonists | |
| Anti-estrogen: | Tamoxifen |
| | Raloxifene |
| | Megestrol |
| LHRH agonists: | Goserelin |
| | Leuprolide acetate |
| Anti-androgens: | Flutamide |
| | Bicalutamide |
| Retinoids/Deltoids | |
| | Cis-retinoic acid |
| Vitamin A derivative: | All-trans retinoic acid (ATRA-IV) |
| Vitamin D3 analogs: | EB 1089 |
| | CB 1093 |
| | KH 1060 |
| Photodynamic therapies: | Vertoporfin (BPD-MA) |
| | Phthalocyanine |
| | Photosensitizer Pc4 |
| | Demethoxy-hypocrellin A |
| | (2BA-2-DMHA) |
| Cytokines: | Interferon-α |
| | Interferon-β |
| | Interferon-γ |
| | Tumor necrosis factor |
| | Interleukin-2 |
| Angiogenesis Inhibitors: | Angiostatin (plasminogen fragment) |
| | antiangiogenic antithrombin III |
| | Angiozyme |
| | ABT-627 |
| | Bay 12-9566 |
| | Benefin |
| | Bevacizumab |
| | BMS-275291 |
| | cartilage-derived inhibitor (CDI) |
| | CAI |
| | CD59 complement fragment |
| | CEP-7055 |
| | Col 3 |
| | Combretastatin A-4 |
| | Endostatin (collagen XVIII fragment) |
| | Fibronectin fragment |
| | Gro-beta |
| | Halofuginone |
| | Heparinases |
| | Heparin hexasaccharide fragment |
| | HMV833 |
| | Human chorionic gonadotropin (hCG) |
| | IM-862 |
| | Interleukins |
| | Kringle 5 (plasminogen fragment) |
| | Marimastat |
| | Metalloproteinase inhibitors |
| | 2-Methoxyestradiol |
| | MMI 270 (CGS 27023A) |
| | MoAb IMC-1C11 |
| | Neovastat |
| | NM-3 |
| | Panzem |
| | PI-88 |
| | Placental ribonuclease inhibitor |
| | Plasminogen activator inhibitor |
| | Platelet factor-4 (PF4) |
| | Prinomastat |
| | Prolactin 16 kD fragment |
| | Proliferin-related protein (PRP) |
| | PTK 787/ZK 222594 |
| | Retinoids |
| | Solimastat |
| | Squalamine |
| | SS 3304 |
| | SU 5416 |
| | SU6668 |
| | SU11248 |
| | Tetrahydrocortisol-S |
| | Tetrathiomolybdate |
| | Thalidomide |
| | Thrombospondin-1 (TSP-1) |
| | TNP-470 |
| | Transforming growth factor-beta |
| | (TGF-β) |
| | Vasculostatin |
| | Vasostatin (calreticulin fragment) |
| | ZD6126 |
| | ZD 6474 |
| | farnesyl transferase inhibitors (FTI) |
| | Bisphosphonates |
| Antimitotic agents: | Allocolchicine |
| | Halichondrin B |
| | Colchicine |
| | colchicine derivative |
| | dolastatin 10 |
| | Maytansine |
| | Rhizoxin |
| | Thiocolchicine |
| | trityl cysteine |
| Others: | |
| Isoprenylation inhibitors: | |
| Dopaminergic neurotoxins: | 1-methyl-4-phenylpyridinium ion |
| Cell cycle inhibitors: | Staurosporine |
| Actinomycins: | Actinomycin D |
| | Dactinomycin |
| Bleomycins: | Bleomycin A2 |
| | Bleomycin B2 |
| | Peplomycin |
| Anthracyclines: | Daunorubicin |
| | Doxorubicin |
| | Idarubicin |
| | Epirubicin |
| | Pirarubicin |
| | Zorubicin |
| | Mitoxantrone |
| MDR inhibitors: | Verapamil |
| Ca²⁺ATPase inhibitors: | Thapsigargin |

Additional suitable other anticancer agents useful in the methods and compositions of the present invention include, but are not limited to abiraterone, acivicin, aclarubicin, acodazole, acronine, acylfulvene, adecypenol, adozelesin, aldesleukin, an ALL-TK antagonist, altretamine, ambamustine, ambomycin, ametantrone, amidox, amifostine, aminoglutethimide, aminolevulinic acid, amrubicin, amsacrine, anagrelide, anastrozole, andrographolide, an angiogenesis inhibitor, antarelix, anthramycin, an apoptosis gene modulator, apurinic acid, ara-CDP-DL-PTBA, arginine deaminase, L-asparaginase, asperlin, asulacrine, atamestane, atrimustine, axinastatin 1, axinastatin 2, axinastatin 3, azacitidine, azasetron, azatoxin, azetepa, azatyrosine, azotomycin, batimastat, benzodepa, bisantrene, bisnafide, bizelesin, brequinar, bropirimine, balanol, a BCR/ABL antagonist, beta-alethine, betaclamycin B, betulinic acid, bisaziridinylspermine, bisnafide, bistratene A, bizelesin, calcipotriol, calphostin C, calusterone, canarypox IL-2, carubicin, carboxyamidotriazole, CaRest M3, CARN 700, carzelesin, castanospermine, cecropin B, cetrorelix, chloroquinoxaline, cicaprost, cirolemycin, cladribine, clotrimazole, collismycin A, collismycin B, conagenin, crambescidin 816, crisnatol, cryptophycin 8, cryptophycin A derivatives, curacin A, cyclopentanthraquinones, cycloplatam, cypemycin, cytostatin, dacliximab, decitabine, dehydrodidemnin B, deslorelin, dexifosfamide, dexormaplatin, dexrazoxane, dexdiaziquone, didemnin B, didox, diethylnorspermine, dihydro-5-acytidine, dihydrotaxol, dioxamycin, diphenyl spiromustine, docosanol, dolasetron, droloxifene, dronabinol, duazomycin, duocarmycin SA, ecomustine, edatrexate, eflornithine, elsamitrucin, enloplatin, enpromate, epipropidine, erbulozole, esorubicin, estramustine, estramustine, an estrogen antagonist, etanidazole, etoprine, exemestane, fadrozole, fazarabine, fenretinide, finasteride, flavopiridol, flezelastine, fluasterone, fluorodaunorunicin, floxuridine, fluorocitabine, forfenimex, formestane, fostriecin, fotemustine, gadolinium texaphyrin, galocitabine, ganirelix, a gelatinase inhibitor, a glutathione inhibitor, hepsulfam, herbimycin A, heregulin, hexamethylene bisacetamide, hypericin, ibandronic acid, idoxifene, idramantone, ilmofosine, ilomastat, imatinib mesylate, imidazoacridones, imiquimod, an IGF-1 inhibitor, iobenguane, iodoipomeanol, iproplatin, irsogladine, isobengazole, isohomohalicondrin B, itasetron, jasplakinolide, leucovorin, levamisole, leuprorelin, liarozole, lissoclinamide 7, lobaplatin, lombricine, lometrexol, lonidamine, losoxantrone, lovastatin, loxoribine, lurtotecan, lutetium texaphyrin, lysofylline, mannostatin A, masoprocol, maspin, a matrix metalloproteinase inhibitor, mechlorethamine, megestrol acetate melphalan, metoclopramide, mifepristone, miltefosine, mirimostim, mitoguazone, mitolactol, mitonafide, mofarotene, molgramostim, mopidamol, a multiple drug resistance gene inhibitor, myriaporone, N-acetyldinaline, nafarelin, nagrestip, napavin, naphterpin, nartograstim, nedaplatin, nemorubicin, neridronic acid, nilutamide, nisamycin, a nitrogen mustard, a nitric oxide modulator, a nitrosourea, nitrullyn, nocodazole, octreotide, okicenone, onapristone, oracin, ormaplatin, osaterone, oxaunomycin, palauamine, palmitoylpamidronic acid, panaxytriol, panomifene, parabactin, pazelliptine, pegaspargase, peldesine, peliomycin, pentamustine, pentosan, pentostatin, pentrozole, peplomycin, perfosfamide, perflubron, perfosfamide, phenazinomycin, a phosphatase inhibitor, picibanil, pilocarpine, pipobroman, piposulfan, piritrexim, placetin A, placetin B, plicamycin, porfiromycin, plomestane, porfimer sodium, porfiromycin, prednimustine, prednisone, prostaglandin J2, microalgal, puromycin, pyrazoloacridine, pyrazofurin, a raf antagonist, raltitrexed, ramosetron, a ras farnesyl protein transferase inhibitor, a ras-GAP inhibitor, retelliptine demethylated, RII retinamide, riboprine, rogletimide, rohitukine, romurtide, roquinimex, rubiginone B1, ruboxyl, safingol, saintopin, SarCNU, sarcophytol A, sargramostim, semustine, a signal transduction modulator, simtrazene, sizofuran, sobuzoxane, solverol, sonermin, sparfosic acid, sparfosate, sparsomycin, spicamycin D, spiromustine, spiroplatin, splenopentin, spongistatin 1, a stem-cell division inhibitor, stipiamide, streptonigrin, a stromelysin inhibitor, sulfinosine, suradista, suramin, swainsonine, talisomycin, tallimustine, tauromustine, tazarotene, tecogalan, tegafur, tellurapyrylium, a telomerase inhibitor, teloxantrone, temoporfin, teroxirone, testolactone, tetrachlorodecaoxide, tetrazomine, thaliblastine, thiamiprine, thiocoraline, thrombopoietin, thymalfasin, thymotrinan, tirapazamine, titanocene, topsentin, toremifene, trestolone, tretinoin, triacetyluridine, triciribine, trimetrexate, triptorelin, tropisetron, tubulozole, turosteride, a tyrosine kinase inhibitor, ubenimex, uracil mustard, uredepa, vapreotide, variolin B, velaresol, veramine, verteporfin, vinxaltine, vinepidine, vinglycinate, vinleurosine, vinrosidine, vinzolidine, vitaxin, vorozole, zanoterone, zeniplatin, zilascorb, zinostatin, and zorubicin.

In various embodiments, the other anticancer agent is an alkylating agent, a platinum-containing agent, an anthracycline, a vinca alkaloid, a taxane, a topoisomerase inhibitor or an angiogenesis inhibitor.

In one embodiment, the other anticancer agent is administered orally.

In another embodiment, the other anticancer agent is administered intravenously.

### 5.6.4 Multi-Therapy for Cancer

The Benzo[c][2,7]naphthyridine Derivatives can be administered to a subject that has undergone or is currently undergoing one or more additional anticancer therapies including, but not limited to, surgery, radiation therapy, or immunotherapy, such as cancer vaccines.

In one embodiment, the invention provides methods for treating or preventing cancer comprising administering to a subject in need thereof (a) an amount of a Benzo[c][2,7]naphthyridine Derivative effective to treat or prevent cancer; and (b) another anticancer therapy including, but not limited to, surgery, radiation therapy, or immunotherapy, such as a cancer vaccine.

In one embodiment, the other anticancer therapy is radiation therapy.

In another embodiment, the other anticancer therapy is surgery.

In still another embodiment, the other anticancer therapy is immunotherapy.

In a specific embodiment, the present methods for treating or preventing cancer comprise administering a Benzo[c][2,7]naphthyridine Derivative and radiation therapy. The radiation therapy can be administered concurrently with, prior to, or subsequent to the Benzo[c][2,7]naphthyridine Derivative, in one embodiment, at least an hour, five hours, 12 hours, a day, a week, a month, or several months (e.g., up to three months), prior or subsequent to administration of the Benzo[c][2,7]naphthyridine Derivatives.

Where the other anticancer therapy is radiation therapy, any radiation therapy protocol can be used depending upon the type of cancer to be treated. For example, but not by way of limitation, X-ray radiation can be administered; in particular, high-energy megavoltage (radiation of greater that 1 MeV energy) can be used for deep tumors, and electron beam and orthovoltage X-ray radiation can be used for skin cancers. Gamma-ray emitting radioisotopes, such as radioactive isotopes of radium, cobalt and other elements, can also be administered.

Additionally, in one embodiment the invention provides methods of treatment of cancer using a Benzo[c][2,7]naphthyridine Derivative as an alternative to chemotherapy or radiation therapy where the chemotherapy or the radiation therapy results in negative side effects in the subject being treated. The subject being treated can, optionally, be treated with another anticancer therapy such as surgery, radiation therapy, or immunotherapy.

The Benzo[c][2,7]naphthyridine Derivatives can also be used in vitro or ex vivo, such as for the treatment of certain cancers, including, but not limited to leukemias and lymphomas, wherein such treatment involves autologous stem cell transplants. This can involve a process in which the subject's autologous hematopoietic stem cells are harvested and purged of all cancer cells, the subject's remaining bone-marrow cell population is then eradicated via the administration of a Benzo[c][2,7]naphthyridine Derivative and/or radiation, and the resultant stem cells are infused back into the subject. Supportive care can be subsequently provided while bone marrow function is restored and the subject recovers.

### 5.6.5 Methods for Treating or Preventing an Autoimmune Disease

An autoimmune can be treated or prevented by administration of an effective amount of a Benzo[c][2,7]naphthyridine Derivative.

Autoimmune diseases that can be treated or prevented by administering an effective amount of a Benzo[c][2,7]naphthyridine Derivative include, but are not limited to, multiple sclerosis (MS), Addison's disease, angiitis, alopecia greata, ankylosing spondylitis, antiphospholipid syndrome, autism, autoimmune haemolytic anaemia, autoimmune hepatitis, Behcet's syndrome, Berger's disease, bullous pemphigoid, cardiomyopathy, coeliac disease, chronic fatigue syndrome (CFS, CFIDS), chronic inflammatory polyneuropathy, Churg-Strauss syndrome, CREST syndrome, Crohn's disease, dermatomyositis, fibromyalgia, giant cell arteritis, Grave's disease, Guillain Barre syndrome, Hashimoto's thyroiditis, idiopathic pulmonary fibrosis, idiopathic thrombocytopenia purpura (ITP), type 1 diabetes, lichen planus, Meniere's disease, mixed connective tissue disease, myasthenia gravis, polyarteritis nodosa, polymyalgia rheumatica, polymyositis, primary biliary cirrhosis, psoriasis, Raynaud's disease, Reiter's syndrome, relapsing polychondritis, rheumatic fever, rheumatoid arthritis (RA), sarcoidosis, scleroderma, Sjögren's syndrome, stiff-man syndrome, systemic lupus erythematosus (SLE), ulcerative colitis, uveitis, vitiligo and Wegener's granulomatosis.

In one embodiment, the autoimmune disease is dependent on PKCθ activity.

### 5.6.6 Modulation of Activity of Protein Kinases

A Benzo[c][2,7]naphthyridine Derivative is useful for modulating activity of one or more protein kinases, which include, but are not limited to PDK-1 kinase and AGC family protein kinases (e.g., PKB (Akt), S6K or PKC).

### 5.6.6.1 Modulation of PDK-1 Activity

A Benzo[c][2,7]naphthyridine Derivative is useful for modulating PDK-1 activity.

PDK-1 is believed to phosphorylate and activate several AGC family protein kinases, including isoforms of protein kinase B (PKB)/Akt, p70 ribosomal S6 kinase (S6K), serum- and glucocorticoid-induced protein kinase (SGK) and protein kinase C(PKC), which are involved in regulating physiological processes relevant to metabolism, growth, proliferation and survival. Modulation of PDK-1 activity can, therefore, effect treatment or prevention of a disease or a condition which results from an increase, decrease, or lack of activity or expression of PDK-1, PKB (Akt), S6K, SGK, PKC or any other protein involved in the AGC kinase signal transduction pathway.

### 5.6.6.2 Modulation of PKB (Akt) Activity

A Benzo[c][2,7]naphthyridine Derivative is useful for modulating PKB (Akt) activity.

A number of cancers possess mutations in genes that result in elevation of cellular levels of PtdIns(3,4,5)P₃, with one of the most common mutations occurring in the PtdIns(3,4,5)P₃ 3-phosphatase PTEN (Phosphatase and tensin homolog) gene. This results in elevation of PKB (Akt) activity, which is believed to function as a driving force in promoting the uncontrolled proliferation and enhanced survival of these cells.

Modulation of PKB (Akt) activity can, therefore, effect treatment or prevention of a disease or a condition which results from an increase, decrease, or lack of activity or expression of a PKB (Akt) isoform. In one embodiment, the disease or condition is cancer.

### 5.6.6.3 Modulation of S6K Activity

A Benzo[c][2,7]naphthyridine Derivative is useful for modulating S6K activity

A number of cancers possess mutations in genes that result in elevation of cellular levels of PtdIns(3,4,5)P₃, with one of the most common mutations occurring in the PtdIns(3,4,5)P₃ 3-phosphatase PTEN (Phosphatase and tensin homolog) gene. This results in elevation of S6K activity, which is believed to function as a driving force in promoting the uncontrolled proliferation and enhanced survival of these cells.

Modulation of S6K activity can, therefore, effect treatment or prevention of a disease or a condition which results from an increase, decrease, or lack of activity or expression of S6K. In one embodiment, the disease or condition is cancer.

### 5.6.6.4 Modulation of PKC Activity

A Benzo[c][2,7]naphthyridine Derivatives is useful for modulating activity one or more isoforms of PKC.

PKC family kinases are involved in regulating physiological processes relevant to cell proliferation, differentiation and survival. Modulation of PKC activity can, therefore, effect treatment or prevention of diseases or conditions which result from an increase, decrease, or lack of activity or expression of an isoform of PKC. PKCθ, for example, is believed to be involved in the regulation of multiple T-cell functions necessary for the development of autoimmune disease. Examples of such a disease or condition include, but are not limited to, a proliferative disorder, such as cancer, and an autoimmune disease.

Examples of an autoimmune disease include, but are not limited to, multiple sclerosis (MS), Addison's disease, angiitis, alopecia greata, ankylosing spondylitis, antiphospholipid syndrome, autism, autoimmune haemolytic anaemia, autoimmune hepatitis, Behcet's syndrome, Berger's disease, bullous pemphigoid, cardiomyopathy, coeliac disease, chronic fatigue syndrome (CFS, CFIDS), chronic inflammatory polyneuropathy, Churg-Strauss syndrome, CREST syndrome, Crohn's disease, dermatomyositis, fibromyalgia, giant cell arteritis, Grave's disease, Guillain Barre syndrome, Hashimoto's thyroiditis, idiopathic pulmonary fibrosis, idiopathic thrombocytopenia purpura (ITP), type 1 diabetes, lichen planus, Meniere's disease, mixed connective tissue disease, myasthenia gravis, polyarteritis nodosa, polymyalgia rheumatica, polymyositis, primary biliary cirrhosis, psoriasis, Raynaud's disease, Reiter's syndrome, relapsing polychondritis, rheumatic fever, rheumatoid arthritis (RA), sarcoidosis, scleroderma, Sjögren's syndrome, stiff-man syndrome, systemic lupus erythematosus (SLE), ulcerative colitis, uveitis, vitiligo and Wegener's granulomatosis.

### 5.7 Therapeutic/Prophylactic Administration

In one embodiment, the invention provides compositions useful for treating or preventing a proliferative disorder or an autoimmune disease. The compositions are suitable for internal use and comprise an effective amount of a Benzo[c][2,7]naphthyridine Derivative and a physiologically acceptable carrier or vehicle.

A Benzo[c][2,7]naphthyridine Derivative can be administered in amounts that are effective to treat or prevent a proliferative disorder in a subject.

A Benzo[c][2,7]naphthyridine Derivative can be administered in amounts that are effective to treat or prevent an autoimmune disease in a subject.

Administration of a Benzo[c][2,7]naphthyridine Derivative can be accomplished via any mode of administration for therapeutic agents. These modes include systemic or local administration such as oral, nasal, parenteral, transdermal, subcutaneous, vaginal, buccal, rectal or topical administration modes. In some instances, administration will result in the release of a Benzo[c][2,7]naphthyridine Derivative into the bloodstream.

In one embodiment, the Benzo[c][2,7]naphthyridine Derivatives are administered orally.

Depending on the intended mode of administration, the compositions can be in solid, semi-solid or liquid dosage form, such as, for example, injectables, tablets, suppositories, pills, time-release capsules, elixirs, tinctures, emulsions, syrups, powders, liquids, suspensions, or the like, preferably in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Illustrative pharmaceutical compositions include tablets and gelatin capsules comprising a Benzo[c][2,7]naphthyridine Derivative and a physiologically acceptable carrier or vehicle. Illustrative carriers or vehicles include a) a diluent, e.g., lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; and/or e) absorbent, colorant, flavorant and sweetener.

Liquid, particularly injectable, compositions can, for example, be prepared by dissolution, dispersion, etc. For example, the Benzo[c][2,7]naphthyridine Derivative is dissolved in or mixed with a pharmaceutically acceptable solvent such as, for example, water, saline, aqueous dextrose, glycerol, ethanol, and the like, to thereby form an injectable isotonic solution or suspension.

The Benzo[c][2,7]naphthyridine Derivatives can be also formulated as a suppository that can be prepared from fatty emulsions or suspensions; using polyalkylene glycols such as propylene glycol, as the carrier.

The Benzo[c][2,7]naphthyridine Derivatives can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

Benzo[c][2,7]naphthyridine Derivatives can also be delivered by the use of monoclonal antibodies as individual carriers to which the Benzo[c][2,7]naphthyridine Derivative molecules are coupled. The Benzo[c][2,7]naphthyridine Derivatives can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the Benzo[c][2,7]naphthyridine Derivatives can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Parental injectable administration can be used for subcutaneous, intramuscular or intravenous injections and infusions. Injectables can be prepared in conventional forms, either as liquid solutions or suspensions or solid forms suitable for dissolving in liquid prior to injection.

One embodiment, for parenteral administration employs the implantation of a slow-release or sustained-released system, according to U.S. Pat. No. 3,710,795, incorporated herein by reference.

The compositions can be sterilized or contain non-toxic amounts of adjuvants, such as preserving, stabilizing, wetting or emulsifying agents, solution promoters, salts for regulating the osmotic pressure pH buffering agents, and other substances, including, but not limited to, sodium acetate or triethanolamine oleate. In addition, they can also contain other therapeutically valuable substances.

Compositions can be prepared according to conventional mixing, granulating or coating methods, respectively, and the present pharmaceutical compositions can contain from about 0.1% to about 99%, preferably from about 1% to about 70% of the Benzo[c][2,7]naphthyridine Derivative by weight or volume.

The dosage regimen utilizing the Benzo[c][2,7]naphthyridine Derivative can be selected in accordance with a variety of factors including type, species, age, weight, sex and medical condition of the subject; the severity of the condition to be treated; the route of administration; the renal or hepatic function of the subject; and the particular Benzo[c][2,7]naphthyridine Derivative employed. A person skilled in the art can readily determine and prescribe the effective amount of the drug useful for treating or preventing a proliferative disorder.

Effective dosage amounts of the Benzo[c][2,7]naphthyridine Derivatives, when administered to a subject, range from about 0.05 to about 1000 mg of Benzo[c][2,7]naphthyridine Derivative per day. Compositions for in vivo or in vitro use can contain about 0.5, 1.0, 2.5, 5.0, 10.0, 15.0, 25.0, 50.0, 100.0, 250.0, 500.0 or 1000.0 mg of Benzo[c][2,7]naphthyridine Derivative. In one embodiment, the compositions are in the form of a tablet that can be scored. Effective plasma levels of the Benzo[c][2,7]naphthyridine Derivatives can range from about 0.002 mg to about 50 mg per kg of body weight per day. The amount of a Benzo[c][2,7]naphthyridine Derivative that is effective in the treatment or prevention of cancer can be determined by clinical techniques that are known to those of skill in the art. In addition, in vitro and in vivo assays can optionally be employed to help identify optimal dosage ranges. The precise dose to be employed can also depend on the route of administration, and the seriousness of the proliferative disorder being treated and can be decided according to the judgment of the practitioner and each subject's circumstances in view of, e.g., published clinical studies. Suitable effective dosage amounts, however, can range from about 10 micrograms to about 5 grams about every 4 h, although they are typically about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g, every 4 hours. Equivalent dosages can be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered; that is, if more than one Benzo[c][2,7]naphthyridine Derivative is administered, the effective dosage amounts correspond to the total amount administered.

The dosage regimen utilizing the Benzo[c][2,7]naphthyridine Derivative can be selected in accordance with a variety of factors including type, species, age, weight, sex and medical condition of the subject; the severity of the proliferative disorder to be treated; the route of administration; the renal or hepatic function of the subject; and the particular Benzo[c][2,7]naphthyridine Derivative employed. A person skilled in the art can readily determine and prescribe the effective amount of the drug required to prevent, counter or arrest the progress of the proliferative disorder.

Benzo[c][2,7]naphthyridine Derivatives can be administered in a single daily dose, or the total daily dosage can be administered in divided doses of two, three or four times daily. Furthermore, Benzo[c][2,7]naphthyridine Derivatives can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of Benzo[c][2,7]naphthyridine Derivative ranges from about 0.1% to about 15%, w/w or w/v.

In one embodiment, the compositions comprise an amount of each of a Benzo[c][2,7]naphthyridine Derivative and another anticancer agent which together are effective to treat or prevent cancer. In another embodiment, the amount of Benzo[c][2,7]naphthyridine Derivative and another anticancer agent is at least about 0.01% of the combined combination chemotherapy agents by weight of the composition. When intended for oral administration, this amount can be varied from about 0.1% to about 80% by weight of the composition. Some oral compositions can comprise from about 4% to about 50% of a Benzo[c][2,7]naphthyridine Derivative and another anticancer agent. Other compositions of the present invention are prepared so that a parenteral dosage unit contains from about 0.01% to about 2% by weight of the composition.

The Benzo[c][2,7]naphthyridine Derivatives can be assayed in vitro or in vivo for the desired therapeutic or prophylactic activity prior to use in humans. Animal model systems can be used to demonstrate safety and efficacy.

The present methods for treating or preventing cancer in a subject in need thereof can further comprise administering another prophylactic or therapeutic agent to the subject being administered a Benzo[c][2,7]naphthyridine Derivative. In one embodiment the other prophylactic or therapeutic agent is administered in an effective amount. The other prophylactic or therapeutic agent includes, but is not limited to, an anti-inflammatory agent, an anti-renal failure agent, an anti-diabetic agent, and anti-cardiovascular disease agent, an antiemetic agent, a hematopoietic colony stimulating factor, an anxiolytic agent, and an analgesic agent.

In a further embodiment, the Benzo[c][2,7]naphthyridine Derivative can be administered prior to, concurrently with, or after an antiemetic agent, or on the same day, or within 1 hour, 2 hours, 12 hours, 24 hours, 48 hours or 72 hours of each other.

In another embodiment, the Benzo[c][2,7]naphthyridine Derivative can be administered prior to, concurrently with, or after a hematopoietic colony stimulating factor, or on the same day, or within 1 hour, 2 hours, 12 hours, 24 hours, 48 hours, 72 hours, 1 week, 2 weeks, 3 weeks or 4 weeks of each other.

In still embodiment, the Benzo[c][2,7]naphthyridine Derivative can be administered prior to, concurrently with, or after an opioid or non-opioid analgesic agent, or on the same day, or within 1 hour, 2 hours, 12 hours, 24 hours, 48 hours or 72 hours of each other.

In yet another embodiment, the Benzo[c][2,7]naphthyridine Derivative can be administered prior to, concurrently with, or after an anxiolytic agent, or on the same day, or within 1 hour, 2 hours, 12 hours, 24 hours, 48 hours or 72 hours of each other.

Effective amounts of the other therapeutic agents are well known to those skilled in the art. However, it is well within the skilled artisan's purview to determine the other therapeutic agent's optimal effective amount range. In one embodiment of the invention, where, another therapeutic agent is administered to a subject, the effective amount of the Benzo[c][2,7]naphthyridine Derivative is less than its effective amount would be where the other therapeutic agent is not administered. In this case, without being bound by theory, it is believed that the Benzo[c][2,7]naphthyridine Derivative and the other therapeutic agent act synergistically to treat or prevent cancer.

Antiemetic agents useful in the methods of the present invention include, but are not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, and tropisetron.

Hematopoietic colony stimulating factors useful in the methods of the present invention include, but are not limited to, filgrastim, sargramostim, molgramostim and epoietin alfa.

Opioid analgesic agents useful in the methods of the present invention include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene.

Non-opioid analgesic agents useful in the methods of the present invention include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

Anxiolytic agents useful in the methods of the present invention include, but are not limited to, buspirone, and benzodiazepines such as diazepam, lorazepam, oxazapam, chlorazepate, clonazepam, chlordiazepoxide and alprazolam.

### 5.8 Kits

The invention encompasses kits that can simplify the administration of a Benzo[c][2,7]naphthyridine Derivative to a subject.

A typical kit of the invention comprises a unit dosage form of a Benzo[c][2,7]naphthyridine Derivative. In one embodiment the unit dosage form is a container, which can be sterile, containing an effective amount of a Benzo[c][2,7]naphthyridine Derivative and a physiologically acceptable carrier or vehicle. The kit can further comprise a label or printed instructions instructing the use of the Benzo[c][2,7]naphthyridine Derivative to treat or prevent cancer. The kit can also further comprise a unit dosage form of another prophylactic or therapeutic agent, for example, a container containing an effective amount of another prophylactic or therapeutic agent or another anticancer agent. In one embodiment the kit comprises a container containing an effective amount of a Benzo[c][2,7]naphthyridine Derivative and an effective amount of another prophylactic or therapeutic agent. Examples of other prophylactic or therapeutic agents and other anticancer agents include, but are not limited to, those listed above.

Kits of the invention can further comprise a device that is useful for administering the unit dosage forms. Examples of such a device include, but are not limited to, a syringe, a drip bag, a patch, an inhaler, and an enema bag. The invention is further described in the following examples, which do not limit the scope of the invention described in the claims.

The following examples illustrate the synthesis of illustrative Benzo[c][2,7]naphthyridine Derivatives and demonstrate their usefulness for treating or preventing a proliferative disorder or an autoimmune disease.

### 6. EXAMPLES

### Example 1

### Synthesis of Compound Ia-62

### (9-Methoxy-8-(2-methoxyethoxy)-2-pyrrolidin-1-ylbenzo[c]-2,7-naphthyridin-4-amine)

### Step A: Preparation of 3-Methoxy-4-(2-methoxy-ethoxy)-benzoic acid methyl ester

A mixture of 4-hydroxy-3-methoxy-benzoic acid methyl ester (Aldrich Chemical Co.) (100 g, 549 mmol), 1-bromo-2-methoxy-ethane (76.3, 549 mmol), Aliquat-336 (Aldrich Chemical Co.), (2 g), and potassium carbonate (113.8, 823 mmol) is stirred and refluxed in 1 L of acetone for 18 hours. The mixture is filtered and the solvent is removed. The residue is re-dissolved in ethyl acetate and the solution is washed with a dilute base. The solution is dried (over MgSO₄) and filtered through a pad of hydrous magnesium silicate (Brand-Nu Laboratories). Solvent is removed to provide the title compound (128 g).

### Step B: Preparation of 5-Methoxy-4-(2-methoxy-ethoxy)-2-nitro-benzoic acid methyl ester

3-Methoxy-4-(2-methoxy-ethoxy)-benzoic acid methyl ester (128 g, 532.8 mmol) is dissolved in 364 ml of acetic acid to which 145 ml of concentrated nitric acid is added dropwise over 35 minutes. The mixture is stirred for 20 hours and then poured into water. The solid product is collected and washed with water and hexanes to provide the title compound after drying (149 g).

### Step C: Preparation of 2-Amino-5-methoxy-4-(2-methoxy-ethoxy)-benzoic acid methyl ester

A mixture of 5-methoxy-4-(2-methoxy-ethoxy)-2-nitro-benzoic acid methyl ester (149 g, 522.2 mmol) powdered iron (116.7 g, 2 mmol), and ammonium chloride (139.7 g, 2.61 mmol) in 500 ml of water and 1500 ml of methanol is stirred at reflux for 2 hours. The hot mixture is filtered and the organic solvents are removed. A solution of sodium bicarbonate is added to the residue and the mixture is extracted with ethyl acetate. The extract is dried (over MgSO₄) and filtered through a pad of hydrous magnesium silicate. The solution is concentrated to about 500 ml and then diluted with about 500 ml of hexanes. The mixture is cooled and the product solid is collected to provide 86.8 g of the title compound.

### Step D: Preparation of 2-(dimethylamino-methyleneamino)-5-methoxy-4-(2-methoxy-ethoxy)-benzoic acid methyl ester

A mixture of 2-amino-5-methoxy-4-(2-methoxy-ethoxy)-benzoic acid methyl ester (86.8 g, 340 mmol) and DMF-acetal (60.8, 67.8 mmol) is refluxed for 2 hours. The excess reagent is removed by distillation to provide 98.2 g of the title compound.

### Step E: Preparation of 4-Hydroxy-6-methoxy-7-(2-methoxy-ethoxy)-quinoline-3-carbonitrile

To 135.3 ml (338.3 mmol) of 2.5 M n-butyl lithium in hexanes at −78° C., a solution of acetonitrile in 450 ml of THF is added dropwise over 45 minutes. After another 45 minutes, a solution of 2-(dimethylamino-methyleneamino)-5-methoxy-4-(2-methoxy-ethoxy)-benzoic acid methyl ester (50 g, 161.1 mmol) in 450 ml of THF is added over 1 hour. The mixture is warmed slowly to −55° C. and 46 ml of acetic acid is added. The mixture is warmed to room temperature and the solvent is removed. The residue is diluted with water and the resulting solid is collected, washed with water and hexanes and dried in vacuum to provide 44 g of the title compound.

### Step F: Preparation of 4-Chloro-6-methoxy-7-(2-methoxy-ethoxy)-quinoline-3-carbonitrile

A mixture of hydroxy-6-methoxy-7-(2-methoxy-ethoxy)-quinoline-3-carbonitrile (40.2 g, 146.6 mmol), oxalyl chloride (83.7 g, 4.5 mmol), and DMF (1 g, 14.5 mmol) in 400 ml of methylene chloride is refluxed for 3.5 hours. The mixture is filtered and the solvent is removed. The residue is re-dissolved in methylene chloride and the solution is washed with ice-cold sodium bicarbonate solution. The solution is dried (over MgSO₄) and filtered through a pad of hydrous magnesium silicate. Solvent is removed. The residue is dissolved in warm ethyl acetate and then diluted with hexanes and cooled. Product solid is collected to provide 29.4 g of the title compound.

### Step G: Preparation of tert-Butyl cyano[3-cyano-6-methoxy-7-(2-methoxyethoxy)quinolin-4-yl]acetate

To a suspension of sodium hydride (112.7 mmol) in 70 ml of DMF, a solution of t-butyl cyanoacetate (7.23 g, 51.2 mmol) in 60 ml DMF is added dropwise. After stirring for 15 minutes, the solid 4-chloro-6-methoxy-7-(2-methoxy-ethoxy)-quinoline-3-carbonitrile (10 g, 34.2 mmol) is added. The mixture is stirred at 83° C. for 40 min and then poured into water (400 ml). The mixture is made acidic with aqueous acetic acid and the resulting solid is collected, washed with water, and hexanes. The solid is dissolved in chloroform, the water layer is separated, and the solution is dried (over MgSO₄) and filtered through a pad of hydrous magnesium silicate using ethyl acetate. The solvent is removed and the residue is mixed with ether. The solid is collected to provide 10.8 g of the title compound as a yellow-orange solid.

### Step H: Preparation of 4-(cyanomethyl)-6-methoxy-7-(2-methoxyethoxy)quinoline-3-carbonitrile

A mixture of tert-Butyl cyano[3-cyano-6-methoxy-7-(2-methoxyethoxy)quinolin-4-yl]acetate (9 g, 22.65 mmol) is heated at 175° C. in 90 ml of dichlorobenzene for 35 minutes, at which time a gas evolved. After the reaction is complete, the mixture is cooled and diluted with ether. The solid is collected to provide 6.3 g of the title compound.

### Step I: Preparation of 9-Methoxy-8-(2-methoxyethoxy)-2-pyrrolidin-1-ylbenzo[c]-2,7-naphthyridin-4-amine (Compound Ia-62)

A mixture of 4-(cyanomethyl)-6-methoxy-7-(2-methoxyethoxy)quinoline-3-carbonitrile (242 mg, 0.81 mmol) and pyrrolidine (2.3 g, 32.6 mmol) in 5 ml of toluene is refluxed for 40 hours. After cooling, the solid is collected and washed with ether. The solid is dissolved in chloroform and chromatographed on silica gel eluting with chloroform-methanol mixtures to provide 0.115 g of the title compound. MS: m/e 369.3 (M+H)⁺¹.

6-Bromo-4-(cyanomethyl)quinoline-3-carbonitrile, which can be used for preparation of other compounds described herein, is prepared using the methods of Steps G and H of this example.

### Example 2

### Synthesis of Compound Ia-57

### (9-Methoxy-8-(2-methoxyethoxy)-2-morpholin-4-ylbenzo[c]-2,7-naphthyridin-4-amine)

A mixture of 4-(cyanomethyl)-6-methoxy-7-(2-methoxyethoxy)quinoline-3-carbonitrile (300 mg, 1.0 mmol), morpholine (175.8 g, 2 mmol), and lanthanum trifluoromethanesulfonate (295.7 mg, 0.5 mmol) in 6 ml of THF is placed in a pressure vessel and heated with stirring at 135° C. for 3.6 days. The reaction mixture is then cooled, poured into water and extracted with chloroform. The extract is poured onto a column of hydrous magnesium silicate and the product is eluted with chloroform-methanol mixtures. The product fractions are combined and the solvent is removed. The residue is recrystallized from isopropanol to provide 0.27 g of the title compound. MS: m/e 385.2 (M+H)⁺¹.

### Example 3

### Synthesis of Compound Ia-61

### (2-Chloro-9-methoxy-8-(2-methoxyethoxy)benzo[c]-2,7-naphthyridin-4-amine)

A solution of 4-(cyanomethyl)-6-methoxy-7-(2-methoxyethoxy)quinoline-3-carbonitrile (3 g, 10.1 mmol) is prepared in 60 ml of ethanol-free chloroform and 2 ml of DMF. The mixture is stirred and HCl gas is bubbled in twice a day for three days. The mixture is diluted with ether and the solid is collected. The solid is washed with sodium bicarbonate solution and dissolved in a mixture of ethyl acetate and THF. This solution is dried (over MgSO₄) and filtered through hydrous magnesium silicate. The solvent is removed to provide 2.8 g of the title compound. MS: m/e 334.1 (M+H)⁺¹.

### Example 4

### Synthesis of Compound Ia-60

### (9-Methoxy-8-(2-methoxyethoxy)-N²,N²-dimethylbenzo[c]-2,7-naphthyridine-2,4-diamine)

A mixture of 2-chloro-9-methoxy-8-(2-methoxyethoxy)benzo[c]-2,7-naphthyridin-4-amine (from Example 3) and 8.1 ml of 2M dimethylamine in THF is stirred in a sealed tube and heated at 135° C. for 5 days. The mixture is then diluted with water. The solid is collected and recrystallized from isopropanol to provide 0.2 g of the title compound. MS: m/e 343.2 (M+H)⁺¹.

### Example 5

### Synthesis of certain other Benzo[c][2,7]naphthyridine Derivatives

Compounds described in Table 3 are prepared by reacting an appropriate commercially available aliphatic or heterocyclic amine, with 4-(cyanomethyl)-6-methoxy-7-(2-methoxyethoxy)quinoline-3-carbonitrile or 4-(cyanomethyl)-6,7-dimethoxyquinoline-3-carbonitrile (prepared using the methods of Steps G and H of Example 1), using methods described in Step I of Example 1 and Example 2. Compounds are then characterized using mass spectrometry (MS).

TABLE 3

| | | MS: m/e | MS: m/e |
|---|---|---|---|
| Compound | Compound name | (M + H)⁺¹ | (M + 2H⁾⁺² |
| I-3 | 9-methoxy-8-(2-methoxyethoxy)-N,N′- | 497.2 | 249.1 |
| | bis(pyridin-3-ylmethyl)benzo[c]-2,7- | | |
| | naphthyridine-2,4-diamine | | |
| I-4 | N,N′-bis[(1-ethylpyrrolidin-2-yl)methyl]-9- | 537.3 | 269.1 |
| | methoxy-8-(2-methoxyethoxy)benzo[c]- | | |
| | 2,7-naphthyridine-2,4-diamine | | |
| Ia-43 | 9-methoxy-8-(2-methoxyethoxy)-2-(2- | 397.3 | |
| | methylpiperidin-1-yl)benzo[c]-2,7- | | |
| | naphthyridin-4-amine | | |
| Ia-44 | N²-(1,3-dioxolan-2-ylmethyl)-9-methoxy-8- | 415.3 | 208.1 |
| | (2-methoxyethoxy)-N²-methylbenzo[c]-2,7- | | |
| | naphthyridine-2,4-diamine | | |
| Ia-45 | N²-isopropyl-9-methoxy-8-(2- | 371.3 | |
| | methoxyethoxy)-N²-methylbenzo[c]-2,7- | | |
| | naphthyridine-2,4-diamine | | |
| Ia-49 | 9-methoxy-8-(2-methoxyethoxy)-N²-(2- | | |
| | piperidin-1-ylethyl)benzo[c]-2,7- | | |
| | naphthyridine-2,4-diamine | | |
| Ia-50 | 9-methoxy-8-(2-methoxyethoxy)-2-(4- | 397.2 | |
| | methylpiperidin-1-yl)benzo[c]-2,7- | | |
| | naphthyridin-4-amine | | |
| Ia-51 | 2-{1-[4-amino-9-methoxy-8-(2- | 427.2 | |
| | methoxyethoxy)benzo[c]-2,7-naphthyridin- | | |
| | 2-yl]piperidin-4-yl}ethanol | | |
| Ia-52 | 9-methoxy-8-(2-methoxyethoxy)-N2- | 412.2 | 206.6 |
| | methyl-N²-(1-methylpyrrolidin-3- | | |
| | yl)benzo[c]-2,7-naphthyridine-2,4-diamine | | |
| Ia-53 | 1-[4-amino-9-methoxy-8-(2- | 399.2 | |
| | methoxyethoxy)benzo[c]-2,7-naphthyridin- | | |
| | 2-yl]piperidin-4-ol | | |
| Ia-54 | 9-methoxy-8-(2-methoxyethoxy)-N²-(2- | 387.2 | 194.1 |
| | methoxyethyl)-N²-methylbenzo[c]-2,7- | | |
| | naphthyridine-2,4-diamine | | |
| Ia-55 | 9-methoxy-8-(2-methoxyethoxy)-N²-(2- | 373.2 | 187.1 |
| | methoxyethyl)benzo[c]-2,7-naphthyridine- | | |
| | 2,4-diamine | | |
| Ia-56 | 9-methoxy-8-(2-methoxyethoxy)-2-[(2R)- | 413.3 | 207.1 |
| | 2-(methoxymethyl)pyrrolidin-1- | | |
| | yl]benzo[c]-2,7-naphthyridin-4-amine | | |
| Ib-64 | 8,9-dimethoxy-N²-methyl-N²-(pyridin-3- | 367.2 | 209.1 |
| | ylmethyl)benzo[c]-2,7-naphthyridine-2,4- | | |
| | diamine | | |
| Ib-65 | (3R)-1-(4-amino-8,9-dimethoxybenzo[c]- | 341.2 | |
| | 2,7-naphthyridin-2-yl)pyrrolidin-3-ol | | |
| Ib-66 | 2-[(3R)-3-(dimethylamino)pyrrolidin-1-yl]- | 368.2 | 184.6 |
| | 8,9-dimethoxybenzo[c]-2,7-naphthyridin-4- | | |
| | amine | | |
| Ib-67 | 2-[(3S)-3-(dimethylamino)pyrrolidin-1-yl]- | 368.2 | 184.6 |
| | 8,9-dimethoxybenzo[c]-2,7-naphthyridin-4- | | |
| | amine | | |
| Ib-68 | (3S)-1-(4-amino-8,9-dimethoxybenzo[c]- | 341.2 | |
| | 2,7-naphthyridin-2-yl)pyrrolidin-3-ol | | |

### Example 6

### Synthesis of Compound Ia-48

### (2-(1H-imidazol-1-yl)-9-methoxy-8-(2-methoxyethoxy)benzo[c]-2,7-naphthyridin-4-amine)

A mixture of 4-(cyanomethyl)-6-methoxy-7-(2-methoxyethoxy)quinoline-3-carbonitrile (330 mg, 1.11 mmol) and imidazole (2.04 g, 30 mmol) is heated at 120° C. for 5 hours. The mixture is cooled and diluted with water. Solid is collected, washed with water, and recrystallized from isopropanol to provide 0.13 g of the title compound. MS: m/e 366.2 (M+H)⁺¹, m/e 183.6 (M+2H)⁺²

### Example 7

### Synthesis of Compound Ia-42

### (9-Methoxy-8-(2-methoxyethoxy)-2-(4-methyl-1H-imidazol-1-yl)benzo[c]-2,7-naphthyridin-4-amine)

The title compound is prepared from 4-(cyanomethyl)-6-methoxy-7-(2-methoxyethoxy)quinoline-3-carbonitrile and 4-methyl-1H-imidazole using the method of Example 6. MS: m/e 380.2 (M+H)⁺¹.

### Example 8

### Synthesis of Compound Ib-74

### (2-(1H-imidazol-1-yl)-8,9-dimethoxybenzo[c]-2,7-naphthyridin-4-amine)

The title compound is prepared from 4-(cyanomethyl)-6,7-dimethoxyquinoline-3-carbonitrile and imidazole using the method of Example 6. MS: m/e 322.1 (M+H)⁺¹.

### Example 9

### Synthesis of Compound Ia-41

### (9-Methoxy-8-(2-methoxyethoxy)-2-(4-phenyl-1H-imidazol-1-yl)benzo[c]-2,7-naphthyridin-4-amine)

A mixture of 4-(cyanomethyl)-6-methoxy-7-(2-methoxyethoxy)quinoline-3-carbonitrile (330 mg, 1.11 mmol) and 4-phenylimidazole (320 mg, 2.22 mmol) in 1 ml of dimethoxyethane is heated at 140° C. in a sealed tube for 18 hours. The mixture is poured into water and extracted with chloroform. The extract is purified by chromatography by elution with ethyl acetate-methanol mixture to provide 0.116 g of the title compound. The regiochemistry is confirmed with ¹H-NMR using an NOE experiment. MS: m/e 442.2 (M+H)⁺¹.

### Example 10

### Synthesis of Compound Ib-69

### ([1-(4-Amino-8,9-dimethoxybenzo[c]-2,7-naphthyridin-2-yl)-1H-imidazol-4-yl]methanol)

The title compound is prepared from 4-(cyanomethyl)-6,7-dimethoxyquinoline-3-carbonitrile (0.4 g, 1.58 mmol) and 4-hydroxymethyl imidazole (1.08 g, 11.06 mmol) in 3 ml of THF by heating in a sealed tube at 140° C. for 4 days. The mixture is worked up as described in Example 9. MS: m/e 352.1 (M+H)⁺¹, m/e 176.6 (M+2H)⁺².

### Example 11

### Synthesis of Compound Ia-37

### (8-Bromo-2-(1H-imidazol-1-yl)benzo[c]-2,7-naphthyridin-4-amine)

A mixture of tert-butyl (7-bromo-3-cyanoquinolin-4-yl)(cyano)acetate (19.6 g, 52.7 mmol), imidazole (89.6 g, 1.32 mmol), and pyridine hydrochloride (6.1 g, 52.7 mmol) is heated at 140° C. for 8 hours. The mixture is dissolved in hot water and the solid is collected and washed with water. The residue is boiled in isopropanol, cooled and the solid is collected to provide 12.0 g of the title compound. MS: m/e 341.0 (M+H)⁺¹.

### Example 12

### Synthesis of Compound Ia-36

### (8-[3-(Dimethylamino)prop-1-yn-1-yl]-2-(1H-imidazol-1-yl)benzo[c]-2,7-naphthyridin-4-amine)

A mixture of 8-bromo-2-(1H-imidazol-1-yl)benzo[c]-2,7-naphthyridin-4-amine (330 mg, 0.97 mmol), dimethyl-prop-2-ynyl-amine (322.6 mg, 3.88 mmol), Pd(Cl)₂(Ph₃P)₂ (20.4 mg, 0.29 mmol), triphenylphosphine (30.5 mg, 0.12 mmol), and cuprous iodide (5.5 mg, 0.029 mmol) in 4 ml of as N-methylpyrrolidinone and 6 ml of triethylamine is refluxed for 9 hours. The mixture is diluted with water. The solid is collected and washed with water and hexanes. The residue is dissolved in a mixture of chloroform and THF and poured over a column of silica gel. The column is eluted with chloroform and then with ethyl acetate-methanol-triethylamine (70:30:1). The solvent is removed from the product fractions and the residue is recrystallized from isopropanol to provide 0.175 g of the title compound. MS: m/e 343.2 (M+H)⁺¹, m/e 172.1 (M+2H)⁺²

### Example 13

### Synthesis of Compound Ia-35

### (8-[4-(4-ethylpiperazin-1-yl)but-1-yn-1-yl]-2-(1H-imidazol-1-yl)benzo[c]-2,7-naphthyridin-4-amine)

The title compound is prepared from 8-bromo-2-(1H-imidazol-1-yl)benzo[c]-2,7-naphthyridin-4-amine and 1-but-3-ynyl-4-ethyl-piperazine using the method of Example 11. MS: m/e 426.3 (M+H)⁺¹, m/e 213.6 (M+2H)⁺².

### Example 14

### Synthesis of Compound Ia-30

### (2-(1H-imidazol-1-yl)-8-[4-(1H-imidazol-1-yl)but-1-yn-1-yl]benzo[c]-2,7-naphthyridin-4-amine)

The title compound is prepared from 8-bromo-2-(1H-imidazol-1-yl)benzo[c]-2,7-naphthyridin-4-amine and 1-But-3-ynyl-1H-imidazole using the method of Example 11. MS: m/e 190.6 (M+2H)⁺².

### Example 15

### Synthesis of Compound Ia-18

### (9-[4-(4-Ethylpiperazin-1-yl)but-1-yn-1-yl]-2-(1H-imidazol-1-yl)benzo[c]-2,7-naphthyridin-4-amine)

The title compound is prepared from 9-bromo-2-(1H-imidazol-1-yl)benzo[c]-2,7-naphthyridin-4-amine and 1-but-3-ynyl-4-ethyl-piperazine using the method of Example 11. MS: m/e 426.3 (M+H)⁺¹, m/e 213.6 (M+2H)⁺².

### Example 16

### Synthesis of Compound Ia-24

### (2-(1H-imidazol-1-yl)-8-[(1E)-4-pyrrolidin-1-ylbut-1-en-1-yl]benzo[c]-2,7-naphthyridin-4-amine)

Step A: A mixture of pyrrolidine (6.05 g, 85 mmol) and methanesulfonic acid but-3-ynyl ester (6 g, 40.5 mmol) is refluxed overnight, poured into 1N NaOH and extracted with ether to provide 3.76 g of 1-but-3-ynyl-pyrrolidine.

Step B: 3.76 g of 1-but-3-ynyl-pyrrolidine, 5.19 g of pinicol borane, and 340 mg of bis(cyclopentadienyl)zirconium is stirred overnight. The crude product, 1-[4-(4,4,5,5-tetramethyl-[1,3,2]dioxaborolan-2-yl)-but-3-enyl]-pyrrolidine, is used without additional purification.

Step C: A mixture of 8-bromo-2-(1H-imidazol-1-yl)benzo[c]-2,7-naphthyridin-4-amine (500 mg, 1.47 mmol), 1-[4-(4,4,5,5-tetramethyl-[1,3,2]dioxaborolan-2-yl)-but-3-enyl]-pyrrolidine (738.4 mg, 2.94 mmol), potassium carbonate (609 mg, 4.41 mmol), and tetrakis(triphenylphosphine)palladium (169.8 mg, 0.15 mmol) in 3 ml of ethanol, 3 ml of water, and 30 ml of toluene is stirred at reflux for 3 hours. The mixture is diluted with ethyl acetate-THF, boiled, and water is added. The organic layer is dried (over MgSO₄) and placed on a silica gel column. The product is eluted with ethyl acetate-methanol-triethylamine (70:20:1) to provide 0.16 g of the title compound. MS: m/e 385.2 (M+H)⁺¹, m/e 193.1 (M+2H)⁺².

### Example 17

### Synthesis of Compound Ia-25

### (8-[(1E)-4-(4-ethylpiperazin-1-yl)but-1-en-1-yl]-2-(1H-imidazol-1-yl)benzo[c]-2,7-naphthyridin-4-amine)

The title compound is prepared from 1-but-3-ynyl-4-ethyl-piperazine and 8-bromo-2-(1H-imidazol-1-yl)benzo[c]-2,7-naphthyridin-4-amine using the method of example 14 above. MS: m/e 428.3 (M+H)⁺¹, m/e 214.7 (M+2H)⁺².

### Example 18

### Synthesis of 6,7-Dimethoxy-4-methyl-2-oxo-1,2-dihydro-quinoline-3-carbonitrile

To a 1 L flask provided with mechanical stirrer, condenser, nitrogen trap and drying tube are added in order: ethyl-cyanoacetate (88.2 g, 83.1 ml, 0.78 mol, 2 eq.), 2-amino-4,5 dimethoxyacetophenone (75 g, 0.390 mol, 1 eq.), and ammonium acetate (15 g, 0.195 mol, 0.5 eq.) under vigorous stirring. The mixture is heated to 165 C.° in an oil bath. The reaction mixture is kept under reflux for 10 hours, cooled to room temperature, and diluted with 500 ml cold water. After 1 hour the suspension is filtered, solid precipitate collected, washed 3×200 ml of water, dried in vacuum oven at 60 C.° overnight. The title compound (60.5 g. 64.5%) is obtained as a light yellow solid. MS: m/e 243.3 (M+H)⁺¹

### Example 19

### Synthesis of 2-Chloro-6,7-dimethoxy-4-methyl-quinoline-3-carbonitrile hydrochloride

To a 500 ml flask provided with magnetic stirrer, condenser, nitrogen trap and drying tube 25 g (0.102 mol) 6,7-dimethoxy-4-methyl-2-oxo-1,2-dihydro-quinoline-3-carbonitrile is suspended in 350 ml of phosphorusoxychloride and under vigorous stirring the reaction mixture is refluxed for 12 hours. The reaction mixture is cooled to room temperature and 300 ml of carbon tetrachloride is added and kept at room temperature for 4 hours. The deposited solid filtered off, washed with hexane (100 ml), dried in air at room temperature to provide 16.4 g of the title compound. The filtrate is portion-wise evaporated in vacuo to almost dryness in a Buchi rotavapor, residue is dissolved in 100 ml of carbon tetrachloride, left at room temperature overnight, filtered, dried, to provide additional 4.2 g. of the title compound as an off-white solid, hydrochloride salt (overall yield 20.6 g, 67.5%). MS: m/e 263.2 (M+H)⁺¹.

### Example 20

### Synthesis of 6,7-dimethoxy-4-methyl-quinoline-3-carbonitrile

2-Chloro-6,7-dimethoxy-4-methyl-quinoline-3-carbonitrile hydrochloride (12 g, 0.04 mol) is suspended in 1000 ml THF, and 6 g (0.044 mol, 1.1 eqs.) of potassium carbonate (dissolved in 10 ml of water) is added. The reaction mixture is stirred vigorously using a mechanical stirrer for 6 hours. The THF clear extract (filtrate) is hydrogenated for 10 hours (50 PSI) in a 2-liter bottle in the presence of 3 g 10% Pd/C and 6 g of potassium carbonate dissolved in 10 ml of water, until no more starting material is present. The reaction mixture is filtered through a pad of diatomaceous earth, filtrate evaporated to dryness, residue dissolved in 500 ml methylenechloride, washed with water, dried, and evaporated to a light yellowish solid. Crude product is flash-chromatographed through a short silica gel column, using methylenechloride:ethyl acetate (0 to 10%) as eluant. Collected fractions evaporated to provide 8.9 g (97%) of title compound as a white solid. MS: m/e 229.2 (M+H)⁺¹

### Example 21

### Synthesis of Compound Ia-58

### (2,9-Dimethoxy-8-(2-methoxyethoxy)benzo[c]-2,7-naphthyridin-4-amine)

A solution of 2-chloro-9-methoxy-8-(2-methoxyethoxy)benzo[c]-2,7-naphthyridin-4-amine (300 mg, 1.01 mmol) in 9 ml of methanol into which sodium (116 mg, 5 mmol) is predissolved is refluxed for 4.5 hours. The mixture is poured into water and extracted with methylenechloride. The solution is dried (over MgSO₄) and the solvent is removed. The residue is recrystallized from isopropanol to provide the title compound. MS: m/e 339.2 (M+H)⁺¹.

### Example 22a

### Synthesis of Compounds Ia-11, Ib-24, Ib-28, Ib-29, Ib-41, Ib-44, Ib-52, Ib-55, Ib-56, and Ib-62

Step A: To a stirred solution at 0° C. of 16.74 ml of BH₃ in THF (1.0 M solution, 16.74 mmol) is added (S)-Boc-2-chloro-phenylalanine (2508 mg, 8.37 mmol) in 5 ml dry THF over 30 minutes. The reaction is held at 0° C. for 3 hours and then stopped with 25 ml of 10% acetic acid in methanol. The solution is concentrated in vacuo and the residue is dissolved in 100 ml ethyl acetate and washed with 100 ml of 1N HCl, water, and 1M NH₄HCO₃. The organic layer is dried over MgSO₄ and concentrated in vacuo to provide tert-butyl [(1S)-1-(2-chlorobenzyl)-2-hydroxyethyl]carbamate (1.790 g, 75%). MS: 330.0 [M+FA-H].

Using similar methodology, the following compounds are prepared:

| Compound name | MS: m/e |
|---|---|
| tert-butyl [(1S)-1-(3,4-dichlorobenzyl)-2- | 363.9 |
| hydroxyethyl]carbamate | |
| tert-butyl [(1S)-1-(2-chlorobenzyl)-2- | 330 |
| hydroxyethyl]carbamate | |
| tert-butyl [(1R)-1-(4-fluorobenzyl)-2- | 314.1 |
| hydroxyethyl]carbamate | |
| tert-butyl [(1S)-1-(3-chlorobenzyl)-2- | 330 |
| hydroxyethyl]carbamate | |
| benzyl (4-{(2S)-2-[(tert- | 399.2 |
| butoxycarbonyl)amino]-3- | |
| hydroxypropyl}phenyl)carbamate | |
| tert-butyl [(1S)-1-(4-fluorobenzyl)-2- | 314.1 |
| hydroxyethyl]carbamate | |
| tert-butyl {(1S)-2-hydroxy-1-[3- | 320.2 |
| trifluoromethyl)benzyl]ethyl}carbamate | |
| tert-butyl {(1R)-2-hydroxy-1-[3- | 320.2 |
| trifluoromethyl)benzyl]ethyl}carbamate | |

Step B: Method 1: Tert-butyl [(1S)-1-(2-chlorobenzyl)-2-hydroxyethyl]carbamate (4013 mg, 14.04 mmol), PPh₃ (4419 mg, 14.04 mmol), and 5-hydroxynicotinic acid methyl ester (2121 mg, 14.04 mmol) are dissolved in 60 ml of dry THF and added DEAD (2.65 ml, 16.85 mmol) in 12 ml of dry THF over 10 minutes. The solution is stirred at room temperature for 3 hours then concentrated in vacuo. The residue is dissolved in Et₂O, and stirred for 1 hour and then filtered. The filtrate is washed with 3×200 ml of 0.5N NaOH and the organic layer is dried over MgSO₄ and concentrated in vacuo. The residue is purified by silica gel chromatography using hexanes and ether with a gradient elution of 10-50% to provide methyl 5-{[(2S)-2-[(tert-butoxycarbonyl)amino]-3-(2-chlorophenyl)propyl]oxy}nicotinate (1.780 g, 56%). MS: 421.2 (M+H)⁺¹.

Using this methodology, the following compounds are prepared:

| | MS: m/e |
|---|---|
| Compound name | (M + H)⁺¹ |
| methyl 5-({(2R)-2-[(tert-butoxycarbonyl)amino]-3- | 387.3 |
| phenylpropyl}oxy)nicotinate | |
| methyl 5-{[(2R)-2-[(tert-butoxycarbonyl)amino]-3- | 426.4 |
| (1H-indol-3-yl)propyl]oxy}nicotinate | |
| methyl 5-({(2S)-3-[4-(benzyloxy)phenyl]-2-[(tert- | 493.5 |
| butoxycarbonyl)amino]propyl}oxy)nicotinate | |
| methyl 5-{[(2S)-2-[(tert-butoxycarbonyl)amino]-3- | 421.2 |
| (2-chlorophenyl)propyl]oxy}nicotinate | |
| methyl 5-{[(2R)-2-[(tert-butoxycarbonyl)amino]-3- | 405.3 |
| (4-fluorophenyl)propyl]oxy}nicotinate | |
| methyl 5-{[(2S)-2-[(tert-butoxycarbonyl)amino]-3- | 426.3 |
| (1H-indol-3-yl)propyl]oxy}nicotinate | |
| methyl 5-{[(2S)-2-[(tert-butoxycarbonyl)amino]-3- | 405.2 |
| (4-fluorophenyl)propyl]oxy}nicotinate | |
| methyl 5-{[(2S)-2-[(tert-butoxycarbonyl)amino]-3- | 421.1 |
| (3-chlorophenyl)propyl]oxy}nicotinate | |
| methyl 5-{[(2S)-2-[(tert-butoxycarbonyl)amino]-3- | 455 |
| (3,4-dichlorophenyl)propyl]oxy}nicotinate | |
| methyl 5-({(2S)-3-(4- | 536.2 |
| {[(benzyloxy)carbonyl]amino}phenyl)-2-[(tert- | |
| butoxycarbonyl)amino]propyl}oxy)nicotinate | |
| methyl 5-{[(2S)-2-[(tert-butoxycarbonyl)amino]-3- | 387.1 |
| (phenyl)propyl]oxy}nicotinate | |

Method 2: To a stirred solution of tert-butyl{(1S)-2-hydroxy-1-[3-(trifluoromethyl)benzyl]ethyl}carbamate (1000 mg, 3.13 mmol) in dry methyl chloride, triethylamine (0.65 ml, 4.70 mmol) is added followed by dropwise addition of methanesulfonyl chloride (0.26 ml, 3.29 mmol). The reaction is stirred for 30 minutes and concentrated in vacuo. The residue is dissolved in 150 ml of ethyl acetate and washed with 100 ml each of water, NaHCO₃, brine, and dried over Na₂SO₄. The organic layer is passed through a pad of silica gel and concentrated in vacuo. To a stirred solution of the residue and 5-hydroxynicotinic acid methyl ester (479 mg, 3.13 mmol) in 10 ml of dry DMF, CsCO₃ (2040 mg, 6.26 mmol) is added and the resulting mixture is heated at 60° C. for 18 hours. The mixture is concentrated in vacuo and the residue is dissolved in 200 ml Et₂O and partitioned between 150 ml 0.1N NaOH, dried over Na₂SO₄, and purified by silica gel chromatography using a gradient elution 20-100% ethyl acetate/hexanes to provide methyl 5-({(2S)-2-[(tert-butoxycarbonyl)amino]-3-[3-trifluoromethyl)phenyl]propyl}oxy)nicotinate (339 mg, 24%).

Using this methodology, the following compounds are prepared:

| | MS: m/e |
|---|---|
| Compound Name | (M + H)⁺¹ |
| methyl 5-({(2S)-2-[(tert-butoxycarbonyl)amino]-3-[3- | 455.3 |
| (trifluoromethyl)phenyl]propyl}oxy)nicotinate | |
| methyl 5-({(2R)-2-[(tert-butoxycarbonyl)amino]-3-[3- | 455.3 |
| (trifluoromethyl)phenyl]propyl}oxy)nicotinate | |

Step C:

Method 1: To a stirred solution of methyl 5-{[(2S)-2-[(tert-butoxycarbonyl)amino]-3-(2-chlorophenyl)propyl]oxy}nicotinate (1.020 g, 2.42 mmol) and 6,7-dimethoxy-4-methyl-quinoline-3-carbonitrile (553 mg, 2.42 mmol) in 20 ml of dry THF at −78° C. 7.74 ml of LiHMDS (1.0 M solution in THF, 7.74 mmol) is added dropwise. The reaction is stirred at −78° C. for 15 minutes then slowly warmed to room temperature over 2 hours then stirred for an additional 30 minutes. Crushed CO₂ and then water are added to stop the reaction. The mixture is extracted with 3×150 ml of ethyl acetate, dried over Na₂SO₄, and concentrated in vacuo. The residue is further dried by concentrating in vacuo with toluene. The residue is dissolved in 30 ml of acetic acid and sublimed NH₄OAc (3.731 g, 48.4 mmol) is added and the mixture is refluxed for 20 minutes. The acetic acid is removed by concentrating in vacuo and 30 ml of 2N HCl in 100 ml of THF is added. The reaction is stirred at 60° C. for 1 h then cooled and poured into 5N NaOH and extracted with 3×150 ml of ethyl acetate, dried over Na₂SO₄, and concentrated in vacuo. The residue is purified by silica gel chromatography using 80:20:1.5 ethyl acetate/methanol/Et₃N to provide 2-(5-{[(2S)-2-amino-3-(2-chlorophenyl)propyl]oxy}pyridin-3-yl)-8,9-dimethoxybenzo[c]-2,7-naphthyridin-4-amine (128 mg, 10% for 3 steps). MS: 516.3 (M+H)⁺¹

Using this methodology, the following Benzo[c][2,7]naphthyridine Derivatives are prepared starting with either 6,7-dimethoxy-4-methyl-quinoline-3-carbonitrile or 6-methoxy-7-(2-methoxy-ethoxy)-4-methyl-quinoline-3-carbonitrile:

TABLE 4

| | | MS: m/e |
|---|---|---|
| Compound | Compound Name | (M + H)⁺¹ |
| Ia-11 | 2-(5-{[(2S)-2-amino-3- | 526.4 |
| | phenylpropyl]oxy}pyridin-3-yl)-9-methoxy- | |
| | 8-(2-methoxyethoxy)benzo[c]-2,7- | |
| | naphthyridin-4-amine | |
| Ib-44 | 2-(5-{[(2S)-2-amino-3-(3,4- | 550.2 |
| | dichlorophenyl)propyl]oxy}pyridin-3-yl)- | |
| | 8,9-dimethoxybenzo[c]-2,7-naphthyridin-4- | |
| | amine | |
| Ib-55 | 2-(5-{[(2R)-2-amino-3-(1H-indol-3- | 521.4 |
| | yl)propyl]oxy}pyridin-3-yl)-8,9- | |
| | dimethoxybenzo[c]-2,7-naphthyridin-4- | |
| | amine | |
| Ib-56 | 2-(5-{[(2R)-2-amino-3- | 482.3 |
| | phenylpropyl]oxy}pyridin-3-yl)-8,9- | |
| | dimethoxybenzo[c]-2,7-naphthyridin-4- | |
| | amine | |
| Ib-62 | 2-(5-{[(2S)-2-amino-3- | 482.1 |
| | phenylpropyl]oxy}pyridin-3-yl)-8,9- | |
| | dimethoxybenzo[c]-2,7-naphthyridin-4- | |
| | amine | |

Method 2: To a stirred solution of methyl 5-{[(2S)-2-[(tert-butoxycarbonyl)amino]-3-(4-fluorophenyl)propyl]oxy}nicotinate (1.035 g, 2.56 mmol) and 6,7-dimethoxy-4-methyl-quinoline-3-carbonitrile (584 mg, 2.56 mmol) in 20 ml of dry THF at −78° C. 7.68 ml of LiHMDS (1.0 M solution in THF, 7.68 mmol) is added dropwise. The reaction is stirred at −78° C. for 15 minutes then slowly warmed to room temperature over 2 hours then stirred for an additional 30 minutes. Crushed CO₂ and then water are added to stop the reaction. The mixture is extracted with 3×150 ml of ethyl acetate, dried over Na₂SO₄, and concentrated in vacuo. The residue is further dried by concentrating in vacuo with toluene. The residue is dissolved in 20 ml of hot phenol and sublimed NH₄OAc (3946 mg, 51.20 mmol) is added to the mixture, which is then heated at 140° C. for 1 hour. The reaction is cooled and 5 ml of TFA is added. The reaction is stirred at room temperature for 2 hours and concentrated in vacuo. The liquid is diluted with ethyl acetate and passed through a silica gel pad with 500 ml ethyl acetate. The compound is eluted off the pad with 80:15:1.5 ethyl acetate/methanol/Et₃N to provide 2-(5-{[(2S)-2-amino-3-(4-fluorophenyl)propyl]oxy}pyridin-3-yl)-8,9-dimethoxybenzo[c]-2,7-naphthyridin-4-amine (312 mg, 24% for 3 steps).

Using this methodology, Benzo[c][2,7]naphthyridine Derivatives in Table 5 are prepared starting with 6,7-dimethoxy-4-methyl-quinoline-3-carbonitrile.

TABLE 5

| Compound | Compound name | MS: m/z |
|---|---|---|
| Ib-24 | 2-[5-({(2S)-2-amino-3-[3- | 550.2 |
| | (trifluoromethyl)phenyl]propyl}oxy)pyridin-3- | |
| | yl]-8,9-dimethoxybenzo[c]-2,7-naphthyridin-4- | |
| | amine | |
| Ib-28 | 2-[5-({(2R)-2-amino-3-[3- | 550.4 |
| | (trifluoromethyl)phenyl]propyl}oxy)pyridin-3- | |
| | yl]-8,9-dimethoxybenzo[c]-2,7-naphthyridin-4- | |
| | amine | |
| Ib-29 | 2-(5-{[(2S)-2-amino-3-(3- | 516.2 |
| | chlorophenyl)propyl]oxy}pyridin-3-yl)-8,9- | |
| | dimethoxybenzo[c]-2,7-naphthyridin-4-amine | |
| Ib-41 | 2-(5-{[(2S)-2-amino-3-(4- | 500.2 |
| | fluorophenyl)propyl]oxy}pyridin-3-yl)-8,9- | |
| | dimethoxybenzo[c]-2,7-naphthyridin-4-amine | |

### Example 22b

### Synthesis of Compound Ib-54

### (2-[5-({(2S)-2-amino-3-[4-(benzyloxy)phenyl]propyl}oxy)pyridin-3-yl]-8,9-dimethoxybenzo[c]-2,7-naphthyridin-4-amine)

The title compound is prepared using the methods outlined in Example 22a. MS: 588.4 (M+H)⁺¹

### Example 23

### Synthesis of Compound Ib-53

### (4-((2S)-2-Amino-3-{[5-(4-amino-8,9-dimethoxybenzo[c]-2,7-naphthyridin-2-yl)pyridin-3-yl]oxy}propyl)phenol)

The title compound is prepared as in Example 22a with the addition of the following step: To a medium pressure glass vessel under a N₂ atmosphere in 30 mg of 10% Pd/C is added. 2-[5-({(2S)-2-amino-3-[4-(benzyloxy)phenyl]propyl}oxy)pyridin-3-yl]-8,9-dimethoxybenzo[c]-2,7-naphthyridin-4-amine (162 mg, 0.28 mmol) is dissolved in 25 ml in 1:1 THF/EtOH with 5 drops of concentrated HCl and added to the vessel. The vessel is shaken under 50 PSI pressure of H₂ for 7 days (2 days at 40° C.). The mixture is filtered through diatomaceous earth and concentrated in vacuo. The residue is purified by reverse phase HPLC using a gradient elution of 0-90% CH₃CN/water 0.02% TFA to provide 4-((2S)-2-amino-3-{[5-(4-amino-8,9-dimethoxybenzo[c]-2,7-naphthyridin-2-yl)pyridin-3-yl]oxy}propyl)phenol (15 mg, 11%). MS: 498.3 (M+H)⁺¹.

### Example 24

### Synthesis of Compound Ib-19

### (2-(5-{[(2S)-2-amino-3-(4-aminophenyl)propyl]oxy}pyridin-3-yl)-8,9-dimethoxybenzo[c]-2,7-naphthyridin-4-amine)

The title compound is prepared as in Example 22a with the addition of the following step: to a medium pressure glass vessel under a N₂ atmosphere 100 mg 10% of Pd/C is added. Benzyl [4-((2S)-2-amino-3-{[5-(4-amino-8,9-dimethoxybenzo[c]-2,7-naphthyridin-2-yl)pyridin-3-yl]oxy}propyl)phenyl]carbamate (208 mg, 0.33 mmol) is dissolved in 50:1 ml of EtOH/acetic acid and added to vessel. The vessel is shaken under 50 PSI pressure of H₂ for 36 hours at room temperature. The mixture is filtered through diatomaceous earth and concentrated in vacuo. The residue is purified by reverse phase HPLC using a gradient elution of 10-50% CH₃CN/water 20 mM triethylamine to provide 2-(5-{[(2S)-2-amino-3-(4-aminophenyl)propyl]oxy}pyridin-3-yl)-8,9-dimethoxybenzo[c]-2,7-naphthyridin-4-amine (10 mg, 6%). MS: 497.4 (M+H)⁺¹.

### Example 25

### Synthesis of Compound Ib-13

### (2-(5-{[(2S)-2-amino-3-(1H-indol-3-yl)propyl]oxy}pyridin-3-yl)-8,9-dimethoxybenzo[c]-2,7-naphthyridin-4-amine)

The title compound is prepared as in Example 22a with the addition of Step B1 and modification of Step C:

Step B1: To a stirred solution of methyl 5-{[(2S)-2-[(tert-butoxycarbonyl)amino]-3-(1H-indol-3-yl)propyl]oxy}nicotinate (8.083 g, 19.0 mmol) in 100 ml of dry CH₃CN Boc₂O (4.67 ml, 20.33 mmol) and 300 mg of DMAP is added. The reaction is stirred for 18 hours and then concentrated in vacuo. The residue is dissolved in 300 ml of ethyl acetate and washed with 4×200 ml of 0.2M citric acid, 200 ml NaHCO₃, 200 ml brine, then dried over MgSO₄, and concentrated in vacuo. The residue is purified by silica gel chromatography to provide 3-[2-tert-butoxycarbonylamino-3-(5-methoxycarbonyl-pyridin-3-yloxy)-propyl]-indole-1-carboxylic acid tert-butyl ester (6020 mg, 60%). MS: 526.4 (M+H)⁺¹.

Step C: To a stirred solution of 3-[2-tert-butoxycarbonylamino-3-(5-methoxycarbonyl-pyridin-3-yloxy)-propyl]-indole-1-carboxylic acid tert-butyl ester (2.668 g, 5.08 mmol) and 6,7-dimethoxy-4-methyl-quinoline-3-carbonitrile (1159 mg, 5.08 mmol) in 20 ml of dry THF at −78° C. 40.64 ml of LiHMDS (1.0 M solution in THF, 40.64 mmol) is added dropwise. The reaction is stirred at −78° C. for 15 minutes then slowly warmed to room temperature over 2 hours then stirred for an additional 30 minutes. Crushed CO₂ and then water are added to stop the reaction. The mixture is extracted with 3×150 ml of ethyl acetate, dried over Na₂SO₄ and concentrated in vacuo. The residue is further dried by concentrating in vacuo with toluene. The residue is dissolved in 40 ml of hot phenol and sublimed NH₄OAc (3207 mg, 100 mmol) is added and the mixture is then heated at 140° C. for 1 hour. The reaction is cooled and 5 ml of TFA is added. The reaction is stirred at room temperature for 2 hours and concentrated in vacuo. The liquid is diluted with ethyl acetate and passed through a silica gel pad with 500 ml ethyl acetate. The compound is eluted off the pad with 80:15:1.5 ethyl acetate/methanol/Et₃N then crystallized from CH₃CN to provide 2-(5-{[(2S)-2-amino-3-(1H-indol-3-yl)propyl]oxy}pyridin-3-yl)-8,9-dimethoxybenzo[c]-2,7-naphthyridin-4-amine (200 mg, 8% for 3 steps). MS: 521.4 (M+H)⁺¹

### Example 26

### Synthesis of Compound I-2

### (2-(5-{[(2S)-2-amino-3-(3-chlorophenyl)propyl]oxy}pyridin-3-yl)-N-hydroxy-8,9-dimethoxybenzo[c]-2,7-naphthyridin-4-amine)

The title compound is prepared as in Example 22a with the modification of Step C as follows: to a stirred solution of methyl 5-{[(2S)-2-[(tert-butoxycarbonyl)amino]-3-(3-chlorophenyl)propyl]oxy}nicotinate (474 mg, 1.13 mmol) and 6,7-dimethoxy-4-methyl-quinoline-3-carbonitrile (257 mg, 1.13 mmol) in 20 ml of dry THF at −78° C. 7.35 ml of LiHMDS (1.0 M solution in THF, 7.35 mmol) is added dropwise. The reaction is stirred at −78° C. for 15 minutes then slowly warmed to room temperature over 2 hours then stirred an additional 30 minutes. Crushed CO₂ and then water are added to stop the reaction. The mixture is extracted with 3×100 ml of ethyl acetate, dried over Na₂SO₄ and concentrated in vacuo. The residue is further dried by concentrating in vacuo with toluene. The residue is dissolved in 10 ml of DMF and excess NH. HCl is added then heated at 40° C. for 18 hours. The reaction is concentrated in vacuo and the residue dissolved in 5 ml of methylene chloride and 2 ml of triethylamine. The reaction is stirred at room temperature for 2 hours and concentrated in vacuo. The residue is dissolved in ethyl acetate and washed with saturated Na₂HCO₃, dried over Na₂SO₄, and concentrated in vacuo. The residue is purified by reverse phase HPLC using a gradient elution of 10-40% CH₃CN/water 0.02% TFA to provide 2-(5-{[(2S)-2-amino-3-(3-chlorophenyl)propyl]oxy}pyridin-3-yl)-N-hydroxy-8,9-dimethoxybenzo[c]-2,7-naphthyridin-4-amine (20 mg, 3% for 3 steps). MS: 532.2 (M+H)⁺¹.

### Example 27

### Synthesis of Compound I-1

### (2-(5-{[(2S)-2-amino-3-(1H-indol-3-yl)propyl]oxy}pyridin-3-yl)-N-hydroxy-8,9-dimethoxybenzo[c]-2,7-naphthyridin-4-amine)

The title compound is prepared as for Example 22a with the following modification of Step C:

To a stirred solution of methyl 3-[2-tert-butoxycarbonylamino-3-(5-methoxycarbonyl-pyridin-3-yloxy)-propyl]-indole-1-carboxylic acid tert-butyl ester 678, 1.29 mmol) and 6,7-dimethoxy-4-methyl-quinoline-3-carbonitrile (294 mg, 1.29 mmol) in 20 ml of dry THF at −78° C. 7.74 ml of LiHMDS (1.0 M solution in THF, 7.74 mmol) is added dropwise. The reaction is stirred at −78° C. for 15 minutes then slowly warmed to room temperature over 2 hours and then stirred for an additional 30 minutes. Crushed CO₂ and then water are added to stop the reaction. The mixture is extracted with 3×100 ml of ethyl acetate, dried over Na₂SO₄, and concentrated in vacuo. The residue is further dried by concentrating in vacuo with toluene. The residue is dissolved in 20 ml of hot phenol, NH₂OH.HCl (448 mg, 6.45 mmol) is added and the mixture is then heated at 45° C. for 2 hours, cooled, and then 2 ml of TFA is added. The reaction is stirred for 2 hours and then poured into saturated Na₂HCO₃ and the solids are collected and crystallized from ethyl acetate to provide 2-(5-{[(2S)-2-amino-3-(1H-indol-3-yl)propyl]oxy}pyridin-3-yl)-N-hydroxy-8,9-dimethoxybenzo[c]-2,7-naphthyridin-4-amine (329 mg, 46% for 3 steps). MS: 537.4 (M+H)⁺¹.

### Example 28

### Synthesis of Compound Ib-49

### (2-(5-{[(2S)-2-amino-3-(1,3-dihydro-2H-isoindol-2-yl)propyl]oxy}pyridin-3-yl)-8,9-dimethoxybenzo[c]-2,7-naphthyridin-4-amine)

Step A: To a stirred mixture of S-[2-(tert-butyl-dimethyl-silanyloxy)-1-formyl-ethyl]-carbamic acid tert-butyl ester (5 g, 16.48 mmol) and 2,3-dihydro-1H-isoindole (1.96 g, 16.48 mmol) in 55 ml of methanol sodium cyanoborohydride (1.29 g, 20.6 mmol) and acetic acid (1.09 g, 18.12 mmol) are added. The mixture is stirred overnight. The solvent is removed and the residue is mixed with methylene chloride and sodium bicarbonate solution. The organic layer is dried (over MgSO₄) and the solution is passed through a pad of hydrous magnesium silicate. The solvent is removed to provide 6.6 g of R-[2-(tert-butyl-dimethyl-silanyloxy)-1-(1,3-dihydro-isoindol-2-ylmethyl)-ethyl]-carbamic acid tert-butyl ester.

Step B: R-[2-(tert-butyl-dimethyl-silanyloxy)-1-(1,3-dihydro-isoindol-2-ylmethyl)-ethyl]-carbamic acid tert-butyl ester (6.6 g, 16.23 mmol) is placed in 55 ml of THF and tri-N-butyl ammonium fluoride (17.85 ml, 1M solution in THF) is added. The mixture is stirred for several hours and concentrated. Water is added and the mixture is extracted with ether-hexanes. The solvent is removed and the residue is chromatographed on silica gel eluting with hexane-ether mixtures to provide 2.3 g of tert-butyl [(1R)-2-(1,3-dihydro-2H-isoindol-2-yl)-1-(hydroxymethyl)ethyl]carbamate.

Step C: To a mixture of tert-butyl [(1R)-2-(1,3-dihydro-2H-isoindol-2-yl)-1-(hydroxymethyl)ethyl]carbamate (2.2 g, 7.51 mmol), methyl 3-hydroxy nicotinate (1.15 g, 7.51 mmol), and triphenylphosphine (2.36 g, 9.01 mmol) in 40 ml of THF a 40% solution of diethyl azodicarboxylate (DEAD) in toluene (3.92 g) is added. The mixture is stirred for 3 hours or until completion of the reaction. The solvent is removed and ether is added. The solution is stirred overnight and the liquid is filtered from the precipitated solid. The solvent is removed and the residue is dissolved in methylene chloride. This solution is extracted with ice cold 0.2 N HCl. The aqueous extract is washed with a small amount of ethyl acetate, which is then discarded, and then made basic with sodium hydroxide solution. The mixture is extracted with ethyl acetate and dried (over MgSO₄). The solvent is removed and the product is purified by chromatography on silica gel amount eluted with hexanes-ether mixtures to provide 0.75 g of R-5-[2-tert-Butoxycarbonylamino-3-(1,3-dihydro-isoindol-2-yl)-propoxy]-nicotinic acid methyl ester.

Step D: To a stirred solution of R-5-[2-tert-butoxycarbonylamino-3-(1,3-dihydro-isoindol-2-yl)-propoxy]-nicotinic acid methyl ester (758.5 mg, 1.77 mmol) and 6,7-dimethoxy-4-methyl-quinoline-3-carbonitrile (405 mg, 1.77 mmol) in 34 ml of THF under N₂ at −78° C., a solution lithium hexamethyldisilazane (LiHMDS) in THF (6.03 ml, 6.03 mmol, 1 molar) is added. The mixture is placed in a bath at −50° C. and stirred for 1 hour. The mixture is slowly allowed to warm to room temperature and then poured into saturated ammonium chloride solution. The mixture is extracted multiple times with ethyl acetate. The extract is dried (over MgSO₄) and the solvent is removed. Ammonium acetate (2.74 g) and acetic acid (23 ml) are added. The mixture is refluxed for 15 minutes. Acetic acid is removed under vacuum and the residue is stirred with 50 ml of THF and 17 ml of 2N HCl at 65° C. for 2.5 hours. The mixture is made basic with sodium hydroxide solution and extracted several times with ethyl acetate. The extract is dried (over MgSO₄) and the solvent is removed. The residue is chromatographed on silica gel by eluting with ethyl acetate-methanol mixtures and finally with ethyl acetate-methanol (4:1) containing 0.2% triethylamine to provide 0.14 g of the title compound. MS: 537.2 (M+H)⁺¹, 269.1 (M+2H)⁺².

Using methods described above, either enantiomer of [2-(tert-butyl-dimethyl-silanyloxy)-1-formyl-ethyl]-carbamic acid tert-butyl ester is reacted with an appropriate aliphatic or heterocyclic amine, which after removal of the silyl protecting group provided an alcohol. This is coupled with methyl 3-hydroxy nicotinate as described and the product of this reaction is condensed with 6,7-dimethoxy-4-methyl-quinoline-3-carbonitrile to provide illustrative Benzo[c][2,7]naphthyridine Derivatives listed in Table 6. Compound Ib-35 is isolated, by chromatography, as a side-product in the preparation of the compound of Ib-36. In the preparation of compound Ib-45, N-methyl ammonium acetate is used instead of ammonium acetate in the cyclization step (Step D). Additionally, DMF at 100° C. or phenol at 140° C. can be used in place of acetic acid for the cyclization reaction in Step D.

TABLE 6

| | | MS: m/e | MS: m/e |
|---|---|---|---|
| Compound | Compound Name | (M + H)⁺¹ | (M + 2H)⁺² |
| Ib-20 | 2-(5-{[(2S)-2-amino-3-(4-pyridin-2- | 567.5 | 284.2 |
| | ylpiperazin-1-yl)propyl]oxy}pyridin-3- | | |
| | yl)-8,9-dimethoxybenzo[c]-2,7- | | |
| | naphthyridin-4-amine | | |
| Ib-21 | (2S)-3-{[5-(4-amino-8,9- | 540.4 | 270.7 |
| | dimethoxybenzo[c]-2,7-naphthyridin-2- | | |
| | yl)pyridin-3-yl]oxy}-N1-ethyl-N1- | | |
| | (pyridin-4-ylmethyl)propane-1,2- | | |
| | diamine | | |
| Ib-27 | (2S)-3-{[5-(4-amino-8,9- | 525.5 | 263.2 |
| | dimethoxybenzo[c]-2,7-naphthyridin-2- | | |
| | yl)pyridin-3-yl]oxy}-N1-benzyl-N1- | | |
| | methylpropane-1,2-diamine | | |
| Ib-34 | (2S)-3-{[5-(4-amino-8,9- | 555.4 | 278.2 |
| | dimethoxybenzo[c]-2,7-naphthyridin-2- | | |
| | yl)pyridin-3-yl]oxy}-N1-(2- | | |
| | methoxyethyl)-N1-phenylpropane-1,2- | | |
| | diamine | | |
| Ib-35 | (4S)-4-({[5-(4-amino-8,9- | 541.4 | 271.2 |
| | dimethoxybenzo[c]-2,7-naphthyridin-2- | | |
| | yl)pyridin-3-yl]oxy}methyl)-1-(3- | | |
| | fluorophenyl)imidazolidin-2-one | | |
| Ib-36 | (2S)-3-{[5-(4-amino-8,9- | 515.4 | 258.2 |
| | dimethoxybenzo[c]-2,7-naphthyridin-2- | | |
| | yl)pyridin-3-yl]oxy}-N1-(3- | | |
| | fluorophenyl)propane-1,2-diamine | | |
| Ib-37 | 4-((2S)-2-amino-3-{[5-(4-amino-8,9- | 504.2 | 252.6 |
| | dimethoxybenzo[c]-2,7-naphthyridin-2- | | |
| | yl)pyridin-3-yl]oxy}propyl)piperazin-2- | | |
| | one | | |
| Ib-38 | (2S)-3-{[5-(4-amino-8,9- | | |
| | dimethoxybenzo[c]-2,7-naphthyridin-2- | | |
| | yl)pyridin-3-yl]oxy}-N1-(2- | | |
| | methoxyethyl)-N1-methylpropane-1,2- | | |
| | diamine | | |
| Ib-45 | 2-(5-{[(2S)-2-amino-3-morpholin-4- | 491.5 | 246.2 |
| | ylpropyl]oxy}pyridin-3-yl)-8,9- | | |
| | dimethoxybenzo[c]-2,7-naphthyridin-4- | | |
| | amine | | |
| Ib-46 | 2-(5-{[(2S)-2-amino-3-(2,3-dihydro-1H- | 523.4 | 262.2 |
| | indol-1-yl)propyl]oxy}pyridin-3-yl)-8,9- | | |
| | dimethoxybenzo[c]-2,7-naphthyridin-4- | | |
| | amine | | |
| Ib-48 | 2-(5-{[(2R)-2-amino-3-(1,3-dihydro-2H- | 523.2 | 262.1 |
| | isoindol-2-yl)propyl]oxy}pyridin-3-yl)- | | |
| | 8,9-dimethoxybenzo[c]-2,7- | | |
| | naphthyridin-4-amine | | |
| Ib-50 | 2-(5-{[(2S)-2-amino-3-(3,4- | 537.2 | 269.1 |
| | dihydroisoquinolin-2(1H)- | | |
| | yl)propyl]oxy}pyridin-3-yl)-8,9- | | |
| | dimethoxybenzo[c]-2,7-naphthyridin-4- | | |
| | amine | | |

### Example 29

### Synthesis of Compound Ib-47

### (2-(5-{[(2R)-2-amino-3-(4-fluorophenyl)propyl]oxy}pyridin-3-yl)-8,9-dimethoxybenzo[c]-2,7-naphthyridin-4-amine)

The title compound is prepared in a manner analogous to the preparation of its enantiomer, compound Ib-41 of Example 22a. MS: 500.3 (M+H)⁺¹, MS: 250.7 (M+2H)⁺².

### Example 30

### Synthesis of Methyl 5-[((2R)-2-[(tert-butoxycarbonyl)amino]-3-{[tert-butyl(dimethyl)silyl]oxy}propyl)oxy]nicotinate

To a stirring solution of (R)-(+)-N-(tert butoxycarbonyl)-O-(tert-butyldimethylsilyl)serinol (26.2 g, 85.76 mmol), methyl 3-hydroxy nicotinate (13.13 g, 85.76 mmol), and triphenylphosphine (24.74 g, 94.34 mmol) at 0° C. is added 40% DEAD in toluene (44.8 g). After stirring at room temperature for 2 hr, the mixture is stirred at 60° C. overnight. The solvent is removed and the residue is extracted 4 times with hot hexanes (1 L total volume). The insoluble material is discarded. The solution is stirred and a solid separated which is removed by filtration. The solvent is removed from the filtrate and the residue is chromatographed on silica gel eluting with hexanes-ether mixtures to provide 23.7 g of the title compound.

### Example 31

### Synthesis of Compound Ib-25

### (tert-butyl [(1S)-2-{[5-(4-amino-8,9-dimethoxybenzo[c]-2,7-naphthyridin-2-yl)pyridin-3-yl]oxy}-1-(hydroxymethyl)ethyl]carbamate)

Step A: To a stirred solution of methyl 5-[((2R)-2-[(tert-butoxycarbonyl)amino]-3-{[tert-butyl(dimethyl)silyl]oxy}propyl)oxy]nicotinate (5.69 g, 12.92 mmol) and 6,7-dimethoxy-4-methyl-quinoline-3-carbonitrile (2.95 mg, 12.92 mmol) in 100 ml of THF under N₂ at −78° C. a solution of lithium hexamethyldisilazane (LiHMDS) in THF (58.2 ml, 58.2 mmol, 1 molar) is added. The mixture is placed in a bath at −50° C. and stirred for 1 hour. The mixture is slowly allowed to warm to room temperature over 1.5 hours and then poured into saturated ammonium chloride solution. The mixture is extracted several times with ethyl acetate and then with chloroform. The extracts are combined and dried (over MgSO₄). The solvent is removed and the residue is stirred in ether-hexanes 4:1 overnight. The resulting solid is collected and added to a mixture of 9.96 g of ammonium acetate and 1.91 g of ammonium fluoride in 59 ml of phenol. The mixture is stirred at 140° C. for 30 minutes and the phenol is removed by distillation at reduced pressure. The residue is mixed with a dilute sodium hydroxide solution and the solid is collected by filtration. The filtrate is then dissolved in boiling acetone, filtered, and concentrated. After cooling, the solid is collected to provide 3.75 g of tert-butyl [(1S)-2-{[5-(4-amino-8,9-dimethoxybenzo[c]-2,7-naphthyridin-2-yl)pyridin-3-yl]oxy}-1-(hydroxymethyl)ethyl]carbamate.

Step B: (S)-{2-[5-(4-amino-8,9-dimethoxy-benzo[c][2,7]naphthyridin-2-yl)-pyridin-3-yloxy]-1-hydroxymethyl-ethyl}-carbamic acid tert-butyl ester (330 mg) is mixed with 30 ml of ethyl acetate and 1 ml of concentrated hydrochloric acid. The mixture is stirred at 62° C. for 15 min and at room temperature for 30 min. The resulting solid is collected, washed with ether, and dried in vacuum to provide (2S)-2-amino-3-{[5-(4-amino-8,9-dimethoxybenzo[c]-2,7-naphthyridin-2-yl)pyridin-3-yl]oxy}propan-1-ol as the hydrochloride salt. MS: 422.4 (M+H)⁺¹, MS: 211.7 (M+2H)⁺².

### Example 32

### Synthesis of Compound Ib-10

### ((2S)-2-amino-3-{[5-(4-amino-8,9-dimethoxybenzo[c]-2,7-naphthyridin-2-yl)pyridin-3-yl]oxy}propan-1-ol)

(S)-{2-[5-(4-amino-8,9-dimethoxy-benzo[c][2,7]naphthyridin-2-yl)-pyridin-3-yloxy]-1-hydroxymethyl-ethyl}-carbamic acid tert-butyl ester (330 mg) is mixed with 30 ml of ethyl acetate and 1 ml of concentrated hydrochloric acid. The mixture is stirred at 62° C. for 15 minutes and then at room temperature for 30 minutes. The resulting solid is collected, washed with ether, and dried in vacuum to provide (2S)-2-amino-3-{[5-(4-amino-8,9-dimethoxybenzo[c]-2,7-naphthyridin-2-yl)pyridin-3-yl]oxy}propan-1-ol as the hydrochloride salt. MS: 422.4 (M+H)⁺¹, MS: 211.7 (M+2H)⁺².

### Example 33

### Synthesis of Compound Ib-26

### ((2R)-3-{[5-(4-amino-8,9-dimethoxybenzo[c]-2,7-naphthyridin-2-yl)pyridin-3-yl]oxy}-2-[(tert-butoxycarbonyl)amino]propyl methanesulfonate)

(S)-{2-[5-(4-amino-8,9-dimethoxy-benzo[c][2,7]naphthyridin-2-yl)-pyridin-3-yloxy]-1-hydroxymethyl-ethyl}-carbamic acid tert-butyl ester (3.75 g, 7.19 mmol) is dissolved in 60 ml of pyridine and cooled in an ice bath while methane sulfonyl chloride (2.06 g, 17.97 mmol) is added. The mixture is then stirred for 4.5 hours at room temperature and then poured into water. (2R)-3-{[5-(4-amino-8,9-dimethoxybenzo[c]-2,7-naphthyridin-2-yl)pyridin-3-yl]oxy}-2-[(tert-butoxycarbonyl)amino]propyl methanesulfonate is collected, washed with water, and dried at reduced pressure without heating. The compound is stirred with 225 ml of ethyl acetate and 7 ml of concentrated HCl for 1.5 hours at 55° C. The solvent is removed. Toluene is added and removed several times to remove the water. The residue is stirred in ethyl acetate and the suspended solid is collected via filtration to provide 4.1 g of (2R)-2-amino-3-{[5-(4-amino-8,9-dimethoxybenzo[c]-2,7-naphthyridin-2-yl)pyridin-3-yl]oxy}propyl methanesulfonate as a hydrochloride salt. MS: 500.4 (M+H)⁺¹.

### Example 34

### Synthesis of Compound Ib-7

### ((2R)-2-Amino-3-{[5-(4-amino-8,9-dimethoxybenzo[c]-2,7-naphthyridin-2-yl)pyridin-3-yl]oxy}propyl methanesulfonate)

(2R)-3-{[5-(4-amino-8,9-dimethoxybenzo[c]-2,7-naphthyridin-2-yl)pyridin-3-yl]oxy}-2-[(tert-butoxycarbonyl)amino]propyl methanesulfonate is stirred with 225 ml of ethyl acetate and 7 ml of concentrated hydrochloric acid for 1.5 hours at 55° C. Ethyl acetate is removed and toluene is added and removed several times to remove the water. The residue is stirred in ethyl acetate and the suspended solid is collected via filtration to provide 4.1 g of (2R)-2-amino-3-{[5-(4-amino-8,9-dimethoxybenzo[c]-2,7-naphthyridin-2-yl)pyridin-3-yl]oxy}propyl methanesulfonate as a hydrochloride salt. MS: 500.4 (M+H)⁺¹.

### Example 35

### Synthesis of Compound Ib-6

### ((2S)-3-{[5-(4-amino-8,9-dimethoxybenzo[c]-2,7-naphthyridin-2-yl)pyridin-3-yl]oxy}-N1-methyl-N1-(pyridin-3-ylmethyl)propane-1,2-diamine)

(2R)-2-Amino-3-{[5-(4-amino-8,9-dimethoxybenzo[c]-2,7-naphthyridin-2-yl)pyridin-3-yl]oxy}propyl methanesulfonate hydrochloride salt (400 mg, 0.66 mmol), N-methyl-N-(3-pyridylmethyl)amine (321 mg, 2.63 mmol), sodium iodide (78.8 mg, 0.53 mmol), and diisopropylethylamine (254.7 mg, 1.97 mmol) in 3 ml of DMF are stirred at 50° C. for 4.5 hours. DMF is then evaporated off at 70° C. under vacuum. Sodium bicarbonate solution is added to the residue, which is then extracted with hot chloroform-THF. The solution is dried (over MgSO₄) and placed on a silica gel column, which is eluted first with a chloroform-methanol (4:1) mixture. Product is eluted with chloroform-methanol (4:1)-1% triethylamine. Solvent is removed from the product fractions and the residue is stirred at 60° C. with ethyl acetate. The mixture is cooled and the solid is collected to provide 0.145 g of the title compound. MS: 526.5 (M+H)⁺¹, MS: 263.8 (M+2H)⁺².

### Example 36

### Synthesis of Compound Ib-8

### ((2S)-3-{[5-(4-amino-8,9-dimethoxybenzo[c]-2,7-naphthyridin-2-yl)pyridin-3-yl]oxy}-N1-(3-fluorobenzyl)-N1-methylpropane-1,2-diamine)

The title compound is prepared from (2R)-2-Amino-3-{[5-(4-amino-8,9-dimethoxybenzo[c]-2,7-naphthyridin-2-yl)pyridin-3-yl]oxy}propyl methanesulfonate hydrochloride salt and 3-fluoro-n-methylbenzylamine using methodology of Example 35. MS: 543.5 (M+H)⁺¹, MS: 272.3 (M+2H)⁺².

### Example 37

### Synthesis of Compound Ib-5

### (2-(5-{[(2S)-2-amino-3-(4,4-diethoxypiperidin-1-yl)propyl]oxy}pyridin-3-yl)-8,9-dimethoxybenzo[c]-2,7-naphthyridin-4-amine)

The title compound is prepared from (2R)-2-Amino-3-{[5-(4-amino-8,9-dimethoxybenzo[c]-2,7-naphthyridin-2-yl)pyridin-3-yl]oxy}propyl methanesulfonate hydrochloride salt and 4,4-diethoxy-piperidine as using methodology of Example 35. MS: 577.5 (M+H)⁺¹.

### Example 38

### Synthesis of Compound Ib-4

### (1-[(2S)-2-amino-3-{[5-(4-amino-8,9-dimethoxybenzo[c]-2,7-naphthyridin-2-yl)pyridin-3-yl]oxy}propyl]piperidin-4-one)

2-(5-{[(2S)-2-amino-3-(4,4-diethoxypiperidin-1-yl)propyl]oxy}pyridin-3-yl)-8,9-dimethoxybenzo[c]-2,7-naphthyridin-4-amine (230 mg, 0.4 mmol) is stirred with 2 ml of 6 N hydrochloric acid for 30 minutes. Toluene is added and removed 6 times to remove the water. The resulting solid is washed with ether and dried in vacuum to provide 0.23 g of the title compound as a hydrochloride salt. MS: 503.4 (M+H)⁺¹, MS: 252.2 (M+2H)⁺².

### Example 39

### Synthesis of Compound Ib-9

### (2-(5-{[(2S)-2-amino-3-(1H-imidazol-1-yl)propyl]oxy}pyridin-3-yl)-8,9-dimethoxybenzo[c]-2,7-naphthyridin-4-amine)

(2R)-2-Amino-3-{[5-(4-amino-8,9-dimethoxybenzo[c]-2,7-naphthyridin-2-yl)pyridin-3-yl]oxy}propyl methanesulfonate hydrochloride salt (600 mg, 0.99 mmol) and imidazole (670.8 mg, 9.85 mmol) are heated at 153° C. for 1.5 hours, before dilute sodium bicarbonate solution is added. The aqueous layer is decanted from the semi-solid, which is dissolved in chloroform-methanol and place onto a silica gel column. The column is washed first with chloroform-methanol mixtures and finally with chloroform-methanol-triethylamine (40:10:1) to elute the product. The solvent is removed from the product fraction and the residue is stirred with ethyl acetate. The solid is collected to provide 0.112 g of the title compound. MS: 472.4 (M+H)⁺¹, MS: 236.7 (M+2H)⁺².

### Example 40

### Synthesis of Compound Ia-29

### (N-[4-amino-8-ethoxy-2-(1H-imidazol-1-yl)benzo[c]-2,7-naphthyridin-9-yl]acetamide)

According to Step 6 of Example 1, N-(4-chloro-3-cyano-7-ethoxy-quinolin-6-yl)-acetamide is converted to (6-acetylamino-3-cyano-7-ethoxy-quinolin-4-yl)-cyano-acetic acid tert-butyl ester using t-butyl cyanoacetate and sodium hydride. To 4.3 g, 10.9 mmol of the product imidazole (18.55 g, 272.5 mmol) and pyridine hydrochloride (1.26 g, 10.9 mmol) are added. The mixture is heated at 127° C. for 10 hours. Water is added and the solid is collected, washed with water, and with ether. The solid is then boiled in isopropanol for 30 minutes. The solution is cooled and the title compound is collected to provide 2.4 g of a powder. MS: 363.2 (M+H)⁺¹, MS: 182.1 (M+2H)⁺².

### Example 41

### Synthesis of Compound Ia-28

### (8-Ethoxy-2-(1H-imidazol-1-yl)benzo[c]-2,7-naphthyridine-4,9-diamine)

A solution of N-[4-amino-8-ethoxy-2-(1H-imidazol-1-yl)benzo[c]-2,7-naphthyridin-9-yl]acetamide is refluxed in 25 ml of ethanol and 4 ml of concentrated hydrochloric acid for 3.5 hours. The mixture is poured into water and the solution is washed with ether. The aqueous layer is made basic with NaOH and the resulting solid is collected. The residue is dissolved in THF-methanol (100:5) and filtered through a pad of hydrous magnesium silicate. The solvent is removed and the residue is boiled in ethyl acetate. After cooling, 1.4 g of title compound is collected. MS: 321.2 (M+H)⁺¹, MS: 161.1 (M+2H)⁺².

### Example 42

### Synthesis of Compound Ia-27

### (N-[4-amino-8-ethoxy-2-(1H-imidazol-1-yl)benzo[c]-2,7-naphthyridin-9-yl]-N′-[4-(dimethylamino)phenyl]urea)

A mixture of 8-ethoxy-2-(1H-imidazol-1-yl)benzo[c]-2,7-naphthyridine-4,9-diamine (250 mg, 0.78 mmol) and (4-Isocyanato-phenyl)-dimethyl-amine (139 mg, 0.86 mmol) is heated at 100° C. for 2 hours. Water is added, the solid is collected and is washed with water. The solid is dissolved in THF-methanol (200:25) and filtered through hydrous magnesium silicate. The solvent is removed and the residue is boiled in ethyl acetate. After cooling, 0.255 g of the title compound is collected. MS: 483.32 (M+H)⁺¹, MS: 242.1 (M+2H)⁺².

### Example 43

### Synthesis of Compound Ia-26

### (N-[4-amino-8-ethoxy-2-(1H-imidazol-1-yl)benzo[c]-2,7-naphthyridin-9-yl]-N′-(2-morpholin-4-ylethyl)thiourea)

The title compound is prepared from 8-ethoxy-2-(1H-imidazol-1-yl)benzo[c]-2,7-naphthyridine-4,9-diamine and 4-(2-Isothiocyanato-ethyl)-morpholine using methodology of Example 42. MS: 493.22 (M+H)⁺¹, MS: 247.1 (M+2H)⁺²

### Example 44

### Synthesis of Compound Ib-61

### (8,9-dimethoxy-2-{5-[(2R)-pyrrolidin-2-ylmethoxy]pyridin-3-yl}benzo[c]-2,7-naphthyridin-4-amine)

Methyl 5-{[(2R)-1-(tert-butoxycarbonyl)pyrrolidin-2-yl]methoxy}nicotinate is prepared from methyl 3-hydroxy nicotinate and (R)-2-Hydroxymethyl-pyrrolidine-1-carboxylic acid tert-butyl ester using Step B, Method 1, of Example 22a. This is reacted with 6,7-dimethoxy-4-methyl-quinoline-3-carbonitrile using Step C, Method 1, of Example 22a to provide the title compound. MS: 432.1 (M+H)⁺¹, MS: 216.4 (M+2H)⁺².

### Example 45

### Synthesis of Compound Ib-59

### (8,9-dimethoxy-2-{5-[(3S)-pyrrolidin-3-yloxy]pyridin-3-yl}benzo[c]-2,7-naphthyridin-4-amine)

Methyl 5-{[(3S)-1-(tert-butoxycarbonyl)pyrrolidin-3-yl]oxy}nicotinate is prepared from methyl 3-hydroxy nicotinate and (R)-3-Hydroxy-pyrrolidine-1-carboxylic acid tert-butyl ester according to methods described in Step B, Method 1, of Example 22a. This is reacted with 6,7-dimethoxy-4-methyl-quinoline-3-carbonitrile according to methods described in Step C, Method 1, of Example 22a to provide the title compound. MS: 418.31 (M+H)⁺¹, MS: 209.6 (M+2H)⁺²

### Example 46

### Synthesis of Compound Ib-58

### (8,9-dimethoxy-2-{5-[(3R)-pyrrolidin-3-yloxy]pyridin-3-yl}benzo[c]-2,7-naphthyridin-4-amine)

Methyl 5-{[(3R)-1-(tert-butoxycarbonyl)pyrrolidin-3-yl]oxy}nicotinate is prepared from methyl 3-hydroxy nicotinate and (S)-2-Hydroxymethyl-pyrrolidine-1-carboxylic acid tert-butyl ester according to methods described in Step B, Method 1, of Example 22a. The product is reacted with 6,7-dimethoxy-4-methyl-quinoline-3-carbonitrile according to methods described in Step C, Method 1, of Example 22a to provide the title compound. MS: 418.3 (M+H)⁺¹.

### Example 47

### Synthesis of Compound Ib-57

### (5-(4-Amino-8,9-dimethoxybenzo[c]-2,7-naphthyridin-2-yl)pyridin-3-ol)

3-Trityloxy-benzoic acid methyl ester and 6,7-dimethoxy-4-methyl-quinoline-3-carbonitrile are condensed using methods described in Step C, Method 1, of Example 22a to provide the title compound. The reaction in acetic acid also served to remove the triyl group. MS: 349.2 (M+H)⁺¹.

### Example 48

### Synthesis of Compound Ib-51

### (2-(5-{[(2S)-2-amino-3-(benzyloxy)propyl]oxy}pyridin-3-yl)-8,9-dimethoxybenzo[c]-2,7-naphthyridin-4-amine)

(S)-5-(3-benzyloxy-2-tert-butoxycarbonylamino-propoxy)-nicotinic acid methyl ester is prepared from methyl 3-hydroxy nicotinate and (R)-(1-benzyloxymethyl-2-hydroxy-ethyl)-carbamic acid tert-butyl ester using methods described in Step C, Method 1, of Example 22a. This is reacted with 6,7-dimethoxy-4-methyl-quinoline-3-carbonitrile using the methods described in Step C, Method 1, of Example 22a to provide the title compound. MS: 512.4 (M+H)⁺¹, MS: 256.7 (M+2H)⁺².

### Example 49

### Synthesis of Compound Ib-63

### (8,9-Dimethoxy-2-(1-methyl-1H-imidazol-5-yl)benzo[c]-2,7-naphthyridin-4-amine)

The title compound is prepared by condensation of 6,7-dimethoxy-4-methyl-quinoline-3-carbonitrile and 3-methyl-3H-imidazole-4-carboxylic acid methyl ester using methods described in Step C, Method 1, of Example 22a to provide the title compound. MS: 336.1 (M+H)⁺¹, MS: 168.5 (M+2H)⁺².

### Example 50

### Synthesis of Compound Ia-2

### (9-Methoxy-8-(2-methoxyethoxy)-2-pyridin-3-ylbenzo[c]-2,7-naphthyridin-4-amine)

A degassed solution of 2-chloro-9-methoxy-8-(2-methoxyethoxy)benzo[c]-2,7-naphthyridin-4-amine (50 mg, 0.15 mmol), pyridyl-3-boronic acid (23 mg, 0.19 mmol), and triphenylphosphine (8 mg, 0.03 mmol) in a solvent system comprised of toluene (0.75 ml), N,N-dimethylformamide (0.75 ml) and 40% aqueous potassium carbonate (1.25 ml), is treated with bis(dibenzylideneacetone) palladium (0) (8.6 mg, 0.015 mmol) and the mixture is heated to 100° C. for 16 hours under an atmosphere of nitrogen. The mixture is then cooled to room temperature, diluted with 20 ml ethyl acetate, filtered through a pad of diatomaceous earth, and the filtered solids are washed with ethyl acetate. The pooled filtrate and washings are then washed successively with water, saturated aqueous sodium chloride, and dried over anhydrous sodium sulfate. The drying agent is then removed by filtration, the filtrate is concentrated, and the remaining residue is purified by silica gel chromatography using 10% methanol in ethyl acetate as the eluant to provide the title compound. MS: 377.3 (M+H)⁺¹.

### Example 51

### Synthesis of Compound Ia-34

### (8-(Benzyloxy)-2-(1H-imidazol-1-yl)-9-methoxybenzo[c]-2,7-naphthyridin-4-amine)

Step A: To a mechanically stirred solution of methyl vanillate (103.5 g, 0.568 mol) and benzyl bromide (101.36 ml, 0.852 mol) in acetone (800 ml) at room temperature powdered K₂CO₃ (196.25 g, 1.4 mol) is added The reaction is heated to 45° C. for 3.5 hours, cooled and filtered. The filtrate is concentrated in vacuo and the residue dissolved in EtOAc (300 ml) and washed with water (100 ml) three times saturated. NaHCO₃ (100 ml×2), and brine. The organic layer is dried over Na₂SO₄ and concentrated in vacuo to provide 224.72 g of a white solid. The solid is then triturated in hexane (300 ml) and filtered to provide methyl 4-(benzyloxy)-3-methoxybenzoate (141.45 g, 91%).

Step B: To a suspension of methyl 4-(benzyloxy)-3-methoxybenzoate (141.2 g, 0.519 mol) in acetic acid (400 ml) 160 ml of nitric acid is slowly added. After 20 hours, the reaction is treated with additional nitric acid (100 ml). At 22 hours, the reaction is quenched by addition of ice and then water. The resulting solids are filtered and washed with water three times. Solids are then dried via vacuum oven to provide methyl 4-(benzyloxy)-5-methoxy-2-nitrobenzoate (115 g, 70%).

Step C: Iron powder (46.2 g, 0.828 mol) is added to a suspension of methyl 4-(benzyloxy)-5-methoxy-2-nitrobenzoate (52.5 g, 165.6 mmol) in acetic acid (95 ml) and MeOH (200 ml). The reaction is then heated to reflux for 4 hours. The hot reaction mixture is then filtered through diatomaceous earth with 10% MeOH/CH₂Cl₂. The filtrate is concentrated in vacuo, dissolved in EtOAc, and passed through hydrous magnesium silicate using EtOAc. The organic phase is concentrated in vacuo and triturated with 30% EtOAc/Hexane to provide methyl 2-amino-4-(benzyloxy)-5-methoxybenzoate (31.94 g, 67%).

Step D: A suspension of methyl 2-amino-4-(benzyloxy)-5-methoxybenzoate (33.5 g, 116.5 mmol) in DMF-DMA (23.3 ml, 174.8 mmol) is heated to reflux for 5 hours. The reaction is then concentrated in vacuo and co-evaporated with toluene three times. The resulting solid is triturated with 30% EtOAc/hexane. After filtration, the collected solid is dried in a vacuum oven to provide methyl 4-(benzyloxy)-2-((dimethylamino)methyleneamino)-5-methoxybenzoate (34.57 g, 86%).

Step E: To stirring THF (275 ml) at −78° C. n-BuLi (83.9 ml) is slowly added. Acetonitrile (11 ml, 209.7 mmol) in THF (250 ml) is then added dropwise. After 10 minutes., methyl 4-(benzyloxy)-2-((dimethylamino)methyleneamino)-5-methoxybenzoate (34.2 g, 99.8 mmol) in THF (300 ml) is added dropwise to the white suspension. After 4 hours, the reaction is warmed to −55° C. and quenched by addition of acetic acid (28.6 ml, 495 mmol). The reaction is then warmed to room temperature and concentrated in vacuo. The resulting residue is then treated with water and the resulting solids are collected, and washed with water and hexane to provide 7-(benzyloxy)-4-hydroxy-6-methoxyquinoline-3-carbonitrile (29.87 g, 98%).

Step F: To a stirred suspension of 7-(benzyloxy)-4-hydroxy-6-methoxyquinoline-3-carbonitrile (5.72 g, 18.6 mmol) in CH₂Cl₂ (200 ml) and oxalyl chloride (7.33 ml, 84 mmol) DMF (1.86 mmol) is slowly added and then heated to reflux. After 2 hours, the reaction is concentrated in vacuo and then co-evaporated with toluene three times. The resulting residue is triturated in EtOAc, the solid is collected, and dried in a vacuum oven to provide 7-(benzyloxy)-4-chloro-6-methoxyquinoline-3-carbonitrile (5.63 g, 93%).

Step G: To a solution of 7-(benzyloxy)-4-chloro-6-methoxyquinoline-3-carbonitrile (1.1 g, 3.38 mmol) in DMSO (17 ml) Cs₂CO₃ (1.65 g, 5.08 mmol) and then tert-butyl cyanoacetate (4.06 mmol) are added. The reaction is heated to 100° C. for 4 hours. After cooling to room temperature, the reaction is poured into water (150 ml) and AcOH (2 ml). The solid is collected, washed with water, and then dried in a vacuum oven to provide tert-butyl 2-(7-(benzyloxy)-3-cyano-6-methoxyquinolin-4-yl)-2-cyanoacetate (1.17 g, 81%).

Step H: tert-Butyl 2-(7-(benzyloxy)-3-cyano-6-methoxyquinolin-4-yl)-2-cyanoacetate (1.07 g, 2.49 mmol), imidazole (4.24 g, 62.28 mmol), and pyridine.HCl (287 mg, 2.49 mmol) are melted at 140° C. for 5 h. The reaction is then poured into hot water and the resulting solid is collected. The solid is then washed with water and then Et₂O to provide 8-(benzyloxy)-2-(1H-imidazol-1-yl)-9-methoxybenzo[c][2,7]naphthyridin-4-amine (790.6 mg, 80%). MS: 398.2 (M+H)⁺¹.

### Example 52

### Synthesis of Compound Ia-33

### (4-Amino-2-(1H-imidazol-1-yl)-9-methoxybenzo[c]-2,7-naphthyridin-8-ol)

To a stirred solution of 8-(benzyloxy)-2-(1H-imidazol-1-yl)-9-methoxybenzo[c][2,7]naphthyridin-4-amine (1.06 g, 2.667 mmol) in HOAc (20 ml) 5% Pd/C (500 mg) is added. The air from the reaction is evacuated and filled with hydrogen. The evacuation is repeated three times. After stirring under balloon pressure H₂ for 12 hours, the reaction is filtered through diatomaceous earth with EtOAc and concentrated in vacuo. The residue is then purified via silica gel chromatography with 100% EtOAc-20% MeOH/EtOAc to provide 4-amino-2-(1H-imidazol-1-yl)-9-methoxybenzo[c][2,7]naphthyridin-8-ol (775 mg, 94%). MS: 308.1 (M+H)⁺¹.

### Example 53

### Synthesis of Compound Ia-21

### (8-(2-chloroethoxy)-2-(1H-imidazol-1-yl)-9-methoxybenzo[c][2,7]naphthyridin-4-amine)

To a stirred solution of 4-amino-2-(1H-imidazol-1-yl)-9-methoxybenzo[c][2,7]naphthyridin-8-ol (405 mg, 1.317 mmol) in DMF (7 ml) Cs₂CO₃ (643.6 mg, 1.975 mmol) and 2-chloroethyl-p-toluene sulfonate (262.9 mg, 1.44 mmol) are added. The reaction is stirred at room temperature for 2 hours and is then diluted with water. The resultant solid is collected and triturated with 5% EtOAc/hexane to provide 8-(2-chloroethoxy)-2-(1H-imidazol-1-yl)-9-methoxybenzo[c][2,7]naphthyridin-4-amine (342 mg, 70%). MS: 370.1 (M+H)⁺¹.

### Example 54

### Synthesis of Compound Ia-17

### (8-(3-Chloropropoxy)-2-(1H-imidazol-1-yl)-9-methoxybenzo[c][2,7]naphthyridin-4-amine)

The title compound is prepared in the manner described above in Example 53. MS: 384.1 (M+H)⁺¹.

### Example 55

### Synthesis of Compound Ia-10, Ia-12, Ia-13, Ia-14, Ia-15 and Ia-16

### (2-(1H-imidazol-1-yl)-9-methoxy-8-(2-pyrrolidin-1-ylethoxy)benzo[c]-2,7-naphthyridin-4-amine)

To a stirred solution of 8-(3-chloropropoxy)-2-(1H-imidazol-1-yl)-9-methoxybenzo[c][2,7]naphthyridin-4-amine (83 mg, 0.224 mmol) in NMP (1 ml) NaI (10 mg, 0.0673 mmol) and pyrrolidine (278 mL, 3.36 mmol) are added. The reaction is stirred at 130° C. for 2 hours, after which it is then cooled to room temperature and concentrated in vacuo. The residue is purified by reverse phase chromatography to provide 2-(1H-imidazol-1-yl)-9-methoxy-8-(2-(pyrrolidin-1-yl)ethoxy)benzo[c][2,7]naphthyridin-4-amine (48.2 mg, 59%). MS: 405.3 (M+H)⁺¹.

Using methods and reagents outlined in Examples 51-55, illustrative Benzo[c][2,7]naphthyridine Derivatives listed in Table 7 are prepared.

TABLE 7

| Com- | | MS: m/e |
|---|---|---|
| pound | Compound Name | (M + H)⁺¹ |
| Ia-10 | 2-(1H-imidazol-1-yl)-9-methoxy-8-(2-(4- | 434.4 |
| | methylpiperazin-1- | |
| | yl)ethoxy)benzo[c][2,7]naphthyridin- | |
| | 4-amine | |
| Ia-13 | 2-(1H-imidazol-1-yl)-9-methoxy-8-(2- | 421.3 |
| | morpholinoethoxy)benzo[c][2,7]naphthyridin- | |
| | 4-amine | |
| Ia-14 | 2-(1H-imidazol-1-yl)-9-methoxy-8-(3-(4- | 448.4 |
| | methylpiperazin-1- | |
| | yl)propoxy)benzo[c][2,7]naphthyridin-4-amine | |
| Ia-15 | 2-(1H-imidazol-1-yl)-9-methoxy-8-(3-(pyrrolidin-1- | 419.3 |
| | yl)propoxy)benzo[c][2,7]naphthyridin-4-amine | |
| Ia-16 | 2-(1H-imidazol-1-yl)-9-methoxy-8-(3- | 435.3 |
| | morpholinopropoxy)benzo[c][2,7]naphthyridin- | |
| | 4-amine | |

### Example 56

### Synthesis of (S)-3-Methanesulfonyloxy-piperidine-1-carboxylic acid tert-butyl ester

To a stirred solution of (S)—N-Boc-3-hydroxypiperidine (3 g, 17.1 mmol) and Et₃N (2.62 ml, 18.8 mmol) in CH₂Cl₂ (85 ml) at 0° C. methanesulfonyl chloride (1.33 ml, 17.1 mmol) is added. After stirring at 0° C. for 2 hours, the reaction is concentrated in vacuo and dissolved in ethyl acetate (100 ml). The reaction is washed with saturated NaHCO₃ (50 ml) three times, brine, dried over MgSO₄, filtered, and concentrated in vacuo to provide the title compound (4.25 g, 89%).

### Example 57

### Synthesis of (R)-Methyl 5-(1-(tert-butoxycarbonyl)piperidin-3-yloxy)nicotinate

A suspension of (S)-3-Methanesulfonyloxy-piperidine-1-carboxylic acid tert-butyl ester (2 g, 7.15 mmol), 5-hydroxynicotinic acid methyl ester (2.19 g, 14.32 mmol), and Cs₂CO₃ (4.89 g, 15.03 mmol) in DMF (14 ml) is stirred at room temperature for 1 hour. The reaction is then heated to 40° C. for 70 hours. After cooling to room temperature and concentration in vacuo, the reaction is dissolved in water (100 ml), extracted with EtOAc (50 ml), dried over MgSO₄, filtered, and concentrated in vacuo. The residue is then purified by silica gel chromatography (10-50% EtOAc/Hexanes) to provide (R)-methyl 5-(1-(tert-butoxycarbonyl)piperidin-3-yloxy)nicotinate (643 mg, 27%).

### Example 58

### Synthesis of (R)-5-(1-(tert-butoxycarbonyl)piperidin-3-yloxy)nicotinic acid

To a stirred solution of (R)-methyl 5-(1-(tert-butoxycarbonyl)piperidin-3-yloxy)nicotinate (680 mg, 2.021 mmol) in THF (10 ml) a 5 N solution of NaOH (8 ml, 40.43 mmol) is added. After stirring at room temperature for 3 hours, the reaction is concentrated in vacuo and extracted with EtOAc (30 ml) three times. The aqueous phase is then acidified to pH 5 by addition of concentrated HCl and extracted with EtOAc, dried over Na₂SO₄, filtered, and concentrated in vacuo to provide (R)-5-(1-(tert-butoxycarbonyl)piperidin-3-yloxy)nicotinic acid (2.08 g, 65%).

### Example 59

### Synthesis of (R)-tert-butyl 3-(5-(1H-imidazole-1-carbonyl)pyridin-3-yloxy)piperidine-1-carboxylate

To a stirred solution of (R)-5-(1-(tert-butoxycarbonyl)piperidin-3-yloxy)nicotinic acid (212 mg, 0.657 mmol) in THF (5 ml) 1,1′-carbonyldiimidazole (59.9 mg, 0.986 mmol) at room temperature is added. After stirring for 2 hours, the reaction is concentrated in vacuo, dissolved in EtOAc (40 ml), washed with cold water (20 ml×3), dried over MgSO₄, and concentrated in vacuo to provide (R)-tert-butyl 3-(5-(1H-imidazole-1-carbonyl)pyridin-3-yloxy)piperidine-1-carboxylate (231 mg, 94%).

### Example 60

### Synthesis of (R)-tert-butyl 3-(5-(4-amino-8,9-dimethoxybenzo-[c][2,7]-naphthyridin-2-yl)pyridin-3-yloxy)piperidine-1-carboxylate)

To a suspension of 6,7-dimethoxy-4-methylquinoline-3-carbonitrile (212.3 mg, 0.93 mmol) in THF at −78° C., LiHMDS (6.2 ml, 6.20 mmol, 10M THF) is added and the reaction is stirred for 10 minutes. Next, (R)-tert-butyl 3-(5-(1H-imidazole-1-carbonyl)pyridin-3-yloxy)piperidine-1-carboxylate (231 mg, 0.620 mmol) in THF (10 ml) is added dropwise and the resulting solution is stirred at −78° C. for 2 hours. The reaction is quenched by addition of powdered CO₂ and allowed to warm to room temperature, at which time water (10 ml) is added. The reaction is then extracted with EtOAc (30 ml) three times, dried over Na₂SO₄, filtered, and concentrated in vacuo. The residue is then dissolved in HOAc (15 ml) and treated with freshly sublimed NH₄OAc (956 mg, 12.4 mmol). The solution is heated to reflux for 10 minutes and then concentrated in vacuo. The residue is dissolved in EtOAc (50 ml) and washed with saturated NaHCO₃ (20 ml) three times, dried over Na₂SO₄, filtered and concentrated in vacuo. The residue is then purified by silica gel chromatography with 0-20% MeOH/EtOAc to provide (R)-tert-butyl 3-(5-(4-amino-8,9-dimethoxybenzo-[c][2,7]-naphthyridin-2-yl)pyridin-3-yloxy)piperidine-1-carboxylate (150.4 mg, 46%).

### Example 61

### Synthesis of Compound Ib-42

### (tert-Butyl 4-{[5-(4-amino-8,9-dimethoxybenzo[c]-2,7-naphthyridin-2-yl)pyridin-3-yl]oxy}piperidine-1-carboxylate)

Step A: To a stirred solution of tert-butyl 4-hydroxypiperidine-1-carboxylate (2 g, 9.937 mmol), 5-hydroxynicotinic methyl ester (1.52 g, 9.937 mmol), and triphenyl phosphine (3.9 g, 14.9 mmol) in THF (40 ml) at room temperature, diethyl azodicarboxylate (2.34 ml, 14.9 mmol) is added dropwise. The solution is stirred at room temperature for 12 hours and then concentrated in vacuo. The residue is dissolved in THF (20 ml), treated with NaOH (17.9 ml, 89.43 mmol, 5N), and stirred for 4 hours. The reaction is extracted with CH₂Cl₂ (20 ml) three times, and the aqueous layer acidified to pH 5 by addition of concentrated HCl. The aqueous phase is then extracted with CH₂Cl₂ (20 ml) three times, the organic layer is dried over Na₂SO₄, filtered, and concentrated in vacuo. Solids are then triturated in Et₂O for 12 hours to provide 5-(1-(tert-butoxycarbonyl)piperidin-4-yloxy)nicotinic acid (2.08 g, 65%).

Step B: To a stirred solution of 5-(1-(tert-butoxycarbonyl)piperidin-4-yloxy)nicotinic acid (1.07 g, 3.31 mmol) in THF (16 ml) 1,1′-carbonyldiimidazole (807.5 mg, 4.98 mmol) is added at room temperature. After stirring for 12 hours, the reaction is concentrated in vacuo, dissolved in EtOAc (100 ml), washed with cold water (20 ml) three times, dried over MgSO₄, and concentrated in vacuo to provide tert-butyl 4-(5-(1H-imidazole-1-carbonyl)pyridin-3-yloxy)piperidine-1-carboxylate (1.2 g, 99%).

Step C: To a suspension of 6,7-dimethoxy-4-methylquinoline-3-carbonitrile (500 mg, 2.19 mmol) in THF at −78° C., LiHMDS (8.76 ml, 8.76 mmol, 1.0M THF) is added and the reaction is stirred for 10 minutes. Next, 4-(5-(1H-imidazole-1-carbonyl)pyridin-3-yloxy)piperidine-1-carboxylate (815.7 mg, 2.19 mmol) in THF (20 ml) is added dropwise and the resulting solution is stirred at −78° C. for 2 hours. The reaction is quenched by addition of powdered CO₂ and allowed to warm to room temperature, at which time water (20 ml) is added. The reaction is then extracted with EtOAc (30 ml) three times, dried over Na₂SO₄, filtered, and concentrated in vacuo. The residue is dissolved in HOAc (30 ml) and treated with freshly sublimed NH₄OAc (3.37 g, 43.8 mmol). The solution is heated to reflux for 10 minutes and then concentrated in vacuo. The residue is dissolved in EtOAc (100 ml) and washed with saturated NaHCO₃ (20 ml) three times, dried over Na₂SO₄, filtered and concentrated in vacuo. The residue is then purified by silica gel chromatography with 0-20% MeOH/EtOAc to provide tert-butyl 4-(5-(4-amino-8,9-dimethoxybenzo[c][2,7]naphthyridin-2-yl)pyridin-3-yloxy)piperidine-1-carboxylate (751.9 mg, 65%). MS: 532.3 (M+H)⁺¹.

Using the methods outlined in Examples 56-61, illustrative Benzo[c][2,7]naphthyridine Derivatives listed in Table 8 are prepared.

TABLE 8

| | | (MS: |
|---|---|---|
| | | m/e |
| | | (M + |
| Compound | Compound Name | H)⁺¹ |
| Ia-7 | tert-butyl ((1S)-2-{[5-(4-amino-8-bromobenzo[c]- | 600.2 |
| | 2,7-naphthyridin-2-yl)pyridin-3-yl]oxy}-1- | |
| | benzylethyl)carbamate | |
| Ia-8 | 9-bromo-2-pyridin-3-ylbenzo[c]-2,7-naphthyridin-4- | 351 |
| | amine | |
| Ib-2 | tert-butyl-2-(5-(4-amino-8,9- | 581.2 |
| | dimethoxybenzo[c][2,7]naphthyridin-2-yl)pyridin-3- | |
| | yloxy)ethyl(benzyl)carbamate | |
| Ib-3 | tert-butyl-2-(5-(4-amino-8,9- | 519.2 |
| | dimethoxybenzo[c][2,7]naphthyridin-2-yl)pyridin-3- | |
| | yloxy)ethyl(ethyl)carbamate | |
| Ib-12 | tert-butyl-2-(5-(4-amino-8,9- | 491.2 |
| | dimethoxybenzo[c][2,7]naphthyridin-2-yl)pyridin-3- | |
| | yloxy)ethylcarbamate | |
| Ib-17 | tert-butyl [(1S)-1-({[5-(4-amino-8,9- | 520.4 |
| | dimethoxybenzo[c]-2,7-naphthyridin-2-yl)pyridin-3- | |
| | yl]oxy}methyl)propyl]carbamate | |
| Ib-43 | tert-butyl (3S)-3-{[5-(4-amino-8,9- | 532.3 |
| | dimethoxybenzo[c]-2,7-naphthyridin-2-yl)pyridin-3- | |
| | yl]oxy}piperidine-1-carboxylate | |

### Example 62

### Synthesis of Compound Ib-40

### (8,9-Dimethoxy-2-[5-(piperidin-4-yloxy)pyridin-3-yl]benzo[c]-2,7-naphthyridin-4-amine)

To a stirred solution of (S)-tert-butyl 1-(5-(4-amino-8,9-dimethoxybenzo[c][2,7]naphthyridin-2-yl)pyridin-3-yloxy)butan-2-yl carbamate (184.3 mg, 0.357 mmol) in CH₂Cl₂ (10 ml) TFA (1.36 ml, 17.7 mmol) is added and the solution is stirred at room temperature for 1 hour. The reaction is concentrated in vacuo and then co-evaporated with MeOH three times. The residue is dissolved in MeOH and treated with Et₂O until a solid precipitated from solution. The mixture is stirred for 12 hours and them resulting solid is collected to provide 8,9-dimethoxy-2-[5-(piperidin-4-yloxy)pyridin-3-yl]benzo[c]-2,7-naphthyridin-4-amine (208.3 mg, 77%). MS: 432.2 (M+H)⁺¹.

Using the methods outlined in Example 62, illustrative Benzo[c][2,7]naphthyridine Derivatives listed in Table 9 are prepared.

TABLE 9

| | | (MS: m/e |
|---|---|---|
| Compound | Compound Name | (M + H)⁺¹ |
| Ib-11 | 2-(5-(2-aminoethoxy)pyridin-3-yl)-8,9- | 391.2 |
| | dimethoxybenzo [c][2,7]naphthyridin-4-amine | |
| Ib-14 | 2-(5-{[(2S)-2-aminobutyl]oxy}pyridin-3-yl)- | 420.3 |
| | 8,9-dimethoxybenzo[c]-2,7-naphthyridin-4- | |
| | amine | |
| Ib-18 | 2-(5-{[(2R)-2-aminobutyl]oxy}pyridin-3-yl)- | 420.3 |
| | 8,9-dimethoxybenzo[c]-2,7-naphthyridin-4- | |
| | amine | |
| Ib-33 | 8,9-dimethoxy-2-{5-[(3R)-piperidin-3- | 432.3 |
| | yloxy]pyridin-3-yl}benzo[c]-2,7-naphthyridin- | |
| | 4-amine | |
| Ib-39 | 8,9-dimethoxy-2-{5-[(3S)-piperidin-3- | 432.2 |
| | yloxy]pyridin-3-yl}benzo[c]-2,7-naphthyridin- | |
| | 4-amine | |
| Ib-73 | 2-(5-(2-(ethylamino)ethoxy)pyridin-3-yl)-8,9- | 419.2 |
| | dimethoxybenzo[c][2,7]naphthyridin-4-amine | |
| Ib-75 | 2-(5-(2-(benzylamino)ethoxy)pyridin-3-yl)-8,9- | 481.2 |
| | dimethoxybenzo[c][2,7]naphthyridin-4-amine | |

### Example 63

### Synthesis of Compound Ia-6

### (tert-Butyl-{(1S)-2-[(5-{4-amino-8-[4-(1H-imidazol-1-yl)but-1-yn-1-yl]benzo[c]-2,7-naphthyridin-2-yl}pyridin-3-yl)oxy]-1-benzylethyl}carbamate)

A stirred solution of (S)-tert-butyl 1-(5-(4-amino-8-bromobenzo[c][2,7]naphthyridin-2-yl)pyridin-3-yloxy)-3-phenylpropan-2-ylcarbamate (110 mg, 0.183 mmol) in NMP (2 ml) is degassed by bubbling N₂ for 10 minutes and then treated with 1-(but-3-ynyl)-1H-imidazole (109 mg, 0.195 mmol), PdCl₂(Ph₃)P₂ (3.85 mg, 5.49 Mmol), Ph₃P (5.76 mg, 21.9 Mmol), CuI (1.04 mg, 5.49 Mmol), and Et₃N (128.6 ML, 0.915 Mmol). The solution is heated to 120° C. for 5 hours and then diluted with water (10 ml). The reaction is extracted with EtOAc (20 ml×3), dried over MgSO₄, filtered, and concentrated in vacuo. The residue is then purified by reverse phase chromatography to provide tert-butyl {(1S)-2-[(5-{4-amino-8-[4-(1H-imidazol-1-yl)but-1-yn-1-yl]benzo[c]-2,7-naphthyridin-2-yl}pyridin-3-yl)oxy]-1-benzylethyl}carbamate (59.6 mg, 51%). MS: 640.5 (M+H)⁺¹.

### Example 64

### Synthesis of Compound Ia-5

### (2-(5-{[(2S)-2-Amino-3-phenylpropyl]oxy}pyridin-3-yl)-8-[4-(1H-imidazol-1-yl)but-1-yn-1-yl]benzo[c]-2,7-naphthyridin-4-amine)

To a stirred solution of tert-butyl {(1S)-2-[(5-{4-amino-8-[4-(1H-imidazol-1-yl)but-1-yn-1-yl]benzo[c]-2,7-naphthyridin-2-yl}pyridin-3-yl)oxy]-1-benzylethyl}carbamate (300 mg, 0.46 mmol) in CH₂Cl₂ (5 ml) TFA (723 ML, 9.39 mmol) is added at room temperature. After stirring for 2 hours, the solution is concentrated in vacuo and purified by reverse phase chromatography to provide 2-(5-{[(2S)-2-amino-3-phenylpropyl]oxy}pyridin-3-yl)-8-[4-(1H-imidazol-1-yl)but-1-yn-1-yl]benzo[c]-2,7-naphthyridin-4-amine (203 mg, 82%). MS: 540.4 (M+H)⁺¹.

### Example 65

### Synthesis of Compound Ia-4

### (2-(5-{[(2S)-2-amino-3-phenylpropyl]oxy}pyridin-3-yl)-8-[(1E)-4-pyrrolidin-1-ylbut-1-en-1-yl]benzo[c]-2,7-naphthyridin-4-amine)

A stirred solution of (S)-tert-butyl 1-(5-(4-amino-8-bromobenzo[c][2,7]naphthyridin-2-yl)pyridin-3-yloxy)-3-phenylpropan-2-ylcarbamate (295 mg, 0.491 mmol), (E)-1-(4-(4,4,5,5-tetramethyl-1,3,2-dioxaborolan-2-yl)but-3-enyl)pyrrolidine (246.7 mg, 0.982 mmol), K₂CO₃ (203.6 mg, 1.47 mmol), tetrakis(triphenylphosphine)Pd (56.7 mg, 49.1 ML), EtOH (3 ml), H₂O (3 ml), and toluene (30 ml) is heated to reflux for 3 hours. The solution is then treated with TFA (1 ml) and heated to reflux for 30 minutes. The reaction is concentrated in vacuo and the residue is purified by reverse phase chromatography to provide 2-(5-{[(2S)-2-amino-3-phenylpropyl]oxy}pyridin-3-yl)-8-[(1E)-4-pyrrolidin-1-ylbut-1-en-1-yl]benzo[c]-2,7-naphthyridin-4-amine (33 mg, 6%). MS: 545.4 (M+H)⁺¹.

### Example 66

### Synthesis of Compound Ib-72

### (tert-Butyl-2-(5-(4-amino-8,9-dimethoxybenzo[c][2,7]naphthyridin-2-yl)pyridin-3-yloxy)ethyl(4-fluorobenzyl)carbamate)

Step A: To a stirred solution of tert-butyl 4-fluorobenzyl(2-hydroxyethyl)carbamate (2.2 g, 8.169 mmol), 5-hydroxynicotinic methyl ester (1.25 g, 8.169 mmol), and triphenyl phosphine (6.63 g, 25.32 mmol) in THF (40 ml) at room temperature, diethyl azodicarboxylate (3.85 ml, 24.5 mmol) is added dropwise. The solution is stirred at room temperature for 12 hours and then concentrated in vacuo. The residue is dissolved in EtOAc (50 ml) and extracted with HCl (20 ml, 0.5N) three times. The aqueous phase is neutralized with NaHCO₃, extracted with EtOAc (30 ml) three times, dried over Na₂SO₄, filtered, and concentrated in vacuo. The residue is purified by silica gel chromatography to provide methyl 5-(2-(tert-butoxycarbonyl(4-fluorobenzyl)amino)ethoxy)nicotinate (736.2 mg, 22%).

Step B: To a suspension of 6,7-dimethoxy-4-methylquinoline-3-carbonitrile (361.48 mg, 1.58 mmol) in THF at −78° C., LiHMDS (7.9 ml, 7.91 mmol, 10M THF) is added and the reaction is stirred for 10 minutes. Next, methyl 5-(2-(tert-butoxycarbonyl(4-fluorobenzyl)amino)ethoxy)nicotinate (640.5 mg, 1.58 mmol) in THF (10 ml) is added dropwise and the resulting solution is stirred at −78° C. for 6 hours. The reaction is quenched by addition of powdered CO₂ and allowed to warm to room temperature, at which time water (10 ml) is added. The reaction is extracted with EtOAc (30 ml) three times, dried over Na₂SO₄, filtered, and concentrated in vacuo. The residue is dissolved in HOAc (15 ml) and treated with freshly sublimed NH₄OAc (2.4 g, 31.67 mmol). The solution is heated to reflux for 10 minutes and then concentrated in vacuo. The residue is dissolved in EtOAc (50 ml) and washed with saturated NaHCO₃ (20 ml) three times, dried over Na₂SO₄, filtered, and concentrated in vacuo. The residue is purified by silica gel chromatography with 0-20% MeOH/EtOAc to provide tert-butyl 2-(5-(4-amino-8,9-dimethoxybenzo[c][2,7]naphthyridin-2-yl)pyridin-3-yloxy)ethyl(4-fluorobenzyl)carbamate (646.3 mg, 68%). MS: 599.2 (M+H)⁺¹.

### Example 67

### Synthesis of 2-(5-(2-(4-Fluorobenzylamino)ethoxy)pyridin-3-yl)-8,9-dimethoxybenzo[c][2,7]naphthyridin-4-amine

To a stirred solution of tert-butyl 2-(5-(4-amino-8,9-dimethoxybenzo[c][2,7]naphthyridin-2-yl)pyridin-3-yloxy)ethyl(4-fluorobenzyl)carbamate (551 mg, 0.918 mmol) in CH₂Cl₂ (5 ml), TFA (1.4 ml, 18.37 mmol) is added at room temperature. After stirring for 2 hours, the solution is concentrated in vacuo and purified by reverse phase chromatography to provide 2-(5-(2-(4-fluorobenzylamino)ethoxy)pyridin-3-yl)-8,9-dimethoxybenzo[c][2,7]naphthyridin-4-amine (203 mg, 82%). MS: 499.2 (M+H)⁺¹.

### Example 68

### Synthesis of Compound Ia-3

### (2-(5-{[(2S)-2-Amino-3-phenylpropyl]oxy}pyridin-3-yl)benzo[c]-2,7-naphthyridin-4-amine)

The title compound is prepared using the methods outlined in Example 22a using 3-cyano-4-methyl quinoline as starting material. MS: 422.2 (M+H)⁺¹.

### Example 69

### Synthesis of Compound Ib-31

### (2-(5-{[(2S)-2-aminopropyl]oxy}pyridin-3-yl)-8,9-dimethoxybenzo[c]-2,7-naphthyridin-4-amine)

Step A: To a mixture of (S)-(−)-2-(tert-butoxycarbonylamino)-1-propanol (5.50 g, 31.38 mmol) and triethylamine (3.49 g, 34.52 mmol) in dichloromethane (188 ml) at 0° C. methane sulfonyl chloride (3.74 g, 32.64 mmol) in dichloromethane (62 ml) is added dropwise over 30 minutes. The solvent is evaporated. The residue is partitioned in 600 ml of 3.1 ethyl acetate/water. The organic layer is separated, washed with 5% sodium bicarbonate solution and brine, dried over Na₂SO₄, filtered and evaporated to provide 6.44 g (81%) of a white solid which is characterized as (2S)-2-[(tert-butoxycarbonyl)amino]propyl methanesulfonate. MS (ESI): m/z 298.1.

Step B: A suspension of (2S)-2-[(tert-butoxycarbonyl)amino]propyl methanesulfonate (6.53 g, 25.8 mmol), 5-hydroxynicotinic acid methyl ester (3.96 g, 25.8 mmol) and cesium carbonate (16.81 g, 51.6 mmol) in N,N-dimethylformamide (79 ml) is stirred at 25° C. overnight. N,N-dimethylformamide is removed by rotary evaporator. Ethyl acetate (500 ml) is added and the cesium solid is filtered away. The ethyl acetate layer is washed with 5N sodium hydroxide solution, dried over Na₂SO₄, filtered and evaporated. The resulting residue is dissolved in hot 5:1 hexane/diethyl ether, then filtered through a 7.0 cm column of hydrous magnesium silicate. Fractions are eluted from the column with hexane and diethyl ether=1:1 (300 ml). Fractions 2 and 3 are collected and evaporated to provide 2.69 g (33.6%) of a white solid which is characterized as (5S)-(2-tert-butoxycarbonylamino-propoxy)-nicotinic acid methyl ester. MS (ESI) m/z 311.3.

Step C: To a solution of 6,7-dimethoxy-4-methylquinoline-3-carbonitrile (501 mg, 2.19 mmol) and (5S)-(2-tert-butoxycarbonylamino-propoxy)-nicotinic acid methyl ester (681 mg, 2.19 mmol) in tetrahydrofuran (22 ml) at −78° C., lithium bis(trimethylsilyl)-amide (1.0 M in THF) (10.98 ml, 10.98 mmol) is added over 15 minutes. The reaction mixture is stirred at −78° C. for 1 hour, at −50° C. for 1 hour, and at −20° C. for 1 hour. Saturated ammonium chloride solution (40 ml) is added. The yellow precipitate is filtered and washed with tetrahydrofuran (20 ml) and hexane (20 ml). It is dried in vacuum oven to provide 1.26 g of the ketone intermediate. To the solution of ketone (1.26 g, 2.49 mmol) in phenol (19 ml) ammonium acetate (2.72 g, 35.28 mmol) is added. The reaction mixture is stirred at 110° C. for 1 hour. Phenol is removed at 145° C. under vacuum distillation. The resulting residue is stirred in 2N hydrochloric acid (2.0 ml) and tetrahydrofuran (50 ml) at 65° C. for 2 hours. Brine and 5N sodium hydroxide are added to the reaction mixture, which is extracted with tetrahydrofuran and then with chloroform. The combined organic extracts are dried over Na₂SO₄, filtered and concentrated. The crude mixture is purified in a silica gel column, by elution with chloroform and 10% methanol/chloroform. Fractions are collected, washed with 10% methanol/chloroform (5.0 ml) and methanol/isopropanol=1:1 (5.0 ml) and filtered to provide a red solid. The red solid is stirred in hot acetonitrile and filtered. The filtrate is allowed to sit at room temperature until a yellow precipitate formed, then filtered to provide 78.1 mg (8.8%) of yellow solid which is characterized as 2-(5-{[(2S)-2-aminopropyl]oxy}pyridin-3-yl)-8,9-dimethoxybenzo[c]-2,7-naphthyridin-4-amine. MS: 406.3 (M+H)⁺¹, MS: 203.7 (M+2H)⁺².

### Example 70

### Synthesis of Compound Ib-30

### (2-(5-{[(2R)-2-aminopropyl]oxy}pyridin-3-yl)-8,9-dimethoxybenzo[c]-2,7-naphthyridin-4-amine)

Step A: Mesylation of (R)-(+)-2-(tert-butoxycarbonylamino)-1-propanol (5.50 g, 31.38 mmol) with methane sulfonyl chloride (3.74 g, 32.64 mmol) and triethylamine (3.49 g, 34.52 mmol) in dichloromethane (188 ml) at 0° C., following the procedure in Step A of Example 69, provided 7.89 g (99%) of A white solid which is characterized as (2R)-2-[(tert-butoxycarbonyl)amino]propyl methanesulfonate. MS (ESI) m/z 298.2.

Step B: Coupling of (2R)-2-[(tert-butoxycarbonyl)amino]propyl methanesulfonate (7.78 g, 30.72 mmol) to the 5-hydroxynicotinic acid methyl ester (4.7 g, 30.72 mmol) in the presence of cesium carbonate (20.01 g, 61.4 mmol) in N,N-dimethylformamide (93 ml) at 25° C., following the procedure in Step B of Example 69 provided 1.68 g (16.8%) of a white solid which is characterized as (5R)-(2-tert-butoxycarbonylamino-propoxy)-nicotinic acid methyl ester. MS (ESI) m/z 311.3.

Step C: The title compound is synthesized from 6,7-dimethoxy-4-methylquinoline-3-carbonitrile (500 mg, 2.19 mmol) and (5R)-(2-tert-butoxycarbonylamino-propoxy)-nicotinic acid methyl ester (681 mg, 2.19 mmol) by treating with lithium bis(trimethylsilyl)-amide (1.0 M in THF) (10.98 ml, 10.98 mmol) in tetrahydrofuran (22 ml) at −78° C., following the procedure in Step C of Example 69, to provide 1.8 g of the ketone intermediate, which is cyclized, deprotected and purified according to the same procedure as in Step C of Example 69 to provide 75.0 mg (8.8%) of a yellow solid, which is characterized as 2-(5-{[(2R)-2-aminopropyl]oxy}pyridin-3-yl)-8,9-dimethoxybenzo[c]-2,7-naphthyridin-4-amine. MS: 406.3 (M+H)⁺¹, 203.7 (M+2H)⁺².

### Example 71

### Synthesis of Compound Ib-32

### (2-(5-{3-[Benzyl(methyl)amino]prop-1-yn-1-yl}pyridin-3-yl)-8,9-dimethoxybenzo[c]-2,7-naphthyridin-4-amine)

To a solution of 2-(5-bromopyridin-3-yl)-8,9-dimethoxybenzo[c]-2,7-naphthyridin-4-amine (505 mg, 1.23 mmol), N-methyl-N-ropargylbenzylamine (215 mg, 1.35 mmol), triphenylphosphine (32.3 mg, 0.123 mmol) and tetrakis(triphenylphosphine)-palladium(0) (71.1 mg, 0.06 mmol) in hot, degassed piperidine (14 ml), copper iodide (23.42 mg, 0.123 mmol) is added. The reaction mixture is heated at reflux under nitrogen for 45 minutes. The solvent is evaporated. The residue is washed with ethyl acetate and the solid is filtered. The solid is stirred in hot isopropanol, filtered and dried in a vacuum oven to provide a yellow solid. Recrystallization of the yellow solid in hot ethyl acetate to provide 280 mg (46.6%) of a light yellow solid, which is identified as 2-(5-{3-[benzyl(methyl)amino]prop-1-yn-1-yl}pyridin-3-yl)-8,9-dimethoxybenzo[c]-2,7-naphthyridin-4-amine. MS: 490.4 (M+H)⁺¹.

### Example 72

### Synthesis of Compound Ib-22

### (8,9-Dimethoxy-2-[5-(3-{methyl[(1R)-1-methyl-2-phenylethyl]amino}prop-1-yn-1-yl)pyridin-3-yl]benzo[c]-2,7-naphthyridin-4-amine)

The title compound is synthesized by reacting 2-(5-bromopyridin-3-yl)-8,9-dimethoxybenzo[c]-2,7-naphthyridin-4-amine (276.1 mg, 0.671 mmol) with R-(−)deprenyl hydrochloride (165.2 mg, 0.738 mmol) in the presence of triphenylphosphine (17.6 mg, 0.067 mmol), tetrakis(triphenylphosphine)-palladium(0) (38.77 mg, 0.03 mmol) and copper iodide (12.78 mg, 0.067 mmol) in hot, degassed piperidine (10 ml) according to the procedure in example 71 to provide 33.8 mg (10%) of a yellow solid, which is identified as 8,9-dimethoxy-2-[5-(3-{methyl[(1R)-1-methyl-2-phenylethyl]amino}prop-1-yn-1-yl)pyridin-3-yl]benzo[c]-2,7-naphthyridin-4-amine. MS: 518.4 (M+H)⁺¹, 259.7 (M+2H)⁺².

### Example 73

### Synthesis of Compound Ib-1

### (2-{5-[(3S)-3-amino-4-phenylbut-1-yn-1-yl]pyridin-3-yl}-8,9-dimethoxybenzo[c]-2,7-naphthyridin-4-amine)

Step A: To a solution of N-(tert-butoxycarbonyl)-L-phenylalaine methyl ester (1.0 g, 3.58 mmol) in dichloromethane (10 ml) at −78° C., diisobutylaluminium (1.0 M in toluene) (7.87 ml, 7.87 mmol) is added dropwise over 15 minutes. The reaction mixture is stirred at −78° C. for 5 hours, quenched with acetic acid/toluene (1:5, 20 ml) at −78° C., warmed to room temperature, and poured into diluted hydrochloric acid (20 ml). The organic layer is separated. The aqueous layer is extracted with ethyl acetate (2×40 ml). The combined organic extracts are washed with brine, dried over Na₂SO₄, filtered and concentrated to provide 891 mg (100%) of a white solid, which is identified as (S)-(1-formyl-2-phenyl-ethyl)-carbamic acid tert-butyl ester: MS (ESI) m/z 248.2, MS (ESI) m/z 294.2; HRMS (high-resolution MS): calculated for C₁₄H₁₉NO₃⁺Na⁺, 272.12571, found (ESI-FTMS, [M+Na]¹⁺), 272.125.

Step B: To a suspension of carbon tetrabromide (1.9 g, 5.73 mmol), zinc (380 mg, 5.73 mmol) and triphenyl phosphine (1.5 mg, 5.73 mmol) in dichloromethane (30 ml), which is previously stirred for 16 hours at 25° C., (S)-(1-Formyl-2-phenyl-ethyl)-carbamic acid tert-butyl ester (714 mg, 2.86 mmol) in dichloromethane (30 ml) is added. The reaction mixture is stirred at 25° C. for 72 hours. Pentane (60 ml) is added and allowed to sit at room temperature for 15 minutes. The pentane layer is passed through a 3 inch silica gel plug, eluting with hexane, CH₂Cl₂/hexane (1:2) and CH₂Cl₂/hexane 1:1. Fractions contained desired product are collected and concentrated to provide 660 mg (57%) white solid, which is identified as tert-butyl [(1S)-1-benzyl-3,3-dibromoprop-2-en-1-yl]carbamate. MS (ESI) m/z 448.1; MS (ESI) m/z 438.1; HRMS: calculated for C₁₅H₁₉Br₂NO₂+H+, 403.98553; found (ESI-FTMS, [M+H]¹⁺), 403.98717.

Step C: To a solution of tert-butyl [(1S)-1-benzyl-3,3-dibromoprop-2-en-1-yl]carbamate (2.52 g, 6.23 mmol) in tetrahydrofuran (375 ml) at −78° C., n butyllithium (1.6M in hexane) (11.7 ml, 18.7 mmol) is added dropwise over 20 minutes. The reaction mixture is stirred at −78° C. for 1 hour, then hydrolyzed with 0.01M NaOH (30 ml) at −78° C., and extracted with diethyl ether. The ether extract is washed two times with brine, dried over Na₂SO₄, filtered and concentrated to provide a light yellow solid. The solid is washed with hot petroleum ether/hexane (20 ml), cooled to room temperature and filtered to provide 555 mg (36.3%) of a white solid, which is identified as tert-butyl [(1S)-1-benzylprop-2-yn-1-yl]carbamate. MS (ESI) m/z 246.2; MS (ESI) m/z 263.2; MS (ESI) m/z 491.3; HRMS: calculated for C₁₅H₁₉NO₂+H+, 246.14885; found (ESI-FTMS, [M+H]¹⁺), 246.14776.

Step D: To a solution of 2-(5-bromopyridin-3-yl)-8,9-dimethoxybenzo[c]-2,7-naphthyridin-4-amine (406.7 mg, 0.989 mmol), tert-butyl [(1S)-1-benzylprop-2-yn-1-yl]carbamate (266.9 mg, 1.09 mmol), triphenylphosphine (25.94 mg, 0.1 mmol) and tetrakis (triphenylphosphine)-palladium(0) (57.8 mg, 0.05 mmol) in hot, degassed piperidine (15 ml), copper iodide (18.8 mg, 0.1 mmol) is added. The reaction mixture is heated at reflux under nitrogen for 1 hour, then cooled to room temperature and then refrigerated. Hexane (10 ml) is added. The solid precipitate is filtered, is washed with dichloromethane and filtered again. The filtrate is passed through a silica gel plug, eluting with CH₂Cl₂, 3-7% MeOH/CH₂Cl₂. Fractions are collected and concentrated to provide 383 mg (67%) of a yellow solid, which is characterized as {3-[5-(4-amino-8,9-dimethoxy-benzo[c][2,7]naphthyridin-2-yl)-pyridin-3-yl]-1-benzyl-prop-2-ynyl}-carbamic acid tert-butyl ester. MS [M+H]¹⁺: 576.4.

Step E: To a solution of {3-[5-(4-amino-8,9-dimethoxy-benzo[c][2,7]naphthyridin-2-yl)-pyridin-3-yl]-1-benzyl-prop-2-ynyl}-carbamic acid tert-butyl ester (243.7 mg, 0.42 mmol) in tetrahydrofuran (8 ml) at room temperature, 2N hydrochloric acid (1 ml) is added. The reaction mixture is stirred at 65° C. for 2 hours. Brine (3 ml) and 5N NaOH (3 ml) are added. The reaction mixture then extracted with tetrahydrofuran (40 ml) and chloroform (30 ml).

The combined organic extracts are dried over Na₂SO₄, filtered and concentrated. The crude mixture is filtered through a silica gel plug, eluting with chloroform and 5-15% methanol/chloroform. Fractions are collected and concentrated. The solid is washed with hot hexane (5.0 ml) and filtered to yield 120.4 mg (59.9%) of a light yellow solid which is characterized as 2-{5-[(3S)-3-amino-4-phenylbut-1-yn-1-yl]pyridin-3-yl}-8,9-dimethoxybenzo[c]-2,7-naphthyridin-4-amine. MS: 476.4 (M+H)⁺¹, 238.7 (M+2H)⁺².

### Example 74

### Synthesis of 6,7-Diethoxy-4-methylquinoline-3-carbonitrile

A stirred mixture 0.83 g (3.0 mmol) of 4-chloro-6,7-diethoxyquinoline-3-carbonitrile (prepared as described in J. Med. Chem. (2000), 43:3244), 60 mg (0.60 mmol) of CuCl, and 12 ml of THF at 0° is treated with 5.36 (7.5 mmol) of 1.4M methylmagnesium bromide in 3:1 toluene-THF for 15 minutes. After 45 minutes at 0° C., the reaction mixture is partitioned with dichloromethane (DCM) and aqueous ammonium chloride. The organic layer is washed with water, dried, filtered through diatomaceous earth, and concentrated. The residue is chromatographed on silica gel with DCM-ethyl acetate to provide 640 mg of the title compound as a white solid, Mp 140-145° C., MS: m/e 257.2 (M+H)⁺¹.

### Example 75

### Synthesis of 4-[2-(4-bromophenyl)-2-oxoethyl]-6,7-diethoxyquinoline-3-carbonitrile

A solution of 6,7-diethoxy-4-methylquinoline-3-carbonitrile (128 mg, 0.50 mmol) in 2.5 ml of THF at −78° C. is treated with 1.5 ml of 1.0 M lithium hexamethyldisilazide in THF for 2 minutes. After 5 minutes, methyl 4-bromobenzoate (129 mg, 0.60 mmol) is added. The mixture is warmed to 25° C. over 10 minutes, stirred for 60 minutes, and quenched with solid CO₂. The mixture is partitioned with DCM and water. The organic layer is washed with water, dried, and concentrated. The residue is recrystallized from ethanol to provide 95 mg of the title compound as a light tan solid. Mp. 210-213° C., MS: m/e 439.0, 441.0 (M+H)⁺¹.

### Example 76

### Synthesis of Compound Ia-47

### (2-(4-bromophenyl)-8,9-diethoxybenzo[c]-2,7-naphthyridin-4-amine)

A solution of 4-[2-(4-bromophenyl)-2-oxoethyl]-6,7-diethoxyquinoline-3-carbonitrile (157 mg, 0.36 mmol) in 4.0 ml of HOAc is treated with ammonia gas for 1 minute, the resulting mixture is stirred at 130° C. for 30 minutes and evaporated to dryness. The residue is partitioned with DCM and aqueous ammonium hydroxide. The organic layer is washed with water, dried, and concentrated. The residue is chromatographed on silica gel with DCM-EtOAc-methanol to provide 43 mg of the title compound as a tan solid. Mp. 230-235° C., MS: m/e 438.0, 440.0 (M+H)⁺¹.

### Example 77

### Synthesis of Compound Ia-46

### (8,9-Diethoxy-2-pyridin-3-ylbenzo[c]-2,7-naphthyridin-4-amine)

Similar to the method of Example 75, 6,7-diethoxy-4-methylquinoline-3-carbonitrile is treated with LiHMDS and methyl nicotinate in THF to provide 6,7-diethoxy-4-[2-(pyridin-3-yl)-2-oxoethyl]quinoline-3-carbonitrile. In the manner of Example 76, the latter compound is treated with ammonium acetate in HOAc to provide the title compound as a tan solid, mp 241-245° C. MS: m/e 361.1 (M+H)⁺¹.

### Example 78

### 7-Benzyloxy-6-methoxy-4-methylquinoline-3-carbonitrile

Similar to the method of Example 74, 7-benzyloxy-4-chloro-6-methoxyquinoline-3-carbonitrile (c.f. U.S. Pat. No. 6,288,082) is treated with methylmagnesium bromide in the presence of CuCl to provide the title compound as a white solid. MS: m/e 305.2 (M+H)⁺¹.

### Example 79

### Synthesis of Compound Ia-23

### (8-(Benzylamino)-9-methoxy-2-pyridin-3-ylbenzo[c]-2,7-naphthyridin-4-amine)

Similar to the method of Example 75, 7-(benzyloxy)-6-methoxy-4-methylquinoline-3-carbonitrile is treated with LiHMDS and methyl nicotinate in THF to provide 7-(benzyloxy)-6-methoxy-4-[2-(3-pyridin-3-yl)-2-oxoethyl]quinoline-3-carbonitrile. Similar to the method of Example 76, the latter compound is treated with ammonium acetate in HOAc to provide the title compound as a tan solid. Mp. 251-258° C., MS: m/e 409.2 (M+H)⁺¹.

### Example 80

### Synthesis of 3-(1H-Imidazol-1-ylcarbonyl)-2-methylpyridine

To a stirred mixture of 2-methylnicotinic acid (1.37 g, 10 mmol) and 30 ml of THF, 1,1′-carbonyldiimidazole (1.95 g, 12 mmol) is added at 25° C. After 20 minutes the solution is refluxed for 5 minutes, cooled, and concentrated. The residue is partitioned at 0° C. with DCM and water. The organic layer is washed with water at 0° C., dried, and concentrated to provide the title compound as a colorless syrup.

### Example 81

### Synthesis of 6,7-Diethoxy-4-[2-(2-methylpyridin-3-yl)-2-oxoethyl]quinoline-3-carbonitrile

To a stirred mixture of 6,7-diethoxy-4-methylquinoline-3-carbonitrile (114 mg, 0.50 mmol), 3-(1H-imidazol-1-ylcarbonyl)-2-methylpyridine (112 mg, 0.60 mmol), and 2.5 ml of THF at −78° C., 1.75 ml of 1.0 M LiHMDS in THF is added over 5 minutes. The mixture is warmed to 25° C. over 15 minutes, stirred for 10 minutes, and quenched with solid carbon dioxide. The mixture is partitioned with DCM and water. The organic layer is washed with water, dried, and concentrated to provide the title compound as a brown solid, which is used directly in the next step.

### Example 82

### Synthesis of 8,9-Diethoxy-2-(2-methylpyridin-3-yl)benzo[c]-2,7-naphthyridin-4-amine

A mixture of 6,7-diethoxy-4-[2-(2-methylpyridin-3-yl)-2-oxoethyl]quinoline-3-carbonitrile (0.2 g, 0.5 mmol), ammonium acetate (0.8 g, 10 mmol), and 2.5 ml of HOAc is stirred at 130° C. for 30 minutes and concentrated to dryness. The residue is partitioned with DCM and aqueous ammonium hydroxide. The organic layer is washed with water and brine, dried and concentrated. The residue is chromatographed on silica gel with DCM-ethyl acetate-methanol-triethylamine to provide the title compound as a brown solid. MS: m/e 347.2 (M+H)⁺¹.

### Example 83

### Synthesis of (2Z)-2-Cyano-3-[(3-hydroxy-4-methoxyphenyl)amino]-3-(methylthio)acrylamide

A solution of 3-hydroxy-4-methoxyaniline (0.61 g, 4.4 mmol), 2-cyano-3,3-bis(methylthio)acrylamide (0.75 g, 4.0 mmol), and 8.0 ml of 2-ethoxyethanol is refluxed for 30 minutes and concentrated under vacuum. The residue is recrystallized from ethanol to provide the title compound as a white solid. Mp. 160-164° C., MS: m/e 280.2 (M+H)⁺¹.

### Example 84

### Synthesis of 3-[(3-Hydroxy-4-methoxyphenyl)amino]-3-(methylthio)acrylonitrile

A solution of (2Z)-2-cyano-3-[(3-hydroxy-4-methoxyphenyl)amino]-3-(methylthio)acrylamide (7.37 g, 26.4 mmol), triethylamine (14.8 ml, 106 mmol), and 79 ml of DMF is stirred at 140° C. for 3 hours and concentrated under vacuum. The residue is partitioned with DCM and aqueous ammonium chloride. The organic layer is washed with water, dried and concentrated. The residue is chromatographed on silica gel with DCM-ethyl acetate to provide the title compound as an amber syrup.

### Example 85

### Synthesis of 3-[(3-Acetoxy-4-methoxyphenyl)amino]-3-(methylthio)acrylonitrile

A solution of 3-[(3-hydroxy-4-methoxyphenyl)amino]-3-(methylthio)acrylonitrile (2.16 g, 9.1 mmol), acetic anhydride (4.3 ml, 45.5 mmol), pyridine (1.5 ml, 18.2 mmol), and 45 ml of toluene is stirred at 25° C. for 30 minutes. The solution is passed onto a column of silica gel, and the desired product is eluted with 25:1 DCM-ethyl acetate to provide the title compound as a syrup.

### Example 86

### Synthesis of 3-Cyano-6-methoxy-4-methyl-2-(methylthio)quinolin-7-yl acetate

To a stirred solution of 3-[(3-acetoxy-4-methoxyphenyl)amino]-3-(methylthio)acrylonitrile (27.4 g, 96 mmol), phosphorous oxychloride (13.5 ml, 145 mmol), and 289 ml of chlorobenzene at 0° C. N,N-dimethylacetamide (13.5 ml, 145 mmol) is added over 10 minutes. The solution is warmed to 50° C., stirred for 30 minutes, and then refluxed for 1.5 hours. The mixture is cooled, diluted with DCM and water, and cautiously treated with solid potassium carbonate until pH is between 7 and 8. The organic layer is washed with water, dried, and concentrated. The residue is stirred in methanol at 0° C., and the resulting off-white solid is filtered off to provide the title compound. Mp. 225-230° C., MS: m/e 303.1 (M+H)⁺¹.

### Example 87

### 3-Cyano-6-methoxy-4-methyl-2-(methylsulfonyl)quinolin-7-yl acetate

To a stirred mixture of 3-cyano-6-methoxy-4-methyl-2-(methylthio)quinolin-7-yl acetate (9.8 g, 32.4 mmol) and 324 ml of DCM m-chloroperoxybenzoic acid (mCPBA, 77%, 18.1 g, 81 mmol) is added over 5 minutes while cooling at 25-30° C. After 2.5 hours, the mixture is diluted with DCM and stirred with aqueous sodium sulfite while cooling. The organic layer is washed successively with water, sodium bicarbonate solution and water, dried, and concentrated to provide the title compound as a tan solid.

### Example 88

### Synthesis of 7-Hydroxy-6-methoxy-4-methylquinoline-3-carbonitrile)

A stirred mixture of 3-cyano-6-methoxy-4-methyl-2-(methylsulfonyl)quinolin-7-yl acetate (10.4 g, 32 mmol), zinc dust (10.6 g, 162 mg-atoms), and 324 ml of HOAc is heated at 70° C. for 60 minutes. The reaction mixture is cooled and filtered through diatomaceous earth using DCM to wash off the cake. The filtrate is evaporated using a toluene azeotrope to provide a solid. The solid is dissolved in 130 ml of methanol and treated with 16 ml of concentrated ammonium hydroxide while cooling. The solution is stirred for 16 hours, concentrated, and diluted with water. The resultant solid is filtered, washed with water, and crystallized from methanol to provide the title compound as a tan solid. Mp. 260° C. (decomposition). MS: m/e 215.2 (M+H)⁺¹.

### Example 89

### Synthesis of 6-Methoxy-7-(2-methoxyethoxy)-4-methylquinoline-3-carbonitrile

To a stirred mixture of 6-methoxy-7-(2-methoxyethoxy)-4-methylquinoline-3-carbonitrile 107 mg, 0.50 mmol), cesium carbonate (212 mg, 0.65 mmol), and 1.0 ml of DMF, 2-bromoethyl methyl ether (83 mg, 0.60 mmol) is added. After 1 day, additional 2-bromoethyl methyl ether (83 mg, 0.60 mmol) and DMF (1.0 ml) are added, and the mixture is stirred at 50° C. for 30 minutes. The reaction mixture is partitioned with DCM and water. The organic layer is washed with water, dried, and concentrated to provide 126 mg of the title compound as a tan solid. Mp. 188-193° C., MS: m/e 273.1 (M+H)⁺¹.

### Example 90

### Synthesis of (2Z-3-{[4-Benzyloxy)-3-(methoxyphenyl]amino}-2-cyano-3-(methylthio)acrylamide

Similar to the method of Example 83, 4-benzyloxy-3-methoxyaniline and 2-cyano-3,3-bis(methylthio)acrylamide are refluxed in 2-ethoxyethanol to provide the title compound as a white solid. Mp. 166-170° C., MS: m/e 368.3 (M−H)-1.

### Example 91

### Synthesis of 3-[(4-Benzyloxy-3-methoxyphenyl)amino]-3-(methylthio)acrylonitrile

Similar to the method of Example 84, (2Z-3-{[4-benzyloxy)-3-(methoxyphenyl]amino}-2-cyano-3-(methylthio)acrylamide is reacted with triethylamine in DMF at 140° C. to provide the title compound as a syrup, which is used directly in the next step.

### Example 92

### Synthesis of 6-(Benzyloxy)-7-methoxy-4-methyl-2-(methylthio)quinoline-3-carbonitrile

Similar to the method of Example 86, 3-[(4-benzyloxy-3-methoxyphenyl)amino]-3-(methylthio)acrylonitrile is reacted with phosphorous oxychloride and N,N-dimethylacetamide in chlorobenzene to provide the title compound as a tan solid. Mp. 171-174° C., MS: m/e 351.2 (M+H)⁺¹.

### Example 93

### Synthesis of (6-(Benzyloxy)-7-methoxy-4-methylquinoline-3-carbonitrile)

Similar to the method of Example 87, oxidation of 6-(benzyloxy)-7-methoxy-4-methyl-2-(methylthio)quinoline-3-carbonitrile with mCPBA in DCM provided 6-(benzyloxy)-7-methoxy-4-methyl-2-(methylsulfonyl)quinoline-3-carbonitrile. Similar to the method of Example 88, the latter compound is reacted with zinc dust in HOAc to provide the title compound as a tan solid. Mp. 165-172° C., MS: 305.1 (M+H)⁺¹.

### Example 94

### Synthesis of 6-Hydroxy-7-methoxy-4-methylquinoline-3-carbonitrile

A solution of 6-(benzyloxy)-7-methoxy-4-methylquinoline-3-carbonitrile (0.30 g, 1.0 mmol) and 0.59 ml (5.0 mmol) of thioanisole in 4.0 ml of TFA is refluxed for 60 minutes and concentrated under vacuum. The residue is stirred with aqueous ammonium hydroxide and hexane. The resulting tan solid is filtered, washed with water and hexane, and dried to provide the title compound.

### Example 95

### Synthesis of 7-Fluoro-6-methoxy-4-methylquinoline-3-carbonitrile

In the manner of Example 74, 7-fluoro-4-chloro-6-methoxyquinoline-3-carbonitrile (prepared as described in J. Med. Chem. (2004), 47:1599)) is treated with methylmagnesium bromide in the presence of CuCl to provide the title compound as a white solid. Mp. 177-179° C., MS: m/e 217.2 (M+H)⁺¹.

### Example 96

### Synthesis of 6-Methoxy-4-methyl-7-(pyridin-3-ylmethoxy)quinoline-3-carbonitrile

To a stirred mixture of 7-fluoro-6-methoxy-4-methylquinoline-3-carbonitrile (324 mg, 1.5 mmol), 3-pyridylcarbinol (0.41 g, 3.75 mmol), and 7.5 ml of THF at 0° C., 3.0 ml of 1.0 M sodium hexamethyldisilazide (NaHMDS) in THF is added over 5 minutes. The reaction mixture is stirred at 0° C. for 30 minutes, 25° C. for 30 minutes, and 45° C. for 60 minutes, then treated with 0.5 ml of water and concentrated. The residue is stirred in water, and the tan solid is filtered, washed with water, and dried to provide the title compound. Mp. 206-210° C., MS: m/e 306.2 (M+H)⁺¹.

Using similar methodology, the alcohols or thiols listed in Table 10 are reacted with 7-fluoro-6-methoxy-4-methylquinoline-3-carbonitrile in the presence of NaHMDS to provide 7-substituted quinolines.

TABLE 10

| Alcohol or Thiol | 7-Substituted Quinoline |
|---|---|
| (1-methylpiperidin-4-yl)methanol | 6-Methoxy-4-methyl-7-(1- |
| | methylpiperin-4- |
| | ylmethoxy)quinoline-3-carbonitrile |
| (Pyrrolidin-1-yl)-1-propanol | 6-Methoxy-4-methyl-7-(3- |
| | pyrrolidin-1-ylpropoxy)quinolin-3- |
| | carbonitrile |
| Ethanethiol | 7-(Ethylthio)-6-methoxy-4- |
| | methylquinoline-3-carbonitrile |

### Example 97

### Synthesis of 6-Methoxy-7-[(2-methoxyethyl)(methyl)amino]-4-methylquinoline-3-carbonitrile

A solution of 7-fluoro-6-methoxy-4-methylquinoline-3-carbonitrile (324 mg, 1.5 mmol) and (2-methoxyethyl)(methyl)amine (0.40 g, 4.5 mmol) in 0.75 ml of N-methyl-2-pyrrolidinone (NMP) is stirred at 105° C. for 7 hours and concentrated under vacuum. The residue is partitioned with DCM and water. The organic layer is washed with water, dried, and concentrated. The resulting oil is chromatographed on silica gel with DCM-EtOAc-methanol to provide the title compound as a tan solid. Mp. 90-96° C., MS: m/e 286.3 (M+H)⁺¹.

### Example 98

### Synthesis of 6-Methoxy-4-methyl-7-morpholin-4-ylquinoline-3-carbonitrile

Using the methodology of Example 97, morpholine is reacted with 7-fluoro-6-methoxy-4-methylquinoline-3-carbonitrile to provide the title compound.

### Example 99

### Synthesis of 6-Methoxy-4-methyl-7-(4-pyrrolidin-1-ylpiperidin-1-yl)quinoline-3-carbonitrile

Using the methodology of Example 97, 4-(pyrrolidin-1-yl)piperidine is reacted with 7-fluoro-6-methoxy-4-methylquinoline-3-carbonitrile to provide the title compound.

### Example 100

### Synthesis of Compound Ia-38

### (2-(5-{[(2S)-2-amino-3-phenylpropyl]oxy}pyridine-3-yl)-9-methoxy-N⁸-(2-methoxyethyl)-N⁸-methylbenzo[c]-2,7-naphthyridin-4,8-diamine)

To a stirred solution of 6-methoxy-7-[(2-methoxyethyl)(methyl)amino]-4-methylquinoline-3-carbonitrile (143 mg, 0.50 mmol) and methyl 5-({(2S)-2-[tert-butoxycarbonyl)amino]-3-phenylpropyl}oxy)nicotinate (232 mg, 0.60 mmol) in 2.5 ml of THF at −78° C., 4.0 ml of 1.0 M LiHMDS in THF is added over 5 minutes. The mixture is warmed to 0° C., stirred for 30 minutes, and treated with 5.0 ml of HOAc and ammonium acetate (2 g, 26 mmol). The resulting mixture is stirred at 80° C. for 30 minutes and concentrated under vacuum. The residue is partitioned with DCM and aqueous ammonium hydroxide. The organic layer is washed with water, dried, and concentrated. The residue is stirred with a mixture of 0.5 ml of concentrated HCl and 5 ml of EtOAc at 25° C. for 15 minutes. The reaction mixture is partitioned with DCM and aqueous ammonium hydroxide. The organic layer is washed with water, dried and concentrated. The residue is chromatographed on silica gel with DCM-EtOAc-methanol-triethylamine to provide the title compound as a tan solid. MS: m/e 539.5 (M+H)⁺¹.

### Example 101

### Synthesis of Compound Ia-40

### (2-(5-{[(2S)-2-amino-3-phenylpropyl]oxy}pyridin-3-yl)-8-(ethylthio)-9-methoxybenzo[c]-2,7-naphthyridin-4-amine)

Using the method of Example 100, treatment of 7-(ethylthio)-6-methoxy-4-methylquinoline-3-carbonitrile and methyl 5-({(2S)-2-[tert-butoxycarbonyl)amino]-3-phenylpropyl}oxy)nicotinate with LiHMDS, followed by treatment with ammonium acetate in HOAc, followed by treatment with concentrated hydrochloric acid and EtOAc, provided the title compound as a tan solid. MS: m/e 512.5 (M+H)⁺¹.

### Example 102

### Synthesis of Compound Ia-39

### (2-(5-{[(2S)-2-Amino-3-phenylpropyl]oxy}pyridin-3-yl)-9-methoxy-8-morpholin-4-ylbenzo[c]-2,7-naphthyridin-4-amine)

Using the method of Example 100, treatment of 6-methoxy-4-methyl-7-morpholin-4-ylquinoline-3-carbonitrile and methyl 5-({(2S)-2-[tert-butoxycarbonyl)amino]-3-phenylpropyl}oxy)nicotinate with LiHMDS, followed by ammonium acetate in HOAc, followed by concentrated hydrochloric acid and EtOAc provided the title compound as a tan solid. MS: m/e 537.4 (M+H)⁺¹.

### Example 103

### Synthesis of Compound Ia-22

### (9-Methoxy-8-[(1-methylpiperidin-4-yl)methoxy]-2-pyridin-4-ylbenzo[c]-2,7-naphthyridin-4-amine)

Using the method of Example 77, 6-methoxy-4-methyl-7-(1-methylpiperin-4-ylmethoxy)quinoline-3-carbonitrile is treated with LiHMDS and ethyl isonicotinate in THF followed by ammonium acetate in HOAc to provide the title compound as a tan solid. MS: m/e 430.3 (M+H)⁺¹.

### Example 104

### Synthesis of Compound Ia-31

### (9-Methoxy-2-pyridin-3-yl-8-(3-pyrrolidin-1-ylpropoxy)benzo[c]-2,7-naphthyridin-4-amine)

Using the method of Example 77, 6-methoxy-4-methyl-7-(3-pyrrolidin-1-ylpropoxy)quinolin-3-carbonitrile is treated with LiHMDS and methyl nicotinate in THF followed by ammonium acetate in HOAc to provide the title compound as a yellow solid. MS: m/e 430.2 (M+H)⁺¹.

### Example 105

### Synthesis of Compound Ia-20

### (9-methoxy-2-pyridin-3-yl-8-(4-pyrrolidin-1-ylpiperidin-1-yl)benzo[c]-2,7-naphthyridin-4-amine)

Using the method of Example 75, 6-methoxy-4-methyl-7-(4-pyrrolidin-1-ylpiperidin-1-yl)quinoline-3-carbonitrile is treated with LiHMDS and methyl nicotinate in THF followed by ammonium acetate in HOAc to provide the title compound as a yellow solid. MS: m/e 455.2 (M+H)⁺¹.

### Example 106

### Synthesis of 1-Imidazol-1-yl-3-pyridin-3-yl-propan-1-one

Using the method of Example 80, treatment of 3-(3-pyridyl)propionic acid with 1,1′-carbonyldiimidazole provided the title compound.

### Example 107

### Synthesis of Compound Ib-70

### (8,9-dimethoxy-2-(2-pyridin-3-ylethyl)benzo[c]-2,7-naphthyridin-4-amine)

A stirred mixture of 6,7-dimethoxy-4-methylquinoline-3-carbonitrile (114 mg, 0.50 mmol) and 2.5 ml of THF at −78° C. is treated with 1.0 ml of 1.0 M LiHMDS in THF over 2 minutes. After 10 minutes the mixture is treated with a solution of 1-imidazol-1-yl-3-pyridin-3-yl-propan-1-one (120 mg, 0.60 mmol) in 1.0 ml of THF over 30 seconds. The mixture is warmed to 0° C. during 10 minutes, cooled to −78° C., and treated with 1.0 ml of additional 1.0 M LiHMDS in THF. After 5 minutes, the mixture is treated with a solution of additional 1-imidazol-1-yl-3-pyridin-3-yl-propan-1-one (90 mg, 0.45 mmol) in 1.0 ml of THF. The mixture is warmed to 0° C. and treated with 6.0 ml of HOAc and ammonium acetate (2 g, 26 mmol). The resulting mixture is stirred at 80° C. for 30 minutes and concentrated to dryness under vacuum. The residue is partitioned with DCM and aqueous ammonium hydroxide. The organic layer is washed with water, dried, and concentrated. The residue is chromatographed on silica gel with DCM-EtOAc-methanol-triethylamine to provide the title compound as an off-white solid. MS: m/e 361.3 (M+H)⁺¹.

### Example 108

### Synthesis of Compound Ib-71

### (8,9-dimethoxy-2-methylbenzo[c]-2,7-naphthyridin-4-amine)

Using the method of Example 107, reaction of 6,7-dimethoxy-4-methylquinoline-3-carbonitrile and N-acetylimidazole with LiHMDS followed by ammonium acetate in HOAc provided the title compound as a light amber solid. Mp. 244-248° C., MS: m/e 270.2 (M+H)⁺¹.

### Example 109

### Synthesis of Compound Ib-16

### (N-((1R)-2-{[5-(4-amino-8,9-dimethoxybenzo[c]-2,7-naphthyridin-2-yl)pyridin-3-yl]oxy}-1-benzylethyl)formamide)

Compound Ib-62 (0.78 g, 1.62 mmol) is refluxed in ethylformate (2.4 mL) for 18 hours. The hot solution is filtered to provide the title compound (421.1 mg) as an orange solid. MS: 510.4 (M+H)⁺¹, 255.7 (M+2H)⁺².

### Example 110

### Synthesis of Compound Ib-15

### (2-(5-bromopyridin-3-yl)-8,9-dimethoxybenzo[c]-2,7-naphthyridin-4-amine)

To a stirred solution of 6,7-dimethoxy-4-methyl-quinoline-3-carbonitrile (2.78 g, 12.19 mmol) and 5-bromo-nicotinic acid methyl ester (2.63 g, 12.19 mmol) in THF (90 mL), a solution of LiHMDS (1.0 M, 61 mL, 60.93 mmol) is added in portions not to prevent the reaction temperature from exceeding −60° C. The reaction is then allowed to warm gradually to 15° C. over 18 hours. The reaction is then poured onto a saturated solution of ammonium chloride and is stirred for 1 hour. The resultant precipitate is collected and dried in vacuo overnight. The solid is dissolved in hot phenol to a final volume of 100 mL. The solution is treated with ammonium acetate (18.77 g, 243.8 mmol) and is heated to 100° C. for 2 hours. The phenol is removed by vacuum distillation and the resulting yellow solid is suspended in dilute sodium hydroxide. The solid is collected by vacuum filtration, washed with water, and then dried to provide 4.61 g of the title compound as a yellow solid. MS: 411.1 (M+H)⁺¹.

### Examples 111 and 112

### (N1-[5-(4-Amino-8,9-dimethoxy-benzo[c][2,7]naphthyridin-2-yl)-pyridin-3-yl]-propane-1,2-diamine and N²-[5-(4-Amino-8,9-dimethoxy-benzo[c][2,7]naphthyridin-2-yl)-pyridin-3-yl]-propane-1,2-diamine, respectively)

A mixture of 2-(5-bromopyridin-3-yl)-8,9-dimethoxybenzo[c]-2,7-naphthyridin-4-amine (290.7 mg, 0.709 mmol), 1,2-diaminopropane (0.1 ml, 1.06 mmol), copper iodide (3.5 mg, 0.035 mmol), tribasic potassium phosphate (301.5 mg, 1.42 mmol) and N,N-diethylsalicylamide (27.37 mg, 0.142 mmol) in DMF (20 ml) is heated at 100° C. in a shaker for 18 hours. The solution is diluted with 1N sodium hydroxide and is stirred for 1 hour. The aqueous solution is then saturated with sodium chloride and extracted with THF, followed by additional extractions with CHCl₃. The organic layers are combined, dried, and concentrated. The resulting residue is purified by HPLC (MeCN/water) to provide a 50:50 mixture of the two geometric isomers (204.9 mg orange solid). The isomers are then separated on the HLPC with isocratic MeOH. For N²-[5-(4-amino-8,9-dimethoxy-benzo[c][2,7]naphthyridin-2-yl)-pyridin-3-yl]-propane-1,2-diamine: MS: 405.3 (M+H)⁺¹, 203.1 (M+2H)⁺². For N1-[5-(4-amino-8,9-dimethoxy-benzo[c][2,7]naphthyridin-2-yl)-pyridin-3-yl]-propane-1,2-diamine: MS: 405.3 (M+H)⁺¹, 203.2 (M+2H)⁺²

### Example 113

### Synthesis of Compound Ia-19

### (9-methoxy-2-(2-methylpyridin-3-yl)-8-(4-pyrrolidin-1-ylpiperidin-1-yl)benzo[c]-2,7-naphthyridin-4-amine)

Using the method of Example 82, treatment of 6-methoxy-4-methyl-7-(4-pyrrolidin-1-ylpiperidin-1-yl)quinoline-3-carbonitrile and 3-(1H-imidazol-1-ylcarbonyl)-2-methylpyridine with LiHMDS followed by ammonium acetate in HOAc provided the title compound as an orange solid. MS: m/e 469.4 (M+H)⁺¹.

### Example 114

### Synthesis of Compound Ia-9

### (9-Methoxy-2-(2-methylpyridin-3-yl)-8-(3-pyrrolidin-1-ylpropoxy)benzo[c]-2,7-naphthyridin-4-amine)

Using the method of Example 82, treatment of 6-methoxy-4-methyl-7-(3-pyrrolidin-1-ylpropoxy)quinolin-3-carbonitrile and 3-(1H-imidazol-1-ylcarbonyl)-2-methylpyridine with LiHMDS, followed by ammonium acetate in HOAc provided the title compound as a yellow solid. MS: m/e 444.3 (M+H)⁺¹.

### Example 115

### Synthesis of Compound Ia-1

### (2-(5-{[(2S)-2-Amino-3-(4-fluorophenyl)propyl]oxy}pyridin-3-yl)-9-(benzyloxy)-8-methoxybenzo[c]-2,7-naphthyridin-4-amine)

Treatment of 6-(benzyloxy)-7-methoxy-4-methylquinoline-3-carbonitrile methyl 5-{[(2S)-2-[(tert-butoxycarbonyl)amino]-3-(4-fluorophenyl)propyl]oxy}nicotinate with LiHMDS, followed by ammonium acetate in HOAc provided the title compound as an amber solid. MS: m/e 576.4 (M+H)⁺¹.

### Example 116

### Synthesis of Compound Ib-77

### (N-[(2S)-2-amino-3-{[5-(4-amino-8,9-dimethoxybenzo[c][2,7]naphthyridin-2-yl)pyridin-3-yl]oxy}propyl]-N-butylbenzenesulfonamide)

Step A: To a solution of 1.25 g (3.1 mmol) of 2-{5-[(2S)-aziridin-2-ylmethoxy]pyridin-3-yl}-8,9-dimethoxybenzo[c]-2,7-naphthyridin-4-amine in 42 ml of CHCl₃ and 17 ml of methanol, 0.6 g (4.65 mmol) of diisopropylethyl amine and 0.81 g (3.72 mmol) of BOC anhydride is added. The mixture is stirred for 2 hours and the solvent is removed, providing (S)-tert-butyl 2-((5-(4-amino-8,9-dimethoxybenzo[c][2,7]naphthyridin-2-yl)pyridin-3-yloxy)methyl)aziridine-1-carboxylate.

By using this method and starting with 2-{5-[(2R)-aziridin-2-ylmethoxy]pyridin-3-yl}-8,9-dimethoxybenzo[c]-2,7-naphthyridin-4-amine, (R)-tert-butyl 2-((5-(4-amino-8,9-dimethoxybenzo[c][2,7]naphthyridin-2-yl)pyridin-3-yloxy)methyl)aziridine-1-carboxylate can be prepared.

Step B: To a solution of 0.34 g (0.67 mmol) of the compound prepared in Step A in 4 ml of DMF, 0.57 g (2.66 mmol) of N-butylbenzenesulfonamide and 0.54 g (1.66 mmol) of cesium carbonate is added. The mixture is heated to 75° C. for 5 hours. The solvent is removed at reduced pressure with heating. The mixture is dissolved in chloroform. The solution is washed with dilute sodium hydroxide and concentrated. The intermediate is purified by chromatography on silica gel by elution with chloroform-methanol mixtures. The solvent is removed and the residue is mixed with 23 ml of ethyl acetate, to which 1 ml of concentrated hydrochloric acid has been added. The mixture is stirred for 2 hours and the solid is collected to provide 0.44 g of the title compound as a hydrochloride salt. MS (M+H) (m/e) 617.3.

By using the method described above, the compounds shown in the Table below can be prepared.

| | | MS (m/e) |
|---|---|---|
| Compound | Compound name | (M + H⁾⁺¹ |
| Ib-78 | N-[(2S)-2-amino-3-{[5-(4-amino-8,9- | 579.4 |
| | dimethoxybenzo[c][2,7]naphthyridin-2- | |
| | yl)pyridin-3-yl]oxy}propyl]-3- | |
| | fluorobenzenesulfonamid | |
| Ib-79 | N-[(2S)-2-amino-3-{[5-(4-amino-8,9- | 575.4 |
| | dimethoxybenzo[c][2,7]naphthyridin-2- | |
| | yl)pyridin-3-yl]oxy}propyl]-N- | |
| | methylbenzenesulfonamide | |

### Example 117

### Synthesis of Compound Ib-80

### ((2S)-3-{[5-(4-amino-8,9-dimethoxybenzo[c][2,7]naphthyridin-2-yl)pyridin-3-yl]oxy}-N¹-phenylpropane-1,2-diamine)

Step A: To a solution of 1.93 g (4.78 mmol) of 2-{5-[(2S)-aziridin-2-ylmethoxy]pyridin-3-yl}-8,9-dimethoxybenzo[c]-2,7-naphthyridin-4-amine in 200 ml of CHCl₃ and 125 ml of methanol, 2.16 g (16.7 mmol) of diisopropylethyl amine and 1.42 g (5.98 mmol) of diphenylphosphinic chloride is added. The mixture is stirred for 3 hours, the solvent is removed and chromatographed using chloroform-methanol mixtures to provide 2.9 g of 2-(5-{[(2S)-1-(diphenylphosphoryl)aziridin-2-yl]methoxy}pyridin-3-yl)-8,9-dimethoxybenzo[c][2,7]naphthyridin-4-amine.

By using this method and starting with 2-{5-[(2R)-aziridin-2-ylmethoxy]pyridin-3-yl}-8,9-dimethoxybenzo[c]-2,7-naphthyridin-4-amine, 2-(5-{[(2R)-1-(diphenylphosphoryl)aziridin-2-yl]methoxy}pyridin-3-yl)-8,9-dimethoxybenzo[c][2,7]naphthyridin-4-amine can be prepared.

Step B: To a solution of 0.3 g (0.5 mmol) of 2-(5-{[(2S)-1-(diphenylphosphoryl)aziridin-2-yl]methoxy}pyridin-3-yl)-8,9-dimethoxybenzo[c][2,7]naphthyridin-4-amine (see Step A) in 6 ml of methoxyethanol, 66 mg (0.5 mmol) of lithium iodide and 0.46 g (4.97 mmol) of aniline is added. The mixture is refluxed for 17 hours. The mixture is poured onto a column of silica gel and purified by chromatography by elution with chloroform-methanol mixtures. The solvent is removed and the residue is mixed with 50 ml of ethyl acetate to which 0.7 ml of concentrated hydrochloric acid has been added. The mixture is stirred overnight and the solid is collected to provide 0.15 g of the title compound as a hydrochloride salt. MS (M+H) (m/e) 497.4.

By using the method described above, the compounds shown in the Table below can be prepared.

| | | MS (m/e) |
|---|---|---|
| Compound | Compound name | (M + H)⁺¹ |
| Ib-81 | (2S)-3-{[5-(4-amino-8,9-dimethoxybenzo[c][2,7]naphthyridin-2- | 529.4 |
| | yl)pyridin-3-yl]oxy}-N1-(3-fluoro-4-methylphenyl)propane-1,2- | |
| | diamine | |
| Ib-82 | 2-(5-{[(2S)-2-amino-3-(4-iminopyridin-1(4H)- | 498.4 |
| | yl)propyl]oxy}pyridin-3-yl)-8,9- | |
| | dimethoxybenzo[c][2,7]naphthyridin-4-amine | |
| Ib-83 | (2S)-3-{[5-(4-amino-8,9-dimethoxybenzo[c][2,7]naphthyridin-2- | 529.5 |
| | yl)pyridin-3-yl]oxy}-N1-(3-fluorophenyl)-N1-methylpropane-1,2- | |
| | diamine | |
| Ib-84 | (2S)-3-{[5-(4-amino-8,9-dimethoxybenzo[c][2,7]naphthyridin-2- | 549.4 |
| | yl)pyridin-3-yl]oxy}-N1-(3-chloro-4-fluorophenyl)propane-1,2- | |
| | diamine | |
| Ib-85 | (2S)-3-{[5-(4-amino-8,9-dimethoxybenzo[c][2,7]naphthyridin-2- | 533.5 |
| | yl)pyridin-3-yl]oxy}-N1-(2,4-difluorophenyl)propane-1,2-diamine | |
| Ib-86 | (2S)-3-{[5-(4-amino-8,9-dimethoxybenzo[c][2,7]naphthyridin-2- | 477.5 |
| | yl)pyridin-3-yl]oxy}-N1-tert-butylpropane-1,2-diamine | |
| Ib-87 | (2S)-3-{[5-(4-amino-8,9-dimethoxybenzo[c][2,7]naphthyridin-2- | 571.5 |
| | yl)pyridin-3-yl]oxy}-N1-[2-(4-fluorophenyl)-1,1- | |
| | dimethylethyl]propane-1,2-diamine | |
| Ib-88 | 2-(5-{[(2S)-2-amino-3-(2-amino-1H-benzimidazol-1- | 269.3 |
| | yl)propyl]oxy}pyridin-3-yl)-8,9- | (M + 2H) |
| | dimethoxybenzo[c][2,7]naphthyridin-4-amine | |
| Ib-89 | (2S)-3-{[5-(4-amino-8,9-dimethoxybenzo[c][2,7]naphthyridin-2- | 503.4 |
| | yl)pyridin-3-yl]oxy}-N1-cyclohexylpropane-1,2-diamine | |
| Ib-90 | (2S)-3-{[5-(4-amino-8,9-dimethoxybenzo[c][2,7]naphthyridin-2- | 539.4 |
| | yl)pyridin-3-yl]oxy}-N1-(1-methyl-1-phenylethyl)propane-1,2- | |
| | diamine | |
| Ib-91 | 4-{[(2S)-2-amino-3-{[5-(4-amino-8,9- | 519.4 |
| | dimethoxybenzo[c][2,7]naphthyridin-2-yl)pyridin-3- | |
| | yl]oxy}propyl]amino}cyclohexanol | |
| Ib-92 | 2-[5-({(2S)-2-amino-3-[4-(1,3-thiazol-2-yl)-1H-pyrazol-1- | 555.3 |
| | yl]propyl}oxy)pyridin-3-yl]-8,9- | |
| | dimethoxybenzo[c][2,7]naphthyridin-4-amine | |
| Ib-93 | (2S)-3-{[5-(4-amino-8,9-dimethoxybenzo[c][2,7]naphthyridin-2- | 540.4 |
| | yl)pyridin-3-yl]oxy}-N1-(1-methyl-1-pyridin-3-ylethyl)propane- | |
| | 1,2-diamine | |
| Ib-94 | (2S)-3-{[5-(4-amino-8,9-dimethoxybenzo[c][2,7]naphthyridin-2- | 540.4 |
| | yl)pyridin-3-yl]oxy}-N1-(1-methyl-1-pyridin-2-ylethyl)propane- | |
| | 1,2-diamine | |
| Ib-95 | (2S)-3-{[5-(4-amino-8,9-dimethoxybenzo[c][2,7]naphthyridin-2- | 517.4 |
| | yl)pyridin-3-yl]oxy}-N1-(cyclohexylmethyl)propane-1,2-diamine | |
| Ib-96 | (2S)-3-{[5-(4-amino-8,9-dimethoxybenzo[c][2,7]naphthyridin-2- | 505.4 |
| | yl)pyridin-3-yl]oxy}-N1-(tetrahydro-2H-pyran-4-yl)propane-1,2- | |
| | diamine | |
| Ib-97 | (2S)-3-{[5-(4-amino-8,9-dimethoxybenzo[c][2,7]naphthyridin-2- | 526.4 |
| | yl)pyridin-3-yl]oxy}-N1-(2-pyridin-4-ylethyl)propane-1,2-diamine | |
| Ib-98 | 2-[5-({(2S)-2-amino-3-[(2S)-2-(1,3-thiazol-2-yl)pyrrolidin-1- | 558.4 |
| | yl]propyl}oxy)pyridin-3-yl]-8,9- | |
| | dimethoxybenzo[c][2,7]naphthyridin-4-amine | |
| Ib-100 | (2S)-3-{[5-(4-amino-8,9-dimethoxybenzo[c][2,7]naphthyridin-2- | 475.4 |
| | yl)pyridin-3-yl]oxy}-N1-cyclobutylpropane-1,2-diamine | |
| Ib-101 | (2S)-3-{[5-(4-amino-8,9-dimethoxybenzo[c][2,7]naphthyridin-2- | 489.3 |
| | yl)pyridin-3-yl]oxy}-N1-cyclopentylpropane-1,2-diamine | |
| Ib-102 | 2-[5-({(2S)-2-amino-3-[(2R)-2-(1,3-thiazol-2-yl)pyrrolidin-1- | |
| | yl]propyl}oxy)pyridin-3-yl]-8,9- | |
| | dimethoxybenzo[c][2,7]naphthyridin-4-amine | |
| Ib-103 | (S)-3-(5-(4-amino-8,9-dimethoxybenzo[c][2,7]naphthyridin-2- | 613.4 |
| | yl)pyridin-3-yloxy)-N1-((4-phenyltetrahydro-2H-pyran-4- | |
| | yl)methyl)propane-1,2-diamine | |
| Ib-104 | (2S)-3-{[5-(4-amino-8,9-dimethoxybenzo[c][2,7]naphthyridin-2- | 595.4 |
| | yl)pyridin-3-yl]oxy}-N1-[(4-phenyltetrahydro-2H-pyran-4- | |
| | yl)methyl]propane-1,2-diamine | |
| Ib-105 | (2S)-3-{[5-(4-amino-8,9-dimethoxybenzo[c][2,7]naphthyridin-2- | |
| | yl)pyridin-3-yl]oxy}-N1-[(1-phenylcyclopentyl)methyl]propane- | |
| | 1,2-diamine | |
| Ib-106 | (2S)-3-{[5-(4-amino-8,9-dimethoxybenzo[c][2,7]naphthyridin-2- | 551.4 |
| | yl)pyridin-3-yl]oxy}-N1-[(1-phenylcyclopropyl)methyl]propane- | |
| | 1,2-diamine | |
| Ib-107 | 2-(5-{[(2S)-2-amino-3-(4-pyridin-4-ylpiperazin-1- | 567.4 |
| | yl)propyl]oxy}pyridin-3-yl)-8,9- | |
| | dimethoxybenzo[c][2,7]naphthyridin-4-amine | |
| Ib-108 | (2S)-3-{[5-(4-amino-8,9-dimethoxybenzo[c][2,7]naphthyridin-2- | 540.4 |
| | yl)pyridin-3-yl]oxy}-N1-(1-methyl-1-pyridin-4-ylethyl)propane- | |
| | 1,2-diamine | |
| Ib-109 | (2S)-3-{[5-(4-amino-8,9-dimethoxybenzo[c][2,7]naphthyridin-2- | 503.3 |
| | yl)pyridin-3-yl]oxy}-N1-(2,2,2-trifluoroethyl)propane-1,2-diamine | |
| Ib-110 | 2-(5-{[(2S)-2-amino-3-pyrrolidin-1-ylpropyl]oxy}pyridin-3-yl)-8,9- | 475.3 |
| | dimethoxybenzo[c][2,7]naphthyridin-4-amine | |
| Ib-111 | N-[(2S)-2-amino-3-{[5-(4-amino-8,9- | 512.4 |
| | dimethoxybenzo[c][2,7]naphthyridin-2-yl)pyridin-3- | |
| | yl]oxy}propyl]benzene-1,4-diamine | |
| Ib-112 | (2S)-3-{[5-(4-amino-8,9-dimethoxybenzo[c][2,7]naphthyridin-2- | 545.3 |
| | yl)pyridin-3-yl]oxy}-N1-(3-chlorophenyl)-N1-methylpropane-1,2- | |
| | diamine | |
| Ib-113 | (2S)-3-{[5-(4-amino-8,9-dimethoxybenzo[c][2,7]naphthyridin-2- | 513.3 |
| | yl)pyridin-3-yl]oxy}-N1-(4-methylpyrimidin-2-yl)propane-1,2- | |
| | diamine | |

### Example 118

### Synthesis of Compound Ib-114

### (2-(5-{[(5S)-2-anilino-4,5-dihydro-1H-imidazol-5-yl]methoxy}pyridin-3-yl)-8,9-dimethoxybenzo[c][2,7]naphthyridin-4-amine)

A mixture of 0.4 g (0.79 mmol) of (S)-tert-butyl 2-((5-(4-amino-8,9-dimethoxybenzo[c][2,7]naphthyridin-2-yl)pyridin-3-yloxy)methyl)aziridine-1-carboxylate, 1.1 g (5.66 mmol) of phenyl guanidine carbonate, and 1.44 g of diisopropylethylamine in 8 ml of DMF is heated at 75° C. for 48 hours. The solvents are removed at reduced pressure with heating. The residue is dissolved in chloroform and the solution is washed with 1 N sodium hydroxide and dried over magnesium sulfate. The solvent is removed. To the residue, 100 ml of ethyl acetate and 2 ml of concentrated hydrochloric acid is added and the mixture is stirred for 1.5 hours. The solvent is removed at reduced pressure. Toluene is added and evaporated at reduced pressure with heating to remove the remaining water. To the residue, 75 ml of isopropanol and 2 ml of diisopropylethylamine is added. The mixture is refluxed for 2 hours and the solvent is removed at reduced pressure. The residue is extracted with chloroform-methanol mixture (9:1). The solvent is removed and the residue is purified by HPLC to provide 0.11 g of the title compound. MS (M+H) (m/e) 522.3.

### Example 119

### Synthesis of Compounds Ib-141 to Ib-158

### (2-(5-{[(2R)-2-amino-3-(2-fluorophenoxy)propyl]oxy}pyridin-3-yl)-8,9-dimethoxybenzo[c][2,7]naphthyridin-4-amine)

To a solution of 0.34 g (0.67 mmol) of (S)-tert-butyl 2-((5-(4-amino-8,9-dimethoxybenzo[c][2,7]naphthyridin-2-yl)pyridin-3-yloxy)methyl)aziridine-1-carboxylate in 4 ml of DMF, 0.35 g (2.66 mmol) of 2,4-difluorophenol and 0.54 g (1.66 mmol) of cesium carbonate is added. The mixture is heated to 75° C. for 48 hours. The solvent is removed at reduced pressure with heating. The mixture is dissolved in chloroform. The solution is washed with dilute sodium hydroxide, dried over magnesium sulfate, and concentrated. The intermediate is purified by chromatography on silica gel eluting with chloroform-methanol mixtures. The solvent is removed and the residue is mixed with 26 ml of ethyl acetate to which 1.5 ml of concentrated hydrochloric acid has been added. The mixture is stirred for 3 hours and the solid is collected to provide 0.19 g of the title compound as a hydrochloride salt. MS (M+H) (m/e) 516.3.

By using the method described above, the compounds shown in the Table below can be prepared.

| | | MS (m/e) |
|---|---|---|
| Compound | Compound name | (M + H)⁺¹ |
| Ib-142 | 2-(5-{[(2R)-2-amino-3-(2-fluorophenoxy)propyl]oxy}pyridin-3-yl)- | 516.3 |
| | 8,9-dimethoxybenzo[c][2,7]naphthyridin-4-amine | |
| Ib-143 | 2-(5-{[(2S)-2-amino-3-(3,4-difluorophenoxy)propyl]oxy}pyridin-3- | 534.2 |
| | yl)-8,9-dimethoxybenzo[c][2,7]naphthyridin-4-amine | |
| Ib-144 | 2-[5-({(2R)-2-amino-3-[2-fluoro-5- | 584.4 |
| | (trifluoromethyl)phenoxy]propyl}oxy)pyridin-3-yl]-8,9- | |
| | dimethoxybenzo[c][2,7]naphthyridin-4-amine | |
| Ib-145 | 2-(5-{[(2R)-2-amino-3-{[5-(trifluoromethyl)pyridin-2- | 567.4 |
| | yl]oxy}propyl]oxy}pyridin-3-yl)-8,9- | |
| | dimethoxybenzo[c][2,7]naphthyridin-4-amine | |
| Ib-146 | 2-(5-{[(2R)-2-amino-3-(1H-benzimidazol-2- | 538.4 |
| | yloxy)propyl]oxy}pyridin-3-yl)-8,9- | |
| | dimethoxybenzo[c][2,7]naphthyridin-4-amine | |
| Ib-147 | 2-(5-{[(2R)-2-amino-3-(isoquinolin-1-yloxy)propyl]oxy}pyridin-3- | 549.4 |
| | yl)-8,9-dimethoxybenzo[c][2,7]naphthyridin-4-amine | |
| Ib-148 | 2-(5-{[(2R)-2-amino-3-(4,5-dihydro-1H-imidazol-2- | 490.4 |
| | yloxy)propyl]oxy}pyridin-3-yl)-8,9- | |
| | dimethoxybenzo[c][2,7]naphthyridin-4-amine | |
| Ib-149 | 2-(5-{[(2R)-2-amino-3-(pyrimidin-2-yloxy)propyl]oxy}pyridin-3- | 500.4 |
| | yl)-8,9-dimethoxybenzo[c][2,7]naphthyridin-4-amine | |
| Ib-150 | 2-(5-{[(2R)-2-amino-3-(1,3-benzothiazol-2- | 555.3 |
| | yloxy)propyl]oxy}pyridin-3-yl)-8,9- | |
| | dimethoxybenzo[c][2,7]naphthyridin-4-amine | |
| Ib-151 | 2-(5-{[(2S)-2-amino-3-(pyridin-4-yloxy)propyl]oxy}pyridin-3-yl)- | 499.3 |
| | 8,9-dimethoxybenzo[c][2,7]naphthyridin-4-amine | |
| Ib-152 | 2-[5-({(2R)-2-amino-3-[(4-methylpyridin-2- | 513.3 |
| | yl)oxy]propyl}oxy)pyridin-3-yl]-8,9- | |
| | dimethoxybenzo[c][2,7]naphthyridin-4-amine | |
| Ib-153 | 2-[5-({(2R)-2-amino-3-[(3,5-dichloropyridin-2- | 567.3 |
| | yl)oxy]propyl}oxy)pyridin-3-yl]-8,9- | |
| | dimethoxybenzo[c][2,7]naphthyridin-4-amine | |
| Ib-154 | 2-[5-({(2R)-2-amino-3-[(5-chloropyridin-2- | 533.3 |
| | yl)oxy]propyl}oxy)pyridin-3-yl]-8,9- | |
| | dimethoxybenzo[c][2,7]naphthyridin-4-amine | |
| Ib-155 | 3-{[(2S)-2-amino-3-{[5-(4-amino-8,9- | 523.3 |
| | dimethoxybenzo[c][2,7]naphthyridin-2-yl)pyridin-3- | |
| | yl]oxy}propyl]oxy}benzonitrile | |
| Ib-156 | 2-(5-{[(2R)-2-amino-3-(1,3-thiazol-2-yloxy)propyl]oxy}pyridin-3- | 505.2 |
| | yl)-8,9-dimethoxybenzo[c][2,7]naphthyridin-4-amine | |
| Ib-157 | tert-butyl {(1R)-2-{[5-(4-amino-8,9- | 605.4 |
| | dimethoxybenzo[c][2,7]naphthyridin-2-yl)pyridin-3-yl]oxy}-1- | |
| | [(1,3-thiazol-2-yloxy)methyl]ethyl}carbamate | |
| Ib-158 | 3-(4-{[(2S)-2-amino-3-{[5-(4-amino-8,9- | 551.3 |
| | dimethoxybenzo[c][2,7]naphthyridin-2-yl)pyridin-3- | |
| | yl]oxy}propyl]oxy}phenyl)propanenitrile | |

### Example 120

### Synthesis of Compounds Ib-159 to Ib-167

### (2-(5-{[(2R)-2-amino-3-(pyridin-2-ylthio)propyl]oxy}pyridin-3-yl)-8,9-dimethoxybenzo[c][2,7]naphthyridin-4-amine)

To a solution of 0.34 g (0.67 mmol) of (S)-tert-butyl 2-((5-(4-amino-8,9-dimethoxybenzo[c][2,7]naphthyridin-2-yl)pyridin-3-yloxy)methyl)aziridine-1-carboxylate in 4 ml of DMF, 0.295 g (2.66 mmol) of 2-mercapto pyridine and 0.54 g (1.66 mmol) of cesium carbonate is added. The mixture is heated to 70° C. for 3.5 hours. The solvent is removed at reduced pressure with heating. The mixture is dissolved in chloroform. The solution is washed with dilute sodium hydroxide, dried over magnesium sulfate, and concentrated. The intermediate is purified by chromatography on silica gel by elution with chloroform-methanol mixtures. The solvent is removed and the residue is mixed with 23 ml of ethyl acetate, to which 1 ml of concentrated hydrochloric acid has been added. The mixture is stirred for 3 hours and the solid is collected to provide 0.36 g of the title compound has a hydrochloride salt. MS (m/e) 515.3.

By using the method described above, the compounds shown in the Table below can be prepared.

| | | MS (m/e) |
|---|---|---|
| Compound | Compound name | (M + H)⁺¹ |
| Ib-160 | 2-(5-{[(2R)-2-amino-3-(1H-imidazol-2-ylthio)propyl]oxy}pyridin- | 510.2 |
| | 3-yl)-8,9-dimethoxybenzo[c][2,7]naphthyridin-4-amine | |
| Ib-161 | 2-[5-({(2R)-2-amino-3-[(1-methyl-1H-imidazol-2- | 518.3 |
| | yl)thio]propyl}oxy)pyridin-3-yl]-8,9- | |
| | dimethoxybenzo[c][2,7]naphthyridin-4-amine | |
| Ib-162 | 2-(5-{[(2R)-2-amino-3-(phenylthio)propyl]oxy}pyridin-3-yl)-8,9- | 514.4 |
| | dimethoxybenzo[c][2,7]naphthyridin-4-amine | |
| Ib-163 | 2-[5-({(2R)-2-amino-3-[(2,4- | 582.3 |
| | dichlorophenyl)thio]propyl}oxy)pyridin-3-yl]-8,9- | |
| | dimethoxybenzo[c][2,7]naphthyridin-4-amine | |
| Ib-164 | 2-(5-{[(2R)-2-amino-3-(pyridin-4-ylthio)propyl]oxy}pyridin-3-yl)- | 515.4 |
| | 8,9-dimethoxybenzo[c][2,7]naphthyridin-4-amine | |
| Ib-165 | 2-(5-{[(2R)-2-amino-3-(pyrimidin-2-ylthio)propyl]oxy}pyridin-3- | 516.3 |
| | yl)-8,9-dimethoxybenzo[c][2,7]naphthyridin-4-amine | |
| Ib-166 | 2-(5-{[(2R)-2-amino-3-(1,3-thiazol-2-ylthio)propyl]oxy}pyridin-3- | 521.3 |
| | yl)-8,9-dimethoxybenzo[c][2,7]naphthyridin-4-amine | |
| Ib-167 | (2-{[(2R)-2-amino-3-{[5-(4-amino-8,9- | 548.4 |
| | dimethoxybenzo[c][2,7]naphthyridin-2-yl)pyridin-3- | |
| | yl]oxy}propyl]thio}-1-methyl-1H-imidazol-5-yl)methanol | |

### Example 121

### Synthesis of Compound Ib-168

### (2-{5-[(4-Aminopyridin-3-yl)oxy]pyridin-3-yl}-8,9-dimethoxybenzo[c][2,7]naphthyridin-4-amine)

Step A: A mixture of 3 g (13.7 mmol) of 3-bromo-4-nitropyridine 1-oxide, 2.1 g (13.7 mmol) of methyl 5-hydroxynicotinate, and 5.7 g (41.1 mmol) of potassium carbonate in 60 ml of acetone containing 1 drop of Aliquat-336 is stirred at reflux for 6 hours. The mixture is diluted with acetone and filtered through magnesium silicate. The solvent is removed and the 2.6 g of 3-(5-(methoxycarbonyl)pyridin-3-yloxy)-4-nitropyridine 1-oxide is obtained after chromatography.

Step B: A mixture of 2.35 g (8.07 mmol) of 3-(5-(methoxycarbonyl)pyridin-3-yloxy)-4-nitropyridine 1-oxide, 2.7 g (48.4 mmol) of iron, and 2.91 g (48.4 mmol) of acetic acid is stirred at reflux for 2 hours. To the warm solution, 6 ml of NH₄OH is added. The mixture is filtered through diatomaceous earth and hydrous magnesium silicate using a chloroform-methanol mixture (1:1). The solvent is removed from the filtrate and the residue is purified by chromatography using chloroform containing 0.5% methanol to provide 1.8 g of methyl 5-(4-aminopyridin-3-yloxy)nicotinate.

Step C: To a solution of 1.6 g (6.12 mmol) of methyl 5-(4-aminopyridin-3-yloxy)nicotinate and 0.95 g (7.34 mmol) of diisopropylethylamine in 50 ml of tetrahydrofuran at 0° C., 2 g (9.2 mmol) of BOC anhydride is added. The mixture is stirred overnight. The solvent is removed and the product is purified by chromatography using chloroform-methanol mixtures to provide 2.05 g of methyl 5-(4-(tert-butoxycarbonylamino)pyridin-3-yloxy)nicotinate.

Step D: To a solution of 0.7 g (3.07 mmol) of 6,7-dimethoxy-4-methylquinoline-3-carbonitrile and 1.06 g (3.07 mmol) of methyl 5-(4-(tert-butoxycarbonylamino)pyridin-3-yloxy)nicotinate in 30 ml of tetrahydrofuran at −78° C. with stirring, 13.8 ml (13.8 mmol) of 1 M lithium bis(trimethylsilyl)amide in tetrahydrofuran is added. The mixture is then placed in an ice-bath and stirred for 0.5 hours at 0° C. 20 ml of acetic acid and 4.7 g of ammonium acetate is added to the mixture. The mixture is heated at 75° C. for 0.5 hours. The solvent is removed and the residue is dissolved in chloroform. This solution is washed with NH₄OH and dried over magnesium sulfate. The mixture is filtered and concentrated. The residue is purified by chromatography using chloroform-methanol mixtures. The solvent is removed and the residue is mixed with 50 ml of ethyl acetate and 3 ml of concentrated hydrochloric acid. The mixture is stirred at 50° C. for 1 hour and cooled to room temperature. The solid product is collected by filtration to provide 1.54 g of 2-{5-[(4-Aminopyridin-3-yl)oxy]pyridin-3-yl}-8,9-dimethoxybenzo[c][2,7]naphthyridin-4-amine as the hydrochloride salt. MS (M+H) (m/e) 441.3.

### Example 122

### Synthesis of Compound Ib-169

### (2-{5-[(3-Aminopyridin-2-yl)oxy]pyridin-3-yl}-8,9-dimethoxybenzo[c][2,7]naphthyridin-4-amine)

Step A: A solution of 3.1 g (19.6 mmol) of 2-chloro-3-nitropyridine, 2.99 g (19.6 mmol) of methyl 5-hydroxynicotinate, 8.1 g (58.7 mmol) of potassium carbonate, and 1 drop of Aliquat-336 in 86 ml acetone is stirred at reflux for 6 hours. The mixture is diluted with more acetone and filtered through diatomaceous earth. The solvent is removed and the product is purified by chromatography to provide 4.5 g of methyl 5-(3-nitropyridin-2-yloxy)nicotinate.

Step B: A mixture of 2.5 g (9.08 mmol) of methyl 5-(3-nitropyridin-2-yloxy)nicotinate, 3 g (54.5 mmol) of iron, and 3.27 g (54.5 mmol) of acetic acid is stirred at reflux for 2 hours. To the warm solution, 6 ml of NH₄OH is added. The mixture is filtered through diatomaceous earth using a chloroform-methanol mixture (1:1). The solvent is removed from the filtrate and the residue is purified by chromatography using magnesium silicate to provide 2.1 g of methyl 5-(3-aminopyridin-2-yloxy)nicotinate.

Step C: To a solution of 1 g (4.08 mmol) of methyl 5-(3-aminopyridin-2-yloxy)nicotinate and 0.05 g (0.41 mmol) of DMAP in 12 ml of tetrahydrofuran at 0° C., 1.78 g (8.2 mmol) of BOC anhydride is added. The mixture is stirred overnight. The solvent is removed and the product is purified by chromatography using chloroform mixtures to provide 0.7 g of methyl 5-(3-(bis(tert-butoxycarbonyl)amino)pyridin-2-yloxy)nicotinate.

Step D: To a solution of 0.35 g (1.53 mmol) of 6,7-dimethoxy-4-methylquinoline-3-carbonitrile and 0.68 g (1.53 mmol) of methyl 5-(3-(bis(tert-butoxycarbonyl)amino)pyridin-2-yloxy)nicotinate in 15 ml of tetrahydrofuran at −78° C. with stirring, 6.9 ml (6.9 mmol) of 1 M lithium bis(trimethylsilyl)amide in tetrahydrofuran is added. The mixture is then placed in an ice-bath and stirred for 0.5 hours at 0° C. 11 ml of acetic acid and 2.4 g of ammonium acetate is added to the mixture. The mixture is heated at 75° C. for 0.5 hours. The solvent is removed and the residue is dissolved in chloroform. This solution is washed with NH₄OH and dried over magnesium sulfate. The mixture is filtered and concentrated. The residue is purified by chromatography using chloroform-methanol mixtures. The solvent is removed and the residue is mixed with 50 ml of ethyl acetate and 1.5 ml of concentrated hydrochloric acid. The mixture is stirred at 50° C. for 1 hour and cooled to room temperature. The solid product is collected by filtration to provide 0.74 g of 2-{5-[(3-aminopyridin-2-yl)oxy]pyridin-3-yl}-8,9-dimethoxybenzo[c][2,7]naphthyridin-4-amine as the hydrochloride salt. MS (M+H) (m/e) 441.3.

### Example 123

### (2-(5-aminopyridin-3-yl)-8,9-dimethoxybenzo[c][2,7]naphthyridin-4-amine)

Step A: A suspension of 5-aminonicotinic acid (554.7 mg, 4.02 mmol), and hexane-2,5-dione (0.90 mL, 7.64 mmol), in pyridine (15 mL) is refluxed for 2 hours. Upon heating the reaction becomes homogeneous. The pyridine and the water generated are removed via distillation. The resulting oil is triturated with ether, and the black solid generated is collected by vacuum filtration. The filtrate is concentrated to provide a tan solid. The tan solid is heated in acetonitrile, the remaining solid is collected, and the filtrate is concentrated to provide 923.5 mg of 5-(2,5-dimethyl-1H-pyrrol-1-yl)nicotinic acid as an orange solid.

Step B: 5-(2,5-Dimethyl-1H-pyrrol-1-yl)nicotinic acid (1.15 g, 5.31 mmol) is dissolved in THF (40 mL) in the presence of carbonyldiimidazole (1.29 g, 7.96), and is then stirred at room temperature overnight. The reaction is concentrated, dissolved in chloroform, and washed with ice water to remove the imidazole generated in the reaction. The organic layers are combined, dried over sodium sulfate and concentrated to provide 1.43 g of 5-(2,5-dimethyl-1H-pyrrol-1-yl)pyridin-3-yl)(1H-imidazol-1-yl)methanone as a yellow oil.

Step C: A suspension of 6,7-dimethoxy-4-methylquinoline (1.23 g, 5.38 mmol), and (5-(2,5-dimethyl-1H-pyrrol-1-yl)pyridin-3-yl)(1H-imidazol-1-yl)methanone (1.43 g, 5.38 mmol) is cooled to −78° C. and then treated with a solution of LiHMDS (1.0 M in THF, 27.0 mL, 26.88 mmol) over a 10 minute period. The reaction is stirred at −78° C. for 1 hour, then for an additional 2 hours at 0° C. The reaction is then quenched with acetic acid (100 mL) at 0° C. and stirred for 30 minutes. The reaction is then treated with ammonium acetate (8.29 g, 107.6 mmol) and stirred at room temperature overnight. At this time the reaction is treated with hydroxylamine hydrochloride (7.48 g, 107.6 mmol), to cleave the pyrrole-protecting group, and is refluxed for 4 hours. The reaction is then concentrated. The resulting tan solid (3.28 g) exhibits low solubility. It is heated in 20% methanol/chloroform overnight, and then filtered hot. The filtrate is concentrated to provide 534 mg (28%) of 2-(5-aminopyridin-3-yl)-8,9-dimethoxybenzo[c][2,7]naphthyridin-4-amine as a tan solid.

### Example 124

### Synthesis of Compound Ib-170

### (2-{5-[(3-Aminopyridin-4-yl)oxy]pyridin-3-yl}-8,9-dimethoxybenzo[c][2,7]naphthyridin-4-amine)

Step A: A solution of 3.35 g (21.3 mmol) of 4-chloro-3-nitropyridine, 3.24 g (21.3 mmol) of methyl 5-hydroxynicotinate, 8.8 g (63.4 mmol) of potassium carbonate, and 1 drop of Aliquat-336 (Aldrich Chemical Co.) in 93 ml acetone is stirred at reflux for 6 hours. The mixture is diluted with more acetone and filtered through diatomaceous earth. The solvent is removed and the product is purified by chromatography to provide methyl 5-(3-nitropyridin-4-yloxy)nicotinate.

Step B: A mixture of 6.7 g (24.3 mmol) of methyl 5-(3-nitropyridin-4-yloxy)nicotinate, 8.2 g (146.1 mmol) of iron, and 8.77 g (146.1 mmol) of acetic acid is stirred at reflux for 2 hours. To the warm solution, 6 ml of NH₄OH is added. The mixture is filtered through diatomaceous earth using a chloroform-methanol mixture (1:1). The solvent is removed from the filtrate and the residue is purified by chromatography using magnesium silicate to provide 3.8 g of methyl 5-(3-aminopyridin-4-yloxy)nicotinate.

Step C: To a solution of 1 g (4.08 mmol) of methyl 5-(3-aminopyridin-4-yloxy)nicotinate and 0.07 g (0.61 mmol) of DMAP in 20 ml of tetrahydrofuran at 0° C., 2.67 g (12.2 mmol) of BOC anhydride is added. The mixture is stirred overnight. The solvent is removed and the product is purified by chromatography using chloroform mixtures to provide 0.98 g of methyl 5-(3-(bis(tert-butoxycarbonyl)amino)pyridin-4-yloxy)nicotinate.

Step D: To a solution of 0.425 g (1.86 mmol) of 6,7-dimethoxy-4-methylquinoline-3-carbonitrile and 0.83 g (1.86 mmol) of methyl 5-(3-(bis(tert-butoxycarbonyl)amino)pyridin-4-yloxy)nicotinate in 18 ml of tetrahydrofuran at −78° C. with stirring, 8.3 ml (8.3 mmol) of 1 M lithium bis(trimethylsilyl)amide in tetrahydrofuran is added. The mixture is then placed in an ice bath and stirred for 0.5 hours at 0° C. 14 ml of acetic acid and 2.9 g of ammonium acetate is added to the mixture. The mixture is heated at 75° C. for 0.5 hours. The solvent is removed and the residue is dissolved in chloroform. This solution is washed with NH₄OH and dried over magnesium sulfate. The mixture is filtered and concentrated. The residue is purified by chromatography using chloroform-methanol mixtures. The solvent is removed and the residue is mixed with 61 ml of ethyl acetate and 2 ml of concentrated hydrochloric acid. The mixture is stirred at 50° C. for 1 hour and cooled to room temperature. The solid product is collected by filtration to provide 0.51 g of 2-(5-(3-aminopyridin-4-yloxy)pyridin-3-yl)-8,9-dimethoxybenzo[c][2,7]naphthyridin-4-amine as a hydrochloride salt. MS (M+H) (m/e) 441.3.

### Example 125

### Synthesis of Compound Ib-171

### (2-{5-[(6-Aminopyridin-2-yl)oxy]pyridin-3-yl}-8,9-dimethoxybenzo[c][2,7]naphthyridin-4-amine)

Step A: To a solution of 3 g (17.44 mmol) of 6-bromopyridin-2-amine and 8.75 g (40.1 mmol) of BOC anhydride in 56 ml of tetrahydrofuran, 0.21 g (1.7 mmol) of DMAP is added. The mixture is stirred overnight. The solvent is removed and the residue is chromatographed to provide 5.5 g of the (bis(tert-butoxycarbonyl) derivative. To a 2 g (5.37 mmol) sample of this intermediate in 18 ml of DMF, 1.23 g (8.06 mmol) methyl 5-hydroxynicotinate, 0.26 g (1.34 mmol) of CuI, 2.63 g (8.1 mmol) of cesium carbonate, and 0.24 g of 1,10-phenatoline is added. The mixture is heated to 100° C. for 6 hours and then poured into dilute NH₄OH. The mixture is extracted with ether and the ether solution is dried over magnesium sulfate. The solvent is removed and the residue is purified by chromatography to provide 0.39 g of methyl 5-(6-(tert-butoxycarbonylamino)pyridin-2-yloxy)nicotinate.

Step B: To a solution of 0.26 g (1.12 mmol) of 6,7-dimethoxy-4-methylquinoline-3-carbonitrile and 0.39 g (1.12 mmol) of methyl 5-(6-(tert-butoxycarbonylamino)pyridin-2-yloxy)nicotinate in 11 ml of tetrahydrofuran at −78° C. with stirring, 5 ml (5 mmol) of 1 M lithium bis(trimethylsilyl)amide in tetrahydrofuran is added. The mixture is then placed in an ice bath and stirred for 0.5 hours at 0° C. 9 ml of acetic acid and 1.7 g of ammonium acetate is added to the mixture. The mixture is heated at 75° C. for 0.5 hours. The solvent is removed and the residue is dissolved in chloroform. This solution is washed with NH₄OH and dried over magnesium sulfate. The mixture is filtered and concentrated. The residue is purified by chromatography using chloroform-methanol mixtures. The solvent is removed and the residue is mixed with 36 ml of ethyl acetate and 0.5 ml of concentrated hydrochloric acid. The mixture is stirred at 50° C. for 1 hour and cooled to room temperature. The solid product is collected by filtration to provide 0.09 g of 2-{5-[(6-Aminopyridin-2-yl)oxy]pyridin-3-yl}-8,9-dimethoxybenzo[c][2,7]naphthyridin-4-amine as a hydrochloride salt. MS (M+H) (m/e) 441.3.

### Example 126

### Synthesis of Compound Ib-172

### (2-{5-[(6-Aminopyridin-3-yl)oxy]pyridin-3-yl}-8,9-dimethoxybenzo[c][2,7]naphthyridin-4-amine)

Step A: A solution of 3.79 g (18.73 mmol) of 5-bromo-2-nitropyridine, 2.86 g (18.7 mmol) of methyl 5-hydroxynicotinate, 8.52 g (26.14 mmol) of cesium carbonate in 51 ml of DMF is stirred at 100° C. for 3 hours. The mixture is diluted with water and extracted with ethyl acetate. The solvent is removed and the product is purified by chromatography to provide 3.2 g of methyl 5-(6-nitropyridin-3-yloxy)nicotinate.

Step B: A mixture of 3.16 g (11.5 mmol) of methyl 5-(6-nitropyridin-3-yloxy)nicotinate, 3.85 g (68.9 mmol) of iron, and 4.1 g (68.9 mmol) of acetic acid is stirred at reflux for 2 hours. To the warm solution, 6 ml of NH₄OH is added. The mixture is filtered through diatomaceous earth using a chloroform-methanol mixture (1:1). The solvent is removed from the filtrate and the residue is purified by chromatography using magnesium silicate to provide 1.8 g of methyl 5-(6-aminopyridin-3-yloxy)nicotinate.

Step C: To a solution of 1.7 g (6.93 mmol) of methyl 5-(6-aminopyridin-3-yloxy)nicotinate and 0.12 g (1 mmol) of DMAP in 20 ml of tetrahydrofuran at 0° C., 4.2 g (19.1 mmol) of BOC anhydride is added. The mixture is stirred for 3 hours. The solvent is removed and the product is purified by chromatography using chloroform mixtures to provide 1.1 g of methyl 5-(6-(bis(tert-butoxycarbonyl)amino)pyridin-3-yloxy)nicotinate.

Step D: To a solution of 0.5 g (2.19 mmol) of 6,7-dimethoxy-4-methylquinoline-3-carbonitrile and 0.975 g (2.19 mmol) of methyl 5-(6-(bis(tert-butoxycarbonyl)amino)pyridin-3-yloxy)nicotinate in 22 ml of tetrahydrofuran at −78° C. with stirring, 9.86 ml (9.86 mmol) of 1 M lithium bis(trimethylsilyl)amide in tetrahydrofuran is added. The mixture is placed in an ice bath and stirred for 0.5 hours at 0° C. 17 ml of acetic acid and 3.4 g of ammonium acetate is added to the reaction mixture. The mixture is heated at 75° C. for 0.5 hours. The solvent is removed and the residue is dissolved in chloroform. This solution is washed with NH₄OH and dried over magnesium sulfate. The mixture is filtered and concentrated. The residue is purified by chromatography using chloroform-methanol mixtures. The solvent is removed and the residue is mixed with 70 ml of ethyl acetate and 2.4 ml of concentrated hydrochloric acid. The mixture is stirred at 50° C. for 1 hour and cooled to room temperature. The solid product is collected by filtration to provide 0.69 g of 2-{5-[(6-Aminopyridin-3-yl)oxy]pyridin-3-yl}-8,9-dimethoxybenzo[c][2,7]naphthyridin-4-amine as the hydrochloride salt. MS (M+H) (m/e) 441.3.

### Example 127

### (Tert-butyl (6-{[5-(4-amino-8,9-dimethoxybenzo[c][2,7]naphthyridin-2-yl)pyridin-3-yl]oxy}pyridin-3-yl)carbamate)

Step A: A mixture of 2-chloro-5-nitro-pyridine (3.57 g, 22.4 mmol), potassium carbonate (9.29 g, 67.2 mmol), and methyl 5-hydroxynicotinate (3.44 g, 22.4 mmol) in acetone (100 mL) is refluxed for 8 hours. The reaction is cooled to room temperature, diluted with additional acetone, and filtered. The white solid is washed with acetone. The filtrate is concentrated and chromatographed, first through magnesium silicate and then through silica, both of which are eluted with chloroform to provide 5.89 g of a tan solid (methyl 5-(5-nitropyridin-2-yloxy)nicotinate, 96%).

Step B: Methyl 5-(5-nitropyridin-2-yloxy)nicotinate (5.11 g, 18.58 mmol) is dissolved in methanol (300 mL) and acetic acid (6.38 mL, 111.5 mmol) followed by treatment with iron powder (6.38 g) and refluxed for 3 hours. The reaction is treated with ammonium hydroxide (13 mL) while hot and filtered through a pad of diatomaceous earth. The resulting filtrate is concentrated, slurried in chloroform and filtered through a pad of magnesium silicate to provide 4.74 g (>100%) of a tan solid, which is dried further to provide a more accurate weight of 4.39 g (96%) of methyl 5-(5-aminopyridin-2-yloxy)nicotinate.

Step C: A suspension of methyl 5-(5-aminopyridin-2-yloxy)nicotinate (2.30 g, 9.38 mmol), (Boc)₂O (6.13 g, 28.14 mmol) and catalytic dimethylaminopyridine (171.0 mg, 1.41 mmol) in THF (200 mL) is stirred at room temperature overnight. The reaction is concentrated and chromatographed on silica gel (hexane: ethylacetate) to provide 3.98 g (95%, methyl 5-(5-(bis(tert-butoxycarbonyl)amino)pyridin-2-yloxy)nicotinate) of a yellow oil.

Step D: A suspension of 6,7-dimethoxy-4-methylquinoline (2.23 g, 9.72 mmol), and methyl 5-(5-(bis(tert-butoxycarbonyl)amino)pyridin-2-yloxy)nicotinate (3.61 g, 8.10 mmol) is cooled to −78° C. and is then treated with a solution of LiHMDS (1.0 M in THF, 48.6 mL, 48.6 mmol) over a 10 minute period. The reaction is stirred at −78° C. for 1 hour, then for an additional 2 hours at 0° C. The reaction is then quenched with acetic acid (100 mL) at 0° C. and stirred for 30 minutes. The reaction is then treated with ammonium acetate (37.2 g, 486 mmol) and stirred at room temperature overnight, followed by briefly heating the reaction to 50° C. for 1 hour. The reaction is concentrated and the resulting residue is dissolved in chloroform (300 mL) and washed with ammonium hydroxide. The chloroform solution is concentrated and chromatographed on silica gel (eluted with: CHCl₃ to 20% MeOH/CHCl₃). The resultant residue is triturated with methanol to provide 1.36 g (tert-butyl 6-(5-(4-amino-8,9-dimethoxybenzo[c][2,7]naphthyridin-2-yl)pyridin-3-yloxy)pyridin-3-ylcarbamate (26%) of a tan solid. MS: 541.3 (M+H)⁺¹.

### Example 128

### Synthesis of Compound Ib-173

### (2-(5-(5-aminopyridin-2-yloxy)pyridin-3-yl)-8,9-dimethoxybenzo[c][2,7]naphthyridin-4-amine)

Tert-butyl 6-(5-(4-amino-8,9-dimethoxybenzo[c][2,7]naphthyridin-2-yl)pyridin-3-yloxy)pyridin-3-ylcarbamate (208.7 mg, 0.39 mmol) is dissolved in concentrated HCl (3 mL), and stirred for 15 minutes. The reaction is diluted with ethylacetate (100 mL) and stirred at room temperature overnight. The resulting solid is collected by vacuum filtration, washed with ethylacetate and then ether to provide 190.1 mg (2-(5-(5-aminopyridin-2-yloxy)pyridin-3-yl)-8,9-dimethoxybenzo[c][2,7]naphthyridin-4-amine as a bright yellow solid. MS: 241.7 (M+H)⁺¹.

### Example 128

### Synthesis of Compound Ib-174

### (2-{5-[(4-Aminopyridin-2-yl)oxy]pyridin-3-yl}-8,9-dimethoxybenzo[c][2,7]naphthyridin-4-amine)

By using the method described in Example 121 and starting with 2-bromo-4-nitropyridine 1-oxide, 2-{5-[(4-Aminopyridin-2-yl)oxy]pyridin-3-yl}-8,9-dimethoxybenzo[c][2,7]naphthyridin-4-amine can be prepared. MS (M+H) (m/e) 441.2.

### Example 129

### Synthesis of Compound Ib-175

### 2-{5-[(5-Aminopyridin-3-yl)oxy]pyridin-3-yl}-8,9-dimethoxybenzo[c][2,7]naphthyridin-4-amine

Step A: A solution of 6 g (23.7 mmol) of 3,5-dibromopyridine 1-oxide, 5.45 g (35.6 mmol) of methyl 5-hydroxynicotinate, 1 drop of Aliquat-336, and 15.4 g (47.5 mmol) of cesium carbonate in 65 ml DMF is heated at 100° C. for 13 hours. The mixture is cooled and filtered. The solids are washed with toluene and the solvent is removed from the combined filtrate. The residue is chromatographed to provide 3.0 g of 3-bromo-5-(5-(methoxycarbonyl)pyridin-3-yloxy)pyridine 1-oxide.

Step B: A mixture 4.45 g (13.7 ml) of 3-bromo-5-(5-(methoxycarbonyl)pyridin-3-yloxy)pyridine 1-oxide, 1.64 g (15.74 mmol) of t-butylcarbamate, 6.7 g (20.55 mmol) of cesium carbonate, 125 mg (0.14 mmol) of Pd₂(dba)₃, and 237 mg (0.41 mmol) of 4,5-bis(diphenylphosphino)-9,9-dimethylxanthene in 18 ml of dioxane is stirred at 100° C. for 13 hours. The mixture is diluted with chloroform, filtered, concentrated, and placed on a column of silica gel, which is eluted with chloroform-methanol mixtures to provide 0.78 g of 3-(tert-butoxycarbonylamino)-5-(5-(methoxycarbonyl)pyridin-3-yloxy)pyridine 1-oxide.

Step C: A mixture of 0.78 g (2.16 mmol) of 3-(tert-butoxycarbonylamino)-5-(5-(methoxycarbonyl)pyridin-3-yloxy)pyridine 1-oxide, 4.1 g (64.7 mmol) of ammonium formate, and 0.7 g of 10% Pd/C in 40 ml of methanol is heated with stirring at reflux for 4.5 hours. The mixture is filtered, diluted with chloroform, and washed with brine. The solvent is removed and the residue is purified by chromatography to provide 0.34 g of methyl 5-(5-(tert-butoxycarbonylamino)pyridin-3-yloxy)nicotinate.

Step D: To a solution of 0.23 g (1.01 mmol) of 6,7-dimethoxy-4-methylquinoline-3-carbonitrile and 0.345 g (1.01 mmol) of methyl 5-(5-(tert-butoxycarbonylamino)pyridin-3-yloxy)nicotinate in 10 ml of tetrahydrofuran at −78° C. with stirring, 4.53 ml (4.53 mmol) of 1 M lithium bis(trimethylsilyl)amide in tetrahydrofuran is added. The mixture is placed in an ice bath and stirred for 0.5 hours at 0° C. 7 ml of acetic acid and 1.55 g of ammonium acetate is added to the mixture. The mixture is heated at 75° C. for 0.5 hours. The solvent is removed and the residue is dissolved in chloroform. This solution is washed with NH₄OH and dried over magnesium sulfate. The mixture is filtered and concentrated. The residue is purified by chromatography using chloroform-methanol mixtures. The solvent is removed and the residue is mixed with 16 ml of ethyl acetate and 0.5 ml of concentrated hydrochloric acid. The mixture is stirred at 50° C. for 1 hour and cooled to room temperature. The solid product is collected by filtration to provide 0.226 g of 2-{5-[(5-Aminopyridin-3-yl)oxy]pyridin-3-yl}-8,9-dimethoxybenzo[c][2,7]naphthyridin-4-amine as the hydrochloride salt. MS (M+H) (m/e) 441.2.

### Example 130

### Synthesis of Compounds Ib-176, Ib-186, Ib-196, Ib-199, Ib-204, Ib-207, Ib-202, and Ib-203

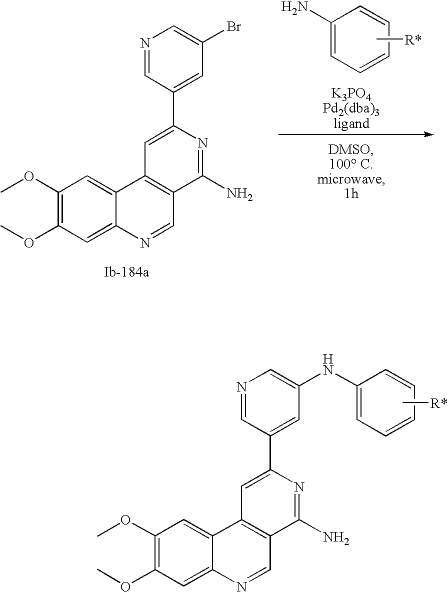

### (8,9-dimethoxy-2-{5-[(3-methoxyphenyl)amino]pyridin-3-yl}benzo[c][2,7]naphthyridin-4-amine)

A mixture of 2-(5-bromopyridin-3-yl)-8,9-dimethoxybenzo[c]-2,7-naphthyridin-4-amine (100 mg, 0.243 mmol, prepared as in Example 154, below), m-anisidine (60 mg, 0.486 mmol), powdered K₃PO₄ (77 mg, 0.360 mmol), tris(dibenzylideneacetone)dipalladium (0) (Pd₂(dba)₃, 21.9 mg, 0.024 mmol), and 2-dicyclohexylphosphino-2′-(N,N-dimethylamino)biphenyl (28.6 mg, 0.073 mmol) is suspended in DMSO (2.5 ml) in a microwave vial. The vial is sealed and the reaction is allowed to take place at 100° C. for 1 hour. The reaction is then cooled to room temperature, filtered, and then purified by HPLC to provide 8,9-dimethoxy-2-{5-[(3-methoxyphenyl)amino]pyridin-3-yl}benzo[c][2,7]naphthyridin-4-amine (38.4 mg, 56%). MS: 454.2 (M+H)⁺¹

Using the method described above the Benzo[c][2,7]naphthyridine Derivatives listed below are prepared.

| | | MS (m/e) |
|---|---|---|
| Compound | Compound Name | (M + H)⁺¹ |
| Ib-176 | 8,9-dimethoxy-2-{5-[(3-methoxyphenyl)amino]pyridin-3- | 454.2 |
| | yl}benzo[c][2,7]naphthyridin-4-amine | |
| Ib-177 | 8,9-dimethoxy-2-{5-[(4-methoxyphenyl)amino]pyridin-3- | 454.2 |
| | yl}benzo[c][2,7]naphthyridin-4-amine | |
| Ib-178 | 2-{[5-(4-amino-8,9-dimethoxybenzo[c][2,7]naphthyridin-2-yl)pyridin- | 449.2 |
| | 3-yl]amino}benzonitrile | |
| Ib-179 | 3-{[5-(4-amino-8,9-dimethoxybenzo[c][2,7]naphthyridin-2-yl)pyridin- | 449.2 |
| | 3-yl]amino}benzonitrile | |
| Ib-180 | 4-{[5-(4-amino-8,9-dimethoxybenzo[c][2,7]naphthyridin-2-yl)pyridin- | 449.2 |
| | 3-yl]amino}benzonitrile | |
| Ib-181 | 8,9-dimethoxy-2-{5-[(2-methoxyphenyl)amino]pyridin-3- | 454.2 |
| | yl}benzo[c][2,7]naphthyridin-4-amine | |
| Ib-182 | N-[5-(4-amino-8,9-dimethoxybenzo[c][2,7]naphthyridin-2-yl)pyridin- | 439.2 |
| | 3-yl]benzene-1,2-diamine | |
| Ib-183 | N-[5-(4-amino-8,9-dimethoxybenzo[c][2,7]naphthyridin-2-yl)pyridin- | 515.2 |
| | 3-yl]-N′-phenylbenzene-1,2-diamine | |
| Ib-184b | 8,9-dimethoxy-2-{5-[(2-morpholin-4-ylphenyl)amino]pyridin-3- | 509.2 |
| | yl}benzo[c][2,7]naphthyridin-4-amine | |
| Ib-185 | N-(2-{[5-(4-amino-8,9-dimethoxybenzo[c][2,7]naphthyridin-2- | 481.2 |
| | yl)pyridin-3-yl]amino}phenyl)acetamide | |
| Ib-186 | 2-{[5-(4-amino-8,9-dimethoxybenzo[c][2,7]naphthyridin-2-yl)pyridin- | 467.2 |
| | 3-yl]amino}benzamide | |
| Ib-187 | 2-{[5-(4-amino-8,9-dimethoxybenzo[c][2,7]naphthyridin-2-yl)pyridin- | 481.2 |
| | 3-yl]amino}-N-methylbenzamide | |
| Ib-188 | 3-{[5-(4-amino-8,9-dimethoxybenzo[c][2,7]naphthyridin-2-yl)pyridin- | 467.3 |
| | 3-yl]amino}benzamide | |
| Ib-189 | 2-(5-{[3-(aminomethyl)phenyl]amino}pyridin-3-yl)-8,9- | 453.2 |
| | dimethoxybenzo[c][2,7]naphthyridin-4-amine | |
| Ib-190 | 4-{[5-(4-amino-8,9-dimethoxybenzo[c][2,7]naphthyridin-2-yl)pyridin- | 539.1 |
| | 3-yl]amino}-2,3,5,6-tetrafluorobenzamide | |
| Ib-191 | 4-{[5-(4-amino-8,9-dimethoxybenzo[c][2,7]naphthyridin-2-yl)pyridin- | 566.3 |
| | 3-yl]amino}-N-[2-(diethylamino)ethyl]benzamide | |
| Ib-192 | 2-(5-{[4-(aminomethyl)phenyl]amino}pyridin-3-yl)-8,9- | 453.2 |
| | dimethoxybenzo[c][2,7]naphthyridin-4-amine | |
| Ib-193 | 4-{[5-(4-amino-8,9-dimethoxybenzo[c][2,7]naphthyridin-2-yl)pyridin- | 543.2 |
| | 3-yl]amino}-N-phenylbenzamide | |
| Ib-194 | (4-{[5-(4-amino-8,9-dimethoxybenzo[c][2,7]naphthyridin-2- | 454.2 |
| | yl)pyridin-3-yl]amino}phenyl)methanol | |
| Ib-195 | (3-{[5-(4-amino-8,9-dimethoxybenzo[c][2,7]naphthyridin-2- | 454.2 |
| | yl)pyridin-3-yl]amino}phenyl)methanol | |
| Ib-196 | (2-{[5-(4-amino-8,9-dimethoxybenzo[c][2,7]naphthyridin-2- | 454.2 |
| | yl)pyridin-3-yl]amino}phenyl)methanol | |
| Ib-197 | 2-(2-{[5-(4-amino-8,9-dimethoxybenzo[c][2,7]naphthyridin-2- | 468.2 |
| | yl)pyridin-3-yl]amino}phenyl)ethanol | |
| Ib-198 | 2-(4-{[5-(4-amino-8,9-dimethoxybenzo[c][2,7]naphthyridin-2- | 468.2 |
| | yl)pyridin-3-yl]amino}phenyl)ethanol | |
| Ib-199 | 8,9-dimethoxy-2-[5-(pyridin-2-ylamino)pyridin-3- | 425.2 |
| | yl]benzo[c][2,7]naphthyridin-4-Amine | |
| Ib-200 | 8,9-dimethoxy-2-[5-(pyridin-3-ylamino)pyridin-3- | 425.2 |
| | yl]benzo[c][2,7]naphthyridin-4-Amine | |
| Ib-201 | 8,9-dimethoxy-2-[5-(pyridin-4-ylamino)pyridin-3- | 425.2 |
| | yl]benzo[c][2,7]naphthyridin-4-Amine | |
| Ib-202 | 2-(5-{[2-(benzyloxy)phenyl]amino}pyridin-3-yl)-8,9- | 530.3 |
| | dimethoxybenzo[c][2,7]naphthyridin-4-amine | |
| Ib-203 | 2-(5-anilinopyridin-3-yl)-8,9-dimethoxybenzo[c][2,7]naphthyridin-4- | 424.3 |
| | amine | |
| Ib-204 | 2-[5-(biphenyl-2-ylamino)pyridin-3-yl]-8,9- | 500.2 |
| | dimethoxybenzo[c][2,7]naphthyridin-4-Amine | |
| Ib-205 | 2-{5-[(2-fluorophenyl)amino]pyridin-3-yl}-8,9- | 442.2 |
| | dimethoxybenzo[c][2,7]naphthyridin-4-amine | |
| Ib-206 | 4-{[5-(4-amino-8,9-dimethoxybenzo[c][2,7]naphthyridin-2-yl)pyridin- | 467.2 |
| | 3-yl]amino}benzamide | |
| Ib-207 | 8,9-dimethoxy-2-(5-{[2-(2-morpholin-4- | 553.2 |
| | ylethoxy)phenyl]amino}pyridin-3-yl)benzo[c][2,7]naphthyridin-4- | |
| | amine | |
| Ib-208 | 8,9-dimethoxy-2-(5-{[2-(2-methoxyethoxy)phenyl]amino}pyridin-3- | 498.2 |
| | yl)benzo[c][2,7]naphthyridin-4-amine | |

### Example 131

### Synthesis of Compound Ib-209

### (2-(5-{[2-(aminomethyl)phenyl]amino}pyridin-3-yl)-8,9-dimethoxybenzo[c][2,7]naphthyridin-4-amine)

A mixture of 2-(5-bromopyridin-3-yl)-8,9-dimethoxybenzo[c]-2,7-naphthyridin-4-amine (300 mg, 0.729 mmol), (2-amino-benzyl)-carbamic acid tert-butyl ester (324 mg, 1.458 mmol), powdered K₃PO₄ (231 mg, 1.09 mmol), tris(dibenzylideneacetone)dipalladium (0) (Pd₂(dba)₃, 66 mg, 0.072 mmol), and 2-dicyclohexylphosphino-2′-(N,N-dimethylamino)biphenyl (87 mg, 0.219 mmol) is suspended in DMSO (5 ml) in a microwave vial. The vial is sealed and the reaction is allowed to take place at 100° C. for 1 hour. The reaction is cooled to room temperature, filtered, and then purified by HPLC to provide a yellow solid. The solid is suspended in ethyl acetate (4 mL) and then concentrated hydrochloric acid (1 mL) is added. The reaction mixture is stirred at room temperature overnight. The resulted yellow solid is filtered, washed with ethyl acetate and dried to provide 2-(5-{[2-(aminomethyl)phenyl]amino}pyridin-3-yl)-8,9-dimethoxybenzo[c][2,7]naphthyridin-4-amine (25 mg, 10%). MS: 453.2 (M+H)⁺¹.

### Example 132

### Synthesis of Compound Ib-210

### (8,9-dimethoxy-2-[5-(4-methoxyphenoxy)pyridin-3-yl]benzo[c][2,7]naphthyridin-4-amine)

A mixture of 2-(5-bromopyridin-3-yl)-8,9-dimethoxybenzo[c]-2,7-naphthyridin-4-amine (100 mg, 0.243 mmol), 4-methoxyphenol (36 mg, 0.291 mmol), sodium t-butoxide (46 mg, 0.486 mmol), tris(dibenzylideneacetone)dipalladium (0) (Pd₂(dba)₃, 22 mg, 0.024 mmol), and 2-di-t-butylphosphino-2′,4′,6′-triisopropyl-1,1′-biphenyl (30 mg, 0.072 mmol) is suspended in DMSO (2.5 ml) in a microwave vial. The vial is sealed and the reaction is allowed to take place at 175° C. for 1 hour. The reaction is then cooled to room temperature, filtered, and purified by HPLC to provide a yellow solid compound, 8,9-dimethoxy-2-[5-(4-methoxyphenoxy)pyridin-3-yl]benzo[c][2,7]naphthyridin-4-amine (6 mg, 5.6%). MS: 455.3 (M+H)⁺¹.

### Example 133

### Synthesis of Compound Ib-211

### (3-(4-Amino-8,9-dimethoxybenzo[c][2,7]naphthyridin-2-yl)benzaldehyde)

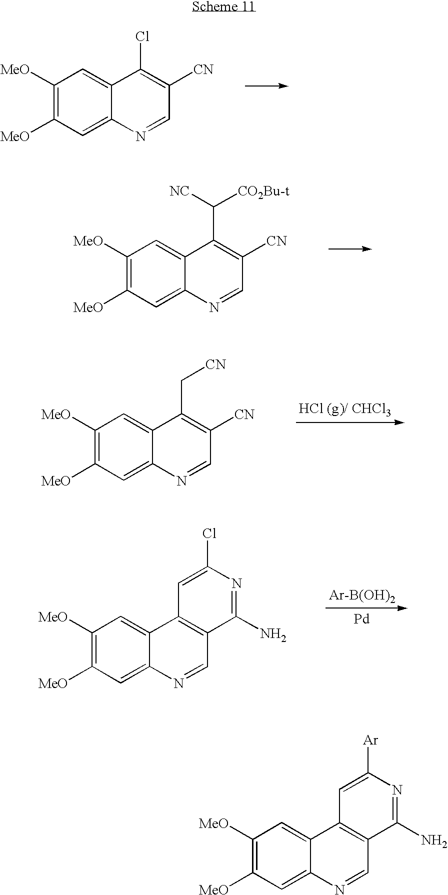

Step A: To NaH (60%, 2.54 g, 63.4 mmol) in DMF (60 mL), tert-butyl 2-cyanoacetate (4.07 g, 28.8 mmol) in DMF (20 mL) at 0° C. is added dropwise over 30 minutes. After stirring at room temperature for 15 minutes, 4-chloro-6,7-dimethoxy-3-quinolinecarbonitrile (5.0 g, 19.2 mmol) is added. The reaction mixture is heated at 80-85° C. for 2 hours, cooled to room temperature, poured into 500 mL of ice water, and acidified with HOAc (4.03 g in 50 mL of water). The solid is collected by filtration, washed with water and dried to provide 6.85 g of tert-butyl 2-cyano-2-(3-cyano-6,7-dimethoxyquinolin-4-yl)acetate as an orange solid, which can be used for the next step without further purification.

Step B: A mixture of tert-butyl 2-cyano-2-(3-cyano-6,7-dimethoxyquinolin-4-yl)acetate (6.6 g, 18.1 mmol) in 1,2-dichlorobenzene (60 mL) is heated at 175° C. for 40 minutes and cooled to room temperature to yield a thick suspension. The solids are collected by filtration and washed thoroughly with hexane to provide 4.1 g of 4-(cyanomethyl)-6,7-dimethoxyquinoline-3-carbonitrile as a yellow solid, which can be used directly for the next step.

Step C: To a stirred mixture of 4-(cyanomethyl)-6,7-dimethoxyquinoline-3-carbonitrile (100 mg, 0.39 mmol) and chloroform (5 mL), gaseous HCl is bubbled in for 5-10 minutes. The reaction mixture is stirred at room temperature for 3 days while gaseous HCl is bubbled in for 5-10 minutes every 10-12 hours. The resulting mixture is diluted with hexane, filtered and washed with hexane to provide a crude solid, which is purified by flash column chromatograph to provide 42 mg of 2-chloro-8,9-dimethoxybenzo[c]-2,7-naphthyridin-4-amine as a light yellow solid. MS 290.2 (M+H)⁺¹.

Step D: A mixture of 2-chloro-8,9-dimethoxybenzo[c]-2,7-naphthyridin-4-amine (100 mg, 0.306 mmol), 3-formylphenylboronic acid (70 mg, 0.450 mmol), and Pd(PPh₃)₄ (18 mg, 0.0153 mmol) in DME (2.0 mL) and saturated aqueous sodium bicarbonate (1.0 mL) is heated at 90° C. for 3 hours. After cooling to room temperature, the reaction mixture is partitioned between water and dichloromethane. The combined organic layers are dried over sodium sulfate, concentrated and purified by HPLC (eluted with a gradient of acetonitrile/water containing TFA) to provide the title compound as an off-white solid. MS: 360.1 (M+H)⁺¹.

Using methods and reagents outlined above, the following illustrative Benzo[c][2,7]naphthyridine Derivatives listed below are prepared.

| Compound | Compound Name | MS m/e [M + 1]⁺¹ |
|---|---|---|
| Ib-212 | 2-(4-amino-8,9-dimethoxybenzo[c][2,7]naphthyridin-2- | 376.1 |
| | yl)benzoic acid | |
| Ib-213 | 4-(4-amino-8,9-dimethoxybenzo[c][2,7]naphthyridin-2- | 360.1 |
| | yl)benzaldehyde | |
| Ib-214 | 2-{6-[3-(dimethylamino)propoxy]pyridin-3-yl}-8,9- | 434.2 |
| | dimethoxybenzo[c][2,7]naphthyridin-4-amine | |
| Ib-215 | 8,9-dimethoxy-2-(6-morpholin-4-ylpyridin-3- | 418.4 |
| | yl)benzo[c][2,7]naphthyridin-4-amine | |
| Ib-216 | 8,9-dimethoxy-2-(2-morpholin-4-ylpyrimidin-5- | 419.4 |
| | yl)benzo[c][2,7]naphthyridin-4-amine | |
| Ib-217 | 5-(4-amino-8,9-dimethoxybenzo[c][2,7]naphthyridin-2- | 366.1 |
| | yl)thiophene-2-carbaldehyde | |
| Ib-218 | 8,9-dimethoxy-2-{6-[(2-morpholin-4-ylethyl)amino]pyridin- | 461.2 |
| | 3-yl}benzo[c][2,7]naphthyridin-4-amine | |
| Ib-219 | 2-(3-ethoxyphenyl)-8,9- | 376.2 |
| | dimethoxybenzo[c][2,7]naphthyridin-4-amine | |
| Ib-220 | 8,9-dimethoxy-2-(3- | 390.2 |
| | propoxyphenyl)benzo[c][2,7]naphthyridin-4-amine | |
| Ib-221 | 8,9-dimethoxy-2-(3-thienyl)benzo[c][2,7]naphthyridin-4- | 338.1 |
| | amine | |
| Ib-222 | 2-(3-furyl)-8,9-dimethoxybenzo[c][2,7]naphthyridin-4- | 322.3 |
| | amine | |
| Ib-223 | 2-(6-fluoropyridin-3-yl)-8,9- | 351.1 |
| | dimethoxybenzo[c][2,7]naphthyridin-4-amine | |
| Ib-224 | 2-(6-aminopyridin-3-yl)-8,9- | 348.3 |
| | dimethoxybenzo[c][2,7]naphthyridin-4-amine | |
| Ib-226 | 2-isoquinolin-4-yl-8,9-dimethoxybenzo[c][2,7]naphthyridin- | 383.1 |
| | 4-amine | |
| Ib-227 | 5-(4-amino-8,9-dimethoxybenzo[c][2,7]naphthyridin-2- | 388.1 |
| | yl)isophthalaldehyde | |
| Ib-228 | 8,9-dimethoxy-2-(6-piperazin-1-ylpyridin-3- | 417.3 |
| | yl)benzo[c][2,7]naphthyridin-4-amine | |

### Example 134

### Synthesis of Compound Ia-63

### (2-(5-{[(2R)-2-aminobutyl]oxy}pyridin-3-yl)-8-(2-chloroethoxy)-9-methoxybenzo[c][2,7]naphthyridin-4-amine)

Step A: A stirred solution of 7-(benzyloxy)-6-methoxy-4-methylquinoline-3-carbonitrile (4.08, 13.4 mmol) and thioanisole (7.87 ml, 67 mmol) in TFA (54 ml) is refluxed for 1.5 hours. The solution is concentrated in vacuo and the residue stirred in 1:1 DCM-hexane and aqueous NaHCO₃. The resulting solid is filtered off and recrystallized from MeOH to provide 7-hydroxy-6-methoxy-4-methylquinoline-3-carbonitrile as a tan solid (1.30 g, 45%).

Step B: To a stirred solution of 7-hydroxy-6-methoxy-4-methylquinoline-3-carbonitrile (1.08 g, 5.06 mmol), methyl 5-({(2R)-2-[(tert-butoxycarbonyl)amino]butyl}oxy)nicotinate (1.97 g, 6.07 mmol) in 25.3 ml of THF at −78° C., 1.0 M LiHMDS in THF (30.4 mmol) is added over 3 minutes. The mixture is warmed to 0° C. and stirred for 30 minutes. The mixture is treated successively with 50 ml of HOAc and 25 g of NH₄OAc, stirred at 90° C. for 30 minutes, and concentrated to dryness. The residue is stirred in aqueous KHCO₃, and the resulting solid is filtered, washed with water, and dried. Flash chromatography of the residue on silica gel with 10:1 DCM-MeOH provides tert-butyl [(1R)-1-({[5-(4-amino-8-hydroxy-9-methoxybenzo[c][2,7]naphthyridin-2-yl)pyridin-3-yl]oxy}methyl)propyl]carbamate as an orange solid (2.15 g, 84%).

Step C: To a stirred mixture of tert-butyl [(1R)-1-({[5-(4-amino-8-hydroxy-9-methoxybenzo[c][2,7]naphthyridin-2-yl)pyridin-3-yl]oxy}methyl)propyl]carbamate (126 mg, 0.25 mmol), 2-chloroethyl tosylate (82 mg, 0.35 mmol), and 0.50 ml of DMF at 25° C. Cs₂CO_{3 (}98 mg, 0.30 mmol) is added. After 20 hours, the DMF is evaporated in vacuo, and the residue is stirred in water. The resulting tan solid is filtered, washed with water, and dried. To a suspension of the solid in 2.5 ml of EtOAc at 25° C., 0.25 ml of concentrated HCl is added. After 30 minutes, the mixture is partitioned with DCM and aqueous NH₄OH. The organic layer is washed with water, dried and concentrated. Flash chromatography of the residue on silica gel with DCM-EtOAc-MeOH-TEA provides 2-(5-{[(2R)-2-aminobutyl]oxy}pyridin-3-yl)-8-(2-chloroethoxy)-9-methoxybenzo[c][2,7]naphthyridin-4-amine as an amber solid (68 mg, 58%). MS: 468.3, 470.3 (M+H)⁺¹.

### Example 135

### Synthesis of Compounds Ia-64 to Ia-68

Using the procedure described in Example 134, Step C, treatment of tert-butyl [(1R)-1-({[5-(4-amino-8-hydroxy-9-methoxybenzo[c][2,7]naphthyridin-2-yl)pyridin-3-yl]oxy}methyl)propyl]carbamate with Cs₂CO₃ and the appropriate alkylating agent, followed by treatment with HCl in EtOAc and water, provides the compounds in the Table below.

| Compound | Compound Name | MS: m/e (M + H)⁺¹ |
|---|---|---|
| Ia-64 | 2-(5-{[(2R)-2-aminobutyl]oxy}pyridin-3-yl)-8-ethoxy- | 434.3 |
| | 9-methoxybenzo[c][2,7]naphthyridin-4-amine | |
| Ia-65 | 2-(5-{[(2R)-2-aminobutyl]oxy}pyridin-3-yl)-8-(2- | 452.3 |
| | fluoroethoxy)-9-methoxybenzo[c][2,7]naphthyridin- | |
| | 4-amine | |
| Ia-66 | 2-(5-{[(2R)-2-aminobutyl]oxy}pyridin-3-yl)-9- | 516.3 |
| | methoxy-8-(4,4,4- | |
| | trifluorobutoxy)benzo[c][2,7]naphthyridin-4-amine | |
| Ia-67 | 2-(5-{[(2R)-2-aminobutyl]oxy}pyridin-3-yl)-8-(3- | 482.3, 484.3 |
| | chloropropoxy)-9-methoxybenzo[c][2,7]naphthyridin- | |
| | 4-amine | |
| Ia-68 | {[4-amino-2-(5-{[(2R)-2-aminobutyl]oxy}pyridin-3- | 464.4 |
| | yl)-9-methoxybenzo[c][2,7]naphthyridin-8- | |
| | yl]oxy}acetic acid | |

### Example 136

### Synthesis of Compounds Ia-69a, Ia-69b and Ia-70

### (Tert-butyl [(1R)-1-({[5-(4-amino-9-hydroxy-8-methoxybenzo[c][2,7]naphthyridin-2-yl)pyridin-3-yl]oxy}methyl)propyl]carbamate)

In the manner described in Example 134, treatment of 6-hydroxy-7-methoxy-4-methylquinoline-3-carbonitrile with methyl 5-({(2R)-2-[(tert-butoxycarbonyl)amino]butyl}oxy)nicotinate and LiHMDS in THF, followed by NH₄OAc in HOAc provides the title compound as an amber solid. MS: 506.4 (M+H)⁺¹.

Using the procedure described in Example 134, treatment of tert-butyl [(1R)-1-({[5-(4-amino-8-hydroxy-9-methoxybenzo[c][2,7]naphthyridin-2-yl)pyridin-3-yl]oxy}methyl)propyl]carbamate with Cs₂CO₃ and the appropriate alkylating agent, followed by treatment with HCl in EtOAc and water, provides the compounds of the Table below.

| | | MS: m/e |
|---|---|---|
| Compound | Compound Name | (M + H)⁺¹ |
| Ia-69b | 2-(5-{[(2R)-2-aminobutyl]oxy}pyridin-3- | 468.3, 470.2 |
| | yl)-9-(2-chloroethoxy)-8-methoxybenzo[c][2, | |
| | 7]naphthyridin-4-amine | |
| Ia-70 | 2-(5-{[(2R)-2-aminobutyl]oxy}pyridin-3- | 452.3 |
| | yl)-9-(2-fluoroethoxy)-8-methoxybenzo[c][2, | |
| | 7]naphthyridin-4-amine | |

### Example 137

### Synthesis of Compound Ia-71a

### (Tert-butyl [(1R)-1-({[5-(4-amino-8,9-dihydroxybenzo[c][2,7]naphthyridin-2-yl)pyridin-3-yl]oxy}methyl)propyl]carbamate)

Step A: A stirred mixture of 6,7-dimethoxy-4-methylquinoline-3-carbonitrile (0.68 g, 3.0 mmol) and 7.5 g of pyridine hydrochloride is heated at 205° C. for 30 minutes and cooled to 25° C. The mixture is stirred with 7.5 ml of concentrated NH₄OH for 30 minutes and concentrated to dryness. The residue is stirred in water, and the resulting solid is filtered, washed with water and dried to provide 6,7-dihydroxy-4-methylquinoline-3-carbonitrile (0.59 g, 98%).

Step B: In the manner described in Example 134, treatment of 6,7-dihydroxy-4-methylquinoline-3-carbonitrile with methyl 5-({(2R)-2-[(tert-butoxycarbonyl)amino]butyl}oxy)nicotinate and LiHMDS in THF, followed by NH₄OAc in HOAc provides the title compound as an amber solid. MS: 492.4 (M+H)⁺¹.

### Example 138

### Synthesis of Compound Ia-71b

### (Tert-butyl [(1R)-1-({[5-(4-amino-9-bromobenzo[c][2,7]naphthyridin-2-yl)pyridin-3-yl]oxy}methyl)propyl]carbamate)

Step A: A mixture of 2-amino-5-bromoacetophenone (17.1 g, 80 mmol), dimethylformamide dimethyl acetal (14.1 ml, 100 mmol), and 40 ml of t-butanol is stirred at 100° C. for 4 hours, cooled to 25° C., and treated with t-butyl cyanoacetate (14.7 g, 104 mmol). After 1.5 hours the mixture is evaporated to dryness, and the residue is filtered through a short column of silica gel as a solution in 10:1 DCM-EtOAc. Evaporation of solvents provides 2-cyano-3-(2′-acetyl-4′-bromophenyl)amino-2-propenoic acid t-butyl ester as an off-white solid (20.1 g, 70%).

Step B: A stirred solution of 2-cyano-3-(2′-acetyl-4′-bromophenyl)amino-2-propenoic acid t-butyl ester (19.9 g, 54.5 mmol) and diisopropylethylamine (0.47 ml, 2.7 mmol) in 272 ml of 1,2-dichlorobenzene is refluxed for 24 hours. The solution is cooled to 10° C. and treated during 1 minute with 1.0 M KOtBu in t-butanol (327 mmol). After 1 hour at 25° C., the mixture is quenched with CO₂ and partitioned with DCM and water. The organic layer is washed with water, dried, and concentrated. Flash chromatography of the residue on silica gel with DCM-EtOAc provides 6-bromo-4-methylquinoline-3-carbonitrile as an off-white solid (12.5 g, 93%).

Step C: In the manner described in Example 134, treatment of 6-bromo-4-methylquinoline-3-carbonitrile with methyl 5-({(2R)-2-[(tert-butoxycarbonyl)amino]butyl}oxy)nicotinate and LiHMDS in THF, followed by NH₄OAc in HOAc, provides the title compound as a light amber solid. MS: 538.3, 540.3 (M+H)⁺¹.

### Example 139

### Synthesis of Compound Ia-73

### (4-amino-2-(5-{[(2R)-2-aminobutyl]oxy}pyridin-3-yl)-8-methoxybenzo[c][2,7]naphthyridin-9-ol)

To a stirred mixture of tert-butyl [(1R)-1-({[5-(4-amino-9-hydroxy-8-methoxybenzo[c][2,7]naphthyridin-2-yl)pyridin-3-yl]oxy}methyl)propyl]carbamate (106 mg, 0.21 mmol) and 2.1 ml of EtOAc at 25° C., 0.21 ml of concentrated HCl is added. After 1.5 hours the mixture is diluted with Et₂O and filtered. The yellow solid is washed with Et₂O and dried in vacuo to provide 4-amino-2-(5-{[(2R)-2-aminobutyl]oxy}pyridin-3-yl)-8-methoxybenzo[c][2,7]naphthyridin-9-ol, trihydrochloride salt (112 mg, 100%). MS: 406.3 (M+H)⁺¹.

### Example 140

### Synthesis of Compound Ia-74

### (2-(5-{[(2R)-2-aminobutyl]oxy}pyridin-3-yl)-8-methoxybenzo[c][2,7]naphthyridin-4-amine)

Step A: In the manner of Example 134, treatment of 7-methoxy-4-methylquinoline-3-carbonitrile with methyl 5-({(2R)-2-[(tert-butoxycarbonyl)amino]butyl}oxy)nicotinate and LiHMDS in THF, followed by NH₄OAc in HOAc, provides tert-butyl [(1R)-1-({[5-(4-amino-8-methoxybenzo[c][2,7]naphthyridin-2-yl)pyridin-3-yl]oxy}methyl)propyl]carbamate as a tan solid (115 mg, 47%).

Step B: In the manner of Example 139, the above treatment of tert-butyl [(1R)-1-({[5-(4-amino-8-methoxybenzo[c][2,7]naphthyridin-2-yl)pyridin-3-yl]oxy}methyl)propyl]carbamate with HCl in EtOAc and water provides 2-(5-{[(2R)-2-aminobutyl]oxy}pyridin-3-yl)-8-methoxybenzo[c][2,7]naphthyridin-4-amine, trihydrochloride salt as a yellow solid. MS: 390.3 (M+H)⁺¹.

### Example 141

### Synthesis of Compound Ia-75

### (2-(5-{[(2R)-2-aminobutyl]oxy}pyridin-3-yl)-9-bromobenzo[c][2,7]naphthyridin-4-amine)

In the manner of Example 139, treatment of tert-butyl [(1R)-1-({[5-(4-amino-9-bromobenzo[c][2,7]naphthyridin-2-yl)pyridin-3-yl]oxy}methyl)propyl]carbamate with HCl in EtOAc and water provides 2-(5-{[(2R)-2-aminobutyl]oxy}pyridin-3-yl)-9-bromobenzo[c][2,7]naphthyridin-4-amine, trihydrochloride salt as a yellow solid. MS: 438.1, 440.1 (M+H)⁺¹.

### Example 142

### Synthesis of Compound Ib-229

### (2-(4-bromophenyl)-8,9-dimethoxybenzo[c][2,7]naphthyridin-4-amine)

In the manner described in Example 134, treatment of 6,7-dimethoxy-4-methylquinoline-3-carbonitrile with methyl 4-bromobenzoate and LiHMDS in THF, followed by NH₄OAc in HOAc provides the title compound as a tan solid. MS: 410.2, 412.2 (M+H)⁺¹.

### Example 143

### Synthesis Compound Ia-76

### (2-(5-{[(2R)-2-aminobutyl]oxy}pyridin-3-yl)-9-quinolin-3-ylbenzo[c][2,7]naphthyridin-4-amine)

Step A: A mixture of tert-butyl [(1R)-1-({[5-(4-amino-9-bromobenzo[c][2,7]naphthyridin-2-yl)pyridin-3-yl]oxy}methyl)propyl]carbamate (269 mg, 0.50 mmol), 3-quinolylboronic acid (130 mg, 0.75 mmol), potassium carbonate (221 mg, 1.6 mmol), tetrakis(triphenylphosphine)palladium(0) (29 mg, 0.025 mmol), DME (1.0 ml), and water (0.50 ml) is stirred at 95° C. for 1 hour. The reaction mixture is cooled, stirred in DCM and water, and filtered to provide tert-butyl [(1R)-1-({[5-(4-amino-9-quinolin-3-ylbenzo[c][2,7]naphthyridin-2-yl)pyridin-3-yl]oxy}methyl)propyl]carbamate 126 mg, 43%).

Step B: In the manner of Example 139, treatment of tert-butyl [(1R)-1-({[5-(4-amino-9-quinolin-3-ylbenzo[c][2,7]naphthyridin-2-yl)pyridin-3-yl]oxy}methyl)propyl]carbamate with HCl in EtOAc and water provides 2-(5-{[(2R)-2-aminobutyl]oxy}pyridin-3-yl)-9-quinolin-3-ylbenzo[c][2,7]naphthyridin-4-amine, trihydrochloride salt as a yellow solid. MS: 487.3 (M+H)⁺¹.

### Example 144

### Synthesis of Compound Ia-77

### (2-(5-{[(2R)-2-aminobutyl]oxy}pyridin-3-yl)-9-(phenylethynyl)benzo[c][2,7]naphthyridin-4-amine)

Step A: A mixture of tert-butyl [(1R)-1-({[5-(4-amino-9-bromobenzo[c][2,7]naphthyridin-2-yl)pyridin-3-yl]oxy}methyl)propyl]carbamate (269 mg, 0.50 mmol), phenylacetylene (102 mg, 1.0 mmol), potassium carbonate (0.34 g, 2.5 mmol), CuI (19 mg, 0.10 mmol), tripenylphosphine (26 mg, 0.10 mmol), dichlorobis(tripenylphosphine)palladium(II) (18 mg, 0.025 mmol), MeOH (0.20 ml), and NMP (1.0 ml) is stirred at 60° C. for 1.5 hours. The reaction mixture is cooled and partitioned with DCM and water. The organic layer is washed with water, dried, and concentrated. Flash chromatography of the residue on silica gel with DCM-EtOAc-MeOH-TEA provides tert-butyl [(1R)-1-({[5-(4-amino-9-(phenylethynyl)lbenzo[c][2,7]naphthyridin-2-yl)pyridin-3-yl]oxy}methyl)propyl]carbamate as an amber solid (257 mg, 92%).

Step B: In the manner of Example 139, treatment of tert-butyl [(1R)-1-({[5-(4-amino-9-(phenylethynyl)benzo[c][2,7]naphthyridin-2-yl)pyridin-3-yl]oxy}methyl)propyl]carbamate with HCl in EtOAc and water provides 2-(5-{[(2R)-2-aminobutyl]oxy}pyridin-3-yl)-9-(phenylethynyl)lbenzo[c][2,7]naphthyridin-4-amine, trihydrochloride salt as an orange solid. MS: 460.0 (M+H)⁺¹.

### Example 145

### Synthesis of Compound Ia-78

### (2-(5-{[(2R)-2-aminobutyl]oxy}pyridin-3-yl)-9-morpholin-4-ylbenzo[c][2,7]naphthyridin-4-amine)

Step A: A mixture of tert-butyl [(1R)-1-({[5-(4-amino-9-bromobenzo[c][2,7]naphthyridin-2-yl)pyridin-3-yl]oxy}methyl)propyl]carbamate (1.08 g, 2.0 mmol), dimethylformamide dimethyl acetal (0.56 ml, 4.0 mmol), and 4.0 ml of toluene is refluxed for 1 hour and subjected to evaporation at 50° C. in vacuo to provide tert-butyl [(1R)-1-({[5-(9-bromo-4-{[(1E)-(dimethylamino)methylene]amino}benzo[c][2,7]naphthyridin-2-yl)pyridin-3-yl]oxy}methyl)propyl]carbamate as a tan solid (1.14 g, 96%).

Step B: A mixture of tert-butyl [(1R)-1-({[5-(9-bromo-4-{[(1E)-(dimethylamino)methylene]amino}benzo[c][2,7]naphthyridin-2-yl)pyridin-3-yl]oxy}methyl)propyl]carbamate (297 mg, 0.50 mmol), morpholine (65 mg, 0.75 mmol), 4,5-bis(diphenylphosphino)-9,9-dimethylxanthene (36 mg, 0.06 mmol), NaOtBu (72 mg, 0.75 mmol), tris(dibenzylidineacetone)dipalladium(0) (18 mg, 0.02 mmol), and toluene (6.0 ml) is stirred at 95° C. for 1.5 hours. The mixture is cooled and partitioned with DCM and water. The organic layer is washed with water, dried, and concentrated. Flash chromatography of the residue on silica gel with DCM-EtOAc-MeOH provides tert-butyl [(1R)-1-({[5-9-(morph Olin-4-yl)-4-{[(1E)-(dimethylamino)methylene]amino}benzo[c][2,7]naphthyridin-2-yl)pyridin-3-yl]oxy}methyl)propyl]carbamate as a yellow solid (161 mg, 54%).

Step C: To a stirred solution of tert-butyl [(1R)-1-({[5-9-(morpholin-4-yl)-4-{[(1E)-(dimethylamino)methylene]amino}benzo[c][2,7]naphthyridin-2-yl)pyridin-3-yl]oxy}methyl)propyl]carbamate (161 mg, 0.27 mmol) in 5.4 ml of EtOAc, 0.54 ml of concentrated HCl is added. The resulting suspension is stirred for 30 minutes and treated with 9 ml of saturated NH₄Cl. The resulting mixture is stirred for 20 hours and partitioned with 20:1 DCM-MeOH and aqueous NaOH. The organic layer is washed with saturated NaCl, dried, and concentrated. Flash chromatography of the residue on silica gel with EtOAc-MeOH-TEA provides 2-(5-{[(2R)-2-aminobutyl]oxy}pyridin-3-yl)-9-morpholin-4-ylbenzo[c][2,7]naphthyridin-4-amine as a yellow solid (117 mg, 98%). MS: 445.2 (M+H)⁺¹.

### Example 146

### Synthesis of Compound Ia-79

### (9-bromobenzo[c][2,7]naphthyridin-4-amine)

Step A: A stirred solution of 6-bromo-4-methylquinoline-3-carbonitrile (4.21 g, 17 mmol), dimethylformamide dimethyl acetal (4.80 ml, 34 mmol), and 8.5 ml of DMF is refluxed for 3 hours and concentrated in vacuo to provide 6-bromo-4-[(E)-2-(dimethylamino)vinyl]quinoline-3-carbonitrile as a tan solid (4.7 g, 100%).

Step B: A stirred mixture of 6-bromo-4-[(E)-2-(dimethylamino)vinyl]quinoline-3-carbonitrile (60 mg, 0.20 mmol) and NH₄OAc (0.4 g, 5 mmol) in 2.0 ml of HOAc is refluxed for 1 hour and concentrated in vacuo. The residue is stirred in dilute NaOH, and the resultant solid is filtered, washed, with water and dried. Digestion of the solid in hot EtOH provides 9-bromobenzo[c][2,7]naphthyridin-4-amine as a tan solid (27 mg, 50%). MS: 274.0, 276.0 (M+H)⁺¹.

### Example 147

### Synthesis of Compound Ib-230

### (8,9-dimethoxybenzo[c][2,7]naphthyridin-4-amine)

Step A: A stirred solution of 6,7-dimethoxy-4-methylquinoline-3-carbonitrile (2.28 g, 10 mmol), dimethylformamide dimethyl acetal (2.80 ml, 20 mmol), and 5.0 ml of DMF is refluxed for 3 hours and concentrated in vacuo to provide 4-[(E)-2-(dimethylamino)vinyl]-6,7-dimethoxyquinoline-3-carbonitrile as a tan solid (2.8 g, 100%).

Step B: A stirred mixture of 4-[(E)-2-(dimethylamino)vinyl]-6,7-dimethoxyquinoline-3-carbonitrile (198 mg, 0.70 mmol) and NH₄OAc (1.1 g, 14 mmol) in 7.0 ml of HOAc is refluxed for 5 hour and concentrated in vacuo. The residue is stirred in dilute NaOH, and the resultant solid is filtered, washed with water and dried. Recrystallization from EtOH provides 8,9-dimethoxybenzo[c][2,7]naphthyridin-4-amine as a light yellow solid (58 mg, 32%). MS: 256.1 (M+H)⁺¹.

### Example 148

### Synthesis of Compound I-6

### (9-bromo-N-(2-methylphenyl)benzo[c][2,7]naphthyridin-4-amine)

Step A: A stirred solution of 6-bromo-4-[(E)-2-(dimethylamino)vinyl]quinoline-3-carbonitrile and 23 ml of concentrated H₂SO₄ in 46 ml of HOAc is refluxed for 30 minutes, cooled, and diluted with water. The mixture is brought to pH 8 by addition of K₂CO₃, and the resultant solid is filtered, washed with water, and dried to provide 9-bromobenzo[c][2,7]naphthyridin-4-ol as an off-white solid (4.61 g, 98%).

Step B: A stirred mixture of 9-bromobenzo[c][2,7]naphthyridin-4-ol (4.13 g, 15 mmol), 0.15 ml of DMF, and 75 ml of POCl₃ is refluxed for 4 hours and concentrated to dryness in vacuo. The residue is partitioned with DCM and aqueous NaHCO₃. The organic layer is washed with water, dried, and concentrated to provide 9-bromo-4-chlorobenzo[c][2,7]naphthyridine as a tan solid (4.18 g, 95%).

Step C: A stirred mixture of 9-bromo-4-chlorobenzo[c][2,7]naphthyridine (235 mg, 0.80 mmol), 2-methylaniline (172 mg, 1.6 mmol), pyridine hydrochloride 92 mg, 0.80 mmol), and 4.8 ml of 2-ethoxyethanol is refluxed for 8 hours, cooled, and partitioned with DCM and aqueous K₂CO₃. The organic layer is washed with water, dried and concentrated. The residue is crystallized from Et₂O-hexane to provide 9-bromo-N-(2-methylphenyl)benzo[c][2,7]naphthyridin-4-amine as a brown solid (208 mg, 72%). MS: 364.1, 366.1 (M+H)⁺¹.

### Example 149

### Synthesis of Compound I-7

### (9-bromo-N-(4-methoxyphenyl)benzo[c][2,7]naphthyridin-4-amine)

In the manner of Example 148, treatment of 9-bromo-4-chlorobenzo[c][2,7]naphthyridine with 4-methoxyaniline and pyridine hydrochloride in 2-ethoxyethanol provides the title compound as a yellow solid. MS: 380.1, 382.1 (M+H)⁺¹.

### Example 150

### Synthesis of Compound I-8 N-(3-bromophenyl)-8,9-dimethoxybenzo[c][2,7]naphthyridin-4-amine

Step A: A stirred solution of 4-[(E)-2-(dimethylamino)vinyl]-6,7-dimethoxyquinoline-3-carbonitrile (1.35 g, 4.75 mmol) and 48 ml of HOAc is saturated with HCl gas at 0-5° C. The solution is stirred at 25° C. for 16 hours and concentrated to dryness in vacuo. The residue is stirred in DCM and aqueous K₂CO₃ and filtered. The organic layer of the filtrate is washed with water, dried and concentrated to provide 4-chloro-8,9-dimethoxybenzo[c][2,7]naphthyridine as an amber solid (0.82 g, 63%).

Step B: In the manner of Example 148, treatment of 4-chloro-8,9-dimethoxybenzo[c][2,7]naphthyridine with 3-bromoaniline and pyridine hydrochloride in 2-ethoxyethanol provides the title compound as a tan solid. MS: 410.0, 412.0 (M+H)⁺¹.

### Example 151

### Synthesis of Compound Ib-232

### (2-(3-Fluoro-2-methylphenyl)-8,9-dimethoxybenzo[c][2,7]naphthyridin-4-amine)

To a stirred solution of 3-fluoro-2-methylbenzoic acid (499.0 mg, 3.24 mmol) in 50 ml anhydrous THF, N,N′-carbonyldiimidazole (526.0 mg, 3.24 mmol) is added and the solution is stirred at room temperature for 15 hours. To this solution 6,7-dimethoxy-4-methylquinoline-3-carbonitrile (748.0 mg, 3.3 mmol) is added and the mixture is cooled to −78° C. Then, LiHMDS (1M/THF, 13 ml, 13 mmol) is added, and the mixture is allowed to warm to room temperature while stirred for 2 hours. The dark solution is cooled to ice temperature followed by the addition of glacial acidic acid (20 ml) in small portions, followed by the addition of ammonium acetate (2.5 g, 32.4 mmol). The mixture is heated to 80° C. and stirred at this temperature under nitrogen for 30 mins. The solvents are reduced and crushed ice is added to the mixture, followed by the addition of aqueous ammonium hydroxide solution (5M). The precipitate is filtered, washed with water and hexanes. Purification on silica using CHCl₃/MeOH (10:1) provides 2-(3-fluoro-2-methylphenyl)-8,9-dimethoxybenzo[c][2,7]naphthyridin-4-amine as a brown solid (238 mg, 20.2%) in sufficient purity. MS: 364 (M+H)⁺¹.

Using methods and reagents outlined above, illustrative Benzo[c][2,7]naphthyridine Derivatives listed in the Table below can be prepared.

| Compound | Compound Name | MS: m/e (M + H)⁺¹ |
|---|---|---|
| Ib-76 | 3-(4-amino-8,9-dimethoxybenzo[c][2,7]naphthyridin- | 376.2 |
| | 2-yl)benzoic acid | |
| Ib-233 | 2-(3-aminophenyl)-8,9- | 347.2 |
| | dimethoxybenzo[c][2,7]naphthyridin-4-amine | |
| Ib-234 | 2-(4-bromo-3-fluorophenyl)-8,9- | 428.2 |
| | dimethoxybenzo[c][2,7]naphthyridin-4-amine | |
| Ib-235 | 2-(2-bromo-1,3-thiazol-5-yl)-8,9- | 417.2 |
| | dimethoxybenzo[c][2,7]naphthyridin-4-amine | |
| Ib-236 | 8,9-dimethoxy-2-(1,3-thiazol-5- | 339.2 |
| | yl)benzo[c][2,7]naphthyridin-4-amine | |
| Ib-237 | 8,9-dimethoxy-2-(6-methoxypyridin-3- | 363.1 |
| | yl)benzo[c][2,7]naphthyridin-4-amine | |
| Ib-238 | methyl 3-(4-amino-8,9- | 468.4 |
| | dimethoxybenzo[c][2,7]naphthyridin-2-yl)benzoate | |
| Ib-239 | 2-(3,5-difluorophenyl)-8,9- | 368.3 |
| | dimethoxybenzo[c][2,7]naphthyridin-4-amine | |
| Ib-240 | 2-(3,4-difluorophenyl)-8,9- | 368.3 |
| | dimethoxybenzo[c][2,7]naphthyridin-4-amine | |
| Ib-241 | 3-(4-amino-8,9-dimethoxybenzo[c][2,7]naphthyridin- | 375.3 |
| | 2-yl)-5-fluorobenzonitrile | |
| Ib-242 | 2-(3-fluoro-5-methoxyphenyl)-8,9- | 380.3 |
| | dimethoxybenzo[c][2,7]naphthyridin-4-amine | |
| Ib-243 | 3-(4-amino-8,9-dimethoxybenzo[c][2,7]naphthyridin- | 409.1 |
| | 2-yl)-5-fluorobenzamide | |
| Ib-244 | 2-(1H-benzimidazol-5-yl)-8,9- | 372.1 |
| | dimethoxybenzo[c][2,7]naphthyridin-4-amine | |
| Ib-245 | 8,9-dimethoxy-2-[3-(1,3-oxazol-5- | 399.2 |
| | yl)phenyl]benzo[c][2,7]naphthyridin-4-amine | |
| Ib-246 | 8,9-dimethoxy-2-[5-(1-methyl-1H-pyrazol-4- | 413.3 |
| | yl)pyridin-3-yl]benzo[c][2,7]naphthyridin-4-amine | |
| Ib-247 | 8,9-dimethoxy-2-{3-[(4-pyridin-2-ylpiperazin-1- | 521.2 |
| | yl)carbonyl]phenyl}benzo[c][2,7]naphthyridin-4- | |
| | amine | |
| Ib-248 | 3-(4-amino-8,9-dimethoxybenzo[c][2,7]naphthyridin- | 466.2 |
| | 2-yl)-N-(pyridin-3-ylmethyl)benzamide | |
| Ib-249 | 2-(1H-indazol-5-yl)-8,9- | 372.1 |
| | dimethoxybenzo[c][2,7]naphthyridin-4-amine | |

### Example 152

### Synthesis of Compound Ib-250

### (2-(3-(3-Aminopropyl)-5-fluorophenyl)-8,9-dimethoxybenzo[c][2,7]naphthyridin-4-amine)

To a solution of tert-butyl allylcarbamate (245 mg, 1.56 mmol) in anhydrous THF (3 ml), a solution of 9-borabicyclo[3.3.1]nonane (1M/THF, 9 ml, 4.5 mmol) is added over a period of 20 minutes via a syringe pump at 0° C. The solution is slowly warmed to room temperature and stirred for 5 hours. This solution is added to a mixture of 2-(3-bromo-5-fluorophenyl)-8,9-dimethoxybenzo[c][2,7]naphthyridin-4-amine (316 mg, 0.74 mmol), cesium carbonate (404 mg, 1.24 mmol) and [1,1′-bis(diphenylphosphino)-ferrocene]dichloropalladium (II) (118 mg, 0.144 mol) in a mixture of DMF (2 ml) and water (1 ml) and the combined mixture is heated to 80° C. under nitrogen for 14 hours. The reaction mixture is quenched with brine, extracted with a 1:1 mixture of EtAc and THF, and dried with sodium sulfate. Flash column chromatography using silica and CHCl₃/MeOH (10:1) provides tert-butyl 3-(3-(4-amino-8,9-dimethoxybenzo-[c][2,7]naphthyridin-2-yl)-5-fluorophenyl)propylcarbamate (253 mg, 67.7%). MS: 507 (M+H)⁺¹.

To a solution of tert-butyl 3-(3-(4-amino-8,9-dimethoxybenzo-[c][2,7]-naphthyridin-2-yl)-5-fluorophenyl)propylcarbamate (466 mg, 0.92 mmol) in anhydrous DCM (10 ml), trifluoroacetic acid (2 ml, 2.96 g, 26.0 mmol) at 0° C. is added and the solution is slowly allowed to warm to room temperature while stirred for 5 hours. The solvent is removed a mixture of 1N aqueous NaOH, THF and EtAc is added to the residue, which is then extracted, washed with brine and dried over sodium sulfate. The resultant semi-solid is dissolved in a mixture of THF/MeOH (5:1, 5 ml total, containing 1% triethylamine) and the solution is loaded onto solid silica. The silica is washed repeatedly with the same solvent combination. Then, the silica is suspended in a mixture of CHCl₃/MeOH (2:1, 5 ml, containing 1% triethylamine) and ultrasonicated for 5 mins. It is filtered, the solvents are removed and the residue is dried in vacuo to provide 2-(3-(3-Aminopropyl)-5-fluorophenyl)-8,9-dimethoxybenzo-[c][2,7]naphthyridin-4-amine in sufficient purity (32 mg, 9%). MS: 407.4 (M+H)⁺¹.

### Example 153

### Synthesis of Compound Ib-251

### ((S)-1-(1-(5-(4-amino-8,9-dimethoxybenzo[c][2,7]naphthyridin-2-yl)pyridin-3-yloxy)butan-2-yl)guanidine)

To a suspension of (S)-2-(5-(2-aminobutoxy)pyridin-3-yl)-8,9-dimethoxybenzo-[c][2,7]naphthyridin-4-amine hydrochloride (223.7 mg, 0.373 mmol) and potassium carbonate (534 mg, 3.86 mmol) in 8 ml anhydrous DMF, tert-butyl (1H-pyrazol-1-yl)methane-diylidenedicarbamate (237 mg, 0.763 mmol) is added and the mixture is stirred at room temperature for 17 hours. The solvent is removed and the residue purified using flash column chromatography (CHCl₃/MeOH 5:1) to provide (S)-tert-butyl (1-(5-(4-amino-8,9-dimethoxybenzo[c][2,7]naphthyridin-2-yl)pyridin-3-yloxy)butan-2-ylamino)(tert-butoxycarbonylamino)methylenecarbamate (15 mg, 6%). MS: 662.0 (M+H)⁺¹.

A mixture of (S)-tert-butyl (1-(5-(4-amino-8,9-dimethoxybenzo-[c][2,7]naphthyridin-2-yl)pyridin-3-yloxy)butan-2-ylamino)(tert-butoxycarbonylamino)-methylenecarbamate (34.40 mg, 0.052 mmol, 3 ml EtAc and 0.35 12 N aqueous HCl solution is stirred for 14 hours at room temperature. The obtained precipitate is dried to provide (S)-1-(1-(5-(4-amino-8,9-dimethoxybenzo[c][2,7]naphthyridin-2-yl)pyridin-3-yloxy)butan-2-yl)guanidine as a hydrochloride salt in sufficient purity (23.5 mg, 70.1%). MS: 462.4 (M+H)⁺¹.

### Example 154

### Synthesis of Compound Ib-184a

### (2-(5-bromopyridin-3-yl)-8,9-dimethoxybenzo[c]-2,7-naphthyridin-4-amine)

To a suspension of dimethoxymethylquinoline (5.50 g, 24.1 mmol) and methyl-5-bromonicotinate (5.20 g, 24.1 mmol) in tetrahydrofuran (200 mL) at −78° C., lithium bis(trimethylsilyl)-amide (1.0 M in THF, 108.45 mL, 108.45 mmol) is added. The reaction mixture is allowed to warm up to room temperature and stirred overnight. The reaction mixture is then cooled to 0° C., and acetic acid (149.6 mL) and ammonia acetate (37.15 g, 482 mmol) are added. The reaction mixture is heated at 100° C. for 2 hours. After cooling to room temperature, the solid is filtered, washed with 1N NaOH, EtOAc and diethyl ether to provide 8.29 g (84%) of a light yellow solid which is characterized as 2-(5-bromopyridin-3-yl)-8,9-dimethoxybenzo[c]-2,7-naphthyridin-4-amine MS (ESI): m/z 411; HRMS, calculated for C₁₉H₁₅BrN₄O₂+H+, 411.04511; found (ESI, [M+H]+ Observed), 411.0443; HRMS: calculated for C₁₉H₁₅BrN₄O₂+H+, 411.04511; found (ESI, [M+H]+ Calculated), 411.0451.

### Example 155

### Synthesis of Compounds Ib-253 to Ib-255

### (2-[5-(3-aminoprop-1-yn-1-yl)pyridin-3-yl]-8,9-dimethoxybenzo[c][2,7]naphthyridin-4-amine)

To a mixture of 2-(5-bromopyridin-3-yl)-8,9-dimethoxybenzo[c]-2,7-naphthyridin-4-amine (891 mg, 2.17 mmol), carbamic acid, 2-propenyl-1,1-dimethylethyl ester (370 mg, 2.38 mmol), tetrakis(triphenyl phosphine)palladium(0) (125.4 mg, 0.11 mmol), and triphenyl phosphine (56.92 mg, 0.22 mmol) in hot, degassed piperidine (25 mL), copper iodide (41.9 mg, 0.22 mmol) is added. The reaction mixture is microwaved at 80° C. for 1 hour. Solvent is evaporated in vacuo, toluene is added and then evaporated. The crude residue is triturated with EtOAc, the solid is filtered, washed with EtOAc, CH₃CN, MeOH and hot EtOH to provide 407 mg (38%) of yellow solid, which is characterized as tert-butyl {3-[5-(4-amino-8,9-dimethoxybenzo[c][2,7]naphthyridin-2-yl)pyridin-3-yl]prop-2-yn-1-yl}carbamate.

Using methods and reagents outlined in the Examples above, illustrative Aminopropynyl-Benzo[c][2,7]naphthyridine Derivatives listed in the Table below are prepared. The intermediate necessary to prepared list below can be prepared according to the procedures described in Reginato, et al., Tetrahedron, 1996, Vol. 52, No. 33:10985-10996, which is incorporated herein by reference in its entirety.

| | | MS: m/e |
|---|---|---|
| Compound | Compound Name | (M + H)⁺¹ |
| Ib-253 | 2-[5-(3-amino-3-methylbut-1-yn-1-yl)pyridin- | 414.4 |
| | 3-yl]-8,9-dimethoxybenzo[c][2,7]naphthyridin- | |
| | 4-amine | |
| Ib-254 | 2-{5-[(1-aminocyclohexyl)ethynyl]pyridin-3- | 454.4 |
| | yl}-8,9-dimethoxybenzo[c][2,7]naphthyridin- | |
| | 4-amine | |
| Ib-255 | tert-butyl {(1R)-3-[5-(4-amino-8,9-dimethoxy | 514.4 |
| | benzo[c][2,7]naphthyridin-2-yl)pyridin-3-yl]- | |
| | 1-ethylprop-2-yn-1-yl}carbamate 124725 | |

### Example 156

### Synthesis of Compound Ib-252

### (2-[5-(3-aminoprop-1-yn-1-yl)pyridin-3-yl]-8,9-dimethoxybenzo[c][2,7]naphthyridin-4-amine)

A suspension of tert-butyl {3-[5-(4-amino-8,9-dimethoxybenzo[c][2,7]naphthyridin-2-yl)pyridin-3-yl]prop-2-yn-1-yl}carbamate (33 mg, 0.07 mmol) in EtOAc (2.3 mL) is treated with concentrated HCl (0.5 mL) and the resulting reaction mixture is stirred at room temperature for 3 hours. The solid is filtered and washed with EtOAc to provide 36 mg (quantitative) of a red solid, characterized as 2-[5-(3-aminoprop-1-yn-1-yl)pyridin-3-yl]-8,9-dimethoxybenzo[c][2,7]naphthyridin-4-amine: MS: 386.3 (M+H)⁺¹.

### Example 157

### Synthesis of Compounds Ib-257 to Ib-268

### (2-[5-(3-aminopropyl)pyridin-3-yl]-8,9-dimethoxybenzo[c][2,7]naphthyridin-4-amine)

To a solution of tert-butyl {3-[5-(4-amino-8,9-dimethoxybenzo[c][2,7]naphthyridin-2-yl)pyridin-3-yl]prop-2-yn-1-yl}carbamate (180 mg, 0.371 mmol) in EtOH (15 mL) and N,N-dimethylformamide (7 mL), 10% Pd/C (150 mg) is added. The reaction mixture is hydrogenated under 50 PSI of pressure at room temperature overnight. It is filtered through diatomaceous earth and rinsed with EtOH. Solvent is evaporated in vacuo to provide 150 mg (82%) of product as a brown solid: {3-{5-(4-Amino-8,9-dimethoxybenzo[c][2,7]naphthyridin-2-yl)-pyridin-3-yl]-propyl}-carbamic acid tert-butyl ester. MS: 490.2 (M+H)⁺¹.

Using methods and reagents outlined in the examples above, illustrative Benzo[c][2,7]naphthyridine Derivatives listed the Table below can be prepared.

| | | MS: m/e |
|---|---|---|
| Compound | Compound Name | (M + H)⁺¹ |
| Ib-257 | 2-[5-(3-amino-3-methylbutyl)pyridin-3-yl]- | 418.4 |
| | 8,9-dimethoxybenzo[c][2,7]naphthyridin-4- | |
| | amine | |
| Ib-258 | 2-{5-[2-(1-aminocyclohexyl)ethyl]pyridin-3- | 458.5 |
| | yl}-8,9- | |
| | dimethoxybenzo[c][2,7]naphthyridin-4- | |
| | amine | |

### Example 158

### Synthesis of Compound Ib-256

Solid {3-{5-(4-Amino-8,9-dimethoxybenzo[c][2,7]naphthyridin-2-yl)-pyridin-3-yl]-propyl}-carbamic acid tert-butyl ester (150 mg, 0.307 mmol) is treated with neat trifluoroacetic acid (2.0 mL). The reaction mixture is stirred at room temperature for 1 hour. Trifluoroacetic acid is removed. The solid is dissolved in MeOH (2.0 mL), and then purified in Gilson reverse phase HPLC. Product fractions are collected and concentrated to provide 25 mg (21%) of brown solid, characterized as 2-[5-(3-aminopropyl)pyridin-3-yl]-8,9-dimethoxybenzo[c][2,7]naphthyridin-4-amine: MS: 390.2 (M+H)⁺¹.

### Example 159

### Synthesis of Compound Ib-259

### (Tert-butyl {(1R)-3-[5-(4-amino-8,9-dimethoxybenzo[c][2,7]naphthyridin-2-yl)pyridin-3-yl]-1-ethylpropyl}carbamate)

To tert-butyl {(1R)-3-[5-(4-amino-8,9-dimethoxy benzo[c][2,7]naphthyridin-2-yl)pyridin-3-yl]-1-ethylprop-2-yn-1-yl}carbamate (318 mg, 0.62 mmol) in EtOH (24.5 mL) and N,N-dimethylformamide (11.5 mL), 10% Pd/C (150 mg) is added. The reaction mixture is hydrogenated under 50 PSI of pressure at room temperature overnight, filtered through diatomaceous earth, and rinsed with Et₂O. The solvent is evaporated in vacuo to provide 144 mg (45%) of yellow solid, characterized as tert-butyl {(1R)-3-[5-(4-amino-8,9-dimethoxybenzo[c][2,7]naphthyridin-2-yl)pyridin-3-yl]-1-ethylpropyl}carbamate: MS: 518.5 (M+H)⁺¹.

### Example 160

### Synthesis of Compound Ib-260

### (2-{5-[(3R)-3-aminopentyl]pyridin-3-yl}-8,9-dimethoxybenzo[c][2,7]naphthyridin-4-amine)

Solid tert-butyl {(1R)-3-[5-(4-amino-8,9-dimethoxybenzo[c][2,7]naphthyridin-2-yl)pyridin-3-yl]-1-ethylpropyl}carbamate (78 mg, 0.15 mmol) in EtOAc (5.2 mL) is treated with concentrated HCl (0.22 mL) and the resulting reaction mixture is stirred at room temperature for 3 hours. The solid is filtered and washed with Et₂O to provide 75 mg (100%) of a red solid, characterized as 2-{5-[(3R)-3-aminopentyl]pyridin-3-yl}-8,9-dimethoxybenzo[c][2,7]naphthyridin-4-amine: MS: 418.4 (M+H)⁺¹.

### Example 161

### Synthesis of Compound Ib-261

### (2-{5-[(3R)-3-aminopent-1-yn-1-yl]pyridin-3-yl}-8,9-dimethoxybenzo[c][2,7]naphthyridin-4-amine)

Solid tert-butyl {(1R)-3-[5-(4-amino-8,9-dimethoxy benzo[c][2,7]naphthyridin-2-yl)pyridin-3-yl]-1-ethylprop-2-yn-1-yl}carbamate (105 mg, 0.21 mmol) in EtOAc (7 mL) is treated with concentrated HCl (0.3 mL), following the procedure of Example 160, to provide 54 mg (63.8%) of an orange solid, characterized as 2-{5-[(3R)-3-aminopent-1-yn-1-yl]pyridin-3-yl}-8,9-dimethoxybenzo[c][2,7]naphthyridin-4-amine: MS: 414.3 (M+H)⁺¹.

### Example 162

### (Tert-butyl [(1R)-3-[5-(4-amino-8,9-dimethoxybenzo[c][2,7]naphthyridin-2-yl)pyridin-3-yl]-1-({[tert-butyl(dimethyl)silyl]oxy}methyl)prop-2-yn-1-yl]carbamate)

Step A: Methyl N-(tert-butoxycarbonyl)-O-[tert-butyl(dimethyl)silyl]-L-serinate. A reaction mixture of N-(tert-butoxycarbonyl)-L-serinate methyl ester (39.0 g, 177.89 mmol), tert-Butyldimethylsilylchloride (29.5 g, 195.67 mmol) and imidazole (30.3 g, 444.73 mmol) in N,N-dimethylformamide (700 mL) is stirred at 65° C. for 2 days. N,N-dimethylformamide is removed in vacuo. H₂O is added to the residue, which is then extracted with hexane. The hexane solution is washed with dilute HCl and brine. It is then dried over MgSO₄, passed through a short column of silica gel, and evaporated solvent to provide 53.9 g (100%) of colorless oil, characterized as methyl N-(tert-butoxycarbonyl)-O-[tert-butyl(dimethyl)silyl]-L-serinate: MS: 334.3 (M+H)⁺¹.

Step B: Tert-butyl [(1S)-2-{[tert-butyl(dimethyl)silyl]oxy}-1-formylethyl]carbamate. To a cold solution of methyl N-(tert-butoxycarbonyl)-O-[tert-butyl(dimethyl)silyl]-L-serinate (53.98 g, 161.86 mmol) CH₂Cl₂ (360 mL) at −78° C., diisobutylaluminium hydride (1.0 M solution in toluene, 323.7 g, 323.7 mmol) is added dropwise over 40 minutes. The reaction mixture is stirred at −78° C. for 4 hours and then quenched with acetic acid/toluene. The resultant solution is poured into diluted HCl and filtered. The organic layer is removed. The aqueous layer is extracted with EtOAc two times. The combined organic extracts are washed with brine, dried over MgSO₄, and then passed through a plug of magnesium silicate. Solvent is removed in vacuum to provide 49.4 g (100%) of colorless oil, characterized as tert-butyl [(1S)-2-{[tert-butyl(dimethyl)silyl]oxy}-1-formylethyl]carbamate: MS: 304.3 (M+H)⁺¹.

Step C: Tert-butyl [(1R)-1-({[tert-butyl(dimethyl)silyl]oxy}methyl)prop-2-yn-1-yl]carbamate. To the solution of tert-butyl [(1S)-2-{[tert-butyl(dimethyl)silyl]oxy}-1-formylethyl]carbamate (26.3 g, 78.08 mmol) in anhydrous MeOH (700 mL), dimethyl (1-diazo-2-oxopropyl)phosphonate (33.0 g, 156.16 mmol) and K₂CO₃ (30 g, 195.2 mmol) is added at 0° C. (see Ohira, S. Synth. Commun. 1989, 19, 561-564, incorporated by reference herein in its entirety.) The reaction mixture is gradually warmed to room temperature overnight, then partitioned between saturated NaHCO₃ solution and CH₂Cl₂. The aqueous layer is extracted with CH₂Cl₂ two times. The combined organic extracts are dried over Na₂SO₄, filtered and concentrated to provide a brown oil crude. Chromatography of the crude using a forisil column, eluted with hexane, hexane/EtOAc=3:1 to 1:1, provides 13.88 g (59%) of an orange solid, characterized as tert-butyl [(1R)-1-({[tert-butyl(dimethyl)silyl]oxy}methyl)prop-2-yn-1-yl]carbamate: MS: 300.4 (M+H)⁺¹.

Step D: Tert-butyl [(1R)-3-[5-(4-amino-8,9-dimethoxybenzo[c][2,7]naphthyridin-2-yl)pyridin-3-yl]-1-({[tert-butyl(dimethyl)silyl]oxy}methyl)prop-2-yn-1-yl]carbamate. The reaction mixture of 2-(5-bromopyridin-3-yl)-8,9-dimethoxybenzo[c]-2,7-naphthyridin-4-amine (8.97 g, 21.89 mmol), tert-butyl [(1R)-1-({[tert-butyl(dimethyl)silyl]oxy}methyl)prop-2-yn-1-yl]carbamate (8.1 mg, 27.04 mmol), tetrakis(triphenyl phosphine)palladium(0) (1.42 mg, 1.2 mmol), triphenyl phosphine (644.8 mg, 2.5 mmol) and copper iodide (468.1 mg, 2.45 mmol) in hot, degassed piperidine (63 mL) and N,N-dimethylformamide (63 mL), is microwaved at 80° C. for 2 hours. The solvent is evaporated in vacuo. Toluene is added and evaporated. The residue is chromatographed using a silica gel column, eluted with CHCl₃, 1% isopropanol/CHCl₃ to 3% isopropanol/CHCl₃. Product fractions are collected and solvent is evaporated to provide 8.01 g (58%) of an orange solid, characterized as tert-butyl [(1R)-3-[5-(4-amino-8,9-dimethoxybenzo[c][2,7]naphthyridin-2-yl)pyridin-3-yl]-1-({[tert-butyl(dimethyl)silyl]oxy}methyl)prop-2-yn-1-yl]carbamate: MS: 630.3 (M+H)⁺¹.

### Example 163

### (Tert-butyl [(1R)-3-[5-(4-amino-8,9-dimethoxybenzo[c][2,7]naphthyridin-2-yl)pyridin-3-yl]-1-({[tert-butyl(dimethyl)silyl]oxy}methyl)propyl]carbamate)

To a solution of tert-butyl [(1R)-3-[5-(4-amino-8,9-dimethoxybenzo[c][2,7]naphthyridin-2-yl)pyridin-3-yl]-1-({[tert-butyl(dimethyl)silyl]oxy}methyl)prop-2-yn-1-yl]carbamate (1.03 mg, 1.64 mmol) in EtOH (60 mL) and N,N-dimethylformamide (40 mL), 10% Pd/C (wet 1.32 g) is added. The reaction mixture is hydrogenated under 50 PSI of pressure at room temperature overnight, filtered through diatomaceous earth, and rinsed with EtOH. The solvent is evaporated in vacuo to provide 285 mg (27%) of product as a yellow solid, characterized as tert-butyl [(1R)-3-[5-(4-amino-8,9-dimethoxybenzo[c][2,7]naphthyridin-2-yl)pyridin-3-yl]-1-({[tert-butyl(dimethyl)silyl]oxy}methyl)propyl]carbamate: MS: 634.5 (M+H)⁺¹.

### Example 164

### Synthesis of Compound Ib-262

### (Tert-butyl [(1R)-3-[5-(4-amino-8,9-dimethoxybenzo[c][2,7]naphthyridin-2-yl)pyridin-3-yl]-1-(hydroxymethyl)propyl]carbamate)

To a suspension of tert-butyl [(1R)-3-[5-(4-amino-8,9-dimethoxybenzo[c][2,7]naphthyridin-2-yl)pyridin-3-yl]-1-({[tert-butyl(dimethyl)silyl]oxy}methyl)propyl]carbamate (2.7 g, 4.26 mmol) in MeOH (55 mL) at room temperature, NH₄F is added, and the resultant reaction mixture is refluxed for 3 hours, and then diluted with ice/water. The solid is collected, washed with water several times, boiled in acetone, cooled, and filtered to provide 1.5 g (67.7%) of a light green solid, characterized as tert-butyl [(1R)-3-[5-(4-amino-8,9-dimethoxybenzo[c][2,7]naphthyridin-2-yl)pyridin-3-yl]-1-(hydroxymethyl)propyl]carbamate: MS: 520.3 (M+H)⁺¹.

### Example 165

### Synthesis of Compound Ib-263

### (2-(5-{2-[(2R)-aziridin-2-yl]ethyl}pyridin-3-yl)-8,9-dimethoxybenzo[c][2,7]naphthyridin-4-amine)

Step A: Methanesulfonic acid 4-[5-(4-amino-8,9-dimethoxy-benzo[c][2,7]naphthyridin-2-yl)-pyridin-3-yl]-2-tert-butoxycarbonylamino-butyl ester. To a mixture of tert-butyl [(1R)-3-[5-(4-amino-8,9-dimethoxybenzo[c][2,7]naphthyridin-2-yl)pyridin-3-yl]-1-(hydroxymethyl)propyl]carbamate (1.62 g, 3.118 mmol) in pyridine (25.8 mL) at 0° C., mesyl chloride (3.57 g, 31.18 mmol) is added. The reaction mixture is stirred at 0° C. for 5 hours, and then diluted with cold H₂O (40 mL). The solid is collected, washed with H₂O three times, washed with ether, and dried to provide 765 mg (41%) of a yellow solid, identified as methanesulfonic acid 4-[5-(4-amino-8,9-dimethoxy-benzo[c][2,7]naphthyridin-2-yl)-pyridin-3-yl]-2-tert-butoxycarbonylamino-butyl ester.

Step B: Methanesulfonic acid 2-amino-4-[5-(4-amino-8,9-dimethoxy-benzo[c][2,7]naphthyridin-2-yl)-pyridin-3-yl]-butyl ester. A solution of methanesulfonic acid 4-[5-(4-amino-8,9-dimethoxy-benzo[c][2,7]naphthyridin-2-yl)-pyridin-3-yl]-2-tert-butoxycarbonylamino-butyl ester (650 g, 2.37 mmol) in EtOAc (39.2 mL) is treated with concentrated HCl (0.5 mL). The resultant reaction mixture is stirred at room temperature for 4 hours. EtOAc is evaporated in vacuo. Toluene is added and evaporated 3 times. EtOAc is added and evaporated. More EtOAc is added and the solid is collected, washed with a small amount of EtOAc and ether to provide 1.4 g (90%) of a yellow solid, identified as methanesulfonic acid 2-amino-4-[5-(4-amino-8,9-dimethoxy-benzo[c][2,7]naphthyridin-2-yl)-pyridin-3-yl]-butyl ester.

Step C: 2-(5-{2-[(2R)-aziridin-2-yl]ethyl}pyridin-3-yl)-8,9-dimethoxybenzo[c][2,7]naphthyridin-4-amine. A solution of methanesulfonic acid 2-amino-4-[5-(4-amino-8,9-dimethoxy-benzo[c][2,7]naphthyridin-2-yl)-pyridin-3-yl]-butyl ester (1.34 g, 2.71 mmol) in H₂O (25 mL), 5N NaOH (1.34 g, 2.71 mmol), CHCl₃ (93 mL) and Hünig's base (0.472 mL, 2.71 mmol) is added. The resulting reaction mixture is stirred at room temperature overnight. The organic layer is separated, and the aqueous layer is extracted with CHCl₃ three times. The combined organic extracts are dried over MgSO₄. The solvent is evaporated to provide 1.04 g (95%) of a yellow solid, characterized as 2-(5-{2-[(2R)-aziridin-2-yl]ethyl}pyridin-3-yl)-8,9-dimethoxybenzo[c][2,7]naphthyridin-4-amine: MS: 402.3 (M+H)⁺¹.

### Example 165

### (2-(5-{2-[(2R)-1-(diphenylphosphoryl)aziridin-2-yl]ethyl}pyridin-3-yl)-8,9-dimethoxybenzo[c][2,7]naphthyridin-4-amine)

To a solution of 2-(5-{2-[(2R)-aziridin-2-yl]ethyl}pyridin-3-yl)-8,9-dimethoxybenzo[c][2,7]naphthyridin-4-amine (1.04 g, 2.6 mmol) in CHCl₃ (107 mL) and MeOH (68 mL) at 0° C., diphenylphosphinic chloride (769.6 g, 3.25 mmol) in CHCl₃ (2.65 mL) is added dropwise over 15 minutes. The reaction mixture is stirred at 0° C. for 1 hour and then at room temperature for 3 hours. The solvent is evaporated in vacuo. The residue is mixed with a CHCl₃/MeOH solution. Saturated NaHCO₃ is added, and the aqueous layer is extracted with CHCl₃/MeOH solution 3 times. The combined organic extracts are dried over Na₂SO₄, filtered, and concentrated. The crude is purified by column chromatography, eluted from 2% MeOH/CHCl₃ to 11% MeOH/CHCl₃. Product fractions are collected, the solvent is evaporated to provide 360 mg (23%) of product as an orange solid, characterized as 2-(5-{2-[(2R)-1-(diphenylphosphoryl)aziridin-2-yl]ethyl}pyridin-3-yl)-8,9-dimethoxybenzo[c][2,7]naphthyridin-4-amine: MS: 602.4 (M+H)⁺¹.

### Example 166

### (N-{(1R)-3-[5-(4-amino-8,9-dimethoxybenzo[c][2,7]naphthyridin-2-yl)pyridin-3-yl]-1-[(1,3-thiazol-2-ylthio)methyl]propyl}-P,P-diphenylphosphinic amide)

A reaction mixture of 2-(5-{2-[(2R)-1-(diphenylphosphoryl)aziridin-2-yl]ethyl}pyridin-3-yl)-8,9-dimethoxybenzo[c][2,7]naphthyridin-4-amine (137 mg, 0.23 mmol), 2-mercaptothiazole (266 mg, 2.28 mmol) and LiI (30.5 mg, 0.23 mmol) in N,N-dimethylformamide (2.0 mL) is stirred at 65° C. overnight. The solvent is evaporated in vacuo. The residue is dissolved in DMSO, purified by Gilson reverse phase HPLC to provide 42 mg (25%) of an orange solid, characterized as N-{(1R)-3-[5-(4-amino-8,9-dimethoxybenzo[c][2,7]naphthyridin-2-yl)pyridin-3-yl]-1-[(1,3-thiazol-2-ylthio)methyl]propyl}-P,P-diphenylphosphinic amide: MS 719.3 (M+H)⁺¹.

### Example 167

### (N-[(1R)-3-[5-(4-amino-8,9-dimethoxybenzo[c][2,7]naphthyridin-2-yl)pyridin-3-yl]-1-{[ethyl(pyridin-4-ylmethyl)amino]methyl}propyl]-P,P-diphenylphosphinic amide)

A reaction mixture of 2-(5-{2-[(2R)-1-(diphenylphosphoryl)aziridin-2-yl]ethyl}pyridin-3-yl)-8,9-dimethoxybenzo[c][2,7]naphthyridin-4-amine (183 mg, 0.304 mmol), 4-(ethylaminomethyl)-pyridine (0.423 mL, 3.04 mmol) and LiI (40.68 mg, 0.304 mmol) in N,N-dimethylformamide (3.6 mL) is allowed to react following the procedure of Example 166 to provide 99 mg (44%) of an orange solid, characterized as N-[(1R)-3-[5-(4-amino-8,9-dimethoxybenzo[c][2,7]naphthyridin-2-yl)pyridin-3-yl]-1-{[ethyl (pyridin-4-ylmethyl)amino]methyl}propyl]-P,P-diphenylphosphinic amide: MS: 738.4 (M+H)⁺¹.

### Example 168

### Synthesis of Compound Ib-264

### (2-{5-[(3R)-3-amino-4-(1,3-thiazol-2-ylthio)butyl]pyridin-3-yl}-8,9-dimethoxybenzo[c][2,7]naphthyridin-4-amine)

A solution of N-{(1R)-3-[5-(4-amino-8,9-dimethoxybenzo[c][2,7]naphthyridin-2-yl)pyridin-3-yl]-1-[(1,3-thiazol-2-ylthio)methyl]propyl}-P,P-diphenylphosphinic amide (see Example 166) (42 mg, 0.06 mmol) in EtOAc (3.0 mL) is treated with concentrated HCl (0.5 mL) and stirred for 30 minutes. EtOAc (25 mL) is added and the resultant reaction mixture is stirred at room temperature overnight. The solid is filtered and washed with EtOAc to provide 29 mg (95%) of a red solid, characterized as 2-{5-[(3R)-3-amino-4-(1,3-thiazol-2-ylthio)butyl]pyridin-3-yl}-8,9-dimethoxybenzo[c][2,7]naphthyridin-4-amine: MS: 519.3 (M+H)⁺¹.

### Example 169

### Synthesis of Compound Ib-265a

### ((2R)-4-[5-(4-amino-8,9-dimethoxybenzo[c][2,7]naphthyridin-2-yl)pyridin-3-yl]-N¹-ethyl-N¹-(pyridin-4-ylmethyl)butane-1,2-diamine)

A solution of N-[(1R)-3-[5-(4-amino-8,9-dimethoxybenzo[c][2,7]naphthyridin-2-yl)pyridin-3-yl]-1-{[ethyl(pyridin-4-ylmethyl)amino]methyl}propyl]-P,P-diphenylphosphinic amide (see Example 167) (78 mg, 0.106 mmol) in EtOAc (3.0 mL) is treated with concentrated HCl (1.0 mL) following the procedure of Example 168 to provide 55 mg (72%) of an orange solid, characterized as (2R)-4-[5-(4-amino-8,9-dimethoxybenzo[c][2,7]naphthyridin-2-yl)pyridin-3-yl]-N¹-ethyl-N¹-(pyridin-4-ylmethyl)butane-1,2-diamine: MS: 538.5 (M+H)⁺¹.

### Example 170

### (Tert-butyl (3-{[5-(4-amino-8,9-dimethoxybenzo[c][2,7]naphthyridin-2-yl)pyridin-3-yl]oxy}pyridin-2-yl)carbamate)

Step A: 3-Fluoro-2-nitro-pyridine. A solution of sodium nitrite (503.4 mg, 73 mmol) in H₂O (1.0 mL) is added dropwise to a stirred mixture of 3-amino-2-nitropyridine (1.05 g, 73 mmol) in 34% fluoroboric acid (3.5 mL). Temperature is maintained between −8° C. and −2° C. during the addition. The reaction mixture is stirred at room temperature for 30 minutes. The suspension is filtered and the solid is washed with 34% fluoroboric acid (20 mL) and ether (10 mL), dried under vacuum overnight to provide a fluoroborate salt. The fluoroborate salt is refluxed in toluene (50 mL) for 3 hours, treated with 10% NaHCO₃, and extracted with CHCl₃ three times. The combined extracts are dried in Na₂SO₄, filtered and concentrated to provide 30 mg (2.8%) of brown oil, identified as 3-Fluoro-2-nitro-pyridine.

Step B: 5-(2-Nitro-pyridin-3-yloxy)-nicotinic acid methyl ester. To a solution of 3-Fluoro-2-nitro-pyridine (183 mg, 1.29 mmol) and 5-hydroxynicotinic acid methyl ester (197.2 mg, 1.29 mmol) in tetrahydrofuran (2.8 mL) at room temperature under nitrogen, NaH (51.52 mg, 1.29 mmol) is added. The reaction mixture is stirred at room temperature for 3 days. The product is partitioned between CHCl₃ and H₂O. The CHCl₃ layer is separated, aqueous layer is extracted with CHCl₃ three times. The combined organic extracts are washed with brine, dried over Na₂SO₄, filtered and concentrated to provide a brown oil crude. The crude is purified in a preparative thin layer chromatography plate, eluted with 1% MeOH/CHCl₃ to provide 224 mg (63.2%) of product as yellow crystals, identified as 5-(2-Nitro-pyridin-3-yloxy)-nicotinic acid methyl ester.

Step C: 5-(2-Amino-pyridin-3-yloxy)-nicotinic acid methyl ester. To a solution of 5-(2-Nitro-pyridin-3-yloxy)-nicotinic acid methyl ester (2.353 mg, 8.55 mmol) in MtOH (8 mL) and EtOAc (38 mL), 10% Pd/C (wet 770 mg) is added. The reaction mixture is hydrogenated under 50 PSI of pressure at room temperature for 1 hour, filtered through diatomaceous earth, and rinsed with MtOH. Solvent is evaporated in vacuo to provide 1.75 g (83%) of product as a white solid, identified as 5-(2-Amino-pyridin-3-yloxy)-nicotinic acid methyl ester.

Step D: Methyl 5-({2-[bis(tert-butoxycarbonyl)amino]pyridin-3-yl}oxy)nicotinate. To a solution of 5-(2-Amino-pyridin-3-yloxy)-nicotinic acid methyl ester (1.75 g, 7.14 mmol) and DMAP (130.8 mg, 1.07 mmol) in tetrahydrofuran (25 mL) at 0° C., di-tert-butyl dicarbonate (4.28 mg, 19.63 mmol) is added. The reaction mixture is stirred at room temperature overnight. Tetrahydrofuran is evaporated in vacuo. The residue is chromatographed using ether/hexane=1:10 to 1:7. Product fractions are combined and evaporated to provide 1.17 g (37%) of product as an oil, identified as methyl 5-({2-[bis(tert-butoxycarbonyl)amino]pyridin-3-yl}oxy)nicotinate.

Step E: tert-butyl (3-{[5-(4-amino-8,9-dimethoxybenzo[c][2,7]naphthyridin-2-yl)pyridin-3-yl]oxy}pyridin-2-yl)carbamate. To a solution of 6,7-dimethoxy-4-methylquinoline-3-carbonitrile (440.6 mg, 1.93 mmol) and methyl 5-({2-[bis(tert-butoxycarbonyl)amino]pyridin-3-yl}oxy)nicotinate (860 mg, 1.94 mmol) in tetrahydrofuran (18 mL) at −78° C. under nitrogen, Lithium bis(trimethylsilyl)-amide (8.69 mL, 8.69 mmol) is added. The reaction mixture is stirred at −78° C. for 10 minutes and then stirred at 0° C. for 1 hour. Acetic acid (12.0 mL) is added, followed by ammonium acetate (2.98 g, 38.65 mmol). The resultant reaction mixture is heated at 75° C. for 1 hour. Tetrahydrofuran is evaporated in vacuo. The residue is dissolved in CHCl₃, washed with NH₄OH, dried over Na₂SO₄, filtered, and concentrated to provide crude residue. Column chromatography of the crude residue in MeOH/CHCl₃ provides 602 mg (57%) of a yellow solid, characterized as tert-butyl (3-{[5-(4-amino-8,9-dimethoxybenzo[c][2,7]naphthyridin-2-yl)pyridin-3-yl]oxy}pyridin-2-yl)carbamate: MS 541.3 (M+H)⁺¹.

### Example 171

### Synthesis of Compound Ib-265b

### (2-{5-[(2-aminopyridin-3-yl)oxy]pyridin-3-yl}-8,9-dimethoxybenzo[c][2,7]naphthyridin-4-amine)

A solution of tert-butyl (3-{[5-(4-amino-8,9-dimethoxybenzo[c][2,7]naphthyridin-2-yl)pyridin-3-yl]oxy}pyridin-2-yl)carbamate (170 mg, 0.34 mmol) in EtOAc (100 mL) is treated with concentrated HCl (8 mL). The reaction mixture is stirred at room temperature for 40 minutes. 100 mL of EtOAc is added and the reaction mixture is stirred at room temperature for 7 hours. The solid is filtered and washed with EtOAc and ether to provide 204 mg (37.1%) of a yellow solid, characterized as 2-{5-[(2-aminopyridin-3-yl)oxy]pyridin-3-yl}-8,9-dimethoxybenzo[c][2,7]naphthyridin-4-amine: MS: 441.3 (M+H)⁺¹.

### Example 172

### Synthesis of Compound Ib-266

### (2-(5-(Allyloxy)pyridin-3-yl)-8,9-dimethoxybenzo[c][2,7]naphthyridin-4-amine)

To a suspension of 5-(4-amino-8,9-dimethoxybenzo[c][2,7]naphthyridin-2-yl)pyridin-3-ol tetrahydroacetate (170.5 mg, 0.32 mmol) and cesium carbonate (1.039 g, 3.19 mmol), allyl bromide (0.27 ml, 386 mg, 3.2 mmol) is added and the mixture is stirred at room temperature for 1 hour. The solvents are reduced and water is added. The resultant precipitate is washed with water and hexanes and further purified by flash column chromatography (Si, CHCl₃/MeOH 10:1) to provide 2-(5-(allyloxy)pyridin-3-yl)-8,9-dimethoxybenzo[c][2,7]naphthyridin-4-amine (68.3 mg, 55%). MS: 507 (M+H)⁺¹.

Using the methods described above, 2-{[5-(4-amino-8,9-dimethoxybenzo[c][2,7]naphthyridin-2-yl)pyridin-3-yl]oxy}-1-phenylethanone can be prepared. MS: 467 (M+H)⁺¹.

### Example 173

### Synthesis of Compounds Ib-268 to Ib-270

### (5-(4-Amino-8,9-dimethoxybenzo[c][2,7]naphthyridin-2-yl)-N-butylnicotinimidamide)

To a solution of 5-(4-amino-8,9-dimethoxybenzo[c][2,7]naphthyridin-2-yl)nicotinonitrile (50 mg, 0.14 mmol) in 5 ml of anhydrous ethanol, propylamine (20 equivalents) is added and the mixture is heated to 80° C. for 24 hours. The solvent is removed and the residue is purified by flash column chromatography (Si—CHCl3/MeOH 10:1, 1% NEt₃) to provide 5-(4-Amino-8,9-dimethoxybenzo[c][2,7]naphthyridin-2-yl)-N-butylnicotinimidamide in sufficient purity (25.6 mg, 43%). MS: 430.2 (M+H)⁺¹.

In a similar manner, the following compounds are prepared:

### 5-(4-Amino-8,9-dimethoxybenzo[c][2,7]naphthyridin-2-yl)-N-isopropylnicotin-imidamide

MS: 417.2 (M+H)⁺¹.

### 5-(4-Amino-8,9-dimethoxybenzo[c][2,7]naphthyridin-2-yl)-N-sec-butylnicotin-imidamide

MS: 431.2 (M+H)⁺¹.

### Example 174

### Synthesis of Compound Ib-271

### ((S)-2-(5-(2-amino-4-(thiazol-2-yl)butoxy)pyridin-3-yl)-8,9-dimethoxybenzo[c][2,7]naphthyridin-4-amine)

Step A: To a solution of 2-methylthiazole (2.6294 g, 26.6 mmol) in 30 ml anhydrous THF, 1.6 M solution of n-BuLi in hexanes (16.6 ml, 26.56 mmol) at −78° C. is added. The mixture is stirred at this temperature for 10 minutes, followed by dropwise addition of (S)-2-(5-((1-(diphenylphosphoryl)aziridin-2-yl)methoxy)pyridin-3-yl)-8,9-dimethoxybenzo[c][2,7]naphthyridin-4-amine (1 g, 1.66 mmol) as a solution in 40 ml anhydrous THF at −78° C. The mixture is allowed to slowly warm to room temperature and is then further stirred for 12 hours. The reaction mixture is then quenched with saturated ammonium chloride solution and extracted using a mixture of THF/EtAc (1:1) and brine. The combined organic layers are washed with brine and dried over sodium sulfate. Flash-column chromatography (Si; CHCl₃/MeOH 10:1, R_{f}=0.45) provided (S)—N-(1-(5-(4-amino-8,9-dimethoxybenzo[c][2,7]naphthyridin-2-yl)pyridin-3-yloxy)-4-(thiazol-2-yl)butan-2-yl)-P,P-diphenylphosphinic amide as an orange solid (284.2 mg, 24.4%). (M+H)⁺¹=703.2.

Step B: To a solution of (S)—N-(1-(5-(4-amino-8,9-dimethoxybenzo[c][2,7]naphthyridin-2-yl)pyridin-3-yloxy)-4-(thiazol-2-yl)butan-2-yl)-P,P-diphenylphosphinic amide (131 mg, 0.19 mmol) in 8 ml anhydrous THF, a 1.2 N aqueous HCl solution (2 ml) is added and the solution is warmed to 40° C. for 23 hours. The solvent is removed and the residue is dried in vacuo for 5 hours. The residue is re-dissolved in 25 ml of MeOH and polymer-bound carbonate (810 mg, 2.43 mmol) is added to the solution. The reaction is stirred for 90 mins. and the mixture is filtered and the solvent is removed. The residue is crystallized from boiling CHCl₃ to provide (S)-2-(5-(2-amino-4-(thiazol-2-yl)butoxy)pyridin-3-yl)-8,9-dimethoxybenzo[c][2,7]-naphthyridin-4-amine as a brown solid (40 mg, 42.7%). (M+H)⁺¹=503.2.

To a solution of 2-methylthiazole (2.6294 g, 26.6 mmol) in 30 ml of anhydrous THF, 1.6 M solution of n-BuLi in hexanes (16.6 ml, 26.56 mmol) at −78° C. is added. The mixture is stirred at this temperature for 10 mins. followed by the dropwise addition of (S)-2-(5-((1-(diphenylphosphoryl)aziridin-2-yl)methoxy)pyridin-3-yl)-8,9-dimethoxybenzo[c][2,7]naphthyridin-4-amine (1 g, 1.66 mmol) as a solution in 40 ml anhydrous THF at −78° C. The mixture is allowed to slowly warm to room temperature and is then further stirred for 12 hours. The reaction mixture is quenched with saturated ammonium chloride solution and extracted using a mixture of THF/EtAc (1:1) and brine. The combined organic layers are washed with brine and dried over sodium sulfate. Flash-column chromatography (Si; CHCl₃/MeOH 10:1, R_{f}=0.45) provides (S)—N-(1-(5-(4-amino-8,9-dimethoxybenzo[c][2,7]naphthyridin-2-yl)pyridin-3-yloxy)-4-(thiazol-2-yl)butan-2-yl)-P,P-diphenylphosphinic amide as an orange solid (284.2 mg, 24.4%). (M+H)⁺¹=703.2.

Step C: To a solution of (S)—N-(1-(5-(4-amino-8,9-dimethoxybenzo[c][2,7]naphthyridin-2-yl)pyridin-3-yloxy)-4-(thiazol-2-yl)butan-2-yl)-P,P-diphenylphosphinic amide (131 mg, 0.19 mmol) in 8 ml of anhydrous THF 1.2 N aqueous HCl solution (2 ml) is added and the resultant solution is warmed to 40° C. for 23 hours. The solvent is removed and the residue is dried in vacuo for 5 hours. The residue is re-dissolved in 25 ml MeOH and polymer-bound carbonate (810 mg, 2.43 mmol) is added to the solution, which is then stirred for 90 mins., filtered, and the solvent is removed. The residue is crystallized from boiling CHCl₃ to provide (S)-2-(5-(2-amino-4-(thiazol-2-yl)butoxy)pyridin-3-yl)-8,9-dimethoxybenzo[c][2,7]-naphthyridin-4-amine as a brown solid (40 mg, 42.7%). (M+H)⁺¹=503.2.

### Example 175

### PDK-1 Kinase Inhibition by Benzo[c][2,7]naphthyridine Derivatives

The ability of illustrative Benzo[c][2,7]naphthyridine Derivatives to inhibit PDK-1 in vitro can be determined using an ELISA assay as described in Kobayashi et al., Biochem. J. 339:319-328 (1999) and Park et al., EMBO J. 18:3024-3033 (1999).

In an in vitro assay, His₆-tagged SGK1 with S422D mutation (formerly Upstate Biotech, now Millipore) on Thr256 is allowed to be phosphorylated by PDK-1 in the presence of a Benzo[c][2,7]naphthyridine Derivative. After 20 minutes, the reaction is quenched via the addition of urea. The His₆-tagged SGK1 is then bound to 96 well Ni-NTA HisSorb plates (Quiagen, Inc., Valencia, Calif.) and phosphorylated SGK1 is quantitatively detected using rabbit derived anti-phospho SGK antibody (Cell Signaling Technology, Danvers, Mass.) as the primary antibody and horseradish peroxidase (HRP)—coupled anti-rabbit antibody (formerly Zymed, now Invitrogen, Carlsbad, Calif.) as the secondary antibody. HRP is then detected using a chemiluminescent substrate (Pierce Chemical Co., Woburn, Mass.).

IC₅₀ values of illustrative Benzo[c][2,7]naphthyridine Derivatives, shown in Table 11, are calculated using LSW Data Analysis package add-in for Excel (Microsoft).

TABLE 11

| Compound | Average IC₅₀ [nm] |
|---|---|
| I-1 | 80 |
| I-2 | >100 |
| I-3 | 8,830 |
| I-4 | >20,000 |
| I-5 | 823 |
| Ia-1 | >100 |
| Ia-2 | 43 |
| Ia-3 | 104 |
| Ia-4 | 27 |
| Ia-5 | 36 |
| Ia-6 | >5,000 |
| Ia-7 | >5,000 |
| Ia-9 | 2,121 |
| Ia-10 | 1,146 |
| Ia-11 | 12 |
| Ia-12 | 508 |
| Ia-13 | 79 |
| Ia-14 | 356 |
| Ia-15 | 498 |
| Ia-16 | 170 |
| Ia-17 | 105 |
| Ia-18 | >3,000 |
| Ia-19 | >5,000 |
| Ia-20 | 893 |
| Ia-21 | 65 |
| Ia-22 | 6,923 |
| Ia-23 | 146 |
| Ia-24 | 97 |
| Ia-25 | 116 |
| Ia-26 | 549 |
| Ia-27 | 617 |
| Ia-28 | 46 |
| Ia-29 | 385 |
| Ia-30 | 179 |
| Ia-31 | 120 |
| Ia-32 | 754 |
| Ia-33 | 90 |
| Ia-34 | 193 |
| Ia-35 | 493 |
| Ia-36 | 531 |
| Ia-37 | 527 |
| Ia-38 | 69.5 |
| Ia-39 | 59 |
| Ia-40 | 44.5 |
| Ia-41 | 2,034 |
| Ia-42 | 108 |
| Ia-43 | 2,448 |
| Ia-44 | 2,679 |
| Ia-45 | 1,430 |
| Ia-46 | 74 |
| Ia-47 | >20,000 |
| Ia-48 | 70 |
| Ia-49 | >20,000 |
| Ia-50 | 2,435 |
| Ia-51 | 3,724 |
| Ia-52 | 3278 |
| Ia-53 | 2,064 |
| Ia-54 | 2,259 |
| Ia-55 | 7,490 |
| Ia-56 | 2,361 |
| Ia-57 | 6,417 |
| Ia-58 | 1,277 |
| Ia-60 | 739 |
| Ia-61 | 2,619 |
| Ia-62 | 1,863 |
| Ib-76 | 524 |
| Ib-4 | 29 |
| Ib-5 | 26 |
| Ib-6 | 4 |
| Ib-7 | 16 |
| Ib-8 | 5 |
| Ib-9 | 20 |
| Ib-10 | 6 |
| Ib-11 | 5 |
| Ib-13 | 6 |
| Ib-14 | 6 |
| Ib-15 | 66 |
| Ib-16 | 38 |
| Ib-18 | 2 |
| Ib-19 | 81 |
| Ib-20 | 66 |
| Ib-21 | 7 |
| Ib-22 | >100 |
| Ib-23 | >100 |
| Ib-24 | 13 |
| Ib-25 | >100 |
| Ib-26 | >100 |
| Ib-27 | 6 |
| Ib-28 | 21 |
| Ib-29 | 8 |
| Ib-30 | 2 |
| Ib-31 | 6 |
| Ib-32 | 850 |
| Ib-33 | 8 |
| Ib-34 | 6 |
| Ib-35 | 206 |
| Ib-36 | 4 |
| Ib-37 | 18 |
| Ib-38 | 9 |
| Ib-39 | 84 |
| Ib-40 | 117 |
| Ib-41 | 3 |
| Ib-44 | 6 |
| Ib-45 | 25 |
| Ib-46 | 8 |
| Ib-47 | 15 |
| Ib-48 | 34 |
| Ib-49 | 36 |
| Ib-50 | 61 |
| Ib-51 | 5 |
| Ib-52 | 8 |
| Ib-53 | 17 |
| Ib-54 | 212 |
| Ib-55 | 15 |
| Ib-56 | 17 |
| Ib-57 | 57 |
| Ib-58 | 66 |
| Ib-59 | 64 |
| Ib-60 | 532 |
| Ib-61 | 43 |
| Ib-62 | 10 |
| Ib-63 | 66 |
| Ib-64 | 3,584 |
| Ib-65 | 4,607 |
| Ib-66 | 4,971 |
| Ib-67 | 9,643 |
| Ib-68 | 4,125 |
| Ib-69 | 205 |
| Ib-70 | 100 |
| Ib-71 | 100 |
| Ib-73 | 46.5 |
| Ib-74 | 100 |
| Ib-77 | 21 |
| Ib-79 | 11 |
| Ib-80 | 3 |
| Ib-81 | 22 |
| Ib-82 | 92 |
| Ib-83 | 5 |
| Ib-84 | 5 |
| Ib-85 | 5 |
| Ib-86 | 77 |
| Ib-87 | 12 |
| Ib-89 | 61 |
| Ib-90 | 7 |
| Ib-91 | 40 |
| Ib-92 | 36 |
| Ib-93 | 6 |
| Ib-94 | 4 |
| Ib-95 | 53 |
| Ib-96 | 26 |
| Ib-97 | 24 |
| Ib-98 | 10 |
| Ib-98 + Ib-102 | 13 |
| Ib-100 | 63 |
| Ib-101 | 92 |
| Ib-102 | 38 |
| Ib-103 | 20 |
| Ib-104 | 25 |
| Ib-105 | 41 |
| Ib-106 | 32 |
| Ib-107 | 42 |
| Ib-108 | 7 |
| Ib-109 | 14 |
| Ib-110 | 14 |
| Ib-111 | 81 |
| Ib-112 | 40 |
| Ib-113 | 20 |
| Ib-114 | 82 |
| Ib-141 | 10 |
| Ib-142 | 10 |
| Ib-143 | 7 |
| Ib-144 | 64 |
| Ib-145 | 4 |
| Ib-146 | 2 |
| Ib-147 | 10 |
| Ib-148 | 9 |
| Ib-149 | 7 |
| Ib-150 | 6 |
| Ib-151 | 10 |
| Ib-152 | 9 |
| Ib-153 | 4 |
| Ib-154 | 4 |
| Ib-155 | 36 |
| Ib-156 | 3 |
| Ib-158 | 55 |
| Ib-159 | 3 |
| Ib-160 | 2 |
| Ib-161 | 3 |
| Ib-162 | 4 |
| Ib-163 | 12 |
| Ib-164 | 10 |
| Ib-166 | 3 |
| Ib-167 | 5 |
| Ib-168 | 100 |
| Ib-169 | 60 |
| Ib-170 | 115 |
| Ib-171 | 153 |
| Ib-172 | 151 |
| Ib-176 | 434 |
| Ib-177 | 383 |
| Ib-178 | 105 |
| Ib-179 | >700 |
| Ib-180 | 600 |
| Ib-181 | 73.7 |
| Ib-182 | 50.7 |
| Ib-183 | 505 |
| Ib-184b | 74 |
| Ib-185 | 37 |
| Ib-186 | 164 |
| Ib-187 | 110 |
| Ib-188 | 39 |
| Ib-189 | 27 |
| Ib-190 | 197 |
| Ib-191 | 81 |
| Ib-192 | 81 |
| Ib-193 | 506 |
| Ib-194 | 142 |
| Ib-195 | 67 |
| Ib-196 | 47 |
| Ib-197 | 24 |
| Ib-198 | 91 |
| Ib-199 | 476 |
| Ib-200 | 158 |
| Ib-201 | 477 |
| Ib-202 | 1000 |
| Ib-203 | 283 |
| Ib-204 | 51 |
| Ib-205 | 183.5 |
| Ib-206 | 183.5 |
| Ib-210 | >700 |
| Ib-233 | 100 |
| Ib-234 | 1000 |
| Ib-235 | 100 |
| Ib-236 | 100 |
| Ib-237 | 50 |
| Ib-238 | 1000 |
| Ib-239 | 1000 |
| Ib-240 | 998 |
| Ib-241 | 1000 |
| Ib-242 | 147 |
| Ib-243 | 658 |
| Ib-244 | 36 |
| Ib-245 | 100 |
| Ib-246 | 100 |
| Ib-247 | 1000 |
| Ib-248 | 1000 |
| Ib-249 | 205 |
| Ib-250 | 34 |
| Ib-251 | 32 |
| Ib-252 | 53 |
| Ib-253 | 100 |
| Ib-254 | 100 |
| Ib-255 | 100 |
| Ib-256 | 27 |
| Ib-257 | 29 |
| Ib-258 | 27 |
| Ib-266 | 18 |
| Ib-267 | 798 |
| Ib-268 | 1000 |
| Ib-269 | 606 |
| Ib-270 | 606 |

The results in Table 11 demonstrate that illustrative Benzo[c][2,7]naphthyridine Derivatives are effective at inhibiting PDK-1. Accordingly, a Benzo[c][2,7]naphthyridine is useful for treating or preventing a proliferative disorder, such as cancer, as well as for modulating PDK-1 activity.

### Example 176

### PKA, Akt1, S6K and PKCθ Kinase Inhibition by Benzo[c][2,7]naphthyridine Derivatives

The ability of illustrative Benzo[c][2,7]naphthyridine Derivatives to inhibit PKA, Akt1, S6K in vitro can be determined using a Fluorescence Resonance Energy Transfer (FRET) assay, such as a Z′-LYTE™ Kinase Assay Platform, commercially available from Invitrogen (c.f. Rodems et al., ASSAY Drug Devel. Technol. 1:9-19 (2002) and Zhang et al., J. Biomol. Screen. 4:67-73 (1999)). Z′-Lyte™ assay is an in vitro FRET-based assay that utilizes differential sensitivity of phosphorylated and non-phosphorylated peptides to proteolytic cleavage. The peptide substrate (also available from Invitrogen for each of PKA, Akt1 and S6K) is labeled with two different fluorophores, a fluorescence donor at one end of the peptide and a fluorescence acceptor at the other end of the peptide. In the primary kinase reaction, the kinase phosphorylates the peptide substrate in the presence of ATP. In the secondary reaction, a site-specific protease recognizes and cleaves non-phosphorylated peptides, but not phosphorylated peptides. Cleavage disrupts FRET between the donor and acceptor fluorophores on the peptide, whereas uncleaved phosphorylated peptides maintain FRET. Based on a formula that uses the ratio of donor emission (at 460 nm) to acceptor emission (at 535 nM) after excitation of the donor fluorophore at 400 nm, the extent of phosphorylation of the peptide substrates is calculated. IC₅₀ values of illustrative Benzo[c][2,7]naphthyridine Derivatives, shown in Table 12, are calculated using LSW Data Analysis package add-in for Excel (Microsoft).

The ability of illustrative Benzo[c][2,7]naphthyridine Derivatives to inhibit PKCθ can be determined using an IMAP Assay (Molecular Devices Corporation, Sunnyvale, Calif.). The materials used include the following: human PKCθ full length enzyme (Panvera, Madison, Wis., now Invitrogen, Carlsbad, Calif., Cat. No. P2996); substrate peptide: 5FAM-RFARKGSLRQKNV-OH (Molecular Devices, Cat. No. RP7032); ATP (Sigma-Aldrich, St. Louis, Mo., Sigma Cat. No. A2383); DTT (e.g., Pierce, now Fisher Scientific, Waltham, Mass., Pierce Cat. No. 20291); 5× kinase reaction buffer (Molecular Devices, Cat. No. R7209); 5× binding buffer A (Molecular Devices, Cat. No. R7282), 5× binding buffer B (Molecular Devices, Cat. No. R7209); IMAP Beads (Molecular Devices, Cat. No. R7284); and 384-well plates (Corning Costar, Acton, Mass., Cat. No. 3710). The reaction buffer is prepared by diluting the 5× stock reaction buffer and adding DTT to obtain a concentration of 3.0 mM. The binding buffer is prepared by diluting the 5× binding buffer A. A master mix solution is prepared using a 90% dilution of the reaction buffer containing 2×ATP (12 μM) and 2× peptide (200 nM). Compounds are diluted in DMSO to 20× of the maximum concentration for the IC50 measurement. 27 μl of the master mix solution for each IC50 curve is added to the first column in a 384-well plate and 3 μl of 20× compound in DMSO is added to each well. The final concentration of compound is 2× and 10% DMSO. DMSO is added to the rest of the master mix to increase the concentration to 10%. 10 μl of the master mix containing 10% DMSO is added to the rest of the wells on the plate except the 2nd column. 20 μl is transferred from the first column to the 2nd column. The compounds are serially diluted in 2:1 ratio starting from the 2nd column. A 2× (2 nM) PKCθ solution is made in the reaction buffer. 10 μl of the PKCθ solution is added to every well to achieve these final concentrations: PKCθ—1 nM; ATP—6 μM; peptide—100 nM; DMSO—5%. Samples are incubated for 25 minutes at room temperature. The binding reagent is prepared by diluting the beads in 1× binding buffer to 800:1. 50 μl of the binding reagent is added to every well and incubated for 20 minutes. Fluorescence polarization is measured using EnVision™ 2100 multilabel reader (PerkinElmer Life Sciences, Wellesley, Mass.). Wells with no ATPs and wells with no enzymes are used as controls. IC₅₀ values of illustrative Benzo[c][2,7]naphthyridine Derivatives are shown in Table 12.

TABLE 12

Assay conditions

| | 100 μM ATP | 20 μM ATP | 100 μM ATP | 100 μM ATP | 6 μM ATP |
|---|---|---|---|---|---|
| Compound | PKA IC₅₀ [nm] | PKA IC₅₀ [nm] | Akt1 IC₅₀ [nm] | S6K IC₅₀ [nm] | PKCθ IC₅₀ [nm] |
| I-5 | >2,000 | >4,000 | | >2,000 | |
| Ia-1 | >2,000 | | | >2,000 | |
| Ia-11 | 390 | 38 | 1 | 34 | 17 |
| Ia-12 | | >10,000 | 225 | | 255 |
| Ia-13 | >2,000 | >10,000 | 70 | 656 | 121 |
| Ia-14 | | >10,000 | 350 | | |
| Ia-15 | | >10,000 | 380 | | |
| Ia-16 | | >10,000 | 380 | | |
| Ia-17 | >2,000 | >10,000 | 280 | >2,000 | |
| Ia-18 | | >10,000 | >10,000 | | |
| Ia-19 | | >10,000 | 10,000 | | |
| Ia-2 | >2,000 | | | 542 | |
| Ia-20 | | >20,000 | 300 | | |
| Ia-21 | >2,000 | >20,000 | 300 | 1,564 | |
| Ia-23 | | >20,000 | 160 | | |
| Ia-24 | >2,000 | 240 | 75 | 1,034 | |
| Ia-25 | | 1,250 | 230 | | |
| Ia-27 | | >20,000 | 16,000 | | |
| Ia-28 | >2,000 | 20,000 | 360 | 1,094 | 135 |
| Ia-29 | | >5,000 | >5,000 | | |
| Ia-3 | 911 | 300 | | 1,202 | |
| Ia-30 | | >20,000 | 15,000 | | |
| Ia-31 | >2,000 | >20,000 | 170 | 422 | |
| Ia-32 | | >20,000 | 250 | | |
| Ia-33 | >2,000 | 2,500 | 2,000 | >2,000 | |
| Ia-34 | | >20,000 | 180 | | |
| Ia-35 | | 6,000 | 2,900 | | |
| Ia-36 | | 4,000 | 2,000 | | |
| Ia-4 | 70 | <32 | | 89 | |
| Ia-40 | | >20,000 | 1,000 | | 525 |
| Ia-41 | | >20,000 | 18,000 | | |
| Ia-42 | | >20,000 | 350 | | |
| Ia-46 | | >20,000 | 200 | | |
| Ia-48 | >2,000 | >20,000 | 200 | 1,385 | 116 |
| Ia-5 | 872 | 340 | 500 | 520 | |
| Ia-6 | | >20,000 | >20,000 | | |
| Ia-7 | | >20,000 | >20,000 | | |
| Ia-9 | | >10,000 | 4,000 | | |
| Ib-23 | >2,000 | | | >2,000 | |
| Ib-24 | 198-219 | | | 169-194 | |
| Ib-25 | >2,000 | | | >2,000 | |
| Ib-26 | >2,000 | | | >2,000 | |
| Ib-27 | 110-217 | | | 37-43 | |
| Ib-28 | 889 | | | 763 | |
| Ib-29 | 78 | | | 76 | |
| Ib-30 | 377 | | | 97 | |
| Ib-31 | 711 | | | 235 | |
| Ib-32 | >2,000 | | | >2,000 | |
| Ib-33 | 368 | | | 138 | |
| Ib-34 | 116 | <32 | | 61-130 | |
| Ib-35 | >2,000 | >4,000 | | 1,736->2,000 | |
| Ib-36 | 223 | <32 | | 30-133 | |
| Ib-37 | >2,000 | 850 | | 523-821 | |
| Ib-38 | 1,036 | 210 | | 213-365 | |
| Ib-39 | >2,000 | >4,000 | | 1,703->2,000 | |
| Ib-40 | >2,000 | >4,000 | | >2,000 | |
| Ib-41 | 183 | <32 | | 21-102 | |
| Ib-44 | 433 | <32 | | 19 | |
| Ib-45 | >2,000 | 650 | | 725 | |
| Ib-46 | 179 | <32 | | 65 | |
| Ib-47 | 251 | 25 | 12 | 93 | |
| Ib-48 | >2,000 | 2,700 | 400 | 405 | |
| Ib-49 | >2,000 | 2,500 | 310 | 260 | |
| Ib-50 | >2,000 | 2,100 | 160 | 220 | |
| Ib-51 | 712 | 340 | <160 | 60 | |
| Ib-52 | 793 | 45 | 6.4 | 106 | |
| Ib-53 | >2,000 | 400 | 15 | 86 | |
| Ib-54 | | >10,000 | 3,000 | | |
| Ib-55 | 1,324 | <80 | 18 | 254 | 3 |
| Ib-56 | 259 | <160 | 3 | 88 | 4 |
| Ib-57 | >2,000 | >20,000 | 300 | >2,000 | 332 |
| Ib-58 | 1,528 | 140 | 60 | 355 | 249 |
| Ib-59 | >2,000 | 2,000 | 290 | 1,244 | 592 |
| Ib-61 | >2,000 | 1,500 | 250 | 7-82 | |
| Ib-62 | 146-497 | 23 | <160 | 98-140 | 29 |
| Ib-63 | >2,000 | >20,000 | 400 | 1,274 | |
| Ib-69 | | >20,000 | 2,800 | | |
| Ib-74 | | >20,000 | 180 | | |

The results in Table 12 demonstrate that the illustrative Benzo[c][2,7]naphthyridine Derivatives are effective at inhibiting PKA, Akt1, S6K or PKCθ. Accordingly, a Benzo[c][2,7]naphthyridine is useful for treating or preventing a proliferative disorder, such as cancer or an autoimmune disease, as well as for modulating PKA-1, Akt1, S6K or PKCθ activity.

The present invention is not to be limited in scope by the specific embodiments disclosed in the examples, which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosures of which have been incorporated herein in their entirety.

What is claimed is:

1. A compound having the formula 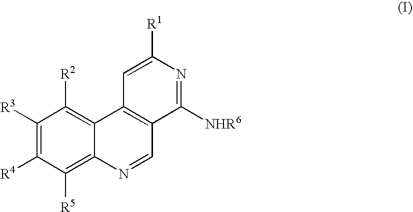
or a pharmaceutically acceptable salt thereof,
wherein
R¹ is —H, -halo, —OH, —NH₂, —CN, —NO₂, —C₁-C₆ alkyl, —O—C₁-C₆ alkyl, —S—C₁-C₆ alkyl, —NH—C₁-C₆ alkyl, —N(C₁-C₆ alkyl)₂, -phenyl, -benzyl, —O-phenyl, —O-benzyl, —NH-phenyl, —NH-benzyl, NR¹²R¹³, —OR¹³, —SR¹³, —N(R¹²)(C(R¹²)₂)ₙ—R¹³, —O—(C(R¹²)₂)ₙ—R¹³, —S—(C(R¹²)₂)ₙ—R¹³, —(C(R¹²)₂)ₙ—R¹³, —(C(R¹²)₂)ₙ—R¹³, —(C(R¹²)₂)ₙ—N(R¹²)(C(R¹²)₂)—R¹³, —(C(R¹²)₂)—N(R¹²)(C(R¹²)₂)ₙ—R¹⁴, —(C(R¹²)₂)ₙ—O—(C(R¹²)₂)ₙ—R¹³, —(C(R¹²)₂)ₙ—O—(C(R¹²)₂)ₙ—R¹⁴, —(C(R¹²)₂)ₙ—S—(C(R¹²)₂)ₙ—R¹³, —(C(R¹²)₂)ₙ—S—(C(R¹²)₂)ₙ—R¹⁴, R¹³, or R¹⁴,
wherein each C₁-C₆ alkyl, -phenyl or -benzyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl, benzoyl, —S(O)₂NH₂, —S(O)₂NH—C₁-C₆ alkyl, or —S(O)₂N(C₁-C₆ alkyl)₂,
wherein n=1-4 at each occurrence, with the proviso that when the carbon chain of length n is between two heteroatoms, n=2-4;
R² and R⁵ are each independently —H, —OH, -halo, —CN, —N₃, —NH₂, —NH—C₁-C₄ alkyl, —N(C₁-C₂ alkyl)₂, —C₁-C₄ alkyl, —O—C₁-C₄ alkyl, or —S—C₁-C₄ alkyl,
wherein each C₁-C₂ alkyl or C₁-C₄ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R³ and R⁴ are each independently —H, —OH, -halo, —C₁-C₆ alkyl, —C₂-C₆ alkenyl, —C₂-C₆ alkynyl, —O—C₁-C₆ alkyl, —O—C₂-C₆ alkenyl, —O—C₂-C₆ alkynyl, —C(O)O—C₁-C₆ alkyl, —C(O)NH—C₁-C₆ alkyl, —C(O)N(C₁-C₆ alkyl)₂, —C₁-C₆-alkylene-O—C₁-C₆ alkyl, —O—C₁-C₆-alkylene-O—C₁-C₆ alkyl, —C₁-C₆-alkylene-C(O)O—C₁-C₆ alkyl, —C₁-C₆ alkylene-C(O)NH—C₁-C₆ alkyl, —OC(O)—C₁-C₆ alkyl, —OC(O)—C₂-C₆ alkenyl, —OC(O)—C₂-C₆ alkynyl, —C₁-C₆ alkylene-OC(O)—C₁-C₆ alkyl, —C₁-C₆ alkylene-OC(O)—C₂-C₆ alkenyl, —C₁-C₆ alkylene-OC(O)—C₂-C₆ alkynyl, —S—C₁-C₆ alkyl, —S(O)—C₁-C₆ alkyl, —S(O)₂—C₁-C₆ alkyl, —S(O)₂NH—C₁-C₆ alkyl, —S(O)₂NH—C₂-C₆ alkenyl, —S(O)₂NH—C₂-C₆ alkynyl, —C(O)-benzyl, —CN, —NO₂, —CHO, —COOH, —C(O)O—C₁-C₆ alkyl, —C(O)—C₁-C₆ alkyl, -phenyl, —O-phenyl, —S-phenyl, -benzyl, —O-benzyl, —CHO, —NHC(O)H, —NH₂, —NHOH, —NH—O—C₁-C₆ alkyl, —NH—C₁-C₆ alkyl, —N(C₁-C₆ alkyl)₂, —NHC(O)—C₁-C₆ alkyl, —N(C₁-C₆ alkyl)(C₂-C₆ alkenyl), —N(C₂-C₆ alkenyl)₂, —NH-phenyl, —NH-benzyl, —NHS(O)₂—C₁-C₆ alkyl, —NHS(O)₂-phenyl, —NHS(O)₂-benzyl, 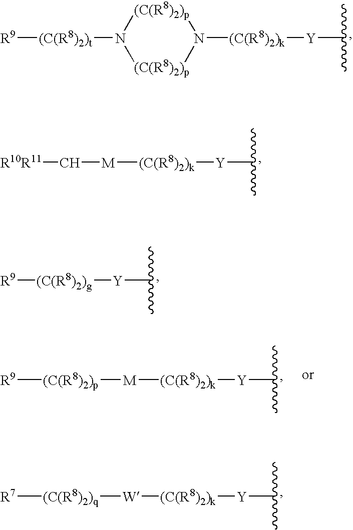
wherein
a=0-1;
g=1-6;
k=0-4;
p=2-4 at each occurrence;
q=0-4;
t=2-4;
M is a bond, —N(R⁸)—, —O—, —N((C(R⁸)₂)ₚN(R₈)₂—, —N((C(R⁸)₂)ₚOR⁸)—, —CC—, —CH═CH—, or phenylene;
W′ is a bond, —N(R⁸)—, —O—, —CC—, —CH═CH— or phenylene;
Y is —(CH₂)ₐ—, —O—, —S—, —C(O)N(R⁸)—, —C(S)N(R⁸)—, or —N(R⁸)—;
when M is phenylene then p=0-4;
when M is —O— and R⁹ is —OR⁸ then p=1-4;
when M is —CC— or —CH═CH— and Y is —S—, —O— or —N(R⁸)— then k=1-4;
when M is —CC— or —CH═CH— then p=1-4;
when W′ is —CC— or —CH═CH— and R⁷ is bonded through a heteroatom then q=1-4;
when W′ is a bond and q=0 and R⁷ is bonded through a nitrogen atom and Y is —S—, —O— or —N(R⁸)— then k=2-4;
when W′ is a bond and k=0 and R⁷ is bonded through a nitrogen atom and Y is —S—, —O— or —N(R⁸)— then q=2-4;
when W′ is not a bond, —CC—, —CH═CH—, or -phenyl- and R⁷ is bonded through a nitrogen atom then q=2-4;
when Y is —N(R⁸)— and R⁹ is —N(R⁸)₂, —N(R⁸)₃⁺ or —NR⁸(OR⁸) then g=2-6;
when Y is —N(R⁸)— and M is —N(R⁸)—, —O—, —N((C(R⁸)₂)ₚN(R⁸)₂)— or —N((C(R⁸)₂)ₚ—OR⁸)— then k=1-4;
when Y is —N(R⁸)— and W′ is —N(R⁸)— or —O— then k=2-4;
when Y is —O— and either M or W′ is —O— then k=2-4;
wherein each —C₁-C₆ alkyl, —C₁-C₆ alkylene, —C₁-C₆ alkylene-O—C₁-C₆ alkyl, —C₂-C₆ alkenyl, —C₂-C₆ alkynyl, -phenyl, -benzyl, or phenylene is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R⁶ is —H, —C₁-C₄ alkyl, —OH, a 5- or 6-membered aromatic monocyclic heterocycle, a 3- to 7-membered non-aromatic monocyclic heterocycle, a —C₁-C₃ alkylene-5- or 6-membered aromatic monocyclic heterocycle or a —C₁-C₃ alkylene-3- to 7-membered non-aromatic monocyclic heterocycle
wherein each 5- or 6-membered aromatic monocyclic heterocycle or 3- to 7-membered non-aromatic monocyclic heterocycle is independently unsubstituted or substituted with one or more of —OH, -halo, —CN, —N₃, —NH₂, and —C₁-C₄ alkyl;
R⁷ is -phenyl, a 5- or 6-membered aromatic monocyclic heterocycle, or a 3- to 7-membered non-aromatic monocyclic heterocycle, wherein, each R⁷ is optionally mono or di-substituted on a carbon atom with —OH, —R⁸, —N(R⁸)₂, —OR⁸, —(C(R⁸)₂)ᵣOR⁸, —(C(R⁸)₂)ᵣN(R⁸)₂, or a 3- to 7-membered monocyclic heterocycle,
wherein r=1-6;
each R⁷ is optionally mono or di-substituted on a saturated carbon atom with —O(C(R⁸)₂)ᵣO—,
wherein r=1-6;
each R⁷ is optionally mono-substituted on a nitrogen atom with R⁸;
R⁸ is each independently —H, —C₁-C₆ alkyl, —C₂-C₆ alkenyl, —C₂-C₆ alkynyl, —C₃-C₈ monocyclic cycloalkyl, —C(O)—C₁-C₆ alkyl, —C(O)O—C₁-C₆ alkyl, or -phenyl, each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R⁹ is -halo, —N(R⁸)₂, —OR⁸, —N(R⁸)₃⁺, or —NR⁸(OR⁸);
R¹⁰ and R¹¹ are each independently —(C(R⁸)₂)ₙN(R⁸)₂ or —(C(R⁸)₂)ₙOR⁸,
wherein s=1-4.
R¹² is each independently —H, —C₁-C₆ alkyl, —C₂-C₆ alkenyl, —C₂-C₆ alkynyl, —C₃-C₈ monocyclic cycloalkyl, —C₃-C₈ monocyclic cycloalkenyl, -phenyl, -benzyl, a 5- or 6-membered aromatic monocyclic heterocycle, or a 3- to 7-membered non-aromatic monocyclic heterocycle, each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R¹³ is a 5- or 6-membered aromatic monocyclic heterocycle, a 3- to 7-membered non-aromatic monocyclic heterocycle, a 8- to 12-membered bicyclic heterocycle, -phenyl, -benzyl, or 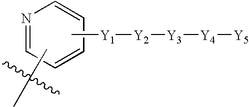
wherein:
Y₁ is a bond, —O—, —N(R¹⁶), —C((R¹⁶)₂)—, —C(R¹⁶)═C(R¹⁶), or —CC—;
Y₂ and Y₄ are each independently —(C(R¹⁶)₂)_{b}—, wherein b=0-4;
Y₃ is —C(R¹⁵R¹⁶)—;
Y₅ is absent, —H, -halo, —C₁-C₆ alkyl, —NH-halo-substituted C₁-C₄ alkyl, —O—C₁-C₆ alkyl, —O—C₁-C₆ alkyl, —OH, —NH₂, —NH—C₁-C₆ alkyl, —N(C₁-C₆ alkyl)₂, —OS(O)₂—C₁-C₆ alkyl, -phenyl, —O-phenyl, —NH-phenyl, -benzyl, —O-benzyl, —NR¹²R²⁸, —OR²⁸, —SR²⁸, —N(R¹²)(C(R¹²)₂)ₘ—R²⁸, —O(C(R¹²)₂)ₘ—R²⁸, —S(C(R¹²)₂)ₘ—R¹², —(C(R¹²)₂)ₘ—R¹², —(C(R¹²)₂)ₘ—R¹⁴, —(C(R¹²)₂)ₘ—N(R¹²)(C(R¹²)₂)ₘ—R¹², —(C(R¹²)₂)ₘ—N(R¹²)(C(R¹²)₂)ₘ—R¹⁴, —(C(R¹²)₂)ₘ—O—(C(R¹²)₂)ₘ—R²⁸, —(C(R¹²)₂)ₘ—O—(C(R¹²)₂)ₘ—R¹⁴, —(C(R¹²)₂)ₘ—S—(C(R¹²)₂)ₘ—R²⁸, —(C(R¹²)₂)ₘ—S—(C(R¹²)₂)ₘ—R¹⁴, R¹⁴, —N(C₁-C₆ alkyl)(S(O)₂R²⁷), a 5- or 6-membered aromatic monocyclic heterocycle, a 3- to 7-membered non-aromatic monocyclic heterocycle, or a 8- to 12-membered bicyclic heterocycle,
wherein m=1-4 with the proviso that when the carbon chain of length m is between two heteroatoms, m=2-4;
when Y₁ is —C((R¹⁶)₂)— the R¹⁶ substituents together with the intervening carbon atom or atoms can form a 3- to 7-membered ring;
when Y₁ is —C(R¹⁶)═C(R¹⁶)— the R¹⁶ substituents together with the intervening carbon atoms can form a 3- to 7-membered ring;
wherein each heterocycle can be substituted on a single saturated carbon atom not adjacent to a nitrogen atom with —O(CH₂)₂O—, —(CH₂)₃O— or —NH—; and
each heterocycle can optionally be benzo-fused; and
each heterocycle can optionally be mono- or di-substituted with —O—, —S—, —NH-benzoyl, R²⁸, —O—R²⁸, —O—C₁₋₄ alkylene-R²⁸, —NH—R²⁸, —NH—C₁-C₄ alkylene-R²⁸, —C(O)—R²⁸, or R¹⁸,
wherein each 5- or 6-membered aromatic monocyclic heterocycle, a 3- to 7-membered non-aromatic monocyclic heterocycle, a 8- to 12-membered bicyclic heterocycle, -phenyl, -benzyl, benzoyl or —C₁-C₆ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —O—C₂-C₆ alkenyl, —S—C₁-C₆ alkyl, —NH—C₁-C₄ alkyl, —N(C₁-C₄ alkyl)₂, -halo-substituted C₁-C₄ alkyl, -amino-substituted C₁-C₄ alkyl, hydroxy-substituted C₁-C₄ alkyl, -cyano-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, —NHC(O)—C₁-C₆ alkyl, —C(O)NH₂, —C(O)NH—C₁-C₆ alkylene-N(C₁-C₄ alkyl)₂, —C(NH)—NH—C₁-C₆ alkyl, —C(NH)—NH₂, -phenyl, -halo-substituted-phenyl, -amino-substituted phenyl, —O-phenyl, —S-phenyl, —NH-phenyl, —C(O)N(H)-phenyl, —O—C₁-C₄ alkylene-C(O)-phenyl, -benzyl, —O-benzyl, —S-benzyl, —NH-benzyl, —C(O)-benzyl, benzoyl, pyridyl or morpholinyl;
R¹⁴ is —N(R¹²)₂, —OR¹², —SR¹², —NR¹²—(C(R¹²)₂)ₙ—N(R¹²)₂, —NR¹²—(C(R¹²)₂)ₙ—OR¹², —N((C(R¹²)₂)ₙ—N(R¹²)₂)₂, —N((C(R¹²)₂)ₙ—OR¹²)₂, or —N((C(R¹²)₂)ₙ—N(R¹²)₂)(C(R¹²)₂)ₙ—OR¹²);
wherein n=1-4 at each occurrence, with the proviso that when the carbon chain of length n is between two heteroatoms, n=2-4;
R¹⁵ is —OH or —N(R¹⁷)₂;
R¹⁶ is each independently an open valence, —H, —C₁-C₈ alkyl, —C₂-C₈ alkenyl, —C₂-C₈ alkynyl, C₃-C₈ monocyclic cycloalkyl, or —C₃-C₈ monocyclic cycloalkenyl, each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R¹⁶ on Y₃ together with R¹⁶ on Y₂ together with the intervening atoms can form a 3- to 7-membered ring;
R¹⁶ on Y₃ together with R¹⁶ on Y₁ together with the intervening atoms can form a 3- to 7-membered ring;
R¹⁶ on Y₁ together with R¹⁶ on Y₂ together with the intervening atoms can form a 3- to 7-membered ring;
R¹⁶ on Y₂ together with R¹⁶ on Y₄ together with the intervening atoms can form a 3- to 7-membered ring;
R¹⁶ on Y₃ together with R¹⁶ on Y₄ together with the intervening atoms can form a 3- to 7-membered ring;
when R¹⁵ is —N(R¹⁷)₂, R¹⁷ on Y₃ together with R¹⁶ on either Y₁, Y₂, or Y₄ together with the intervening atoms can form a 3- to 7-membered ring;
R¹⁷ is each independently an open valence, —H, —C(O)NH—C₁-C₆ alkyl, —C(NH)—NH₂, —C(NH)—NH—C₁-C₆ alkyl, —C(O)—O—C₁-C₆ alkyl, —C(O)—O-phenyl, —C(O)—O—C₁-C₃ alkylene-phenyl, —C(O)—O—C₂-C₃ alkenylene-phenyl, —C(O)—O—C₂-C₃ alkynylene-phenyl, —C(O)H, —C₁-C₉ alkyl, —C₂-C₉ alkenyl, —C₂-C₉ alkynyl, —C₃-C₈ monocyclic cycloalkyl, —C₃-C₈ monocyclic cycloalkenyl, —C₇-C₉ monocyclic cycloalkynyl, -phenyl, —C₁-C₃ alkylene-phenyl, —C₂-C₃ alkenylene-phenyl, or —C₂-C₃ alkynylene-phenyl,
each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R¹⁸ is —H, -halo, —OH, —CN, —NH₂, —N₃, —NO₂, —CHO, —COOH, —C₁-C₆ alkyl, —C₂-C₆ alkenyl, —C₂-C₆ alkynyl, —NHC(O)—C₁-C₆ alkyl, —C₁-C₆ alkylene-O—C₁-C₆ alkyl, —C₁-C₆ alkylene-C(O)O—C₁-C₆ alkyl, —O—C₁-C₆ alkyl, —S—C₁-C₆ alkyl, —C(O)O—C₁-C₆ alkyl, —C(O)—C₁-C₆ alkyl, -phenyl, —O-phenyl, —S-phenyl, -benzoyl, -benzyl, —C₁-C₆ alkylene-C(O)O-phenyl, —NH—C₁-C₆ alkyl, —N(C₁-C₆ alkyl)₂, —NH-phenyl, —NH-benzyl, —NH—C(O)—C₁-C₆ alkyl, —NH—C(O)—C₂-C₆ alkenyl, —NH—C(O)—C₂-C₆ alkynyl, —C₁-C₆ alkylene-COOH, —C₁-C₆ alkylene-CHO, —C₁-C₆ alkylene-NH—C₁-C₆ alkyl, —C₁-C₆ alkylene-N(C₁-C₆ alkyl)₂, —C(O)NH—C₁-C₆ alkyl, —C(O)N(C₁-C₆)₂ alkyl, —O—C₁-C₆ alkylene-NH—C₁-C₆ alkyl, —O—C₁-C₆ alkylene-N(C₁-C₆ alkyl)₂, —OS(O)₂—C₁-C₆ alkyl, or —SH,
wherein each —C₁-C₆ alkyl, —C₁-C₆ alkylene, —C₂-C₆ alkenyl, —C₂-C₆ alkynyl, -phenyl, -benzyl, or benzoyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R²⁷ is C₁-C₆ alkyl, -phenyl, —(C((R¹²)₂)ₚ)-phenyl, a 5- or 6-membered aromatic monocyclic heterocycle, a 3- to 7-membered non-aromatic monocyclic heterocycle, or a 8- to 12-membered bicyclic heterocycle, —(C((R¹²)₂)ₚ)-5- or 6-membered aromatic monocyclic heterocycle, —(C((R¹²)₂)ₚ)-3- to 7-membered non-aromatic monocyclic heterocycle, —(C((R¹²)₂)ₚ)-8- to 12-membered bicyclic heterocycle,
wherein p=1-4 with the proviso that when the carbon chain of length p is between two heteroatoms, p=2-4; and
R²⁸ is a 5- or 6-membered aromatic monocyclic heterocycle, a 3- to 7-membered non-aromatic monocyclic heterocycle, a 8- to 12-membered bicyclic heterocycle, a C₃-C₈ monocyclic cycloalkyl, -phenyl, -benzyl,
wherein each heterocycle can be substituted on a single saturated carbon atom not adjacent to a nitrogen atom with —O(CH₂)₂O—, —(CH₂)₃O— or —NH—;
each heterocycle can optionally be benzo-fused; and
each 5- or 6-membered aromatic monocyclic heterocycle, a 3- to 7-membered non-aromatic monocyclic heterocycle, a 8- to 12-membered bicyclic heterocycle, C₃-C₈ monocyclic cycloalkyl, -phenyl, -benzyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —O—C₂-C₆ alkenyl, —S—C₁-C₆ alkyl, —NH—C₁-C₄ alkyl, —N(C₁-C₄ alkyl)₂, -halo-substituted C₁-C₄ alkyl, -amino-substituted C₁-C₄ alkyl, -hydroxy-substituted-C₁-C₄ alkyl, -cyano-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, —NHC(O)—C₁-C₆ alkyl, —C(O)NH₂, —C(O)NH—C₁-C₆ alkylene-N(C₁-C₄ alkyl)₂, —C(NH)—NH—C₁-C₆ alkyl, —C(NH)—NH₂, -phenyl, -halo-substituted phenyl, -amino-substituted phenyl, —O-phenyl, —S-phenyl, —NH-phenyl, —C(O)N(H)-phenyl, -benzyl, —O-benzyl, —S-benzyl, —NH-benzyl, —C(O)-benzyl, benzoyl, pyridyl or morpholinyl.

2. The compound of claim 1, wherein
R¹ is —H, -halo, —OH, —NH₂, —CN, —NO₂, —C₁-C₆ alkyl, —O—C₁-C₆ alkyl, —S—C₁-C₆ alkyl, —NH—C₁-C₆ alkyl, —N(C₁-C₆ alkyl)₂, -phenyl, -benzyl, —O-phenyl, —O-benzyl, —NH-phenyl, —NH-benzyl, NR¹²R¹³, —OR¹³, —SR¹³, —N(R¹²)(C(R¹²)₂)ₙ—R¹³, —O—(C(R¹²)₂)—R¹³, —S—(C(R¹²)₂)—R¹³, —(C(R¹²)₂)ₙ—R¹³, —(C(R¹²)₂)ₙ—R¹³, —(C(R¹²)₂)ₙ—N(R¹²)(C(R¹²)₂)—R¹³, —(C(R¹²)₂)—N(R¹²)(C(R¹²)₂)ₙ—R¹⁴, —(C(R¹²)₂)ₙ—O—(C(R¹²)₂)ₙ—R¹³, —(C(R¹²)₂)ₙ—O—(C(R¹²)₂)ₙ—R¹⁴, —(C(R¹²)₂)ₙ—S—(C(R¹²)₂)ₙ—R¹³, —(C(R¹²)₂)ₙ—S—(C(R¹²)₂)ₙ—R¹⁴, R¹³, or R¹⁴,
wherein each C₁-C₆ alkyl, -phenyl or -benzyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl, benzoyl, —S(O)₂NH₂, —S(O)₂NH—C₁-C₆ alkyl, or —S(O)₂N(C₁-C₆ alkyl)₂,
wherein n=1-4 at each occurrence, with the proviso that when the carbon chain of length n is between two heteroatoms, n=2-4;
R² and R⁵ are each independently —H, —OH, -halo, —CN, —N₃, —NH₂, —NH—C₁-C₄ alkyl, —N(C₁-C₂ alkyl)₂, —C₁-C₄ alkyl, —O—C₁-C₄ alkyl, or —S—C₁-C₄ alkyl,
wherein each C₁-C₂ alkyl or C₁-C₄ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R³ and R⁴ are each independently —H, —OH, -halo, —C₁-C₆ alkyl, —C₂-C₆ alkenyl, —C₂-C₆ alkynyl, —O—C₁-C₆ alkyl, —O—C₂-C₆ alkenyl, —O—C₂-C₆ alkynyl, —C(O)O—C₁-C₆ alkyl, —C(O)NH—C₁-C₆ alkyl, —C(O)N(C₁-C₆ alkyl)₂, —C₁-C₆-alkylene-O—C₁-C₆ alkyl, —O—C₁-C₆-alkylene-O—C₁-C₆ alkyl, —C₁-C₆-alkylene-C(O)O—C₁-C₆ alkyl, —C₁-C₆ alkylene-C(O)NH—C₁-C₆ alkyl, —OC(O)—C₁-C₆ alkyl, —OC(O)—C₂-C₆ alkenyl, —OC(O)—C₂-C₆ alkynyl, —C₁-C₆ alkylene-OC(O)—C₁-C₆ alkyl, —C₁-C₆ alkylene-OC(O)—C₂-C₆ alkenyl, —C₁-C₆ alkylene-OC(O)—C₂-C₆ alkynyl, —S—C₁-C₆ alkyl, —S(O)—C₁-C₆ alkyl, —S(O)₂—C₁-C₆ alkyl, —S(O)₂NH—C₁-C₆ alkyl, —S(O)₂NH—C₂-C₆ alkenyl, —S(O)₂NH—C₂-C₆ alkynyl, —C(O)-benzyl, —CN, —NO₂, —CHO, —COOH, —C(O)O—C₁-C₆ alkyl, —C(O)—C₁-C₆ alkyl, -phenyl, —O-phenyl, —S-phenyl, -benzyl, —O-benzyl, —CHO, —NHC(O)H, —NH₂, —NHOH, —NH—O—C₁-C₆ alkyl, —NH—C₁-C₆ alkyl, —N(C₁-C₆ alkyl)₂, —NHC(O)—C₁-C₆ alkyl, —N(C₁-C₆ alkyl)(C₂-C₆ alkenyl), —N(C₂-C₆ alkenyl)₂, —NH-phenyl, —NH-benzyl, —NHS(O)₂—C₁-C₆ alkyl, —NHS(O)₂-phenyl, —NHS(O)₂-benzyl, 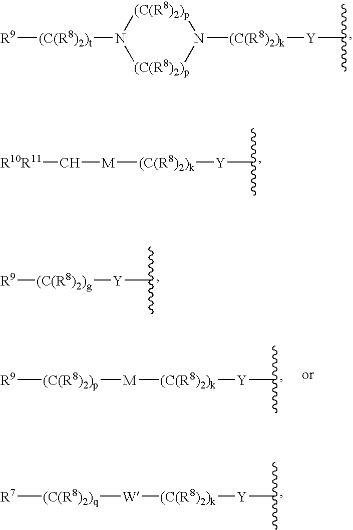
wherein
a=0-1;
g=1-6;
k=0-4;
p=2-4 at each occurrence;
q=0-4;
t=2-4;
M is a bond, —N(R⁸)—, —O—, —N((C(R⁸)₂)ₚN(R₈)₂—, —N((C(R⁸)₂)ₚOR⁸)—, —CC—, —CH═CH—, or phenylene;
W′ is a bond, —N(R⁸)—, —O—, —CC—, —CH═CH— or phenylene;
Y is —(CH₂)ₐ—, —O—, —S—, —C(O)N(R⁸)—, —C(S)N(R⁸)—, or —N(R⁸)—;
when M is phenylene then p=0-4;
when M is —O— and R⁹ is —OR⁸ then p=1-4;
when M is —CC— or —CH═CH— and Y is —S—, —O— or —N(R⁸)— then k=1-4;
when M is —CC— or —CH═CH— then p=1-4;
when W′ is —CC— or —CH═CH— and R⁷ is bonded through a heteroatom then q=1-4;
when W′ is a bond and q=0 and R⁷ is bonded through a nitrogen atom and Y is —S—, —O— or —N(R⁸)— then k=2-4;
when W′ is a bond and k=0 and R⁷ is bonded through a nitrogen atom and Y is —S—, —O— or —N(R⁸)— then q=2-4;
when W′ is not a bond, —CC—, —CH═CH—, or -phenyl- and R⁷ is bonded through a nitrogen atom then q=2-4;
when Y is —N(R⁸)— and R⁹ is —N(R⁸)₂, —N(R⁸)₃⁺ or —NR⁸(OR⁸) then g=2-6;
when Y is —N(R⁸)— and M is —N(R⁸)—, —O—, —N((C(R⁸)₂)ₚN(R⁸)₂)— or —N((C(R⁸)₂)ₚ—OR⁸)— then k=1-4;
when Y is —N(R⁸)— and W′ is —N(R⁸)— or —O— then k=2-4;
when Y is —O— and either M or W′ is —O— then k=2-4;
wherein each —C₁-C₆ alkyl, —C₁-C₆ alkylene, —C₁-C₆ alkylene-O—C₁-C₆ alkyl, —C₂-C₆ alkenyl, —C₂-C₆ alkynyl, -phenyl, -benzyl, or phenylene is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R⁶ is —H, —C₁-C₄ alkyl, —OH, a 5- or 6-membered aromatic monocyclic heterocycle, a 3- to 7-membered non-aromatic monocyclic heterocycle, a —C₁-C₃ alkylene-5- or 6-membered aromatic monocyclic heterocycle or a —C₁-C₃ alkylene-3- to 7-membered non-aromatic monocyclic heterocycle
wherein each 5- or 6-membered aromatic monocyclic heterocycle or 3- to 7-membered non-aromatic monocyclic heterocycle is independently unsubstituted or substituted with one or more of —OH, -halo, —CN, —N₃, —NH₂, and —C₁-C₄ alkyl;
R⁷ is -phenyl, a 5- or 6-membered aromatic monocyclic heterocycle, or a 3- to 7-membered non-aromatic monocyclic heterocycle, wherein, each R⁷ is optionally mono or di-substituted on a carbon atom with —OH, —R⁸, —N(R⁸)₂, —OR⁸, —(C(R⁸)₂)ᵣOR⁸, —(C(R⁸)₂)ᵣN(R⁸)₂, or a 3- to 7-membered monocyclic heterocycle,
wherein r=1-6;
each R⁷ is optionally mono or di-substituted on a saturated carbon atom with —O(C(R⁸)₂)ᵣO—,
wherein r=1-6;
each R⁷ is optionally mono-substituted on a nitrogen atom with R⁸;
R⁸ is each independently —H, —C₁-C₆ alkyl, —C₂-C₆ alkenyl, —C₂-C₆ alkynyl, —C₃-C₈ monocyclic cycloalkyl, —C(O)—C₁-C₆ alkyl, —C(O)O—C₁-C₆ alkyl, or -phenyl, each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R⁹ is -halo, —N(R⁸)₂, —OR⁸, —N(R⁸)₃⁺, or —NR⁸(OR⁸);
R¹⁰ and R¹¹ are each independently —(C(R⁸)₂)ₙN(R⁸)₂ or —(C(R⁸)₂)ₙOR⁸,
wherein s=1-4.
R¹² is each independently —H, —C₁-C₆ alkyl, —C₂-C₆ alkenyl, —C₂-C₆ alkynyl, —C₃-C₈ monocyclic cycloalkyl, —C₃-C₈ monocyclic cycloalkenyl, -phenyl, -benzyl, a 5- or 6-membered aromatic monocyclic heterocycle, or a 3- to 7-membered non-aromatic monocyclic heterocycle, each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R¹³ is a 5- or 6-membered aromatic monocyclic heterocycle, a 3- to 7-membered non-aromatic monocyclic heterocycle, a 8- to 12-membered bicyclic heterocycle, -phenyl, -benzyl, or 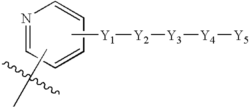
wherein:
Y₁ is a bond, —O—, —N(R¹⁶)—, —C((R¹⁶)₂)—, —C(R¹⁶)═C(R¹⁶), or —CC—;
Y₂ and Y₄ are each independently —(C(R¹⁶)₂)_{b}—, wherein b=0-4;
Y₃ is —C(R¹⁵R¹⁶)—;
Y₅ is absent, —H, -halo, —C₁-C₆ alkyl, —O—C₁-C₆ alkyl, —O—C₁-C₆ alkyl, —OH, —NH₂, —NH—C₁-C₆ alkyl, —N(C₁-C₆ alkyl)₂, —OS(O)₂—C₁-C₆ alkyl, -phenyl, —O-phenyl, —NH-phenyl, -benzyl, —O-benzyl, —NR¹²R²⁸, —OR²⁸, —SR²⁸, —N(R¹²)(C(R¹²)₂)ₘ—R¹², —O(C(R¹²)₂)ₘ—R²⁸, —S(C(R¹²)₂)ₘR²⁸, (C(R¹²)₂)ₘ—R¹⁴, —(C(R¹²)₂)ₘ—R²⁸, —(C(R¹²)₂)ₘ—N(R¹²)(C(R¹²)₂)ₘ—R²⁸, —(C(R¹²)₂)ₘ—N(R¹²)(C(R¹²)₂)ₘ—R¹⁴, —(C(R¹²)₂)ₘ—O—(C(R¹²)₂)ₘ—R¹², —(C(R¹²)₂)ₘ—O—(C(R¹²)₂)ₘ—R²⁸, —(C(R¹²)₂)ₘ—S—(C(R¹²)₂)ₘ—R¹², —(C(R¹²)₂)ₘ—S—(C(R¹²)₂)ₘ—R¹⁴, R¹⁴, —N(C₁-C₆ alkyl)(S(O)₂R²⁷), a 5- or 6-membered aromatic monocyclic heterocycle, a 3- to 7-membered non-aromatic monocyclic heterocycle, or a 8- to 12-membered bicyclic heterocycle,
wherein m=1-4 with the proviso that when the carbon chain of length m is between two heteroatoms, m=2-4;
when Y₁ is —C((R¹⁶)₂)— the R¹⁶ substituents together with the intervening carbon atom or atoms can form a 3- to 7-membered ring;
when Y₁ is —C(R¹⁶)═C(R¹⁶)— the R¹⁶ substituents together with the intervening carbon atoms can form a 3- to 7-membered ring;
wherein each heterocycle can be substituted on a single saturated carbon atom not adjacent to a nitrogen atom with —O(CH₂)₂O— or —(CH₂)₃O—; and
each heterocycle can optionally be benzo-fused; and
each heterocycle can optionally be mono- or di-substituted with —O—, —S—, —NH-benzoyl, R²⁸, —O—R²⁸ or R¹⁸,
wherein each 5- or 6-membered aromatic monocyclic heterocycle, a 3- to 7-membered non-aromatic monocyclic heterocycle, a 8- to 12-membered bicyclic heterocycle, -phenyl, -benzyl, benzoyl or —C₁-C₆ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R¹⁴ is —N(R¹²)₂, —OR¹², —SR¹², —NR¹²—(C(R¹²)₂)ₙ—N(R¹²)₂, —NR¹²—(C(R¹²)₂)ₙ—OR¹², —N((C(R¹²)₂)ₙ—N(R¹²)₂)₂, —N((C(R¹²)₂)ₙ—OR¹²)₂, or —N((C(R¹²)₂)ₙ—N(R¹²)₂)(C(R¹²)₂)ₙ—OR¹²);
wherein n=1-4 at each occurrence, with the proviso that when the carbon chain of length n is between two heteroatoms, n=2-4;
R¹⁵ is —OH or —N(R¹⁷)₂;
R¹⁶ is each independently an open valence, —H, —C₁-C₈ alkyl, —C₂-C₈ alkenyl, —C₂-C₈ alkynyl, C₃-C₈ monocyclic cycloalkyl, or —C₃-C₈ monocyclic cycloalkenyl, each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R¹⁶ on Y₃ together with R¹⁶ on Y₂ together with the intervening atoms can form a 3- to 7-membered ring;
R¹⁶ on Y₃ together with R¹⁶ on Y₁ together with the intervening atoms can form a 3- to 7-membered ring;
R¹⁶ on Y₁ together with R¹⁶ on Y₂ together with the intervening atoms can form a 3- to 7-membered ring;
R¹⁶ on Y₂ together with R¹⁶ on Y₄ together with the intervening atoms can form a 3- to 7-membered ring;
R¹⁶ on Y₃ together with R¹⁶ on Y₄ together with the intervening atoms can form a 3- to 7-membered ring;
when R¹⁵ is —N(R¹⁷)₂, R¹⁷ on Y₃ together with R¹⁶ on either Y₁, Y₂, or Y₄ together with the intervening atoms can form a 3- to 7-membered ring;
R¹⁷ is each independently an open valence, —H, —C(O)NH—C₁-C₆ alkyl, —C(O)—O—C₁-C₆ alkyl, —C(O)—O-phenyl, —C(O)—O—C₁-C₃ alkylene-phenyl, —C(O)—O—C₂-C₃ alkenylene-phenyl, —C(O)—O—C₂-C₃ alkynylene-phenyl, —C(O)H, —C₁-C₉ alkyl, —C₂-C₉ alkenyl, —C₂-C₉ alkynyl, —C₃-C₈ monocyclic cycloalkyl, —C₃-C₈ monocyclic cycloalkenyl, —C₇-C₉ monocyclic cycloalkynyl, -phenyl, —C₁-C₃ alkylene-phenyl, —C₂-C₃ alkenylene-phenyl, or —C₂-C₃ alkynylene-phenyl,
each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R¹⁸ is —H, -halo, —OH, —CN, —NH₂, —N₃, —NO₂, —CHO, —COOH, —C₁-C₆ alkyl, —C₂-C₆ alkenyl, —C₂-C₆ alkynyl, —NHC(O)—C₁-C₆ alkyl, —C₁-C₆ alkylene-O—C₁-C₆ alkyl, —C₁-C₆ alkylene-C(O)O—C₁-C₆ alkyl, —O—C₁-C₆ alkyl, —S—C₁-C₆ alkyl, —C(O)O—C₁-C₆ alkyl, —C(O)—C₁-C₆ alkyl, -phenyl, —O-phenyl, —S-phenyl, -benzoyl, -benzyl, —C₁-C₆ alkylene-C(O)O-phenyl, —NH—C₁-C₆ alkyl, —N(C₁-C₆ alkyl)₂, —NH-phenyl, —NH-benzyl, —NH—C(O)—C₁-C₆ alkyl, —NH—C(O)—C₂-C₆ alkenyl, —NH—C(O)—C₂-C₆ alkynyl, —C₁-C₆ alkylene-COOH, —C₁-C₆ alkylene-CHO, —C₁-C₆ alkylene-NH—C₁-C₆ alkyl, —C₁-C₆ alkylene-N(C₁-C₆ alkyl)₂, —C(O)NH—C₁-C₆ alkyl, —C(O)N(C₁-C₆)₂ alkyl, —O—C₁-C₆ alkylene-NH—C₁-C₆ alkyl, —O—C₁-C₆ alkylene-N(C₁-C₆ alkyl)₂, —OS(O)₂—C₁-C₆ alkyl, or —SH,
wherein each —C₁-C₆ alkyl, —C₁-C₆ alkylene, —C₂-C₆ alkenyl, —C₂-C₆ alkynyl, -phenyl, -benzyl, or benzoyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R²⁷ is C₁-C₆ alkyl, -phenyl, —(C((R¹²)₂)ₚ)-phenyl, a 5- or 6-membered aromatic monocyclic heterocycle, a 3- to 7-membered non-aromatic monocyclic heterocycle, or a 8- to 12-membered bicyclic heterocycle, —(C((R¹²)₂)ₚ)-5- or 6-membered aromatic monocyclic heterocycle, —(C((R¹²)₂)ₚ)-3- to 7-membered non-aromatic monocyclic heterocycle, —(C((R¹²)₂)ₚ)-8- to 12-membered bicyclic heterocycle,
wherein p=1-4 with the proviso that when the carbon chain of length p is between two heteroatoms, p=2-4; and
R²⁸ is a 5- or 6-membered aromatic monocyclic heterocycle, a 3- to 7-membered non-aromatic monocyclic heterocycle, a 8- to 12-membered bicyclic heterocycle, -phenyl, -benzyl,
wherein each heterocycle can be substituted on a single saturated carbon atom not adjacent to a nitrogen atom with —O(CH₂)₂O— or —(CH₂)₃O—;
each heterocycle can optionally be benzo-fused; and
each 5- or 6-membered aromatic monocyclic heterocycle, a 3- to 7-membered non-aromatic monocyclic heterocycle, a 8- to 12-membered bicyclic heterocycle, -phenyl, -benzyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl.

3. The compound of claim 1 or 2, wherein R² and R⁵ are —H, R³ and R⁴ are —O—CH₃, and R⁶ is —H.

4. The compound of claim 1 or 2, wherein R⁴ is C₁-C₆ alkylene-O—C₁-C₆ alkyl, —Y—(C(R⁸)₂)ₖ—W′—C(R⁸)₂)_{q}—R⁷, or —Y—(C(R⁸)₂)_{g}—R⁹.

5. The compound of claim 1 or 2, wherein Y is —O— or —CC—.

6. The compound of claim 1 or 2, wherein Y₁ is —O— and, R¹⁵ is —NH₂.

7. The compound of claim 1 or 2, wherein Y₅ is —N(CH₃)—CH₂-pyridyl, —OS(O)₂—C₁-C₆ alkyl, —C₁-C₆ alkyl, -phenyl, halo-substituted phenyl, or is absent.

8. The compound of claim 1 or 2, wherein R¹ is pyridyl-O-pyrrolidinyl or imidazolyl.

9. The compound of claim 1 or 2, wherein R², R⁵ and R⁶ are —H, and R¹ is

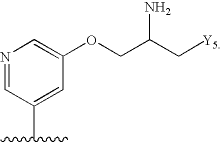

10. A composition comprising an effective amount of the compound or a pharmaceutically acceptable salt of the compound of claim 1 or 2 and a physiologically acceptable carrier or vehicle.

11. A method for treating or preventing a proliferative disorder, the method comprising administering to a subject in need thereof an effective amount of the compound or a pharmaceutically acceptable salt of the compound of claim 1 or 2.

12. The method of claim 11, wherein the proliferative disorder is cancer.

13. The method of claim 12, wherein the subject is a human.

14. A method for modulating activity of PDK-1, PKA, an Akt isoform, a PKC isoform, or S6K comprising administering to a subject in need thereof an effective amount of the compound or a pharmaceutically acceptable salt of the compound of claim 1 or 2, wherein it is known that PDK-1, PKA, the Akt isoform, the PKC isoform, or S6K activity is related to a disease or condition.

15. The method of claim 14, wherein the disease or condition is a proliferative disorder.

16. The method of claim 15, wherein the proliferative disorder is cancer.

17. The method of claim 14, wherein the Akt isoform is Akt1, Akt2, or Akt3.

18. The method of claim 14, wherein the PKC isoform is PKCθ.

19. A method for treating or preventing an autoimmune disease, the method comprising administering to a subject in need thereof an effective amount of the compound or a pharmaceutically acceptable salt of the compound of claim 1 or 2.

20. The method of claim 19, wherein the autoimmune disease is selected from the group consisting of multiple sclerosis, Addison's disease, angiitis, alopecia greata, ankylosing spondylitis, antiphospholipid syndrome, autism, autoimmune haemolytic anaemia, autoimmune hepatitis, Behcet's syndrome, Berger's disease, bullous pemphigoid, cardiomyopathy, coeliac disease, chronic fatigue syndrome, chronic inflammatory polyneuropathy, Churg-Strauss syndrome, CREST Syndrome, Crohn's disease, dermatomyositis, fibromyalgia, giant cell arteritis, Grave's disease, Guillain Barre dyndrome, Hashimoto's thyroiditis, idiopathic pulmonary fibrosis, idiopathic thrombocytopenia purpura, type 1 diabetes, lichen planus, Meniere's disease, mixed connective tissue disease, myasthenia gravis, polyarteritis nodosa, polymyalgia rheumatica, polymyositis, primary biliary cirrhosis, psoriasis, Raynaud's disease, Reiter's syndrome, relapsing polychondritis, rheumatic fever, rheumatoid arthritis, sarcoidosis, scleroderma, Sjögren's syndrome, stiff-man syndrome, systemic lupus erythematosus, ulcerative colitis, uveitis, vitiligo and Wegener's granulomatosis.

21. The process for preparing the compound of the Formula (IIe) 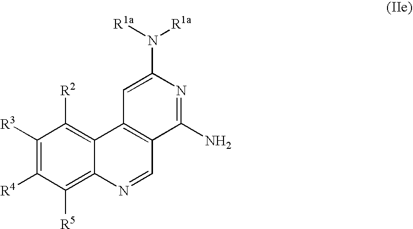
comprising reacting the compound of the Formula (IIc) 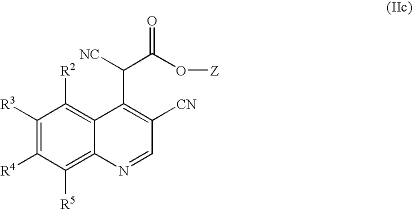
with the compound of the Formula (IId)
(R²¹)₂NH (IId)
wherein
Z is —C(C₁-C₆ alkyl)₃,
each R₂₁ is independently R¹², R¹³ or —(C(R¹²)₂)ₙ—R¹³, wherein n=1-4, with the proviso that when the carbon chain of length n is between two heteroatoms, n=2-4; or
(R²¹)₂N— is —R^{13′},
R² and R⁵ are each independently —H, —OH, -halo, —CN, —N₃, —NH₂, —NH—C₁-C₄ alkyl, —N(C₁-C₂ alkyl)₂, —C₁-C₄ alkyl, —O—C₁-C₄ alkyl, or —S—C₁-C₄ alkyl,
wherein each C₁-C₂ alkyl or C₁-C₄ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R³ and R⁴ are each independently —H, —OH, -halo, —C₁-C₆ alkyl, —C₂-C₆ alkenyl, —C₂-C₆ alkynyl, —O—C₁-C₆ alkyl, —O—C₂-C₆ alkenyl, —O—C₂-C₆ alkynyl, —C(O)O—C₁-C₆ alkyl, —C(O)NH—C₁-C₆ alkyl, —C(O)N(C₁-C₆ alkyl)₂, —C₁-C₆-alkylene-O—C₁-C₆ alkyl, —O—C₁-C₆-alkylene-O—C₁-C₆ alkyl, —C₁-C₆-alkylene-C(O)O—C₁-C₆ alkyl, —C₁-C₆ alkylene-C(O)NH—C₁-C₆ alkyl, —OC(O)—C₁-C₆ alkyl, —OC(O)—C₂-C₆ alkenyl, —OC(O)—C₂-C₆ alkynyl, —C₁-C₆ alkylene-OC(O)—C₁-C₆ alkyl, —C₁-C₆ alkylene-OC(O)—C₂-C₆ alkenyl, —C₁-C₆ alkylene-OC(O)—C₂-C₆ alkynyl, —S—C₁-C₆ alkyl, —S(O)—C₁-C₆ alkyl, —S(O)₂—C₁-C₆ alkyl, —S(O)₂NH—C₁-C₆ alkyl, —S(O)₂NH—C₂-C₆ alkenyl, —S(O)₂NH—C₂-C₆ alkynyl, —C(O)-benzyl, —CN, —NO₂, —CHO, —COOH, —C(O)O—C₁-C₆ alkyl, —C(O)—C₁-C₆ alkyl, -phenyl, —O-phenyl, —S-phenyl, -benzyl, —O-benzyl, —CHO, —NHC(O)H, —NH₂, —NHOH, —NH—O—C₁-C₆ alkyl, —NH—C₁-C₆ alkyl, —N(C₁-C₆ alkyl)₂, —NHC(O)—C₁-C₆ alkyl, —N(C₁-C₆ alkyl)(C₂-C₆ alkenyl), —N(C₂-C₆ alkenyl)₂, —NH-phenyl, —NH-benzyl, —NHS(O)₂—C₁-C₆ alkyl, —NHS(O)₂-phenyl, —NHS(O)₂-benzyl, 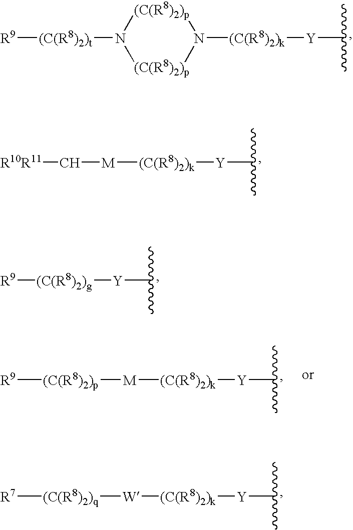
wherein
a=0-1;
g=1-6;
k=0-4;
p=2-4 at each occurrence;
q=0-4;
t=2-4;
M is a bond, —N(R⁸)—, —O—, —N((C(R⁸)₂)ₚN(R⁸)₂)—, —N((C(R⁸)₂)ₚOR⁸)—, —CC—, —CH═CH—, or phenylene;
W′ is a bond, —N(R⁸)—, —O—, —CC—, —CH═CH— or phenylene;
Y is —(CH₂)ₐ—, —O—, —S—, —C(O)N(R⁸)—, —C(S)N(R⁸)—, or —N(R⁸)—;
when M is phenylene then p=0-4;
when M is —O— and R⁹ is —OR⁸ then p=1-4;
when M is —CC— or —CH═CH— and Y is —S—, —O— or —N(R⁸)— then k=1-4;
when M is —CC— or —CH═CH— then p=1-4;
when W′ is —CC— or —CH═CH— and R⁷ is bonded through a heteroatom then q=1-4;
when W′ is a bond and q=0 and R⁷ is bonded through a nitrogen atom and Y is —S—, —O— or —N(R⁸)— then k=2-4;
when W′ is a bond and k=0 and R⁷ is bonded through a nitrogen atom and Y is —S—, —O— or —N(R⁸)— then q=2-4;
when W′ is not a bond, —CC—, —CH═CH—, or -phenyl- and R⁷ is bonded through a nitrogen atom then q=2-4;
when Y is —N(R⁸)— and R⁹ is —N(R⁸)₂, —N(R⁸)₃⁺ or —NR⁸(OR⁸) then g=2-6;
when Y is —N(R⁸)— and M is —N(R⁸)—, —O—, —N((C(R⁸)₂)ₚN(R⁸)₂)— or —N((C(R⁸)₂)ₚ—OR⁸)— then k=1-4;
when Y is —N(R⁸)— and W′ is —N(R⁸)— or —O— then k=2-4;
when Y is —O— and either M or W′ is —O— then k=2-4;
wherein each —C₁-C₆ alkyl, —C₁-C₆ alkylene, —C₁-C₆ alkylene-O—C₁-C₆ alkyl, —C₂-C₆ alkenyl, —C₂-C₆ alkynyl, -phenyl, -benzyl, or phenylene is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R⁷ is -phenyl, a 5- or 6-membered aromatic monocyclic heterocycle, or a 3- to 7-membered non-aromatic monocyclic heterocycle, wherein, each R⁷ is optionally mono or di-substituted on a carbon atom with —OH, —R⁸, —N(R⁸)₂, —OR⁸, —(C(R⁸)₂)ᵣOR⁸, —(C(R⁸)₂)ᵣN(R⁸)₂, or a 3- to 7-membered monocyclic heterocycle,
wherein r=1-6;
each R⁷ is optionally mono or di-substituted on a saturated carbon atom with —O(C(R⁸)₂)ᵣO—,
wherein r=1-6;
each R⁷ is optionally mono-substituted on a nitrogen atom with R⁸;
R⁸ is each independently —H, —C₁-C₆ alkyl, —C₂-C₆ alkenyl, —C₂-C₆ alkynyl, —C₃-C₈ monocyclic cycloalkyl, —C(O)—C₁-C₆ alkyl, —C(O)O—C₁-C₆ alkyl, or -phenyl, each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R⁹ is -halo, —N(R⁸)₂, —OR⁸, —N(R⁸)₃⁺, or —NR⁸(OR⁸);
R¹⁰ and R¹¹ are each independently —(C(R⁸)₂)ₙN(R⁸)₂ or —(C(R⁸)₂)ₙOR⁸,
wherein s=1-4;
R¹² is each independently —H, —C₁-C₆ alkyl, —C₂-C₆ alkenyl, —C₂-C₆ alkynyl, —C₃-C₈ monocyclic cycloalkyl, —C₃-C₈ monocyclic cycloalkenyl, -phenyl, -benzyl, a 5- or 6-membered aromatic monocyclic heterocycle, or a 3- to 7-membered non-aromatic monocyclic heterocycle, each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R¹³ is a 5- or 6-membered aromatic monocyclic heterocycle, a 3- to 7-membered non-aromatic monocyclic heterocycle, a 8- to 12-membered bicyclic heterocycle, -phenyl, -benzyl, or 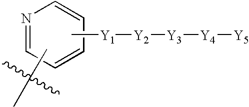
wherein:
Y₁ is a bond, —O—, —N(R¹⁶)—, —C((R¹⁶)₂)—, —C(R¹⁶)═C(R¹⁶), or —CC—;
Y₂ and Y₄ are each independently —(C(R¹⁶)₂)_{b}—, wherein b=0-4;
Y₃ is —C(R¹⁵R¹⁶)—;
Y₅ is absent, —H, -halo, —C₁-C₆ alkyl, —O—C₁-C₆ alkyl, —O—C₁-C₆ alkyl, —OH, —NH₂, —NH—C₁-C₆ alkyl, —N(C₁-C₆ alkyl)₂, —OS(O)₂—C₁-C₆ alkyl, -phenyl, —O-phenyl, —NH-phenyl, -benzyl, —O-benzyl, —NR¹²R²⁸, —OR²⁸, —SR²⁸, —N(R¹²)(C(R¹²)₂)ₘ—R²⁸, —O(C(R¹²)₂)ₘ—R²⁸, —S(C(R¹²)₂)ₘR²⁸, (C(R¹²)₂)ₘ—R²⁸, —(C(R¹²)₂)ₘ—R¹⁴, —(C(R¹²)₂)ₘ—N(R¹²)(C(R¹²)₂)ₘ—R²⁸, —(C(R¹²)₂)ₘ—N(R¹²)(C(R¹²)₂)ₘ—R¹⁴, —(C(R¹²)₂)ₘ—O—(C(R¹²)₂)ₘ—R²⁸, —(C(R¹²)₂)ₘ—O—(C(R¹²)₂)ₘ—R¹⁴, —(C(R¹²)₂)ₘ—S—(C(R¹²)₂)ₘ—R²⁸, —(C(R¹²)₂)ₘ—S—(C(R¹²)₂)ₘ—R¹⁴, R¹⁴, —N(C₁-C₆ alkyl)(S(O)₂R²⁷), a 5- or 6-membered aromatic monocyclic heterocycle, a 3- to 7-membered non-aromatic monocyclic heterocycle, or a 8- to 12-membered bicyclic heterocycle,
wherein m=1-4 with the proviso that m is when between two heteroatoms, m=2-4;
when Y₁ is —C((R¹⁶)₂)— the R¹⁶ substituents together with the intervening carbon atom or atoms can form a 3- to 7-membered ring;
when Y₁ is —C(R¹⁶)═C(R¹⁶)— the R¹⁶ substituents together with the intervening carbon atoms can form a 3- to 7-membered ring;
wherein each heterocycle can be substituted on a single saturated carbon atom not adjacent to a nitrogen atom with —O(CH₂)₂O— or —(CH₂)₃O—; and
each heterocycle can optionally be benzo-fused; and
each heterocycle can optionally be mono- or di-substituted with —O—, —S—, —NH-benzoyl, R²⁸, —O—R²⁸ or R¹⁸,
wherein each 5- or 6-membered aromatic monocyclic heterocycle, a 3- to 7-membered non-aromatic monocyclic heterocycle, a 8- to 12-membered bicyclic heterocycle, -phenyl, -benzyl, benzoyl or —C₁-C₆ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R¹⁴ is —N(R¹²)₂, —OR¹², —SR¹², —NR¹²—(C(R¹²)₂)ₙ—N(R¹²)₂, —NR¹²—(C(R¹²)₂)ₙ—OR¹², —N((C(R¹²)₂)ₙ—N(R¹²)₂)₂, —N((C(R¹²)₂)ₙ—OR¹²)₂, or —N((C(R¹²)₂)ₙ—N(R¹²)₂)(C(R¹²)₂)ₙ—OR¹²);
wherein n=1-4 at each occurrence, with the proviso that when the carbon chain of length n is between two heteroatoms, n=2-4;
R¹⁵ is —OH or —N(R¹⁷)₂;
R¹⁶ is each independently an open valence, —H, —C₁-C₈ alkyl, —C₂-C₈ alkenyl, —C₂-C₈ alkynyl, C₃-C₈ monocyclic cycloalkyl, or —C₃-C₈ monocyclic cycloalkenyl, each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R¹⁶ on Y₃ together with R¹⁶ on Y₂ together with the intervening atoms can form a 3- to 7-membered ring;
R¹⁶ on Y₃ together with R¹⁶ on Y₁ together with the intervening atoms can form a 3- to 7-membered ring;
R¹⁶ on Y₁ together with R¹⁶ on Y₂ together with the intervening atoms can form a 3- to 7-membered ring;
R¹⁶ on Y₂ together with R¹⁶ on Y₄ together with the intervening atoms can form a 3- to 7-membered ring;
R¹⁶ on Y₃ together with R¹⁶ on Y₄ together with the intervening atoms can form a 3- to 7-membered ring;
when R¹⁵ is —N(R¹⁷)₂, R¹⁷ on Y₃ together with R¹⁶ on either Y₁, Y₂, or Y₄ together with the intervening atoms can form a 3- to 7-membered ring;
R¹⁷ is each independently an open valence, —H, —C(O)NH—C₁-C₆ alkyl, —C(O)—O—C₁-C₆ alkyl, —C(O)—O-phenyl, —C(O)—O—C₁-C₃ alkylene-phenyl, —C(O)—O—C₂-C₃ alkenylene-phenyl, —C(O)—O—C₂-C₃ alkynylene-phenyl, —C(O)H, —C₁-C₉ alkyl, —C₂-C₉ alkenyl, —C₂-C₉ alkynyl, —C₃-C₈ monocyclic cycloalkyl, —C₃-C₈ monocyclic cycloalkenyl, —C₇-C₉ monocyclic cycloalkynyl, -phenyl, —C₁-C₃ alkylene-phenyl, —C₂-C₃ alkenylene-phenyl, or —C₂-C₃ alkynylene-phenyl,
each of which other than hydrogen is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R¹⁸ is —H, -halo, —OH, —CN, —NH₂, —N₃, —NO₂, —CHO, —COOH, —C₁-C₆ alkyl, —C₂-C₆ alkenyl, —C₂-C₆ alkynyl, —NHC(O)—C₁-C₆ alkyl, —C₁-C₆ alkylene-O—C₁-C₆ alkyl, —C₁-C₆ alkylene-C(O)O—C₁-C₆ alkyl, —O—C₁-C₆ alkyl, —S—C₁-C₆ alkyl, —C(O)O—C₁-C₆ alkyl, —C(O)—C₁-C₆ alkyl, -phenyl, —O-phenyl, —S-phenyl, -benzoyl, -benzyl, —C₁-C₆ alkylene-C(O)O-phenyl, —NH—C₁-C₆ alkyl, —N(C₁-C₆ alkyl)₂, —NH-phenyl, —NH-benzyl, —NH—C(O)—C₁-C₆ alkyl, —NH—C(O)—C₂-C₆ alkenyl, —NH—C(O)—C₂-C₆ alkynyl, —C₁-C₆ alkylene-COOH, —C₁-C₆ alkylene-CHO, —C₁-C₆ alkylene-NH—C₁-C₆ alkyl, —C₁-C₆ alkylene-N(C₁-C₆ alkyl)₂, —C(O)NH—C₁-C₆ alkyl, —C(O)N(C₁-C₆)₂ alkyl, —O—C₁-C₆ alkylene-NH—C₁-C₆ alkyl, —O—C₁-C₆ alkylene-N(C₁-C₆ alkyl)₂, —OS(O)₂—C₁-C₆ alkyl, or —SH,
wherein each —C₁-C₆ alkyl, —C₁-C₆ alkylene, —C₂-C₆ alkenyl, —C₂-C₆ alkynyl, -phenyl, -benzyl, or benzoyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R^{13′} is a nitrogen-containing 3- to 7-membered non-aromatic monocyclic heterocycle or a nitrogen-containing non-aromatic 8- to 12-membered bicyclic heterocycle, which is not a lactam, which can optionally be mono- or di-substituted with —O—, —S—, —NH-benzoyl, R²⁸, —O—R²⁸, or R¹⁸,
wherein each 5- or 6-membered aromatic monocyclic heterocycle, a 3- to 7-membered non-aromatic monocyclic heterocycle, a 8- to 12-membered bicyclic heterocycle, -phenyl, -benzyl, benzoyl or —C₁-C₆ alkyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl;
R²⁷ is C₁-C₆ alkyl, -phenyl, —(C((R¹²)₂)ₚ)-phenyl, a 5- or 6-membered aromatic monocyclic heterocycle, a 3- to 7-membered non-aromatic monocyclic heterocycle, or a 8- to 12-membered bicyclic heterocycle, —(C((R¹²)₂)ₚ)-5- or 6-membered aromatic monocyclic heterocycle, —(C((R¹²)₂)ₚ)-3- to 7-membered non-aromatic monocyclic heterocycle, —(C((R¹²)₂)ₚ)-8- to 12-membered bicyclic heterocycle,
wherein p=1-4 with the proviso that when the carbon chain of length p is between two heteroatoms, p=2-4; and
R²⁸ is a 5- or 6-membered aromatic monocyclic heterocycle, a 3- to 7-membered non-aromatic monocyclic heterocycle, a 8- to 12-membered bicyclic heterocycle, -phenyl, -benzyl,
wherein each heterocycle can be substituted on a single saturated carbon atom not adjacent to a nitrogen atom with —O(CH₂)₂O— or —(CH₂)₃O—;
each heterocycle can optionally be benzo-fused; and
each 5- or 6-membered aromatic monocyclic heterocycle, a 3- to 7-membered non-aromatic monocyclic heterocycle, a 8- to 12-membered bicyclic heterocycle, -phenyl, -benzyl is independently unsubstituted or substituted with one or more of -halo, —OH, —NH₂, —NO₂, —N₃, —CN, —CF₃, —CHO, —COOH, —C₁-C₆ alkyl, —O—C₁-C₆ alkyl, —S—C₁-C₆ alkyl, —NH—C₁-C₄ alkyl, —N(C₁-C₄ alkyl)₂, -halo-substituted C₁-C₄ alkyl, —C₁-C₄ alkylene-OC(O)—C₁-C₆ alkyl, —C(O)O—C₁-C₆ alkyl, —C(O)NH—C₁-C₆ alkyl, -phenyl, —O-phenyl, —S-phenyl, —NH-phenyl, -benzyl, —O-benzyl, —S-benzyl, —NH-benzyl, —C(O)-benzyl or benzoyl.
